(19) United States
(12) Patent Application Publication
(10) Pub. No.: US 2005/0227301 A1
(43) Pub. Date: Oct. 13, 2005

(54) Cell cycle progression proteins

(75) Inventors: David M. Glover, Cambridge (GB); Graham Bell, Dundee (GB); Lisa M. Frenz, Cambridgeshire (GB); Carol Midgley, Cambridgeshire (GB)

Correspondence Address: PALMER & DODGE, LLP;KATHLEEN M. WILLIAMS, 111 HUNTINGTON AVENUE, BOSTON, MA, 02199, US

(73) Assignee: Polgen

(21) Appl. No.: 10/745,237

(22) Filed: Dec. 23, 2003

Related U.S. Application Data
(60) Provisional application No. 60/468,402, filed on May 6, 2003.
(60) Provisional application No. 60/439,123, filed on Jan. 10, 2003.

(51) Int. Cl. G01N 33/574

(52) U.S. Cl. 435/7.23

(57) ABSTRACT

The invention describes human genes involved in cell cycle progression, including mitosis and meiosis. The invention also relates to the use of these “cell cycle progression” genes and proteins in the modulation of cell cycle progression in cells and methods for identifying modulators of these genes or proteins and hence modulators of mitosis and meiosis.

### RELATED APPLICATIONS

This application claims the benefit of U.S. Provisional Application No. 60/468,402, filed May 6, 2003 and U.S. Provisional Application No. 60/439,123, filed Jan. 10, 2003. The entire teachings of the above applications are incorporated herein by reference.

### BACKGROUND OF THE INVENTION

Proliferative growth of normal cells requires an orderly progression through a series of distinct steps, a process known as the cell cycle. Progression through the cell cycle is modulated by nutrient availability, cell size and growth factors through complex signalling pathways involving phosphorylation cascades and the strictly regulated expression and stability of specific proteins required at each phase of the cell cycle.

The phases of the cell cycle begin with the M phase, where cytoplasmic division (cytokinesis) occurs. The M phase is followed by the G1 phase, in which the cells resume a high rate of biosynthesis and growth. The S phase begins with DNA synthesis, and ends when the DNA content has doubled. The cell then enters the G2 phase, which ends when mitosis starts, signalled by the appearance of condensed chromosomes. Terminally differentiated cells are arrested in the G1 phase, and no longer undergo cell division.

The sequence of cell cycle events is rigorously controlled at specific checkpoints to ensure that each discrete stage in the cell cycle has been completed before the next is initiated. Human diseases associated with abnormal cell proliferation, including cancer, result when these rigorous controls on cell cycle progression are perturbed.

The elucidation of the genes and gene products involved in the cell cycle and its control will provide novel opportunities in the prophylactic, diagnostic and therapeutic management of cancer and other proliferation-related diseases (e.g., atherosclerosis).

On the other hand, it is also sometimes desirable to enhance proliferation of cells in a controlled manner. For example, proliferation of cells is useful in wound healing and where growth of tissue is desirable. Thus, identifying genes, their gene products and modulators which promote, enhance or deter the inhibition of proliferation is desirable.

Despite the desirability of identifying cell cycle components and modulators, there is a deficit of such compounds in the field. Accordingly, it would be advantageous to identify genes and their corresponding protein products whose activity is associated with cell cycle progression.

### SUMMARY OF THE INVENTION

We have now identified a number of human genes involved with cell cycle progression, for example the processes of mitosis and/or meiosis. Discovery of the role of these genes has been through assays configured to identify genes involved in cell cycle progression by knocking down gene expression using RNAi and assessing the resultant phenotype for abnormalities in cell cycle progression.

The invention features a method of identifying an agent that modulates the function of a cell cycle progression polypeptide of SEQ ID NOs:104-205, where the method includes: (a) providing a sample containing a cell cycle progression polypeptide of SEQ ID NOs:104-205, and a candidate agent; (b) measuring the binding of the cell cycle progression polypeptide of SEQ ID NOs:104-205 to the candidate agent in the sample; and (c) comparing the binding of the cell cycle progression polypeptide of SEQ ID NOs:104-205 to the candidate agent in the sample with the binding of the polypeptide of SEQ ID NOs:104-205 to a control agent, where the control agent is known to not bind to the polypeptide of SEQ ID NOs:104-205; where an increase in the binding of the cell cycle progression polypeptide of SEQ ID NOs:104-205 to the candidate agent in the sample relative to the binding of the cell cycle progression polypeptide of SEQ ID NOs:104-205 to the control agent indicates that the candidate agent modulates the function of the cell cycle progression polypeptide of SEQ ID NOs:104-205.

The invention also features a method of detecting the presence in a sample of a cell cycle progression polypeptide of SEQ ID NOs:104-205, where the method includes: (a) bringing the biological sample containing DNA or RNA into contact with a probe comprising a fragment of at least 15 nucleotides of a nucleic acid of SEQ ID NOs:1-103 under hybridizing conditions; and (b) detecting a duplex formed between the probe and nucleic acid in the sample; where detection of a duplex indicates the presence in the sample of a cell cycle progression polypeptide of SEQ ID NOs:104-205.

In another aspect, the invention features a method of detecting the presence in a sample of a cell cycle progression polypeptide of SEQ ID NOs:104-205, where the method includes: (a) providing an antibody capable of binding to the cell cycle progression polypeptide of SEQ ID NOs:104-205; (b) incubating a biological sample with said antibody under conditions which allow for the formation of an antibody-antigen complex; and (c) detecting an antibody-antigen complex comprising said antibody; where detection of an antibody-antigen complex indicates the presence in the sample of a cell cycle progression polypeptide of SEQ ID NOs:104-205.

In a further aspect, the invention features a method of modulating cell cycle progression in a cell, where the method includes: (a) transforming into the cell a double-stranded nucleic acid sequence of SEQ ID NOs:1-103 or a complement thereof, where the nucleic acid sequence is operably linked to a regulatory sequence; and (b) culturing the cell under conditions whereby the nucleic acid sequence is expressed; thereby modulating cell cycle progression in the cell.

The invention also features a method of modulating cell cycle progression in a cell, where the method includes: (a) transforming into the cell a double-stranded nucleic acid sequence encoding a polypeptide of SEQ ID NOs:104-205, where the nucleic acid sequence is operably linked to a regulatory sequence; and (b) culturing the cell under conditions whereby the nucleic acid sequence is expressed; thereby modulating cell cycle progression in the cell.

In an additional aspect, the invention features a method of modulating cell cycle progression in a cell, where the method includes: (a) transforming into the cell a double-stranded nucleic acid sequence encoding a polypeptide having at least 80% sequence identity with a polypeptide of SEQ ID NOs:104-205, where the nucleic acid sequence is operably linked to a regulatory sequence; and (b) culturing the cell under conditions whereby the nucleic acid sequence is expressed; thereby modulating cell cycle progression in the cell.

Another aspect of the invention features a method of modulating cell cycle progression in a cell, where the method includes: (a) transforming into the cell an isolated nucleic acid molecule comprising a regulatory sequence operably linked to a nucleic acid sequence that encodes a ribonucleic acid (RNA) precursor, where the precursor comprises: (i) a first stem portion comprising a 15 to 40 nucleotide long sequence that is identical to 15 to 40 consecutive nucleotides of a sequence of SEQ ID NOs:1-103; (ii) a second stem portion comprising a 15 to 40 nucleotide long sequence that is complementary to 15 to 40 consecutive nucleotides of a sequence of SEQ ID NOs:1-103, and where the first and second stem portions can hybridize with each other to form a duplex stem; and (iii) a loop portion that connects the two stem portions; (b) culturing the cell under conditions whereby the nucleic acid sequence is expressed; thereby modulating cell cycle progression in the cell.

In any of the methods described herein, the nucleic acid sequence is a nucleic acid sequence of SEQ ID NOs:1-103, or a complement thereof. The nucleic acid sequence can encode a polypeptide of SEQ ID NOs:104-205. The nucleic acid sequence can encode a polypeptide having 80% sequence identity to a polypeptide of SEQ ID NOs:104-205.

The methods described herein can be used to decrease cell cycle progression. The decrease can result in a decrease in proliferation of the cell.

The methods described herein can be used to increase cell cycle progression. The increase can result in a increase in proliferation of the cell.

Another feature of the invention is an RNA precursor encoded by a nucleic acid sequence of SEQ ID NOs:1-103. Such an RNA precursor can be included in a composition as a biologically active ingredient. Such an RNA precursor or composition can be used for treating a disease or condition characterized by cell proliferation in mammalian tissue, by contacting the tissue with the RNA precursor or composition. The disease can be cancer.

Another feature of the invention is a host cell transformed by the methods described herein. Such a host cell can contain all or a part of the nucleic acid sequences of SEQ ID NOs:1-103. Such a host cell can express all or a part of the polypeptide sequences of SEQ ID NOs:104-205.

The methods described herein can be used to provide a mammal with an anti-proliferative protein, where the method includes introducing into the mammal a mammalian cell transformed by the methods described herein.

The invention also features a pharmaceutical composition comprising, as an active ingredient, a cell cycle progression nucleic acid sequence of SEQ ID NOs:1-103, and a pharmaceutically-acceptable carrier.

The invention further features a pharmaceutical composition comprising, as an active ingredient, a cell cycle progression polypeptide of SEQ ID NOs:104-205, and a pharmaceutically-acceptable carrier.

The invention additionally features a pharmaceutical composition comprising, as an active ingredient, an antibody to a cell cycle progression nucleic acid sequence of SEQ ID NOs:1-103, and a pharmaceutically-acceptable carrier. Such an antibody can be used in a method for diagnosing a disease or condition characterized by cell proliferation in mammalian tissue, the method comprising contacting the tissue with the antibody, and detecting an antibody/antigen complex, wherein said detection is indicative of said disease or condition.

In another aspect, the invention features a pharmaceutical composition comprising, as an active ingredient, an antibody to a cell cycle progression polypeptide of SEQ ID NOs:104-205, and a pharmaceutically-acceptable carrier. Such an antibody can be used in a method for diagnosing a disease or condition characterized by cell proliferation in mammalian tissue, the method comprising contacting the tissue with the antibody, and detecting an antibody/antigen complex, wherein said detection is indicative of said disease or condition.

A further aspect of the invention features a method for treating a disease or condition characterized by cell proliferation in mammalian tissue, the method comprising contacting the tissue with an antagonist of a cell cycle progression polypeptide of SEQ ID NOs:104-205.

An additional aspect of the invention features a method for treating a disease or condition characterized by cell proliferation in mammalian tissue, the method comprising contacting the tissue with an agonist of a cell cycle progression polypeptide of SEQ ID NOs:104-205.

The invention also features a kit for treating a disease or condition characterized by cell proliferation in mammalian tissue, the kit comprising: (a) a polypeptide encoded by a nucleic acid sequence of SEQ ID NOs:1-103; (b) a nucleic acid having a nucleotide sequence of SEQ ID NOs:1-103; or (c) an antibody recognising an epitope of a polypeptide of (a).

An additional aspect of the invention is an array comprising at least two cell cycle progression genes having nucleic acid sequences of SEQ ID NOs:1-103. The nucleic acid sequences can be DNA sequences. The nucleic acid sequences can be RNA sequences.

Another aspect of the invention is an array comprising at least two cell cycle progression proteins having polypeptide sequences of SEQ ID NOs:104-205. Accordingly, in a first aspect, there is provided a method of modulating cell cycle progression in a cell comprising the step of increasing, decreasing or otherwise altering the functional activity of
i) a polypeptide having an amino acid sequence identified in Table 1;
ii) a polypeptide having an amino acid sequence encoded by a nucleic acid identified in Table 1;
iii) a polypeptide having at least 80% homology with i) or ii);
iv) a nucleic acid having a sequence identified in Table 1 or encoding a polypeptide having the sequence set out in any of i) to iii);
v) a nucleic acid which is capable of selectively hybridising to the sequence set out in iv); or
vi) the complement of iv) or v).

Suitably, the method comprises decreasing gene expression. In a preferred embodiment, the method comprises decreasing the nucleic acid functional activity by introducing a double stranded (dsRNA) corresponding to the nucleic acid, or an antisense RNA corresponding to the nucleic acid, or a fragment thereof, into the cell.

In another embodiment, the method comprises increasing the functional activity.

In a particularly preferred embodiment, the nucleic acid or polypeptide comprises a human nucleic acid or polypeptide as identified in Table 1.

In one embodiment, the method comprises:
a) providing an expression vector comprising a nucleic acid sequence; said nucleic acid sequence being selected from the group consisting of:
i) a nucleic acid having a sequence identified in Table 1;
ii) a nucleic acid which hybridises under stringent conditions to the sequence set out in i); or
iii) the complement of ii); and
b) introducing the expression vector into the cell and maintaining the cell under conditions permitting expression of the encoded polypeptide in the cell.

Knowledge of the genes involved in cell cycle progression allows the development of therapeutic agents for the treatment of medical conditions associated with aberrant cell cycle progression.

Accordingly, in a second aspect of the invention, there is provided a use of a nucleic acid identified in Table 1 or a polypeptide identified in Table 1 or a fragment thereof, in a method of prevention, treatment or diagnosis of a disease in an individual.

Suitably, the nucleic acid or polypeptide comprises a human nucleic acid or polypeptide identified in Table 1.

In one embodiment the nucleic acid or polypeptide is used to identify a substance capable of binding to the polypeptide, which method comprises incubating the polypeptide with a candidate substance under suitable conditions and determining whether the substance binds to the polypeptide.

In another embodiment, the nucleic acid or polypeptide is used to identify a substance capable of modulating the function of the polypeptide, the method comprising the steps of: incubating the polypeptide with a candidate substance and determining whether the activity of the polypeptide is thereby modulated.

Thus, the present invention provides the use of a cell cycle progression polypeptide encoded by a nucleic acid identified in Table 1 in an assay for identifying a substance capable of inhibiting cell cycle progression.

By “cell cycle progression” is meant any of the steps or stages in the cell cycle, for example, formation of the nuclear envelope, exit from the quiescent phase of the cell cycle (G0), G1 progression, chromosome decondensation, nuclear envelope breakdown, START, initiation of DNA replication, progression of DNA replication, termination of DNA replication, centrosome duplication, G2 progression, activation of mitotic or meiotic functions, chromosome condensation, centrosome separation, microtubule nucleation, spindle formation and function, interactions with microtubule motor proteins, chromatid separation and segregation, inactivation of mitotic functions, formation of contractile ring, and cytokinesis functions. Functions of the polynucleotides and polypeptides disclosed herein also include functions such as chromatin binding, formation of replication complexes, replication licensing, phosphorylation or other secondary modification activity, proteolytic degradation, microtubule binding, actin binding, septin binding, microtubule organising centre nucleation activity and binding to components of cell cycle signalling pathways. By saying that the “functional activity” of the polynucleotides and polypeptides disclosed herein is increased or decreased, it is meant that cell cycle progression is increased or decreased as a result of a change in one of these functions. Change in cell cycle progression can be measured by any of a number of standard assays, e.g., mitotic index.

The nucleic acid or polypeptide may be administered to an individual in need of such a treatment. Alternatively, or in addition, the substance identified by the method is administered to an individual in need of such treatment.

Also provided is a substance identified by the above uses. Such substances may be used in a method of therapy, such as in a method of affecting cell cycle progression, for example mitosis and/or meiosis.

The invention also provides a process comprising the steps of: (a) performing one of the above methods; and (b) preparing a quantity of those one or more substances identified as being capable of binding to a polypeptide of the invention.

Also provided is a process comprising the steps of: (a) performing one of the above methods; and (b) preparing a pharmaceutical composition comprising one or more substances identified as being capable of binding to a polypeptide of the invention.

The use may be for a method of diagnosis, in which the presence or absence of a nucleic acid is detected in a biological sample in a method comprising: (a) bringing the biological sample containing DNA or RNA into contact with a probe comprising a fragment of at least 15 nucleotides of the nucleic acid identified in Table 1 under hybridising conditions; and (b) detecting any duplex formed between the probe and nucleic acid in the sample.

Alternatively, or in addition, the absence or presence of a polypeptide is detected in a biological sample in a method comprising: (a) providing an antibody capable of binding to the polypeptide; (b) incubating a biological sample with said antibody under conditions which allow for the formation of an antibody-antigen complex; and (c) determining whether antibody-antigen complex comprising said antibody is formed.

In a particularly preferred embodiment, the disease comprises a disease associated with a defect in the cell cycle and, in particular, a proliferative disease such as cancer.

According to another aspect of the invention, there is provided a pharmaceutical composition comprising any one or more of the following: a polypeptide encoded by a nucleic acid identified in Table 1, or part thereof; a vector comprising a nucleic acid identified in Table 1; an antibody recognising an epitope of a polypeptide encoded by a nucleotide sequence identified in Table 1, together with a pharmaceutically acceptable carrier or diluent.

In one embodiment, the pharmaceutical composition is a vaccine composition.

In a further aspect, there is provided a nucleic acid identified in Table 1 for use in therapy.

In yet another aspect, there is provided a polypeptide encoded by a nucleic acid identified in Table 1 for use in therapy.

In a yet further aspect, there is provided an antibody capable of binding a polypeptide encoded by a nucleic acid identified in Table 1 for use in therapy.

Alternatively, in another aspect of the invention, there is provided a method of treating a patient suffering from a disease associated with enhanced activity of a cell cycle progression protein encoded by a nucleic acid identified in Table 1, which method comprises administering to the patient an antagonist of said cell cycle progression protein.

In another aspect there is provided a method of treating a patient suffering from a disease associated with reduced activity of a cell cycle progression protein encoded by a nucleic acid identified in Table 1, which method comprises administering to the patient an agonist of said cell cycle progression protein.

In an additional aspect, the invention provides kits comprising polynucleotides, polypeptides or antibodies of the invention and methods of using such kits in diagnosing the presence of absence of polynucleotides and polypeptides of the invention including deleterious mutant forms.

Accordingly, there is provided a diagnostic kit for a disease or susceptibility to a disease comprising any one or more of the following: a polypeptide encoded by a nucleic acid sequence identified in Table 1 or part thereof, a nucleic acid having a nucleotide sequence identified in Table 1; and an antibody recognising an epitope of a polypeptide encoded by a nucleic acid having a nucleotide sequence identified in Table 1.

In one embodiment, said diagnostic kit comprises an array, such as a nucleic acid or other microarray, comprising at least two cell cycle progression genes having nucleic acid sequences identified in Table 1, or fragments thereof. The fragments can be 15 nucleotides in length, or longer, up to the full length of the gene.

Suitably the disease or syndrome is one which is associated with abnormal cell cycle or proliferation such as cancer.

### DESCRIPTION OF THE DRAWINGS

FIG. 1 shows the nucleotide sequence for Drosophila gene CG3632 (GI 10728281), which has four transcripts, CT12163 (SEQ ID NO:1), CT13680 (SEQ ID NO:3), CT13700 (SEQ ID NO:5) and CT13718 (SEQ ID NO:7), which encode GI Acc. AAF48583 (SEQ ID NO:2), AAF48584 (SEQ ID NO:4), AAF48582 (SEQ ID NO:6) and AAF48581 (SEQ ID NO:8), respectively.

FIG. 2 shows the nucleotide sequence for Drosophila gene Pp1-87B (GI 7299572) (SEQ ID NO:9), which encodes protein GI Acc. AAF54810 (SEQ ID NO:10).

FIG. 3 shows the nucleotide sequence for Drosophila gene CG3524 (GI 10727365) (SEQ ID NO:11), which encodes protein GI Acc. AAF51149 (SEQ ID NO:12).

FIG. 4 shows the nucleotide sequence for Drosophila gene CG9311 (GI 10727923) (SEQ ID NO:13), which encodes protein GI Acc. AAF49705 (SEQ ID NO:14).

FIG. 5 shows the nucleotide sequence for Drosophila gene CG9092 (GI 7297037) (SEQ ID NO:15), which encodes protein GI Acc. AAF52321 (SEQ ID NO:16).

FIG. 6 shows the nucleotide sequence for Drosophila gene Arr1 (GI 10728850) (SEQ ID NO:17), which encodes protein GI Acc. AAF53644 (SEQ ID NO:18).

FIG. 7 shows the nucleotide sequence for Drosophila gene CG9150 (GI 7297037) (SEQ ID NO:19), which encodes protein GI Acc. AAF52338 (SEQ ID NO:20).

FIG. 8 shows the nucleotide sequence for Drosophila gene CG11102 (GI 10728232) (SEQ ID NO:21), which encodes protein GI Acc. AAF48320 (SEQ ID NO:22).

FIG. 9 shows the nucleotide sequence for Drosophila gene Smr (GI 7292788) (SEQ ID NO:23), which encodes protein GI Acc. AAF48196 (SEQ ID NO:24).

FIG. 10 shows the nucleotide sequence for Drosophila gene CG8045 (GI 7300335), which has three transcripts, CT24072 (SEQ ID NO:25), CT24102 (SEQ ID NO:27) and CT24092 (SEQ ID NO:29), which encode GI Acc. AAF55517 (SEQ ID NO:26), GI Acc. AAF55519 (SEQ ID NO:28) and GI Acc. AAF55523 (SEQ ID NO:30), respectively.

FIG. 11 shows the nucleotide sequence for Drosophila gene CG10420 (GI 7301280) (SEQ ID NO:31), which encodes protein GI Acc. AAF56422 (SEQ ID NO:32).

FIG. 12 shows the nucleotide sequence for Drosophila gene Hsc70-2 (GI 10726497) (SEQ ID NO:33), which encodes protein GI Acc. AAF54899 (SEQ ID NO:34).

FIG. 13 shows the nucleotide sequence for Drosophila gene CG10805 (GI 7297167) (SEQ ID NO:35), which encodes protein GI Acc. AAF52447 (SEQ ID NO:36).

FIG. 14 shows the nucleotide sequence for Drosophila gene eIF-4a (GI 7297037) (SEQ ID NO:37), which encodes protein GI Acc. AAF52317 (SEQ ID NO:38).

FIG. 15 shows the nucleotide sequence for Drosophila gene ACXA (GI 7297983) (SEQ ID NO:39), which encodes protein GI Acc. AAF53228 (SEQ ID NO:40).

FIG. 16 shows the nucleotide sequence for Drosophila gene CG15117 (GI 10727456) (SEQ ID NO:41), which encodes protein GI Acc. AAF57602 (SEQ ID NO:42).

FIG. 17 shows the nucleotide sequence for Drosophila gene BG:DS01759.2 (GI 7298121) (SEQ ID NO:43), which encodes protein GI Acc. AAF53376 (SEQ ID NO:44).

FIG. 18 shows the nucleotide sequence for Drosophila gene TepIII (GI 7297264) (SEQ ID NO:45), which encodes protein GI Acc. AAF52542 (SEQ ID NO:46).

FIG. 19 shows the nucleotide sequence for Drosophila gene Hsc70-4 (GI 10726541) (SEQ ID NO:47), which encodes protein GI Acc. AAF55150 (SEQ ID NO:48).

FIG. 20 shows the nucleotide sequence for Drosophila gene CG7069 (GI 10726692) (SEQ ID NO:49), which encodes protein GI Acc. AAF55980 (SEQ ID NO:50).

FIG. 21 shows the nucleotide sequence for Drosophila gene ACXE (GI 7297983) (SEQ ID NO:51), which encodes protein GI Acc. AAF53229 (SEQ ID NO:52).

FIG. 22 shows the nucleotide sequence for Drosophila gene EG:52C10.5 (GI 10727480) (SEQ ID NO:53), which encodes protein GI Acc. AAF57789 (SEQ ID NO:54).

FIG. 23 shows the nucleotide sequence for Drosophila gene gatA (GI 10726610) (SEQ ID NO:55), which encodes protein GI Acc. AAF55624 (SEQ ID NO:56).

FIG. 24 shows the nucleotide sequence for Drosophila gene CG17149 (GI 10727803), which has two transcripts, CT33310 (SEQ ID NO:57) and CT38086 (SEQ ID NO:59), which encode GI Acc. AAF49052 (SEQ ID NO:58) and GI Acc. AAF49051 (SEQ ID NO:60), respectively.

FIG. 25 shows the nucleotide sequence for Drosophila gene CG2905 (GI 10728163) (SEQ ID NO:61), which encodes protein GI Acc. AAF57342 (SEQ ID NO:62).

FIG. 26 shows the nucleotide sequence for Drosophila gene CG2336 (GI 10727121) (SEQ ID NO:63), which encodes protein GI Acc. AAF54111 (SEQ ID NO:64).

FIG. 27 shows the nucleotide sequence for Drosophila gene TER94 (GI 10727672), which has two transcripts, CT7768 (SEQ ID NO:65) and CT7776 (SEQ ID NO:67), which encode GI Acc. AAF58863 (SEQ ID NO:66) and GI Acc. AAF58864 (SEQ ID NO:68), respectively.

FIG. 28 shows the nucleotide sequence for Drosophila gene CG6313 (GI 10726739) (SEQ ID NO:69), which encodes protein GI Acc. AAF56267 (SEQ ID NO:70).

FIG. 29 shows the nucleotide sequence for Drosophila gene aur (GI 10726473) (SEQ ID NO:71), which encodes protein GI Acc. AAF54723 (SEQ ID NO:72).

FIG. 30 shows the nucleotide sequence for Drosophila gene Pk91C (GI 10799498) (SEQ ID NO:73), which encodes protein GI Acc. AAF55594 (SEQ ID NO:74).

FIG. 31 shows the nucleotide sequence for Drosophila gene Top2 (GI 10728874) (SEQ ID NO:75), which encodes protein GI Acc. AAF53802 (SEQ ID NO:76).

FIG. 32 shows the nucleotide sequence for Drosophila gene alpha-Est1 (GI 10727101) (SEQ ID NO:77), which encodes protein GI Acc. AAG22202 (SEQ ID NO:78).

FIG. 33 shows the nucleotide sequence for Drosophila gene Nrk (GI 10727582) (SEQ ID NO:79), which encodes protein GI Acc. AAF58420 (SEQ ID NO:80).

FIG. 34 shows the nucleotide sequence for Drosophila gene otk (GI 10727617) (SEQ ID NO:81), which encodes protein GI Acc. AAF58596 (SEQ ID NO:82).

FIG. 35 shows the nucleotide sequence for Drosophila gene cad (GI 10799497) (SEQ ID NO:83), which encodes protein GI Acc. AAF53923 (SEQ ID NO:84).

FIG. 36 shows the nucleotide sequence for Drosophila gene Rut (GI 10728252) (SEQ ID NO:85), which encodes protein GI Acc. AAF48388 (SEQ ID NO:86).

FIG. 37 shows the nucleotide sequence for Drosophila gene CG8002 (GI 10728334) (SEQ ID NO:87), which encodes protein GI Acc. AAF48942 (SEQ ID NO:88).

FIG. 38 shows the nucleotide sequence for Drosophila gene CG10335 (GI 10727968) (SEQ ID NO:89), which encodes protein GI Acc. AAF49936 (SEQ ID NO:90).

FIG. 39 shows the nucleotide sequence for Drosophila gene CG8070 (GI 10727693) (SEQ ID NO:91), which encodes protein GI Acc. AAF58986 (SEQ ID NO:92).

FIG. 40 shows the nucleotide sequence for Drosophila gene CG7460 (GI 10727853) (SEQ ID NO:93), which encodes protein GI Acc. AAF49310 (SEQ ID NO:94).

FIG. 41 shows the nucleotide sequence for Drosophila gene CG17735 (GI 10727172) (SEQ ID NO:95), which encodes protein GI Acc. AAF52092 (SEQ ID NO:96).

FIG. 42 shows the nucleotide sequence for Drosophila gene Gycalpha99B (GI 7301790) (SEQ ID NO:97), which encodes protein GI Acc. AAF56917 (SEQ ID NO:98).

FIG. 43 shows the nucleotide sequence for Drosophila gene CG13893 (GI 7291959) (SEQ ID NO:99), which encodes protein GI Acc. AAF47396 (SEQ ID NO:100).

FIG. 44 shows the nucleotide sequence for Drosophila gene CG18176 (GI 10728019) (SEQ ID NO:101), which encodes protein GI Acc. AAF50214 (SEQ ID NO:102).

FIG. 45 shows the nucleotide sequence for Drosophila gene CG8858 (GI 10727617) (SEQ ID NO:103), which encodes protein GI Acc. AAF58554 (SEQ ID NO:104).

FIG. 46 shows the nucleotide sequence for Drosophila gene Ac76E (GI 10733346) (SEQ ID NO:105), which encodes protein GI Acc. AAF49089 (SEQ ID NO:106).

FIG. 47 shows the nucleotide sequence for Drosophila gene CG17010 (GI 7297915) (SEQ ID NO:107), which encodes protein GI Acc. AAF53182 (SEQ ID NO:108).

FIG. 48 shows the nucleotide sequence for Drosophila gene Tkv (GI 7296952) (SEQ ID NO:109), which encodes protein GI Acc. AAF52230 (SEQ ID NO:110).

FIG. 49 shows the nucleotide sequence for Drosophila gene Dnt (GI 10728868) (SEQ ID NO:111), which encodes protein GI Acc. AAF53783 (SEQ ID NO:112).

FIG. 50 shows the nucleotide sequence for Drosophila gene ACXD (GI 10727242) (SEQ ID NO:113), which encodes protein GI Acc. AAF47621 (SEQ ID NO:114).

FIG. 51 shows the nucleotide sequence for Drosophila gene Aats-ala-m (GI 10728137) (SEQ ID NO:115), which encodes protein GI Acc. AAF50804 (SEQ ID NO:116).

FIG. 52 shows the nucleotide sequence for Drosophila gene Gek (GI 7291737) (SEQ ID NO:117), which encodes protein GI Acc. AAF47163 (SEQ ID NO:118).

FIG. 53 shows the nucleotide sequence for Drosophila gene CG3216 (GI 10726992) (SEQ ID NO:119), which encodes protein GI Acc. AAF46649 (SEQ ID NO:120).

FIG. 54 shows the nucleotide sequence for Drosophila gene CG5653 (GI 10728037) (SEQ ID NO:121), which encodes protein GI Acc. AAF50345 (SEQ ID NO:122).

FIG. 55 shows the nucleotide sequence for Drosophila gene CG17740 (GI 10727606) (SEQ ID NO:123), which encodes protein GI Acc. AAF58535 (SEQ ID NO:124).

FIG. 56 shows the nucleotide sequence for Drosophila gene TepI (GI 7298255) (SEQ ID NO:125), which encodes protein GI Acc. AAF53490 (SEQ ID NO:126).

FIG. 57 shows the nucleotide sequence for Drosophila gene for (GI 10727349), which has five transcripts, CT43154 (SEQ ID NO:127), CT42452 (SEQ ID NO:129), CT43152 (SEQ ID NO:131), CT43158 (SEQ ID NO:133) and CT43160 (SEQ ID NO:135), which encode GI Acc. AAF51082 (SEQ ID NO:128), GI Acc. AAG22251 (SEQ ID NO:130), GI Acc. AAG22252 (SEQ ID NO:132), GI Acc. AAG22253 (SEQ ID NO:134) and GI Acc. AAG22254 (SEQ ID NO:136), respectively.

FIG. 58 shows the nucleotide sequence for Drosophila gene Ac13E (GI 10728265) (SEQ ID NO:137), which encodes protein GI Acc. AAF48468 (SEQ ID NO:138).

FIG. 59 shows the nucleotide sequence for Drosophila gene CG2667 (GI 7298935) (SEQ ID NO:139), which encodes protein GI Acc. AAF54154 (SEQ ID NO:140).

FIG. 60 shows the nucleotide sequence for Drosophila gene CG7842 (GI 10727872) (SEQ ID NO:141), which encodes protein GI Acc. AAF49377 (SEQ ID NO:142).

FIG. 61 shows the nucleotide sequence for Drosophila gene CG17486 (GI 7289853) (SEQ ID NO:143), which encodes protein GI Acc. AAF45462 (SEQ ID NO:144).

FIG. 62 shows the nucleotide sequence for Drosophila gene CG6969 (GI 10726705) (SEQ ID NO:145), which encodes protein GI Acc. AAF56043 (SEQ ID NO:146).

FIG. 63 shows the nucleotide sequence for Drosophila gene CG12262 (GI 10728071) (SEQ ID NO:147), which encodes protein GI Acc. AAF50524 (SEQ ID NO:148).

FIG. 64 shows the nucleotide sequence for Drosophila gene Fray (GI 10726601) (SEQ ID NO:149), which encodes protein GI Acc. AAF55567 (SEQ ID NO:150).

FIG. 65 shows the nucleotide sequence for Drosophila gene CG6879 (GI 10726756) (SEQ ID NO:151), which encodes protein GI Acc. AAF56334 (SEQ ID NO:152).

FIG. 66 shows the nucleotide sequence for Drosophila gene CG11594 (GI 10727290) (SEQ ID NO:153), which encodes protein GI Acc. AAF47823 (SEQ ID NO:154).

FIG. 67 shows the nucleotide sequence for Drosophila gene S6kII (GI 10803726) (SEQ ID NO:155), which encodes protein GI Acc. AAF50945 (SEQ ID NO:156).

FIG. 68 shows the nucleotide sequence for Drosophila gene CG11714 (GI 10727982) (SEQ ID NO:157), which encodes protein GI Acc. AAF50053 (SEQ ID NO:158).

FIG. 69 shows the nucleotide sequence for Drosophila gene CG3534 (GI 7300193) (SEQ ID NO:159), which encodes protein GI Acc. AAF55371 (SEQ ID NO:160).

FIG. 70 shows the nucleotide sequence for Drosophila gene CG7335 (GI 10727803) (SEQ ID NO:161), which encodes protein GI Acc. AAF49067 (SEQ ID NO:162).

FIG. 71 shows the nucleotide sequence for Drosophila gene CG11275 (GI 7291355) (SEQ ID NO:163), which encodes protein GI Acc. AAF46814 (SEQ ID NO:164).

FIG. 72 shows the nucleotide sequence for Drosophila gene CG16726 (GI 10728019) (SEQ ID NO:165), which encodes protein GI Acc. AAF50229 (SEQ ID NO:166).

FIG. 73 shows the nucleotide sequence for Drosophila gene CG7514 (GI 10727313) (SEQ ID NO:167), which encodes protein GI Acc. AAF47931 (SEQ ID NO:168).

FIG. 74 shows the nucleotide sequence for Drosophila gene CG17283 (GI 7300241) (SEQ ID NO:169), which encodes protein GI Acc. AAF55416 (SEQ ID NO:170).

FIG. 75 shows the nucleotide sequence for Drosophila gene BcDNA:GH07626 (GI 10727365) (SEQ ID NO:171), which encodes protein GI Acc. AAF51148 (SEQ ID NO:172).

FIG. 76 shows the nucleotide sequence for Drosophila gene CG16752 (GI 10728478) (SEQ ID NO:173), which encodes protein GI Acc. AAF46037 (SEQ ID NO:174).

FIG. 77 shows the nucleotide sequence for Drosophila gene Rpt1 (GI 10727757) (SEQ ID NO:175), which encodes protein GI Acc. AAF59219 (SEQ ID NO:176).

FIG. 78 shows the nucleotide sequence for Drosophila gene Wts (GI 7301969) (SEQ ID NO:177), which encodes protein GI Acc. AAF57085 (SEQ ID NO:178).

FIG. 79 shows the nucleotide sequence for Drosophila gene CG1582 (GI 7292554) (SEQ ID NO:179), which encodes protein GI Acc. AAF47973 (SEQ ID NO:180).

FIG. 80 shows the nucleotide sequence for Drosophila gene CG12289 (GI 10727982) (SEQ ID NO:181), which encodes protein GI Acc. AAF50065 (SEQ ID NO:182).

FIG. 81 shows the nucleotide sequence for Drosophila gene Pepck (GI 10727469) (SEQ ID NO:183), which encodes protein GI Acc. AAF57676 (SEQ ID NO:184).

FIG. 82 shows the nucleotide sequence for Drosophila gene CG5665 (GI 10726906) (SEQ ID NO:185), which encodes protein GI Acc.-AAF51579 (SEQ ID NO:186).

FIG. 83 shows the nucleotide sequence for Drosophila gene CG7285 (GI 10727839) (SEQ ID NO:187), which encodes protein GI Acc. AAF49259 (SEQ ID NO:188).

FIG. 84 shows the nucleotide sequence for Drosophila gene Bt (GI 10726313) (SEQ ID NO:189), which encodes protein GI Acc. AAF59316 (SEQ ID NO:190).

FIG. 85 shows the nucleotide sequence for Drosophila gene CG8795 (GI 10726505) (SEQ ID NO:191), which encodes protein GI Acc. AAF54929 (SEQ ID NO:192).

FIG. 86 shows the nucleotide sequence for Drosophila gene CG10967 (GI 10727955) (SEQ ID NO:193), which encodes protein GI Acc. AAF49878 (SEQ ID NO:194).

FIG. 87 shows the nucleotide sequence for Drosophila gene CG3809 (GI 10726480) (SEQ ID NO:195), which encodes protein GI Acc. AAF54757 (SEQ ID NO:196).

FIG. 88 shows the nucleotide sequence for Drosophila gene Ack (GI 10727290) (SEQ ID NO:197), which encodes protein GI Acc. AAF47839 (SEQ ID NO:198).

FIG. 89 shows the nucleotide sequence for Drosophila gene Abl (GI 10727878) (SEQ ID NO:199), which encodes protein GI Acc. AAF49431 (SEQ ID NO:200).

FIG. 90 shows the nucleotide sequence for Drosophila gene CG7362 (GI 10726534) (SEQ ID NO:201), which encodes protein GI Acc. AAF55096 (SEQ ID NO:202).

FIG. 91 shows the nucleotide sequence for Drosophila gene Cyp9f2 (GI 7299572) (SEQ ID NO:203), which encodes protein GI Acc. AAF54803 (SEQ ID NO:204).

FIG. 92 shows the nucleic acid (SEQ ID NO:205) and amino acid (SEQ ID NO:206) sequence for the human homolog to Drosophila gene CG3632 (SWISS-PROT Ref. No. Q9UEG3).

FIG. 93 shows the nucleic acid (SEQ ID NO:207) and amino acid (SEQ ID NO:208) sequence for the human homolog to Drosophila gene Pp1-87B (SWISS-PROT Ref. No. P36873).

FIG. 94 shows the nucleic acid (SEQ ID NO:209) and amino acid (SEQ ID NO:210) sequence for the human homolog to Drosophila gene CG3524 (SWISS-PROT Ref. No. Q16702).

FIG. 95 shows the nucleic acid (SEQ ID NO:211) and amino acid (SEQ ID NO:212) sequence for the human homolog to Drosophila gene CG9311 (SWISS-PROT Ref. No. Q9H3S7).

FIG. 96 shows the nucleic acid (SEQ ID NO:213) and amino acid (SEQ ID NO:214) sequence for the human homolog to Drosophila gene CG9092 (SWISS-PROT Ref. No. P16278).

FIG. 97 shows the nucleic acid (SEQ ID NO:215) and amino acid (SEQ ID NO:216) sequence for the human homolog to Drosophila gene Arr1 (SWISS-PROT Ref. No. P49407).

FIG. 98 shows the nucleic acid (SEQ ID NO:217) and amino acid (SEQ ID NO:218) sequence for the human homolog to Drosophila gene CG9150 (SWISS-PROT Ref. No. Q9BUC7).

FIG. 99 shows the nucleic acid (SEQ ID NO:219) and amino acid (SEQ ID NO:220) sequence for the human homolog to Drosophila gene CG11102 (SWISS-PROT Ref. No. BAA31634).

FIG. 100 shows the nucleic acid (SEQ ID NO:221) and amino acid (SEQ ID NO:222) sequence for the human homolog to Drosophila gene Smr (SWISS-PROT Ref. No. 075376).

FIG. 101 shows the nucleic acid (SEQ ID NO:223) and amino acid (SEQ ID NO:224) sequence for the human homolog to Drosophila gene CG8045 (SWISS-PROT Ref. No. P42655).

FIG. 102 shows the nucleic acid (SEQ ID NO:225) and amino acid (SEQ ID NO:226) sequence for the human homolog to Drosophila gene CG10420 (SWISS-PROT Ref. No. Q9H173).

FIG. 103 shows the nucleic acid (SEQ ID NO:227) and amino acid (SEQ ID NO:228) sequence for the human homolog to Drosophila gene Hsc70-2 (SWISS-PROT Ref. No. P11142).

FIG. 104 shows the nucleic acid (SEQ ID NO:229) and amino acid (SEQ ID NO:230) sequence for the human homolog to Drosophila gene CG10805 (SWISS-PROT Ref. No. Q9H583).

FIG. 105 shows the nucleic acid (SEQ ID NO:231) and amino acid (SEQ ID NO:232) sequence for the human homolog to Drosophila gene eIF-4a (SWISS-PROT Ref. No. Q96EA8).

FIG. 106 shows the nucleic acid sequences (SEQ ID NO:233 and SEQ ID NO:235, respectively) and the amino acid sequences (SEQ ID NO:234 and SEQ ID NO:236, respectively) for the two human homologs to Drosophila gene ACXA (SWISS-PROT Ref. No. Q08462 and SWISS-PROT Ref. No. P40145).

FIG. 107 shows the nucleic acid (SEQ ID NO:237) and amino acid (SEQ ID NO:238) sequence for the human homolog to Drosophila gene CG15117 (SWISS-PROT Ref. No. P08236).

FIG. 108 shows the nucleic acid (SEQ ID NO:239) and amino acid (SEQ ID NO:240) sequence for the human homolog to Drosophila gene TepIII (SWISS-PROT Ref. No. Q8TDJ3).

FIG. 109 shows the nucleic acid (SEQ ID NO:241) and amino acid (SEQ ID NO:242) sequence for the human homolog to Drosophila gene Hsc70-4 (SWISS-PROT Ref. No. P11142).

FIG. 110 shows the nucleic acid (SEQ ID NO:243) and amino acid (SEQ ID NO:244) sequence for the human homolog to Drosophila gene CG7069 (SWISS-PROT Ref. No. P14786).

FIG. 111 shows the nucleic acid sequences (SEQ ID NO:245 and SEQ ID NO:247, respectively) and amino acid sequences (SEQ ID NO:246 and SEQ ID NO:248, respectively) for the two human homologs to Drosophila gene ACXE (SWISS-PROT Ref. No. P40145 and SWISS-PROT Ref. No. P51828).

FIG. 112 shows the nucleic acid (SEQ ID NO:249) and amino acid (SEQ ID NO:250) sequence for the human homolog to Drosophila gene EG:52C10.5 (SWISS-PROT Ref. No. Q9Y217).

FIG. 113 shows the nucleic acid (SEQ ID NO:251) and amino acid (SEQ ID NO:252) sequence for the human homolog to Drosophila gene gatA (SWISS-PROT Ref. No. Q9HOR6).

FIG. 114 shows the nucleic acid (SEQ ID NO:253) and amino acid (SEQ ID NO:254) sequence for the human homolog to Drosophila gene CG17149 (SWISS-PROT Ref. No. Q9NUH3).

FIG. 115 shows the nucleic acid (SEQ ID NO:255) and amino acid (SEQ ID NO:256) sequence for the human homolog to Drosophila gene CG2905 (SWISS-PROT Ref. No. Q9Y6H4).

FIG. 116 shows the nucleic acid (SEQ ID NO:257) and amino acid (SEQ ID NO:258) sequence for the human homolog to Drosophila gene TER94 (SWISS-PROT Ref. No. P55072).

FIG. 117 shows the nucleic acid (SEQ ID NO:259) and amino acid (SEQ ID NO:260) sequence for the human homolog to Drosophila gene CG6313 (SWISS-PROT Ref. No. BAA31672).

FIG. 118 shows the nucleic acid sequences (SEQ ID NO:261 and SEQ ID NO:263, respectively) and amino acid sequences (SEQ ID NO:262 and SEQ ID NO:264, respectively) for the two human homologs to Drosophila gene aur (SWISS-PROT Ref. No. 060445 and SWISS-PROT Ref. No. 060446).

FIG. 119 shows the nucleic acid (SEQ ID NO:265) and amino acid (SEQ ID NO:266) sequence for the human homolog to Drosophila gene Pk91C (SWISS-PROT Ref. No. Q96SJ5).

FIG. 120 shows the nucleic acid sequences (SEQ ID NO:267 and SEQ ID NO:269, respectively) and amino acid sequences (SEQ ID NO:268 and SEQ ID NO:270, respectively) for the two human homologs to Drosophila gene Top2 (SWISS-PROT Ref. No. P11388 and SWISS-PROT Ref. No. Q02880).

FIG. 121 shows the nucleic acid (SEQ ID NO:271) and amino acid (SEQ ID NO:272) sequence for the human homolog to Drosophila gene alpha-Est1 (SWISS-PROT Ref. No. P22303).

FIG. 122 shows the nucleic acid (SEQ ID NO:273) and amino acid (SEQ ID NO:274) sequence for the human homolog to Drosophila gene Nrk (SWISS-PROT Ref. No. 015146).

FIG. 123 shows the nucleic acid (SEQ ID NO:275) and amino acid (SEQ ID NO:276) sequence for the human homolog to Drosophila gene otk (SWISS-PROT Ref. No. Q13308).

FIG. 124 shows the nucleic acid (SEQ ID NO:277) and amino acid (SEQ ID NO:278) sequence for the human homolog to Drosophila gene cad (SWISS-PROT Ref. No. Q99626).

FIG. 125 shows the nucleic acid (SEQ ID NO:279) and amino acid (SEQ ID NO:280) sequence for the human homolog to Drosophila gene Rut (SWISS-PROT Ref. No. 043306).

FIG. 126 shows the nucleic acid (SEQ ID NO:281) and amino acid (SEQ ID NO:282) sequence for the human homolog to Drosophila gene CG8002 (SWISS-PROT Ref. No. BAC02708).

FIG. 127 shows the nucleic acid (SEQ ID NO:283) and amino acid (SEQ ID NO:284) sequence for the human homolog to Drosophila gene CG10335 (SWISS-PROT Ref. No. P13716).

FIG. 128 shows the nucleic acid (SEQ ID NO:285) and amino acid (SEQ ID NO:286) sequence for the human homolog to Drosophila gene CG8070 (SWISS-PROT Ref. No. Q9NVU7).

FIG. 129 shows the nucleic acid (SEQ ID NO:287) and amino acid (SEQ ID NO:288) sequence for the human homolog to Drosophila gene CG7460 (SWISS-PROT Ref. No. Q96QT3).

FIG. 130 shows the nucleic acid (SEQ ID NO:289) and amino acid (SEQ ID NO:290) sequence for the human homolog to Drosophila gene CG17735 (SWISS-PROT Ref. No. Q14669).

FIG. 131 shows the nucleic acid (SEQ ID NO:291) and amino acid (SEQ ID NO:292) sequence for the human homolog to Drosophila gene Gycalpha99B (SWISS-PROT Ref. No. P33402).

FIG. 132 shows the nucleic acid (SEQ ID NO:293) and amino acid (SEQ ID NO:294) sequence for the human homolog to Drosophila gene CG13893 (SWISS-PROT Ref. No. 076054).

FIG. 133 shows the nucleic acid (SEQ ID NO:295) and amino acid (SEQ ID NO:296) sequence for the human homolog to Drosophila gene CG18176 (SWISS-PROT Ref. No. AAH33918).

FIG. 134 shows the nucleic acid (SEQ ID NO:297) and amino acid (SEQ ID NO:298) sequence for the human homolog to Drosophila gene CG8858 (SWISS-PROT Ref. No. O15074).

FIG. 135 shows the nucleic acid sequences (SEQ ID NO:299 and SEQ ID NO:301, respectively) and amino acid sequences (SEQ ID NO:300 and SEQ ID NO:302, respectively) for the two human homologs to Drosophila gene Ac76E (SWISS-PROT Ref. No. Q08462 and SWISS-PROT Ref. No. P51828).

FIG. 136 shows the nucleic acid (SEQ ID NO:303) and amino acid (SEQ ID NO:304) sequence for the human homolog to Drosophila gene CG17010 (SWISS-PROT Ref. No. Q9H477).

FIG. 137 shows the nucleic acid sequences (SEQ ID NO:305, SEQ ID NO:307 and SEQ ID NO:309, respectively) and amino acid sequences (SEQ ID NO:306, SEQ ID NO:308 and SEQ ID NO:310, respectively) for the three human homologs to Drosophila gene Tkv (SWISS-PROT Ref. No. 000238, SWISS-PROT Ref. No. P36894 and SWISS-PROT Ref. No. Q04771).

FIG. 138 shows the nucleic acid (SEQ ID NO:311) and amino acid (SEQ ID NO:312) sequence for the human homolog to Drosophila gene Dnt (SWISS-PROT Ref. No. P34925).

FIG. 139 shows the nucleic acid sequences (SEQ ID NO:313 and SEQ ID NO:315, respectively) and amino acid sequences (SEQ ID NO:314 and SEQ ID NO:316, respectively) for the two human homologs to Drosophila gene ACXD (SWISS-PROT Ref. No. Q08462 and SWISS-PROT Ref. No. P40145).

FIG. 140 shows the nucleic acid (SEQ ID NO:317) and amino acid (SEQ ID NO:318) sequence for the human homolog to Drosophila gene Aats-ala-m (SWISS-PROT Ref. No. P49588).

FIG. 141 shows the nucleic acid (SEQ ID NO:319) and amino acid (SEQ ID NO:320) sequence for the human homolog to Drosophila gene Gek (SWISS-PROT Ref. No. Q9Y5S2).

FIG. 142 shows the nucleic acid sequences (SEQ ID NO:321 and SEQ ID NO:323, respectively) and amino acid sequence (SEQ ID NO:322 and SEQ ID NO:324, respectively) for the two human homologs to Drosophila gene CG3216 (SWISS-PROT Ref. No. P16066 and SWISS-PROT Ref. No. P20594).

FIG. 143 shows the nucleic acid (SEQ ID NO:325) and amino acid (SEQ ID NO:326) sequence for the human homolog to Drosophila gene CG5653 (SWISS-PROT Ref. No. Q96QT3).

FIG. 144 shows the nucleic acid (SEQ ID NO:327) and amino acid (SEQ ID NO:328) sequence for the human homolog to Drosophila gene CG17740 (SWISS-PROT Ref. No. Q9HCB6).

FIG. 145 shows the nucleic acid (SEQ ID NO:329) and amino acid (SEQ ID NO:330) sequence for the human homolog to Drosophila gene TepI (SWISS-PROT Ref. No. Q8TDJ3).

FIG. 146 shows the nucleic acid (SEQ ID NO:331) and amino acid (SEQ ID NO:332) sequence for the human homolog to Drosophila gene for (SWISS-PROT Ref. No. Q13976).

FIG. 147 shows the nucleic acid sequences (SEQ ID NO:333, and SEQ ID NO:335, respectively) and amino acid sequences (SEQ ID NO:334 and SEQ ID NO:336, respectively) for the two human homologs to Drosophila gene Ac13E (SWISS-PROT Ref. No. 060503 and SWISS-PROT Ref. No. 060266).

FIG. 148 shows the nucleic acid (SEQ ID NO:337) and amino acid (SEQ ID NO:338) sequence for the human homolog to Drosophila gene CG2667 (SWISS-PROT Ref. No. Q16760).

FIG. 149 shows the nucleic acid (SEQ ID NO:339) and amino acid (SEQ ID NO:340) sequence for the human homolog to Drosophila gene CG7842 (SWISS-PROT Ref. No. 095510).

FIG. 150 shows the nucleic acid (SEQ ID NO:341) and amino acid (SEQ ID NO:342) sequence for the human homolog to Drosophila gene CG17486 (SWISS-PROT Ref. No. Q9NWL6).

FIG. 151 shows the nucleic acid (SEQ ID NO:343) and amino acid (SEQ ID NO:344) sequence for the human homolog to Drosophila gene CG6969 (SWISS-PROT Ref. No. Q92626).

FIG. 152 shows the nucleic acid (SEQ ID NO:345) and amino acid (SEQ ID NO:346) sequence for the human homolog to Drosophila gene CG12262 (SWISS-PROT Ref. No. P11310).

FIG. 153 shows the nucleic acid (SEQ ID NO:347) and amino acid (SEQ ID NO:348) sequence for the human homolog to Drosophila gene Fray (SWISS-PROT Ref. No. 095747).

FIG. 154 shows the nucleic acid (SEQ ID NO:349) and amino acid (SEQ ID NO:350) sequence for the human homolog to Drosophila gene CG6879 (SWISS-PROT Ref. No. Q92626).

FIG. 155 shows the nucleic acid (SEQ ID NO:351) and amino acid (SEQ ID NO:352) sequence for the human homolog to Drosophila gene CG11594 (SWISS-PROT Ref. No. Q96C11).

FIG. 156 shows the nucleic acid (SEQ ID NO:353) and amino acid (SEQ ID NO:354) sequence for the human homolog to Drosophila gene S6kII (SWISS-PROT Ref. No. P51812).

FIG. 157 shows the nucleic acid sequences (SEQ ID NO:355 and SEQ ID NO:357, respectively) and amino acid sequences (SEQ ID NO:356 and SEQ ID NO:358, respectively) for the two human homologs to Drosophila gene CG11714 (SWISS-PROT Ref. No. Q9HAB2 and SWISS-PROT Ref. No. 043791).

FIG. 158 shows the nucleic acid (SEQ ID NO:359) and amino acid (SEQ ID NO:360) sequence for the human homolog to Drosophila gene CG3534 (SWISS-PROT Ref. No. 075191).

FIG. 159 shows the nucleic acid (SEQ ID NO:361) and amino acid (SEQ ID NO:362) sequence for the human homolog to Drosophila gene CG7335 (SWISS-PROT Ref. No. P50053).

FIG. 160 shows the nucleic acid sequences (SEQ ID NO:363 and SEQ ID NO:265, respectively) and amino acid sequences (SEQ ID NO:364 and SEQ ID NO:366, respectively) for the two human homologs to Drosophila gene CG11275 (SWISS-PROT Ref. No. Q9HAB2 and SWISS-PROT Ref. No. 043791).

FIG. 161 shows the nucleic acid (SEQ ID NO:367) and amino acid (SEQ ID NO:368) sequence for the human homolog to Drosophila gene CG16726 (SWISS-PROT Ref. No. P34981).

FIG. 162 shows the nucleic acid (SEQ ID NO:369) and amino acid (SEQ ID NO:370) sequence for the human homolog to Drosophila gene CG7514 (SWISS-PROT Ref. No. Q02978).

FIG. 163 shows the nucleic acid (SEQ ID NO:371) and amino acid (SEQ ID NO:372) sequence for the human homolog to Drosophila gene CG17283 (SWISS-PROT Ref. No. P14091).

FIG. 164 shows the nucleic acid (SEQ ID NO:373) and amino acid (SEQ ID NO:374) sequence for the human homolog to Drosophila gene BcDNA:GH07626 (SWISS-PROT Ref. No. Q16702).

FIG. 165 shows the nucleic acid (SEQ ID NO:375) and amino acid (SEQ ID NO:376) sequence for the human homolog to Drosophila gene CG16752 (SWISS-PROT Ref. No. Q8TDU8).

FIG. 166 shows the nucleic acid (SEQ ID NO:377) and amino acid (SEQ ID NO:378) sequence for the human homolog to Drosophila gene Rpt1 (SWISS-PROT Ref. No. P35998).

FIG. 167 shows the nucleic acid sequences (SEQ ID NO:379 and SEQ ID NO:381, respectively) and amino acid sequences (SEQ ID NO:380 and SEQ ID NO:382, respectively) for the two human homologs to Drosophila gene Wts (SWISS-PROT Ref. No. 095835 and SWISS-PROT Ref. No. Q9P2×1).

FIG. 168 shows the nucleic acid (SEQ ID NO:383) and amino acid (SEQ ID NO:384) sequence for the human homolog to Drosophila gene CG1582 (SWISS-PROT Ref. No. AAM73547).

FIG. 169 shows the nucleic acid (SEQ ID NO:385) and amino acid (SEQ ID NO:386) sequence for the human homolog to Drosophila gene CG12289 (SWISS-PROT Ref. No. P50053).

FIG. 170 shows the nucleic acid (SEQ ID NO:387) and amino acid (SEQ ID NO:388) sequence for the human homolog to Drosophila gene Pepck (SWISS-PROT Ref. No. Q16822).

FIG. 171 shows the nucleic acid (SEQ ID NO:389) and amino acid (SEQ ID NO:390) sequence for the human homolog to Drosophila gene CG5665 (SWISS-PROT Ref. No. PO₆₈₅₈).

FIG. 172 shows the nucleic acid (SEQ ID NO:391) and amino acid (SEQ ID NO:392) sequence for the human homolog to Drosophila gene CG7285 (SWISS-PROT Ref. No. Q96TF2).

FIG. 173 shows the nucleic acid (SEQ ID NO:393) and amino acid (SEQ ID NO:394) sequence for the human homolog to Drosophila gene Bt (SWISS-PROT Ref. No. Q8WZ42).

FIG. 174 shows the nucleic acid (SEQ ID NO:395) and amino acid (SEQ ID NO:396) sequence for the human homolog to Drosophila gene CG8795 (SWISS-PROT Ref. No. Q9GZQ4).

FIG. 175 shows the nucleic acid (SEQ ID NO:397) and amino acid (SEQ ID NO:398) sequence for the human homolog to Drosophila gene CGI0967 (SWISS-PROT Ref. No. O75385).

FIG. 176 shows the nucleic acid (SEQ ID NO:399) and amino acid (SEQ ID NO:400) sequence for the human homolog to Drosophila gene CG3809 (SWISS-PROT Ref. No. P55263).

FIG. 177 shows the nucleic acid (SEQ ID NO:401) and amino acid (SEQ ID NO:402) sequence for the human homolog to Drosophila gene Ack (SWISS-PROT Ref. No. Q07912).

FIG. 178 shows the nucleic acid (SEQ ID NO:403) and amino acid (SEQ ID NO:404) sequence for the human homo log to Drosophila gene Abl (SWISS-PROT Ref. No. P00519).

FIG. 179 shows the nucleic acid (SEQ ID NO:405) and amino acid (SEQ ID NO:406) sequence for the human homolog to Drosophila gene CG7362 (SWISS-PROT Ref. No. P14786).

FIG. 180 shows the nucleic acid (SEQ ID NO:407) and amino acid (SEQ ID NO:408) sequence for the human homolog to Drosophila gene Cyp9f2 (SWISS-PROT Ref. No. P08684).

FIG. 181 is a pair of graphs showing FACS profiles for D.Mel-2 cells.

FIG. 182 shows cells transfected with Ect2 siRNA COD1513 (aaGUGGGCUUUGUAAAGAUGG) result in a block in mitosis.

FIG. 183 shows preferred profile after adjustment of voltages on FSC and SSC channels.

FIG. 184 shows the profile after the voltage on FL3 channel is also altered to enable analysis of cells in G1, S and G2/M, setting a gate (G2) to exclude doublets resulting from cell clumping.

FIG. 185 is a histogram showing counts against FL3-H within the gated region (G2) and the associated histogram statistics is generated. Using the histogram statistics, events in G1, in S and in G2/M are calculated, where the number of G1 events was equal to M2×2, the number of G2/M events is equal to M3×2 and the number of S events equated to M1-[(M2×2)+(M3×2)].

FIG. 185 is a histogram showing counts against FL3-H within the gated region (G2) and the associated histogram statistics is generated. Using the histogram statistics, events in G1, in S and in G2/M are calculated, where the number of G1 events was equal to M2×2, the number of G2/M events is equal to M3×2 and the number of S events equated to M1−[(M2×2)+(M3×2)].

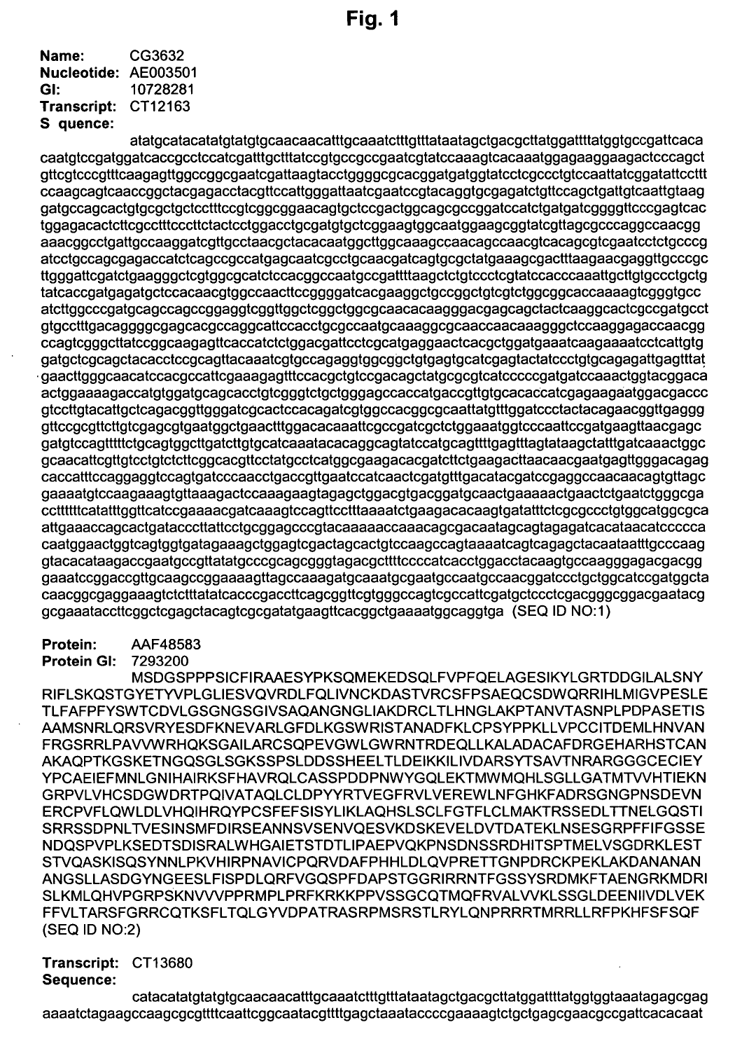

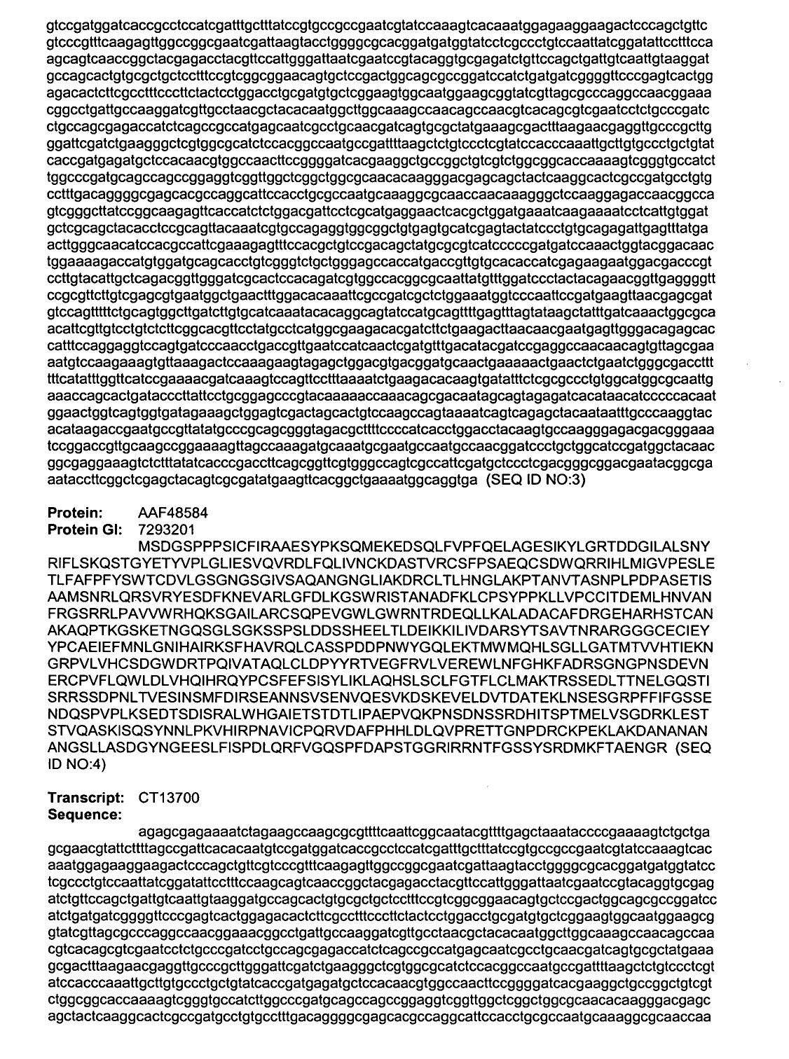

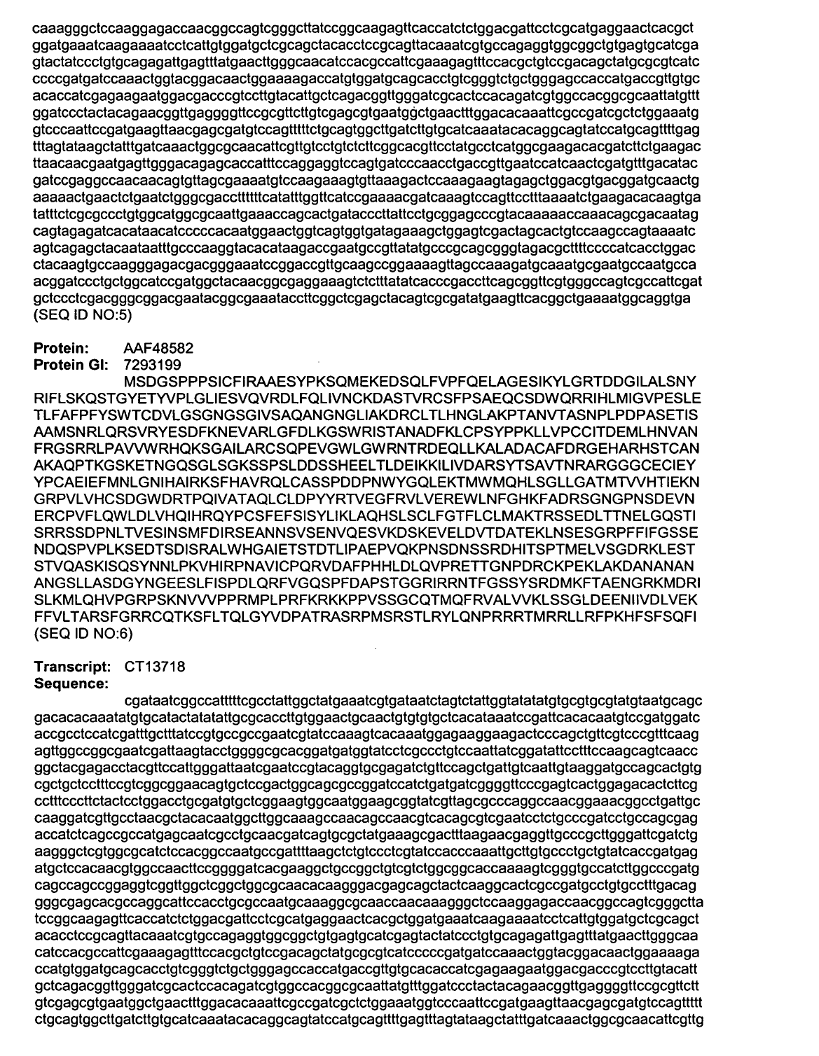

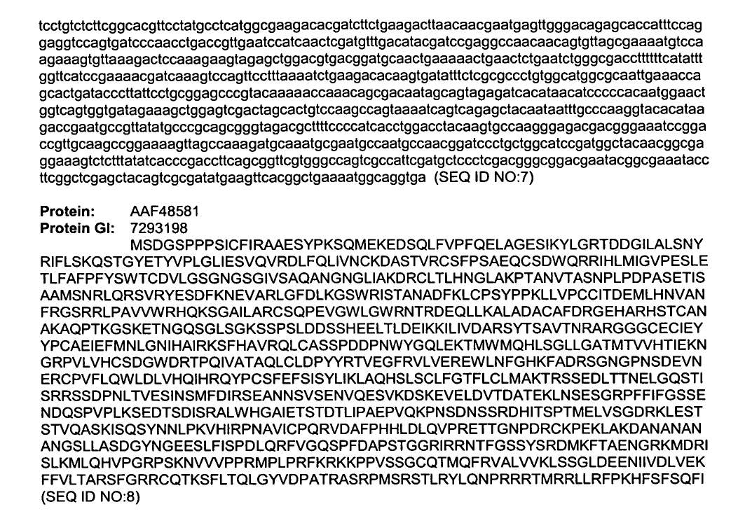

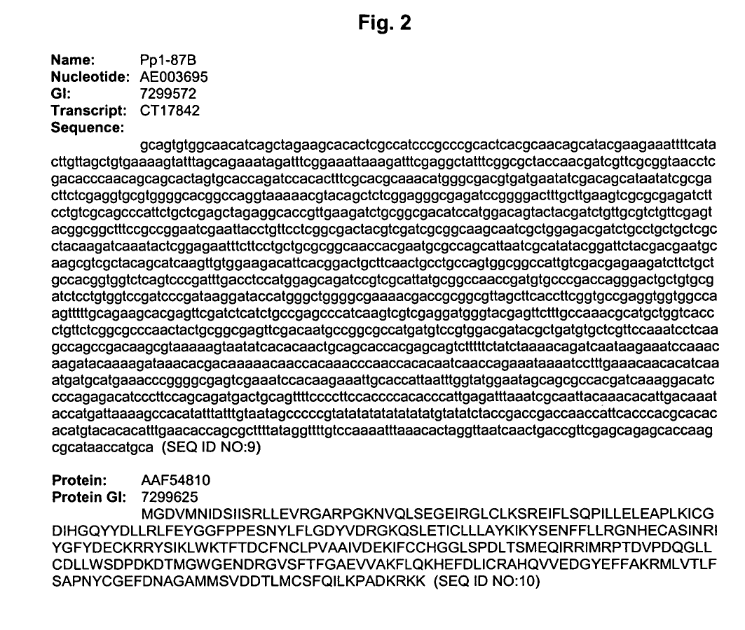

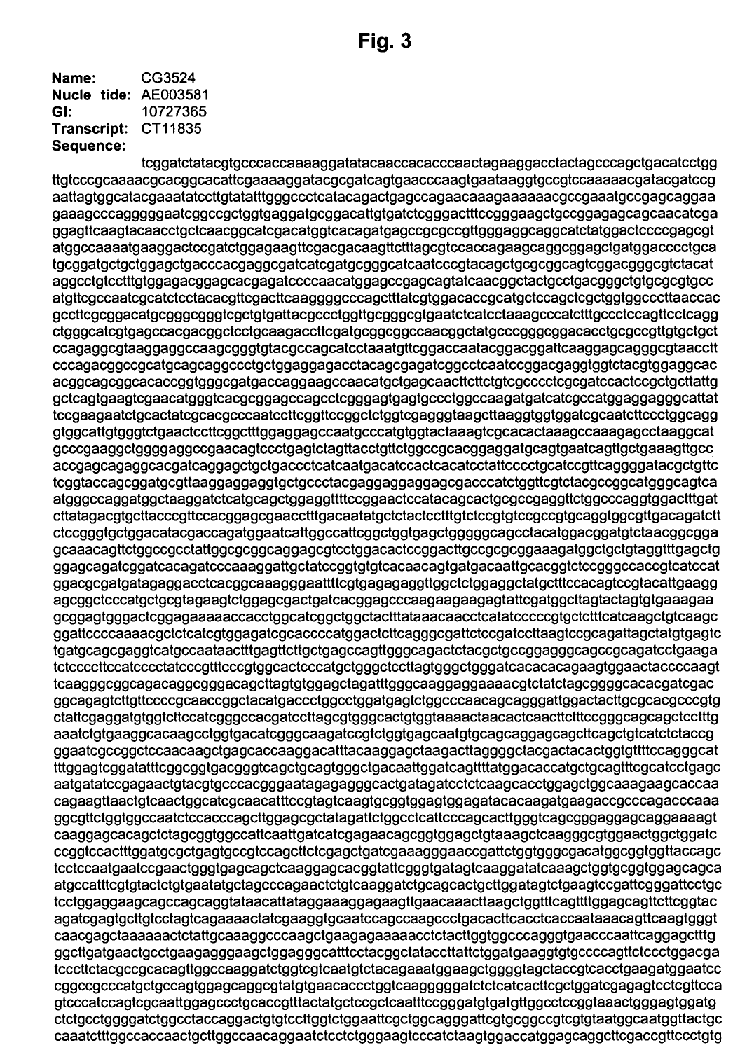

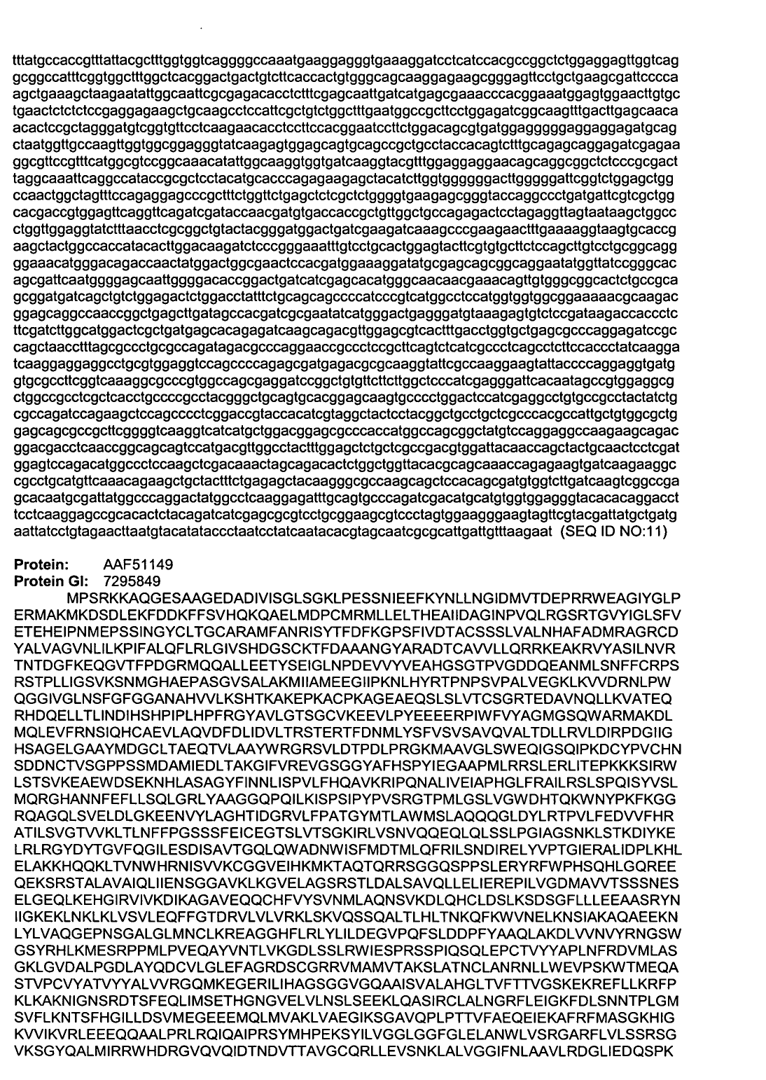

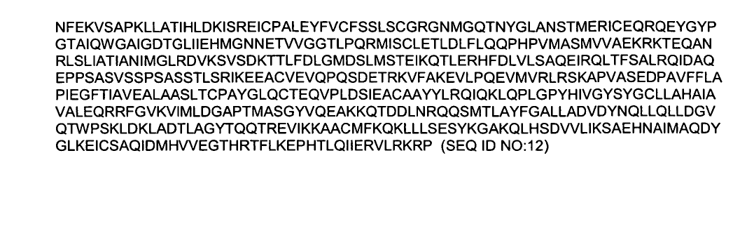

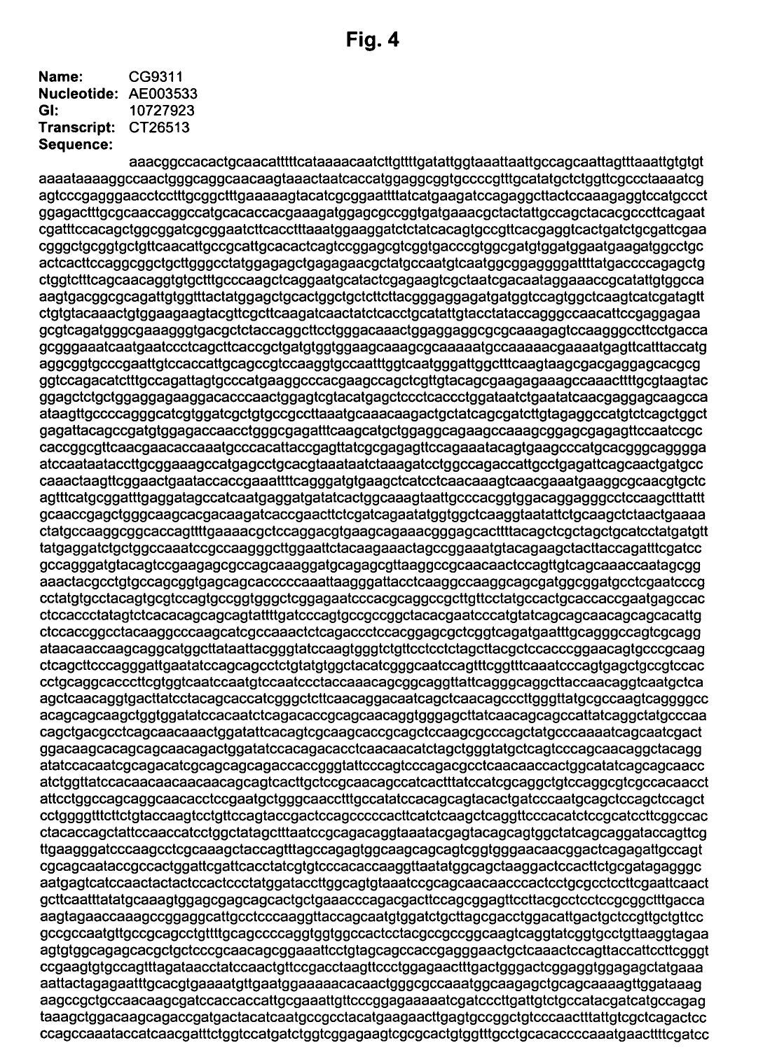

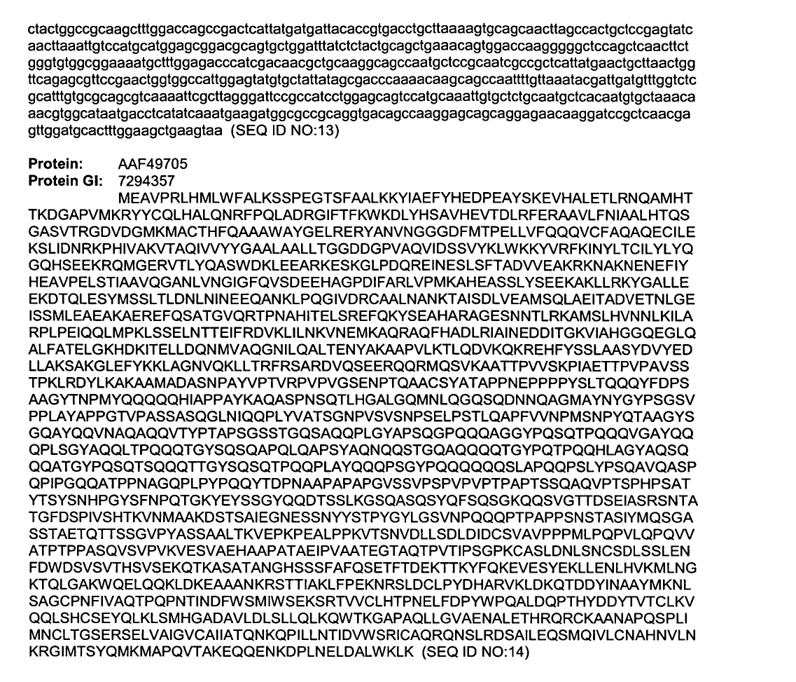

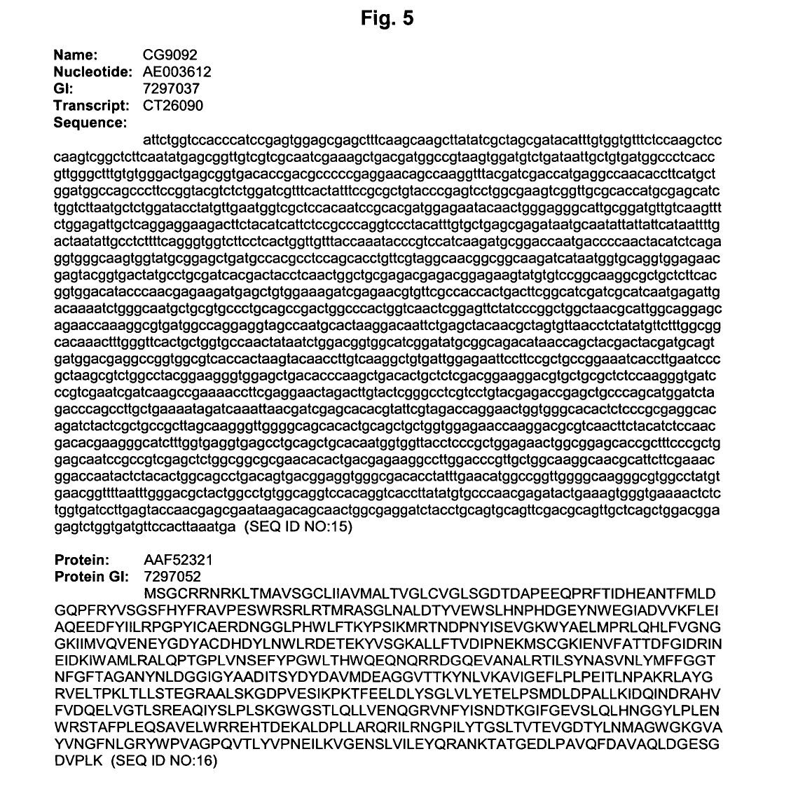

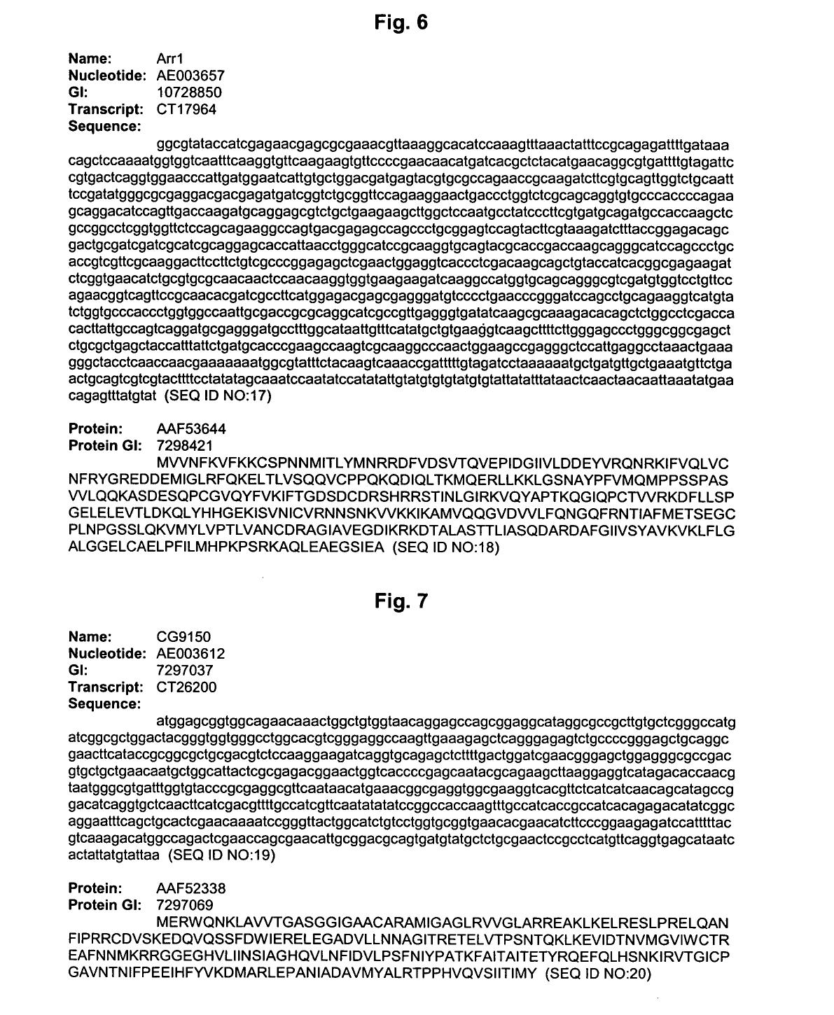

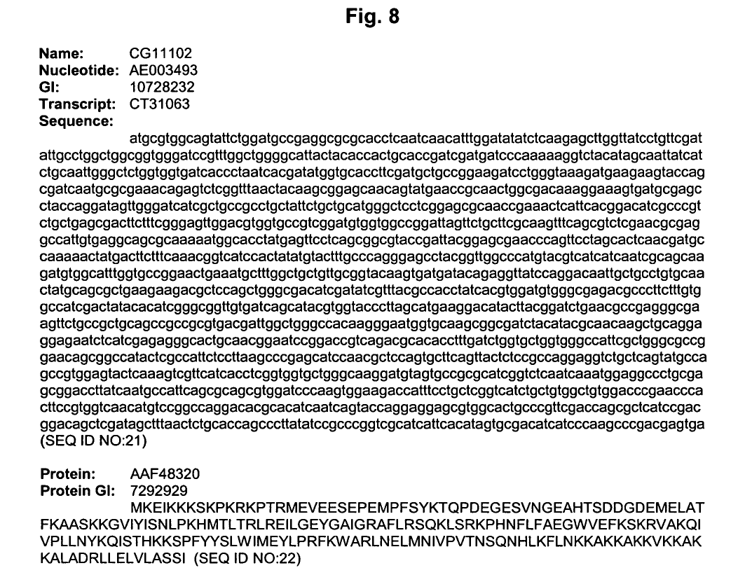

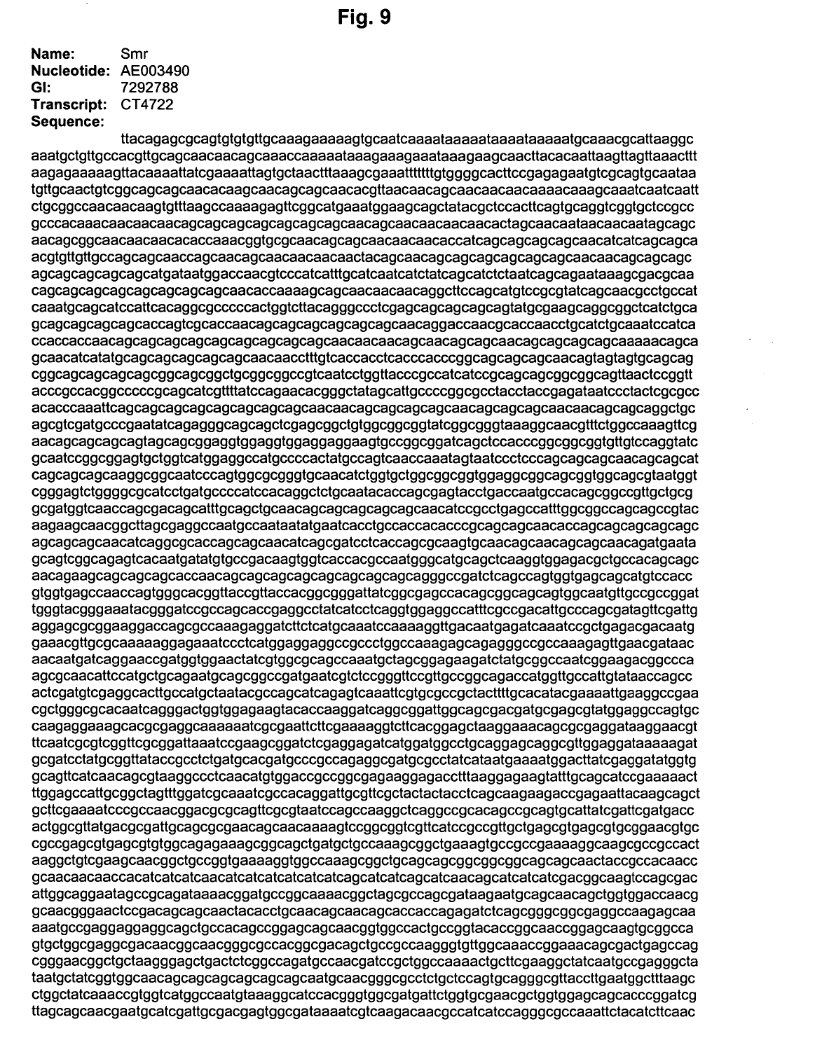

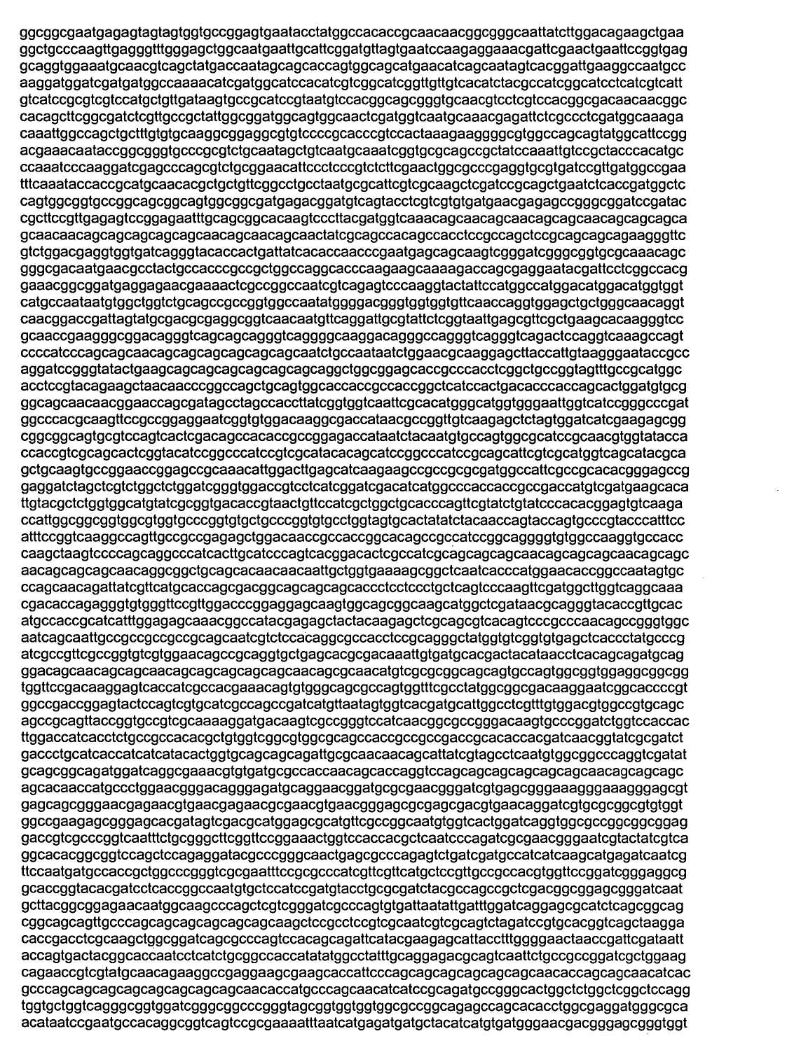

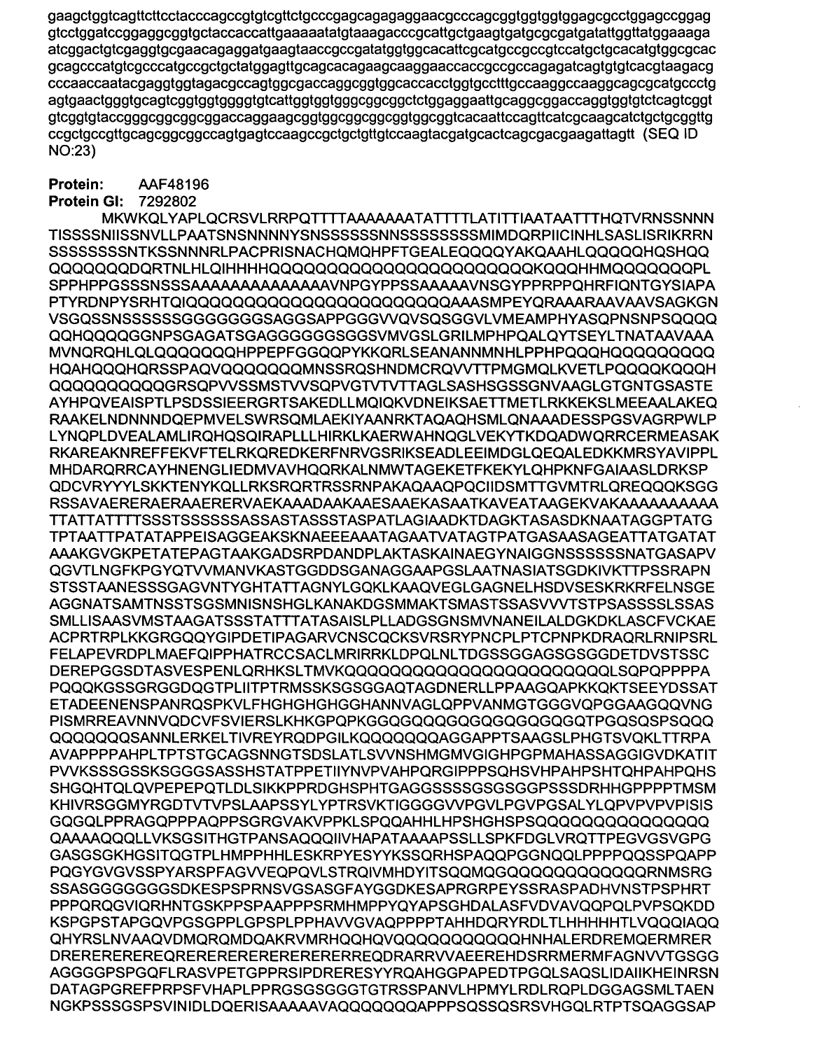

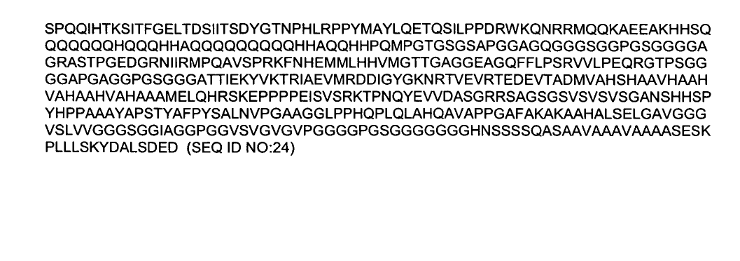

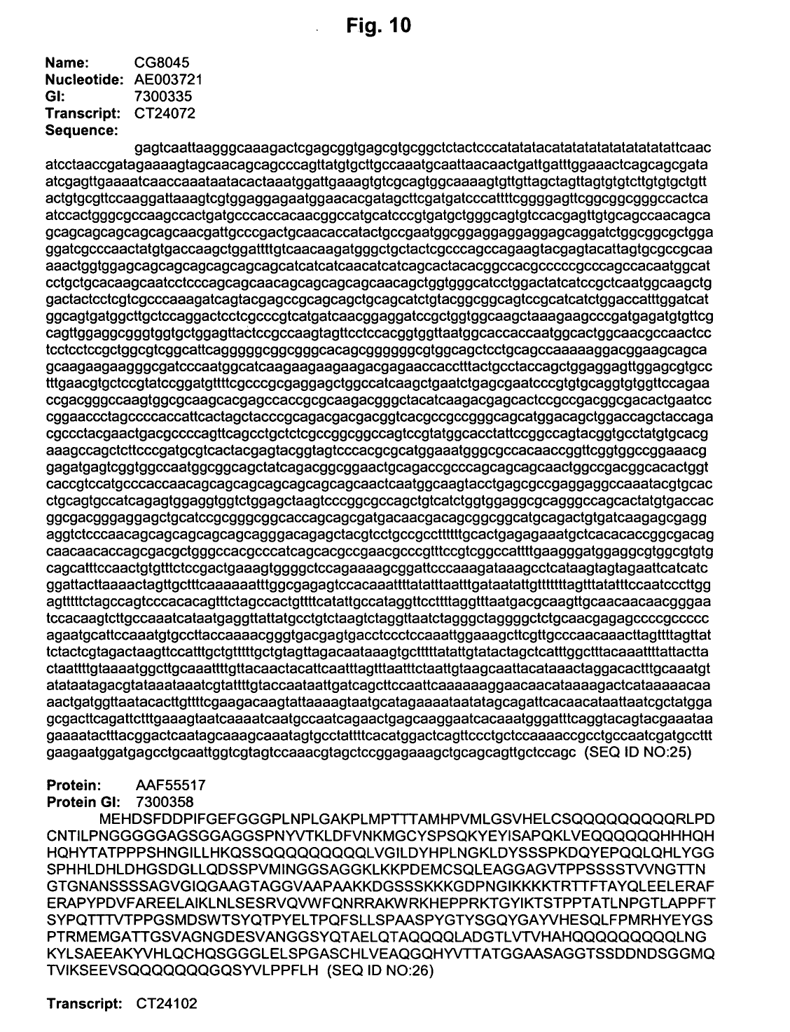

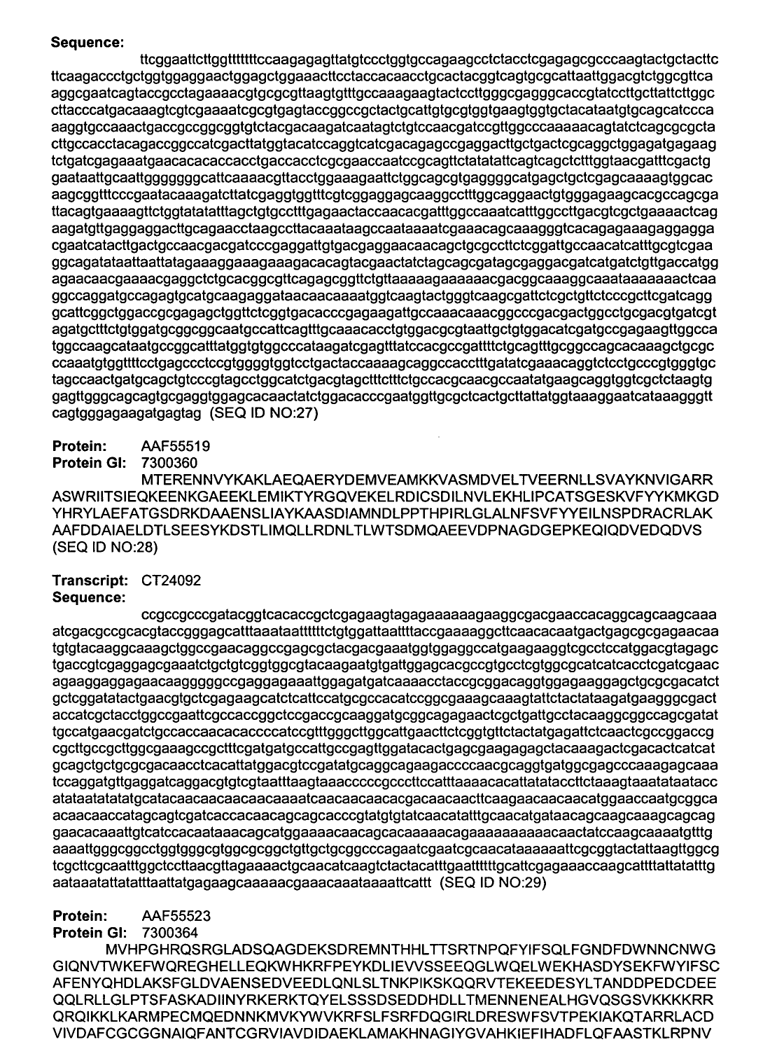

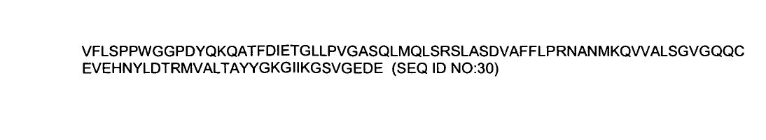

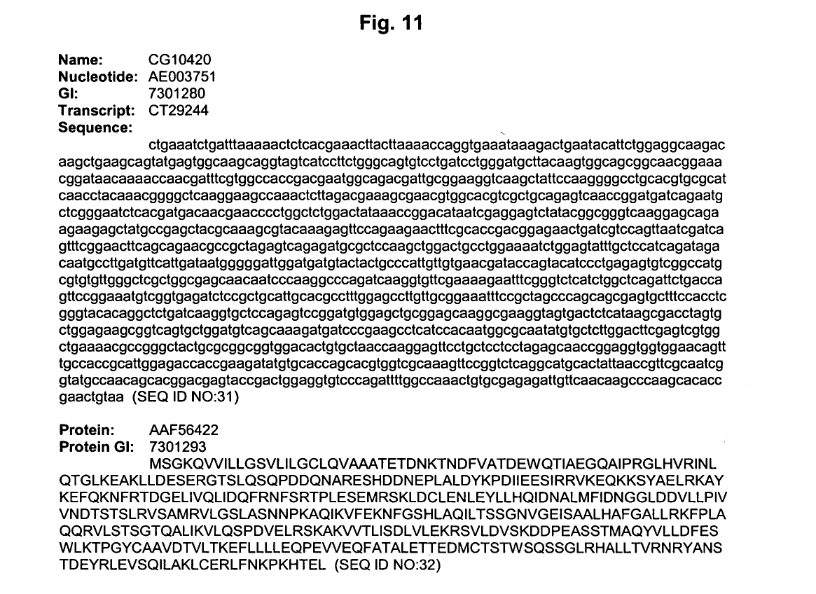

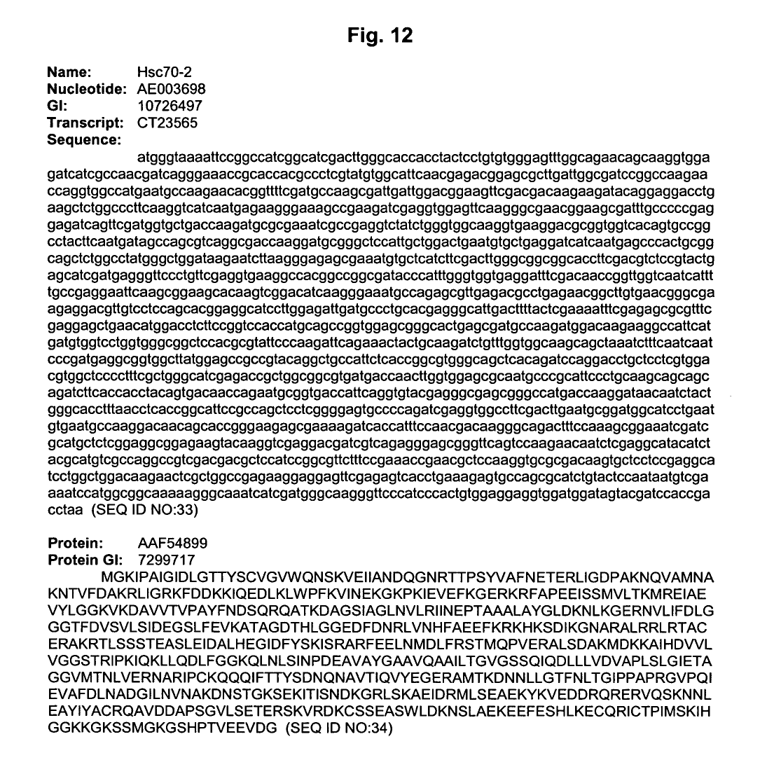

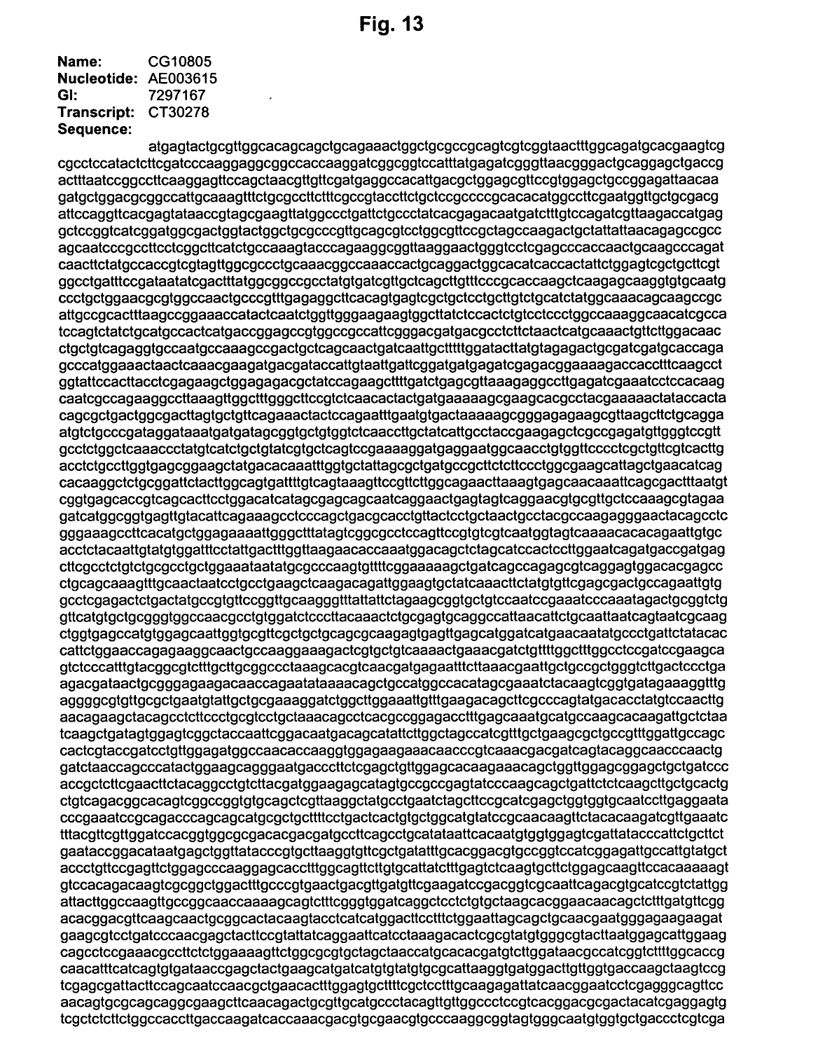

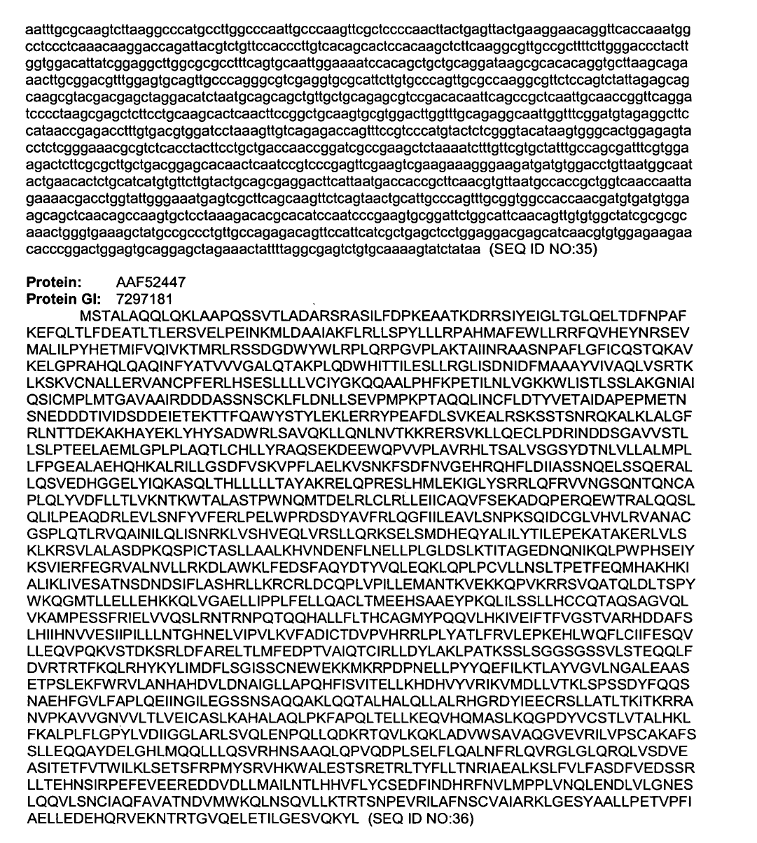

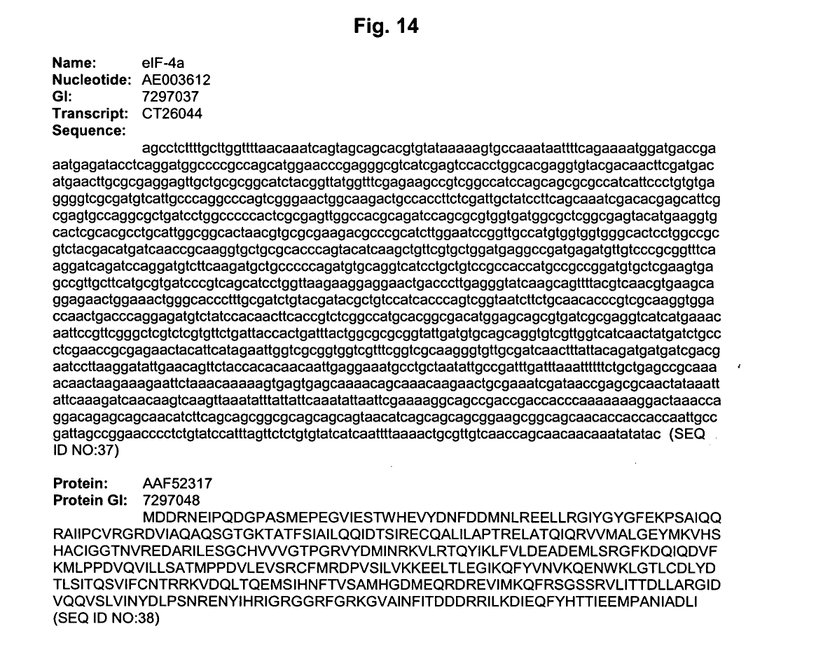

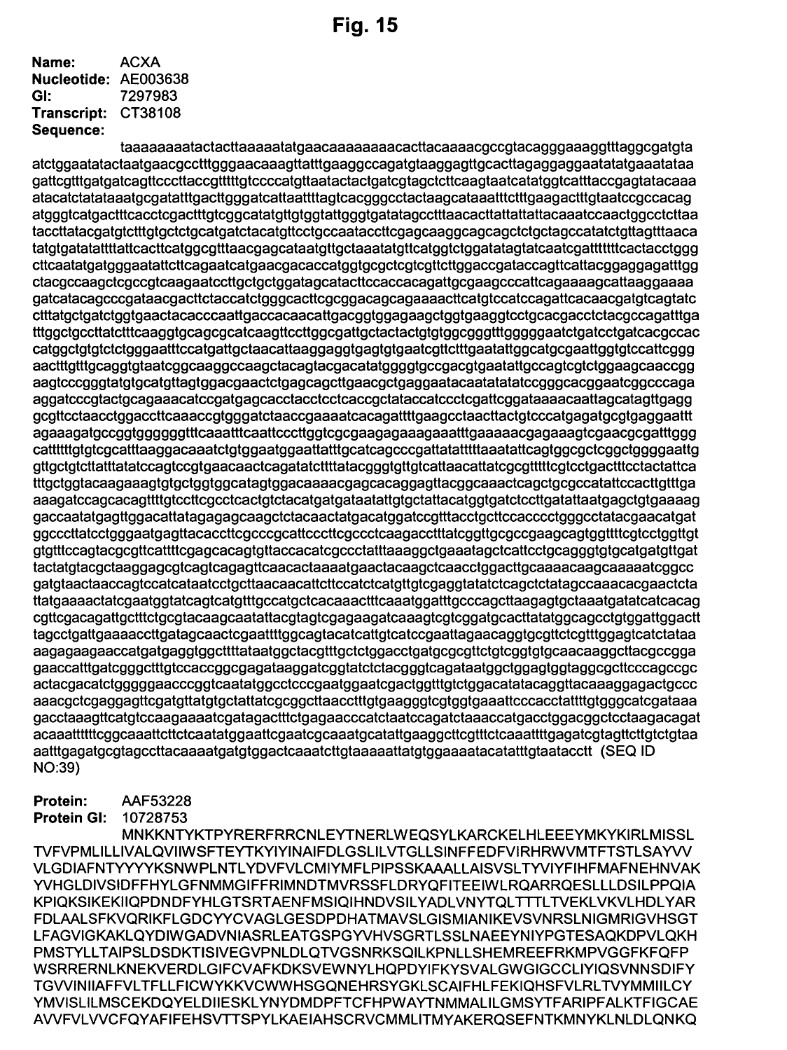

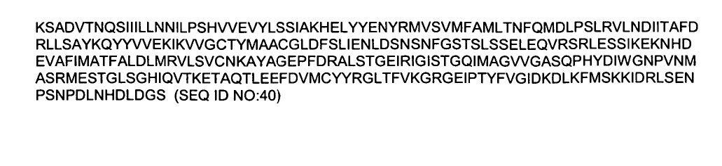

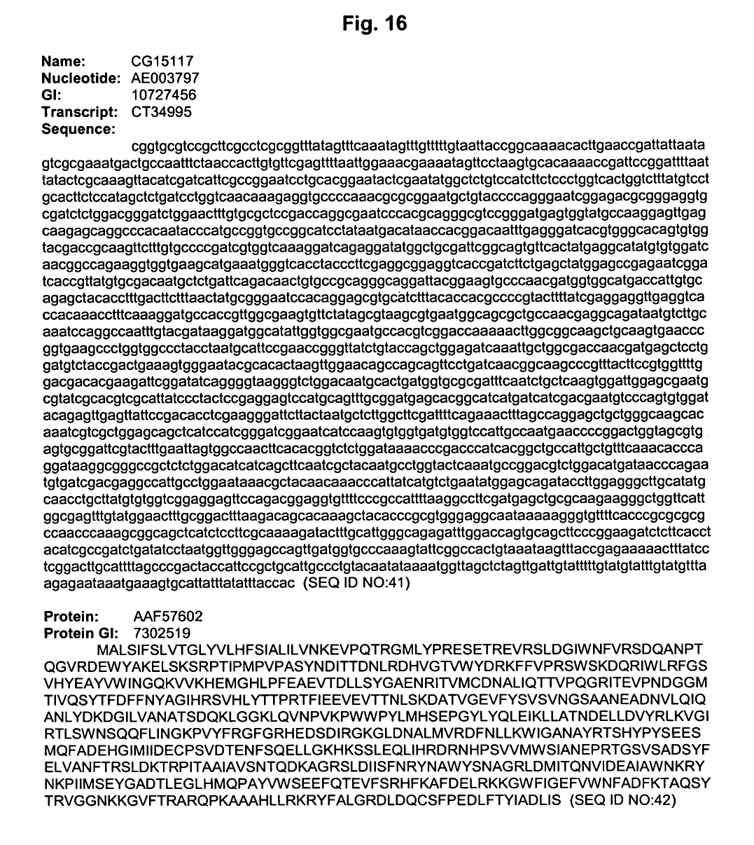

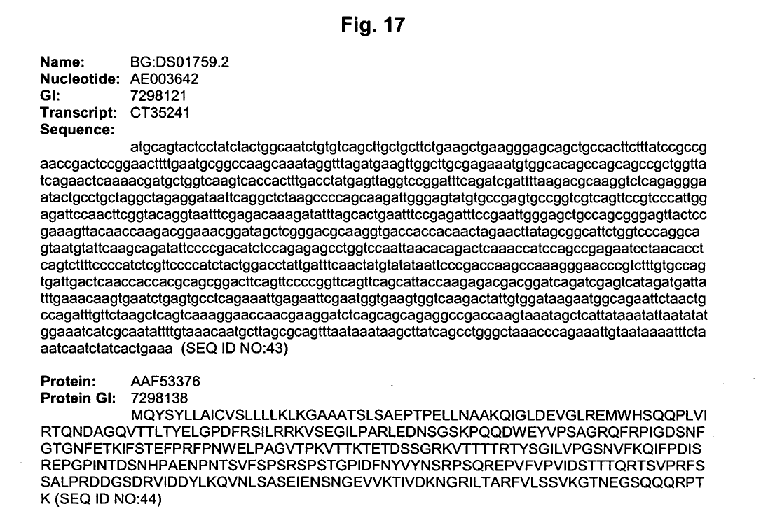

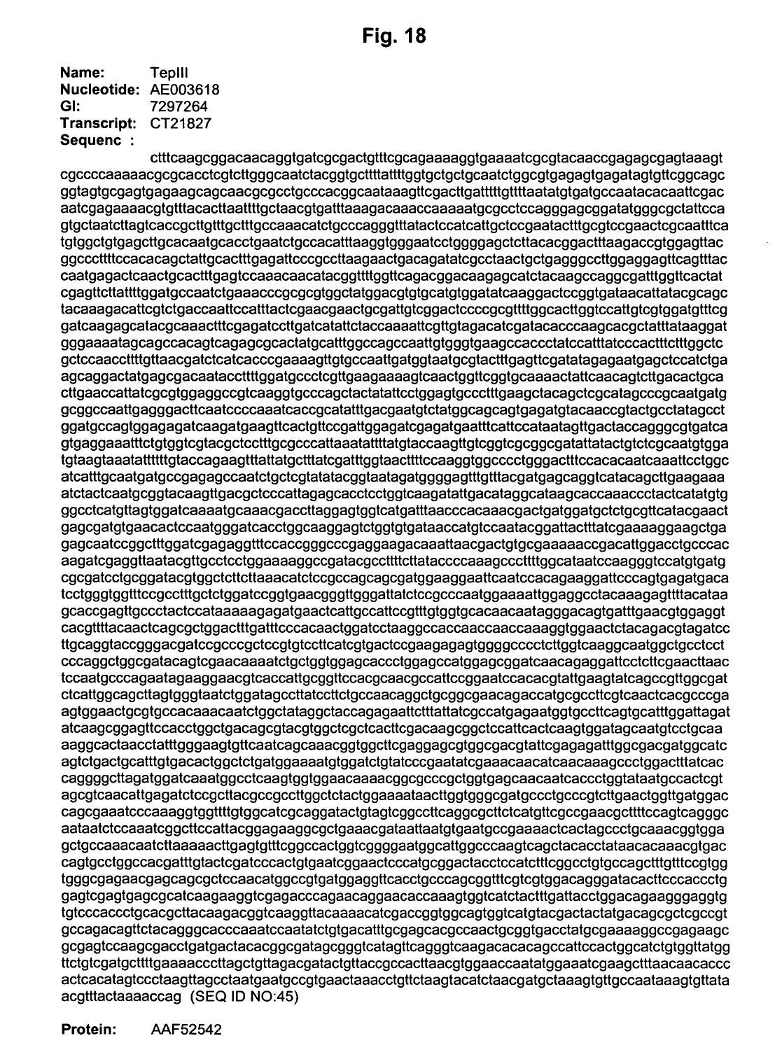

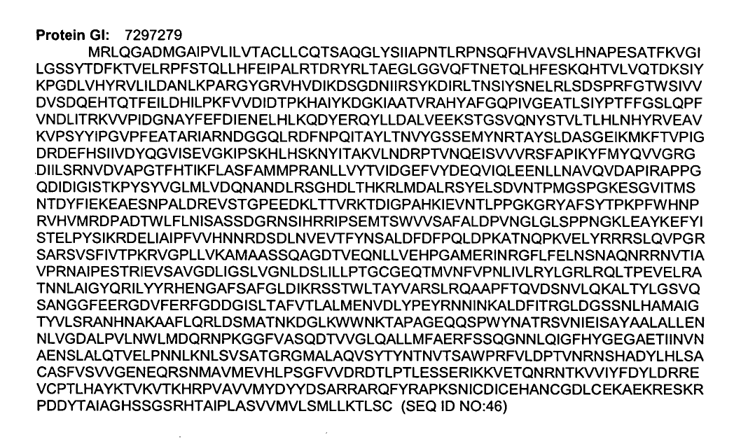

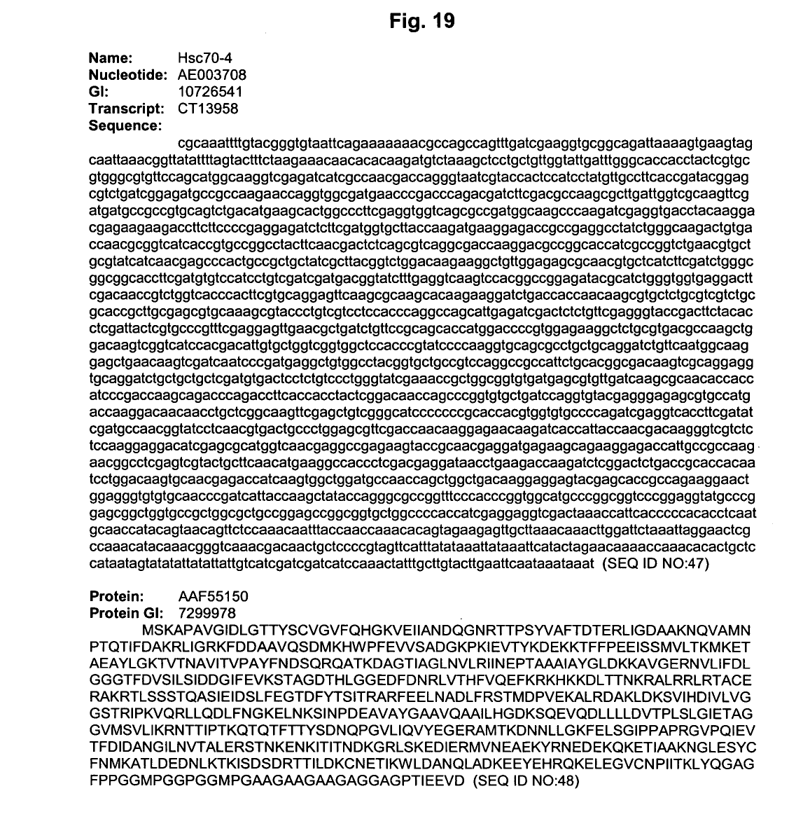

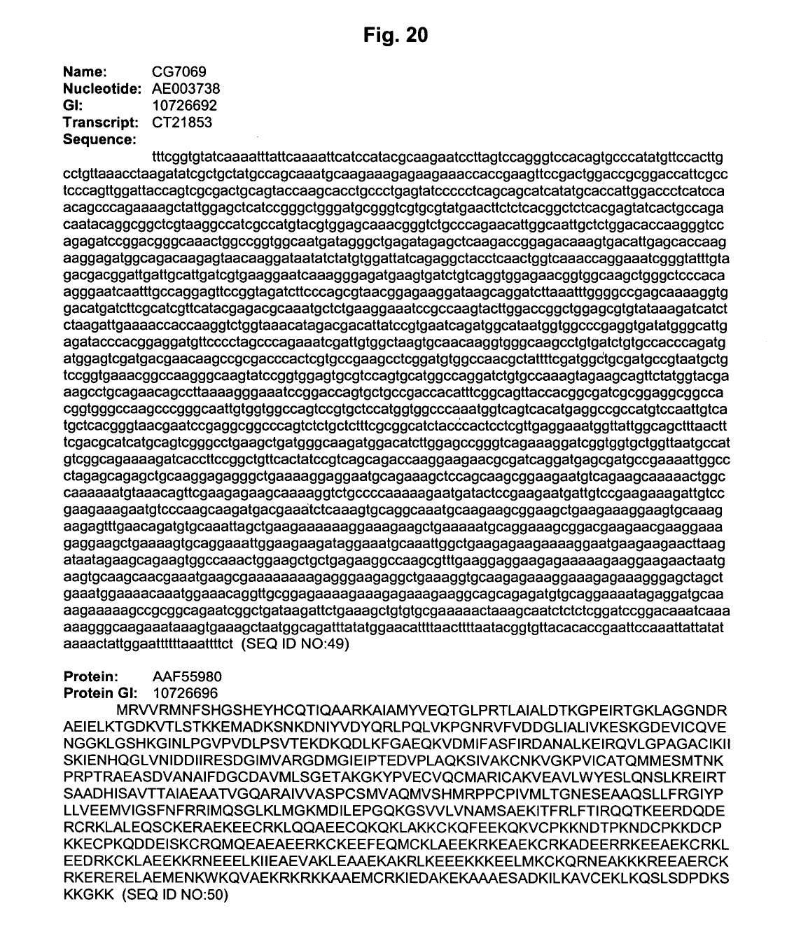

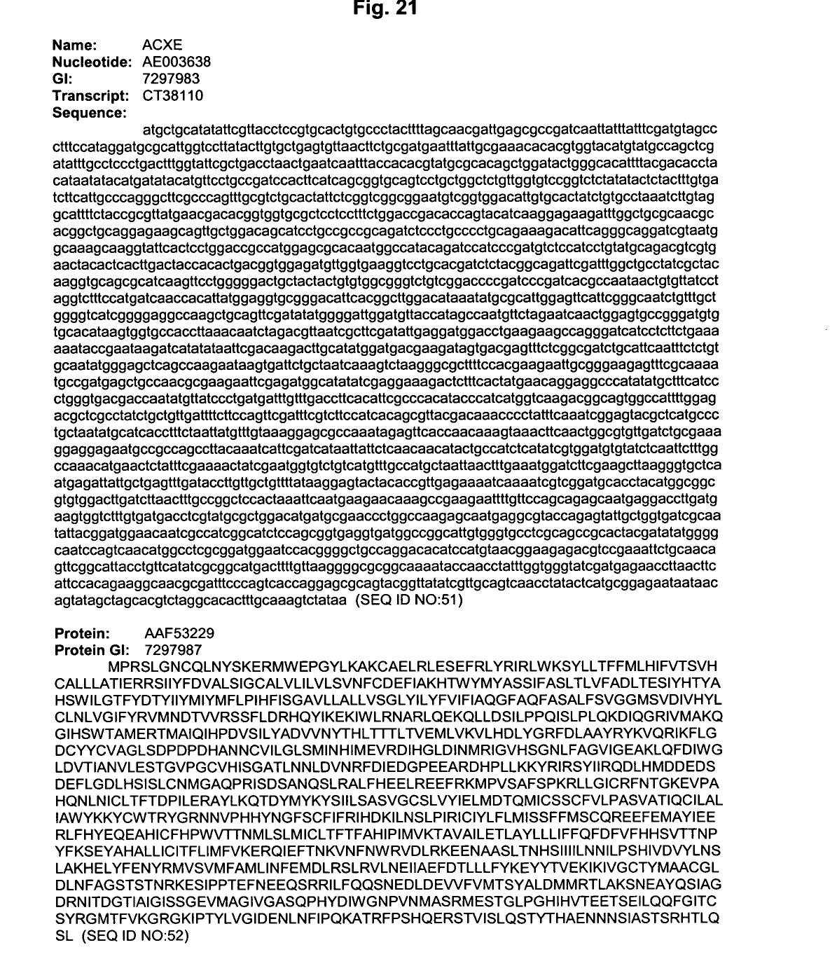

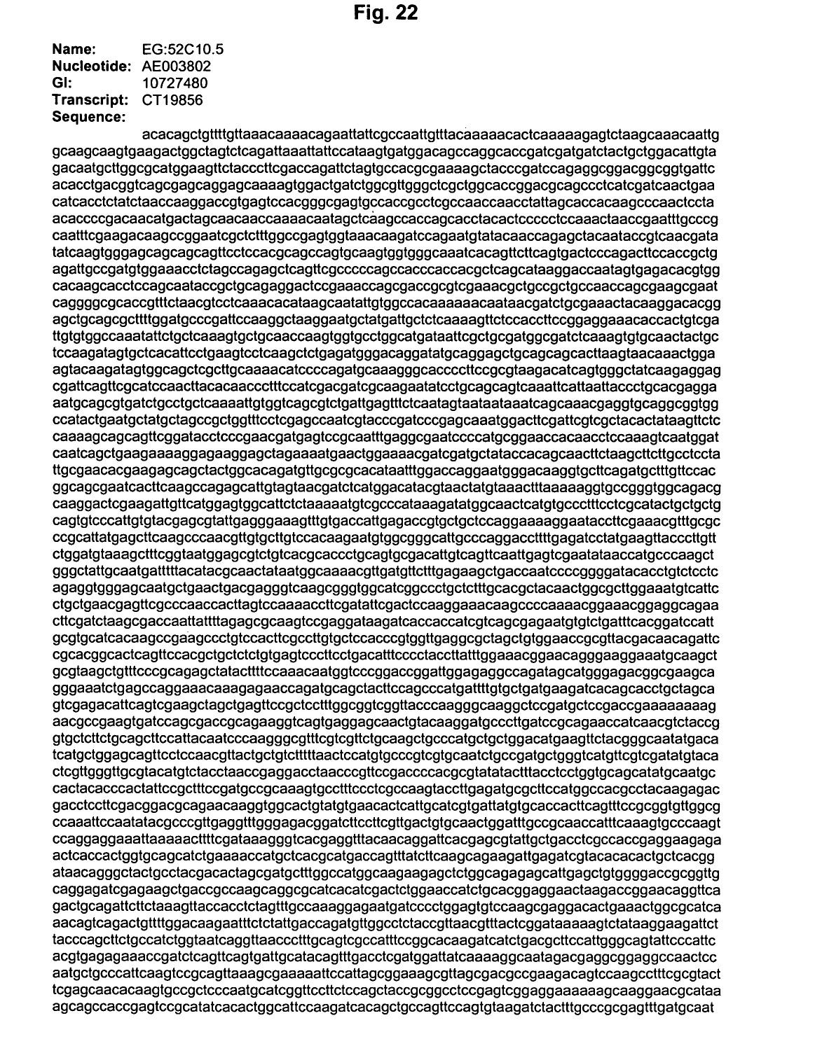

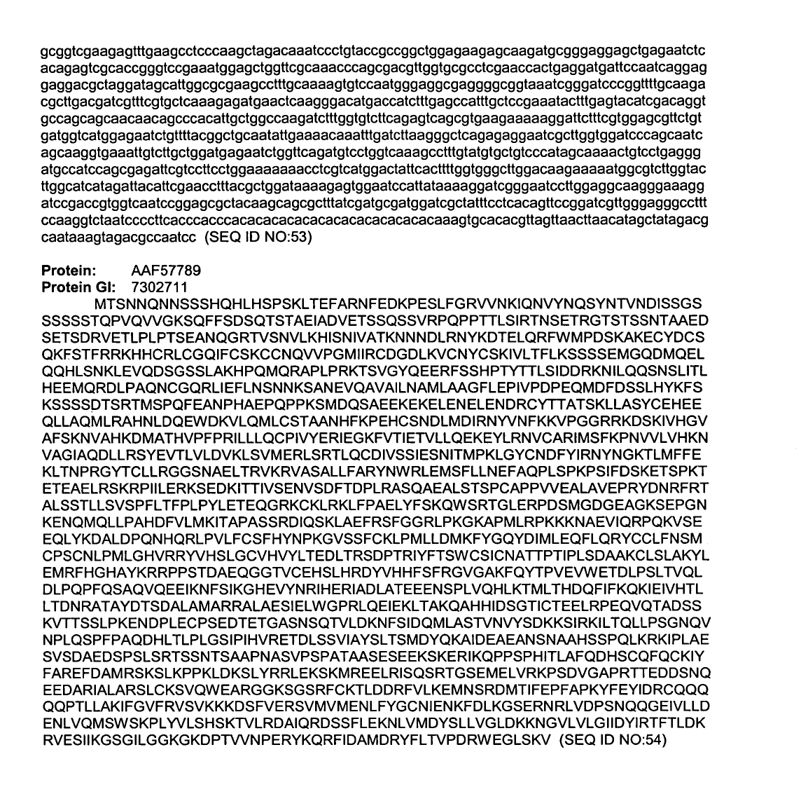

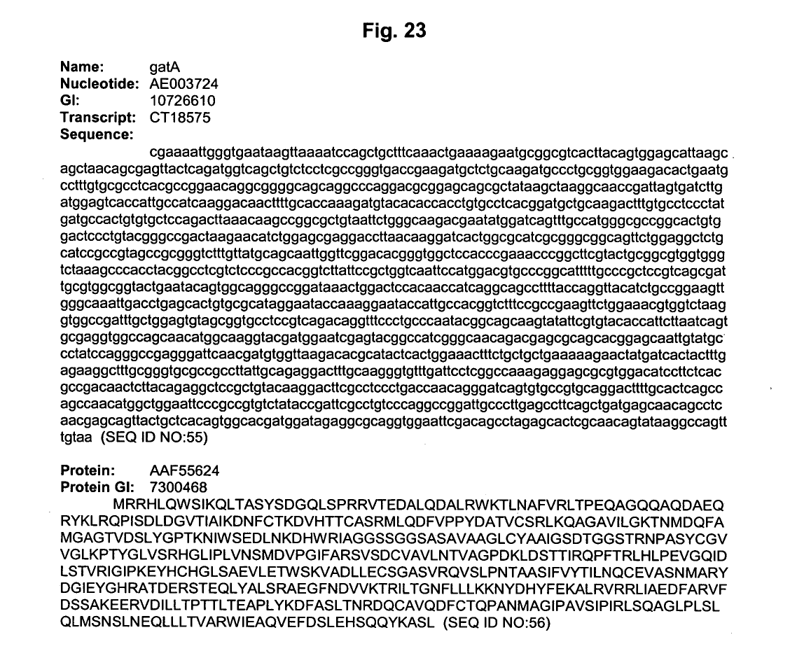

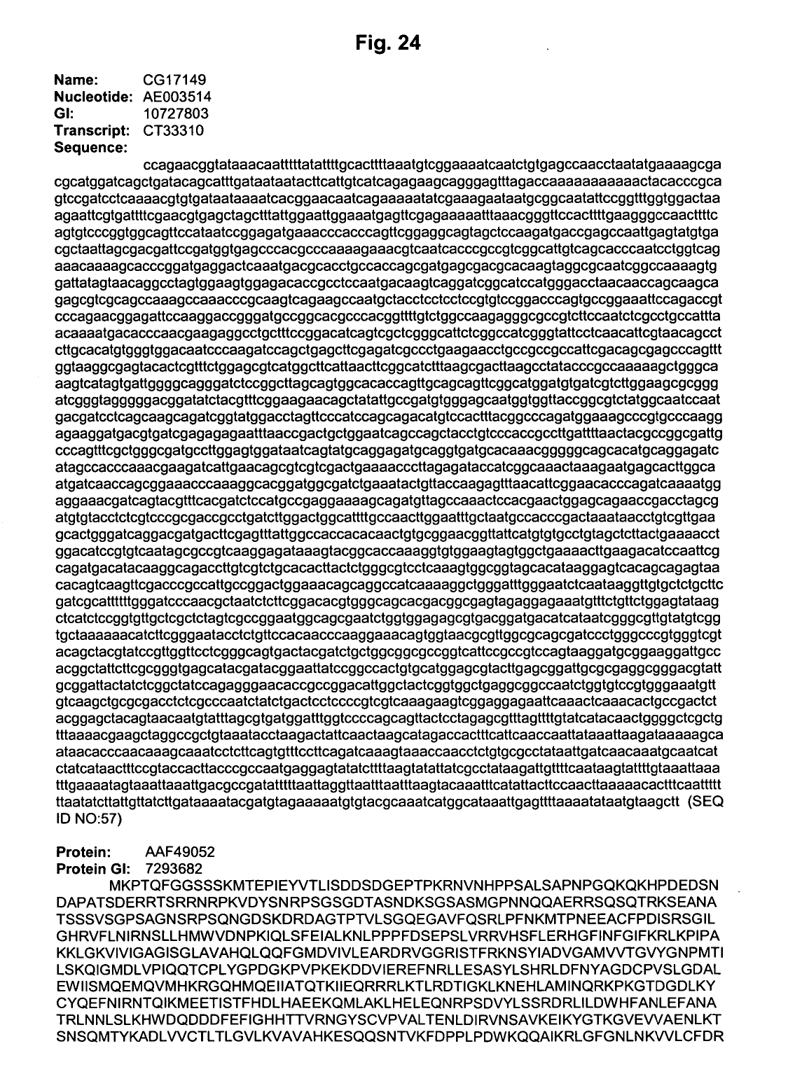

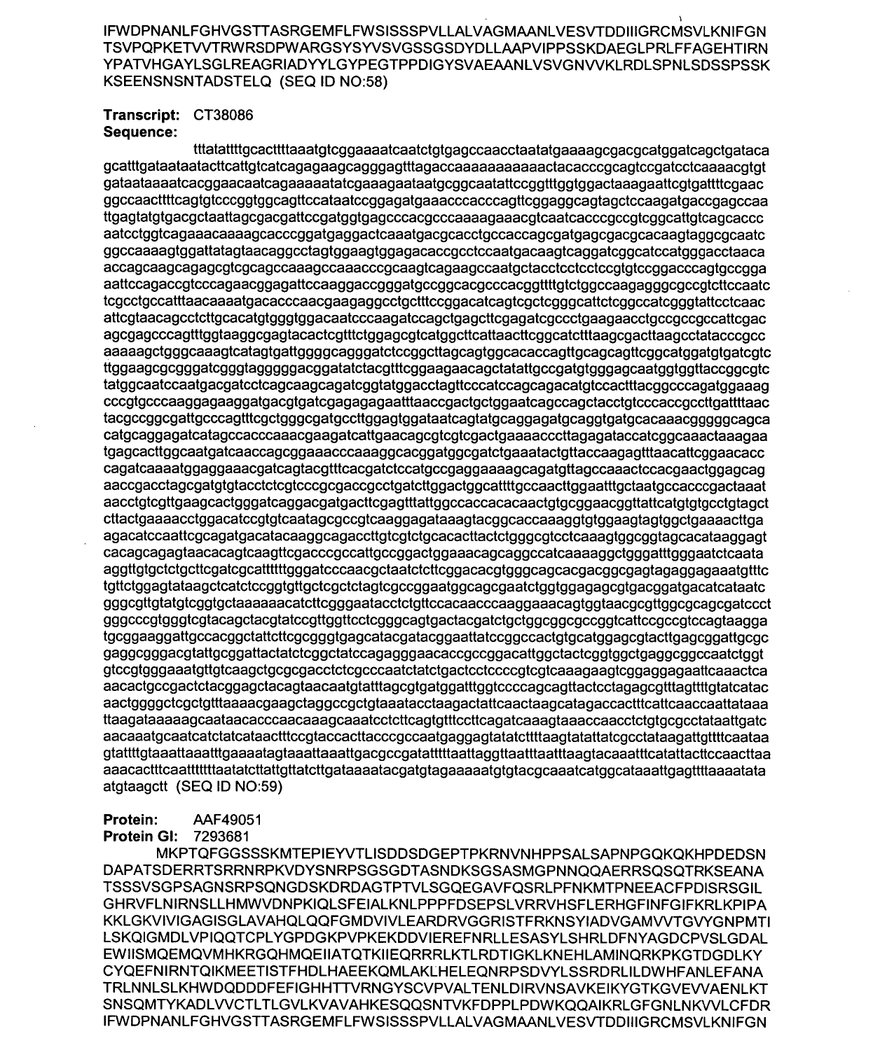

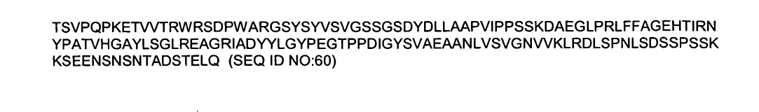

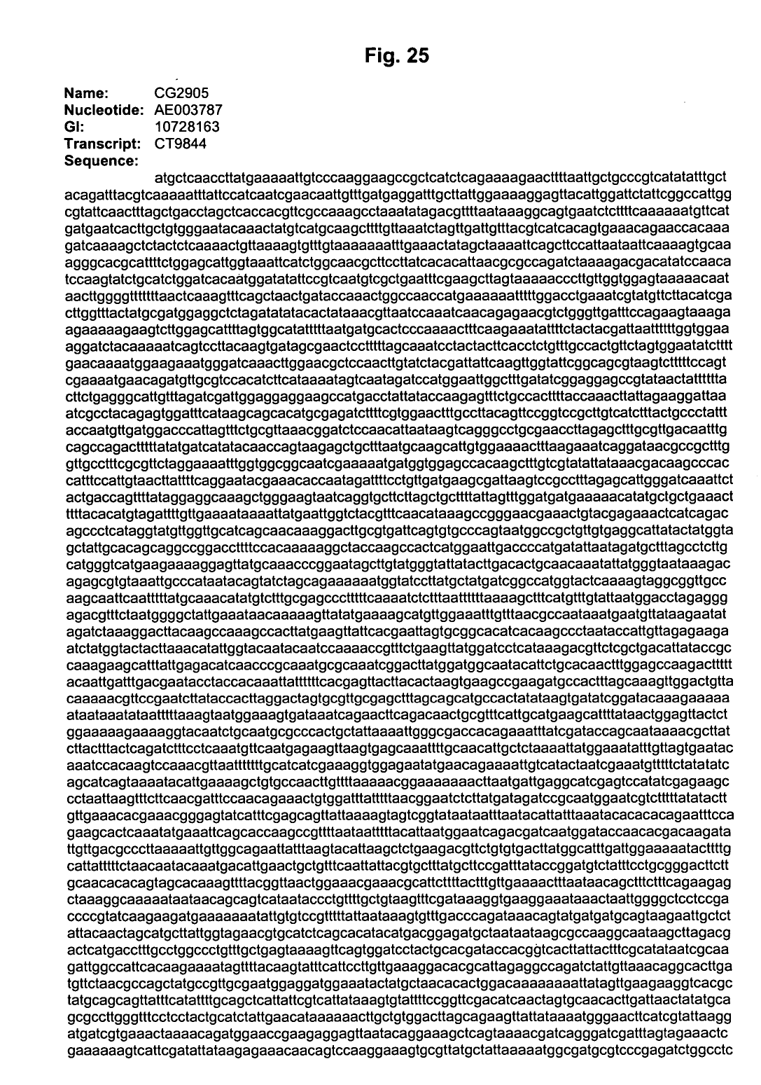

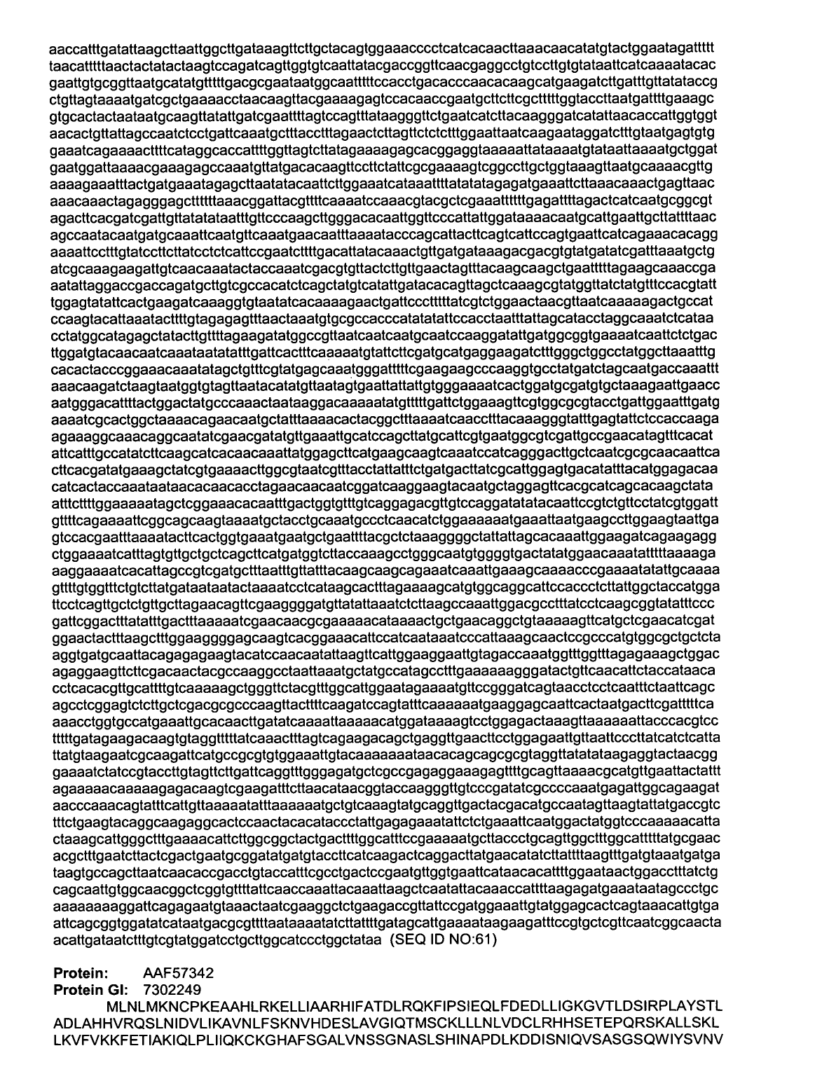

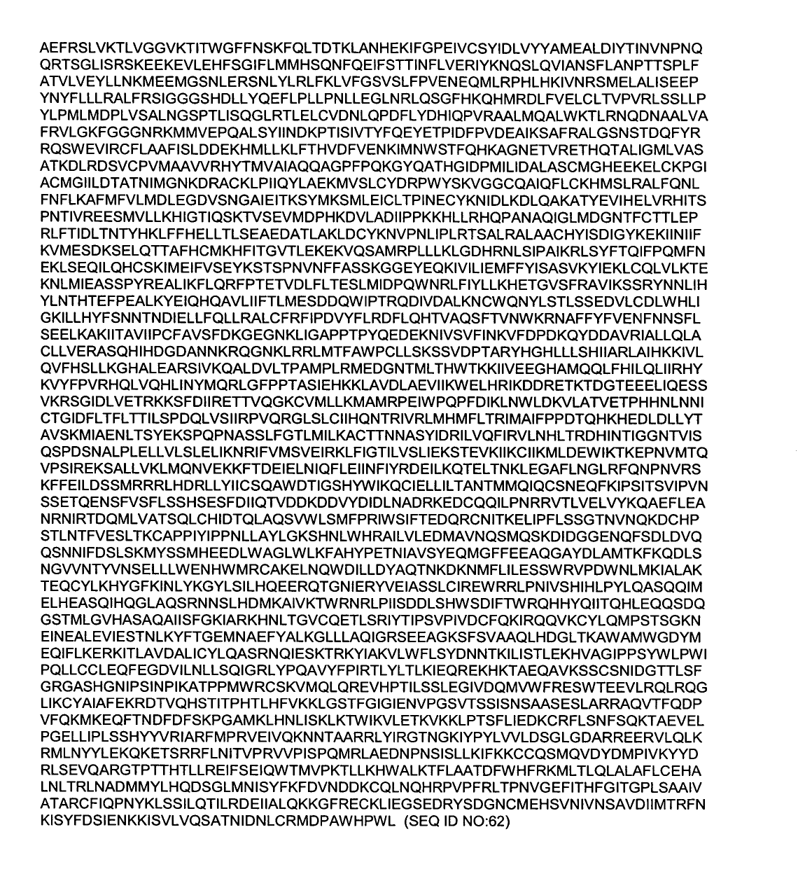

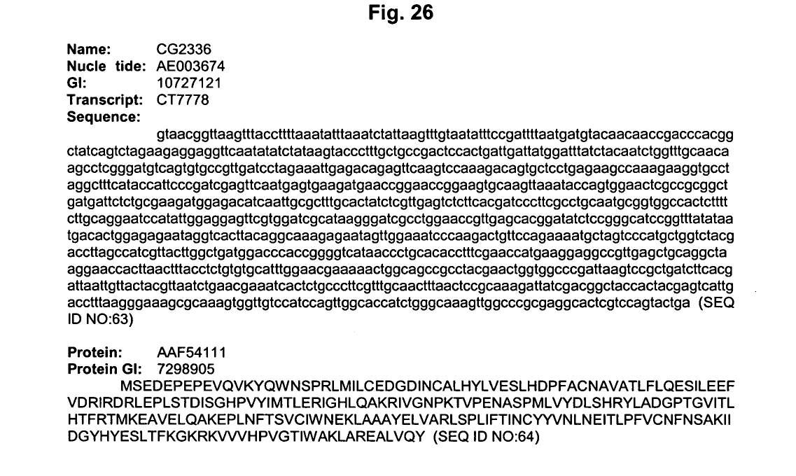

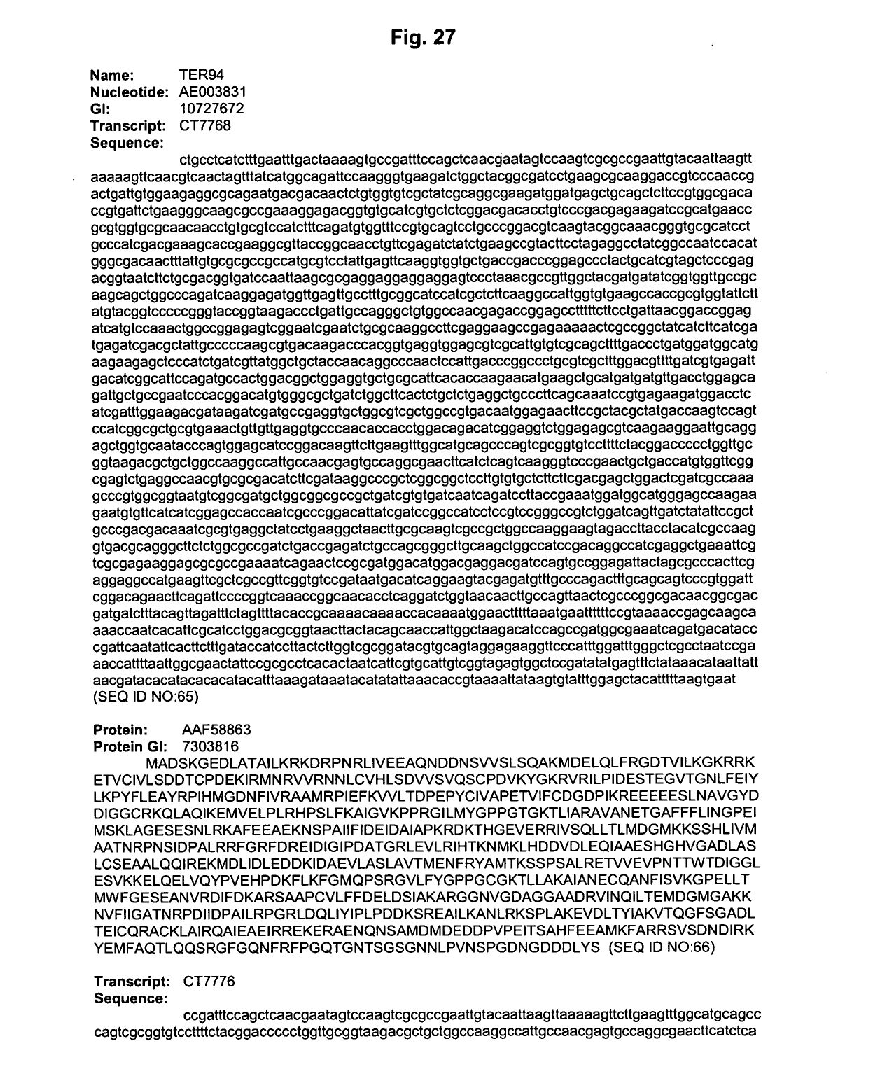

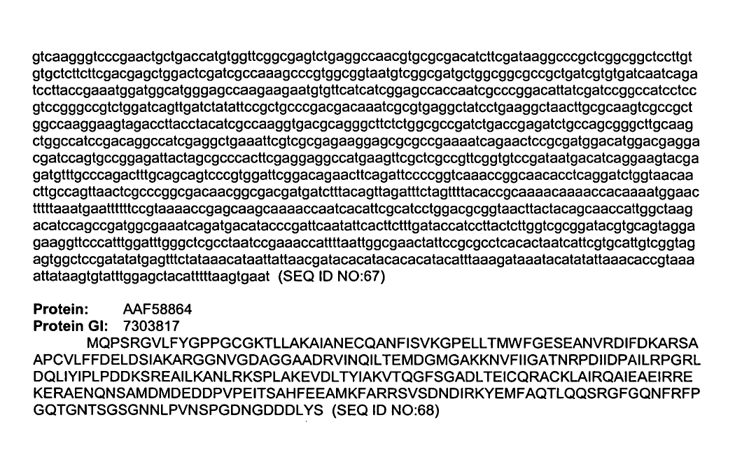

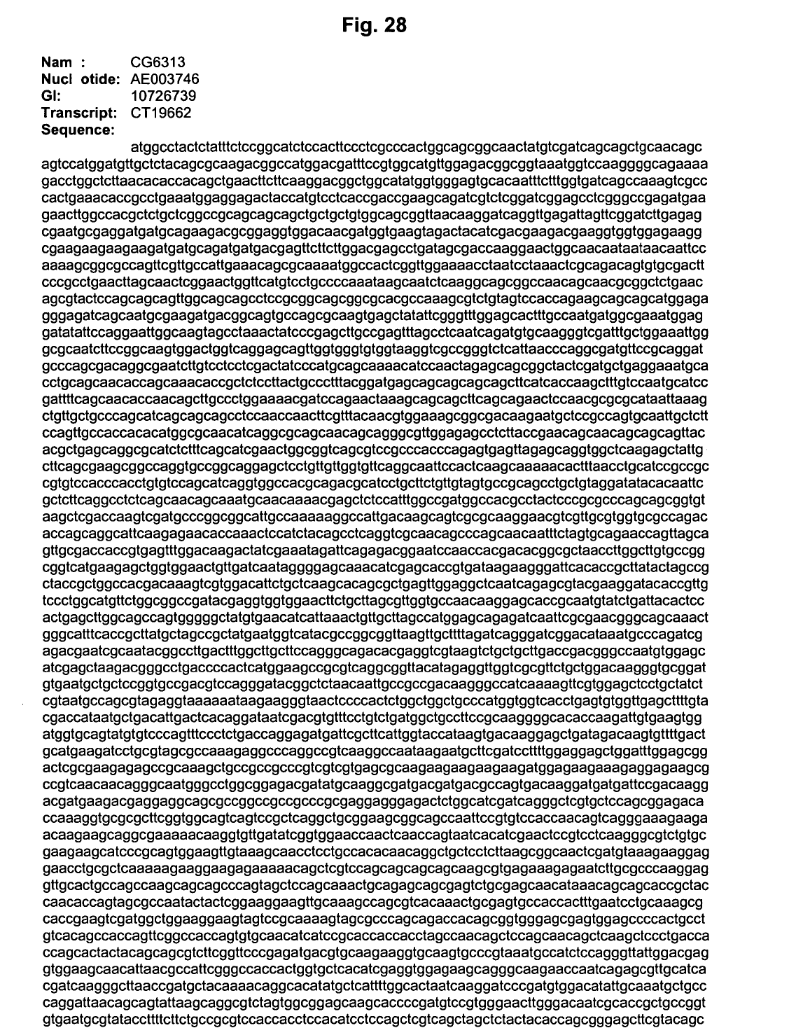

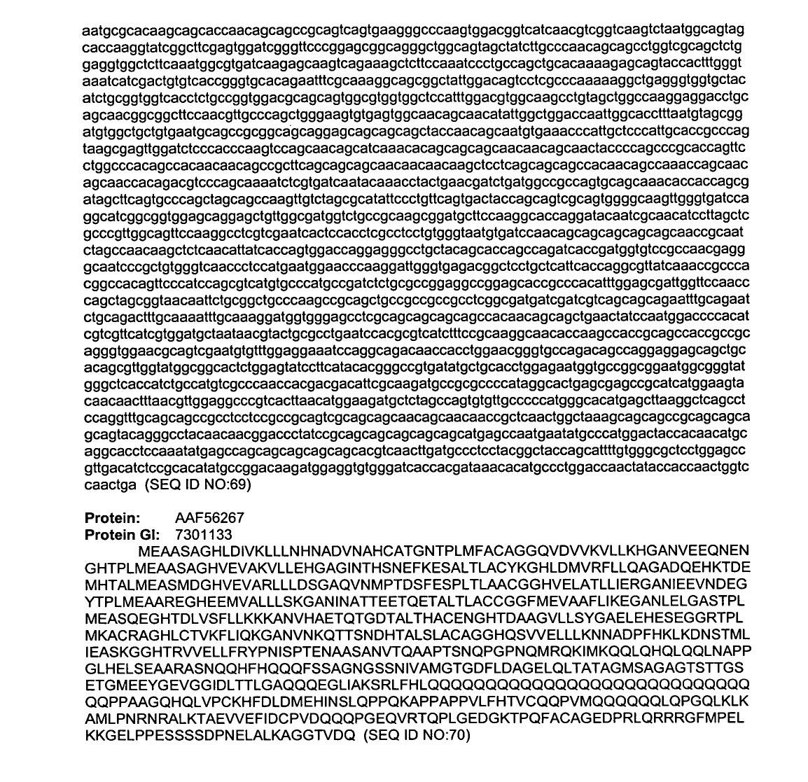

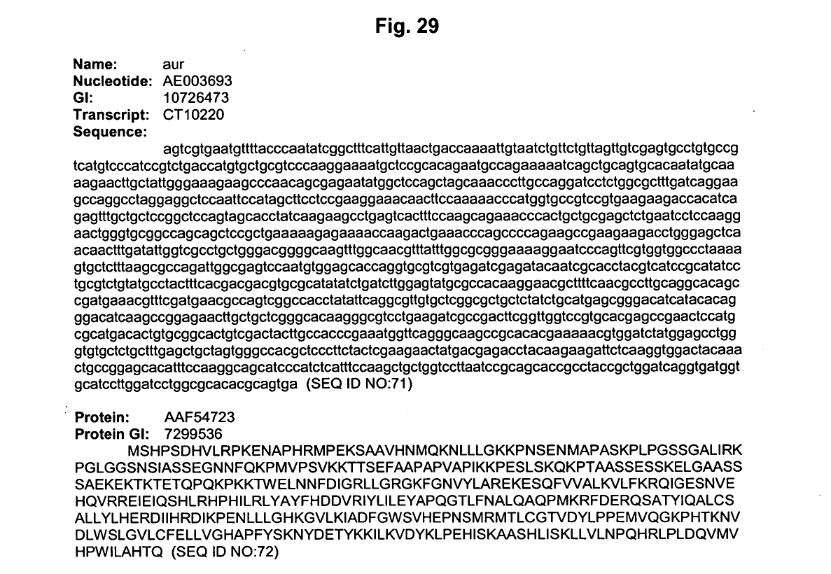

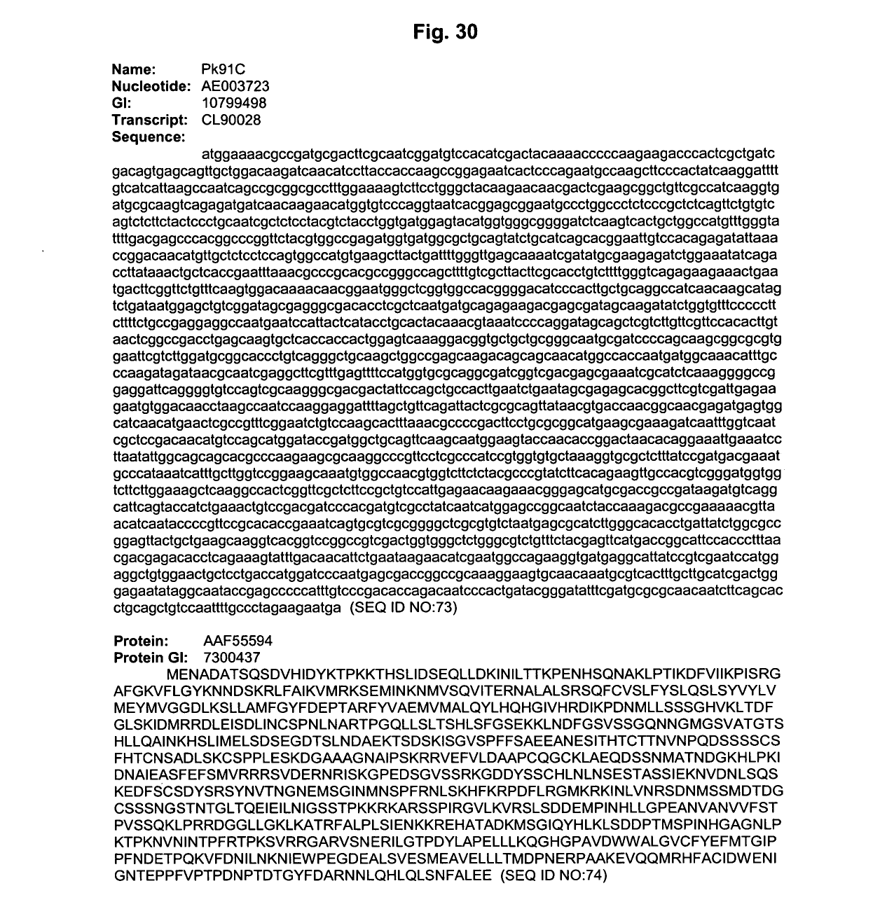

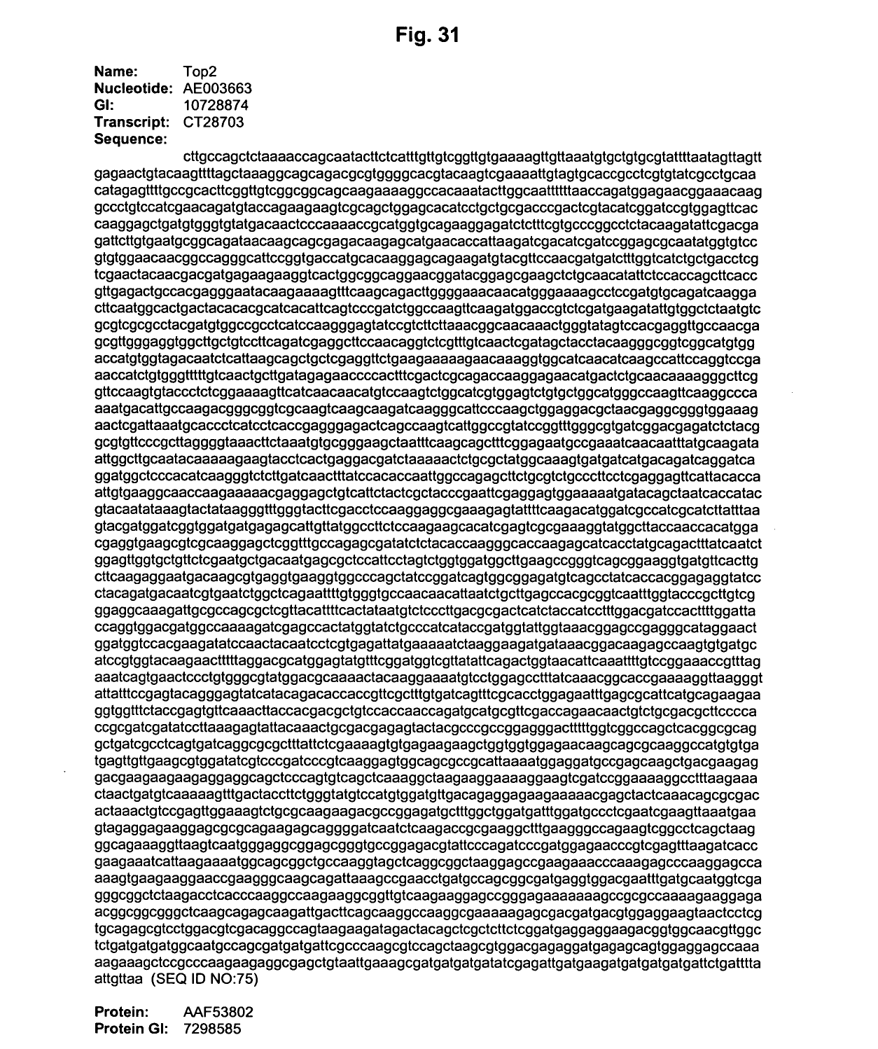

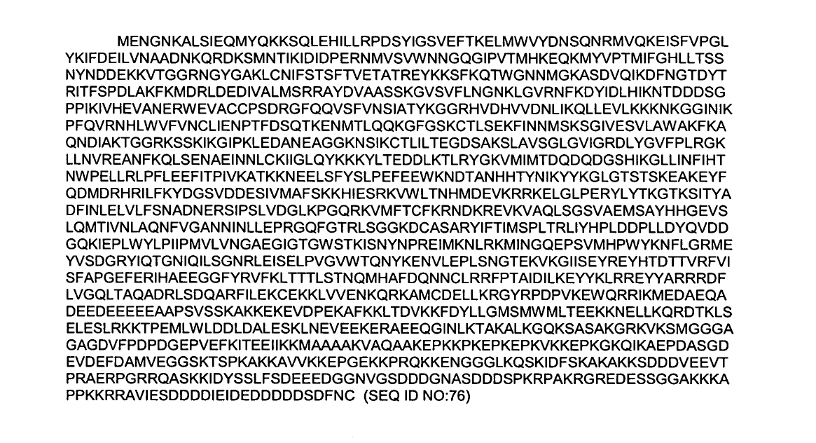

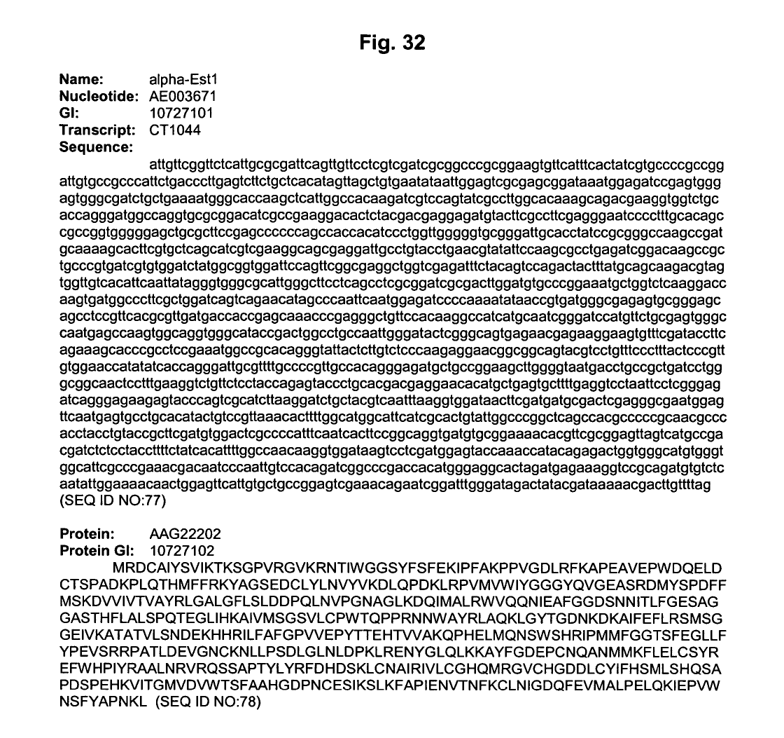

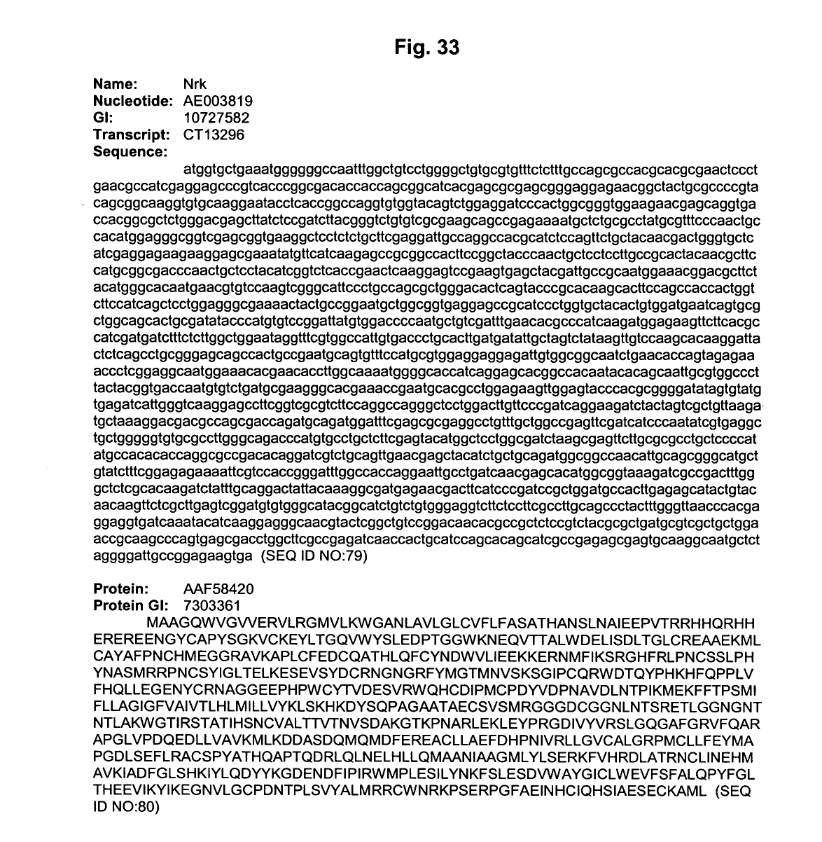

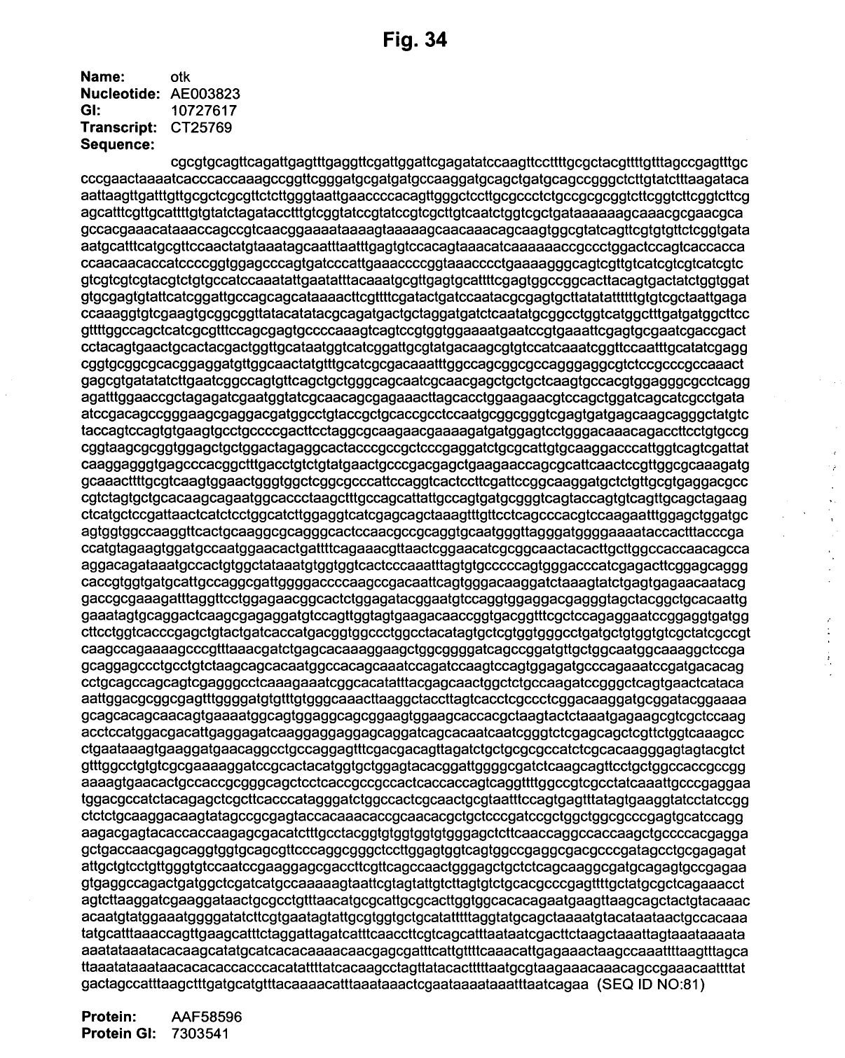

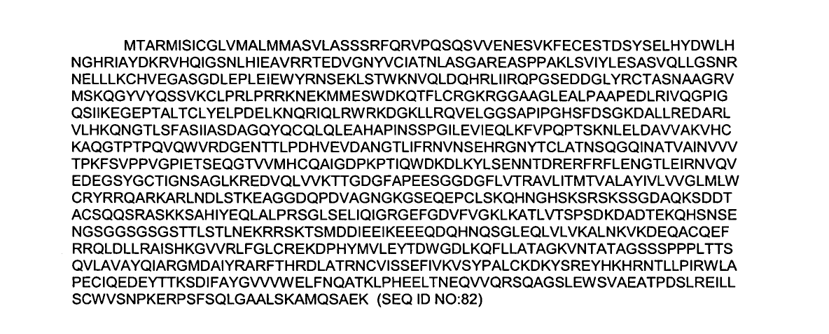

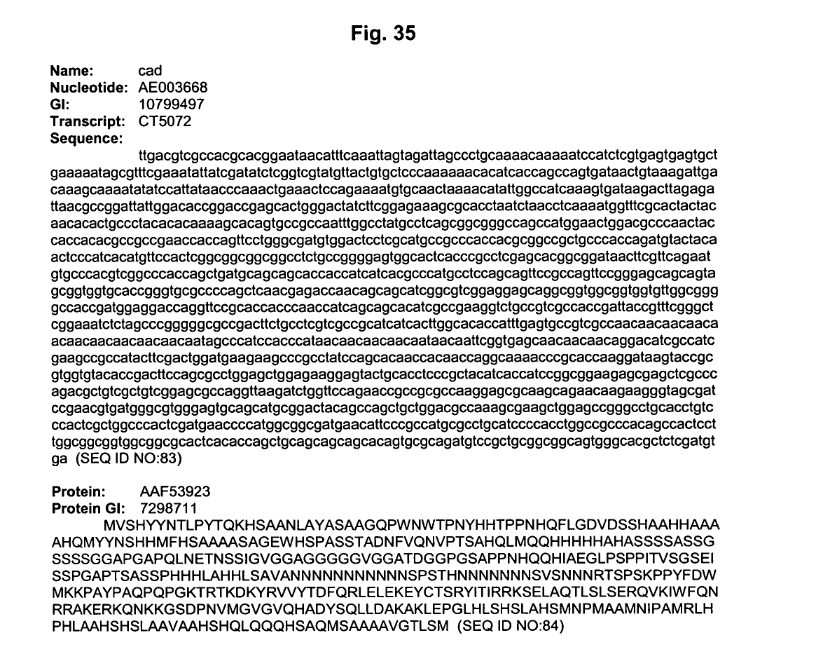

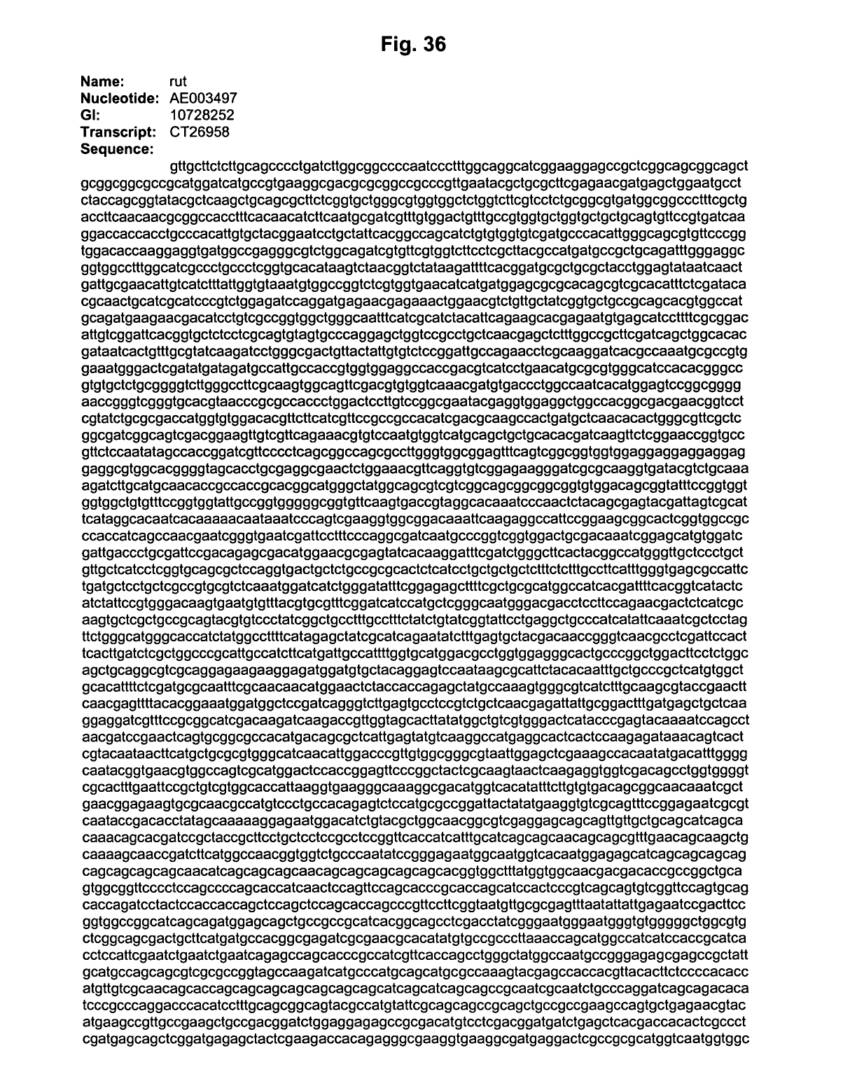

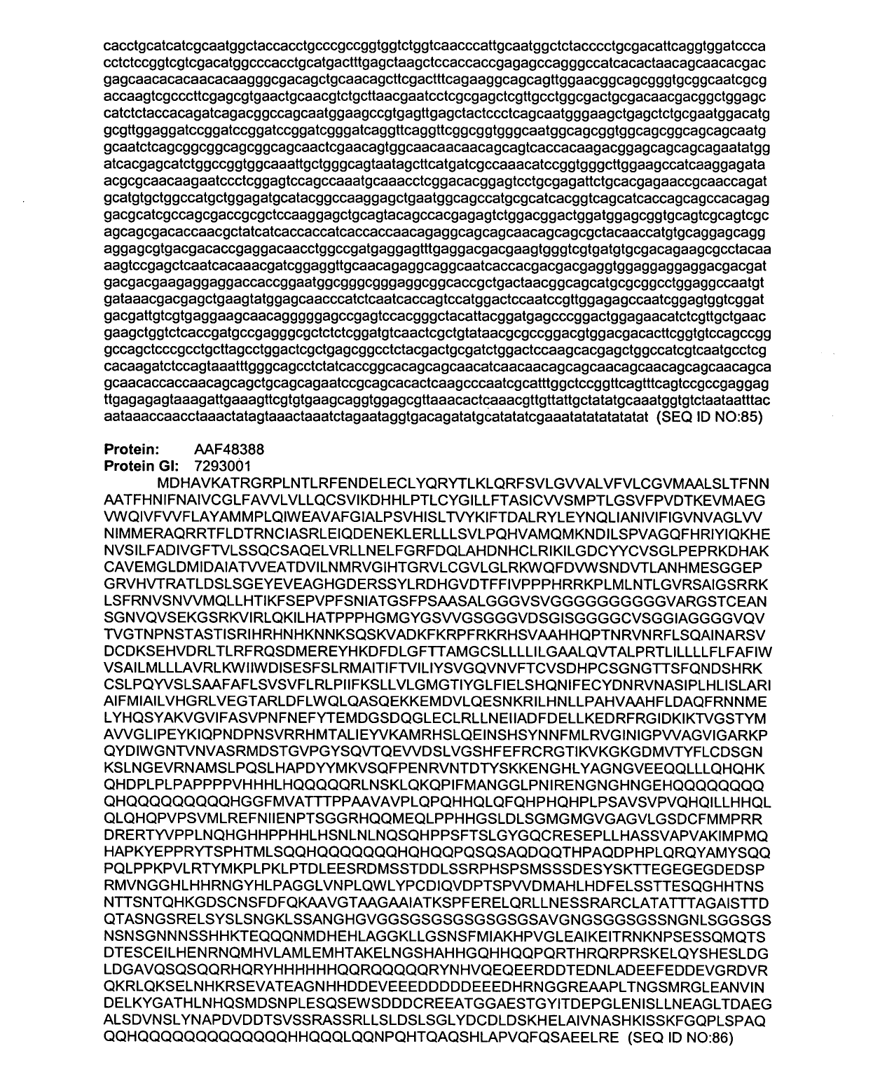

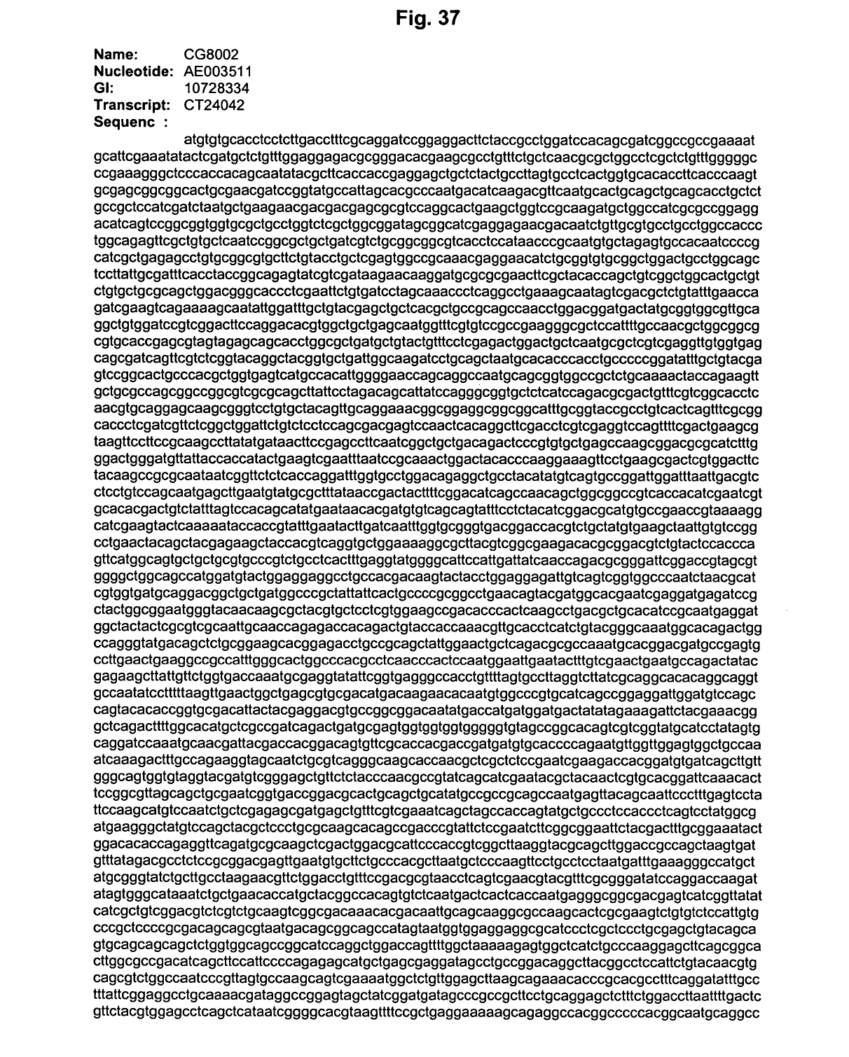

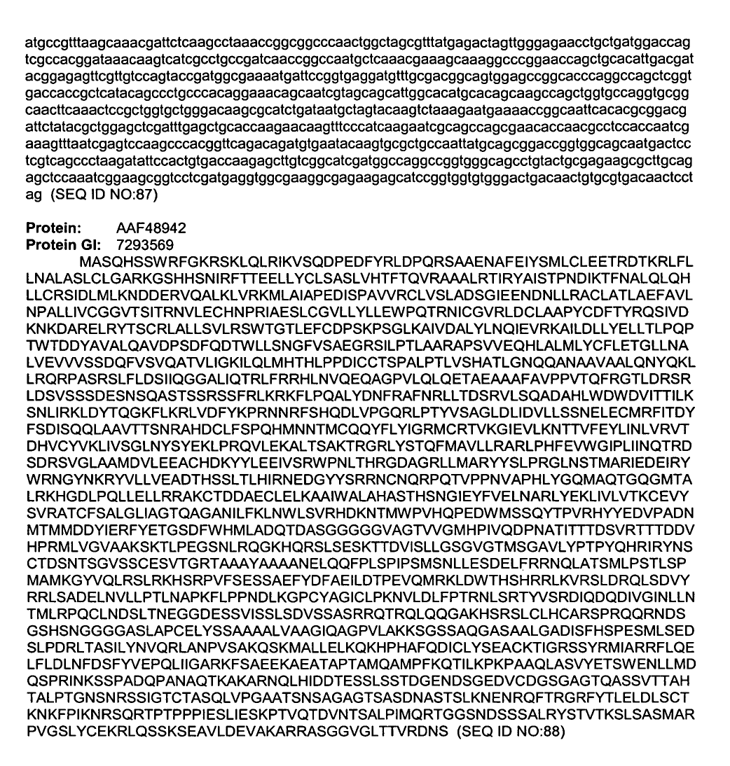

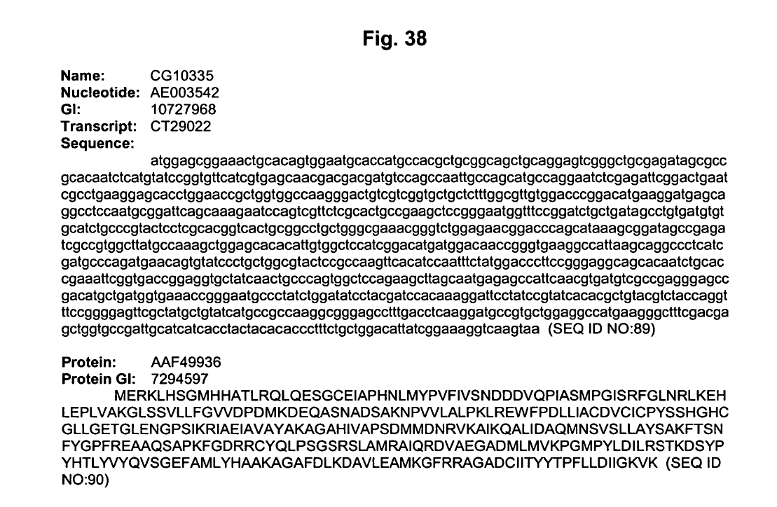

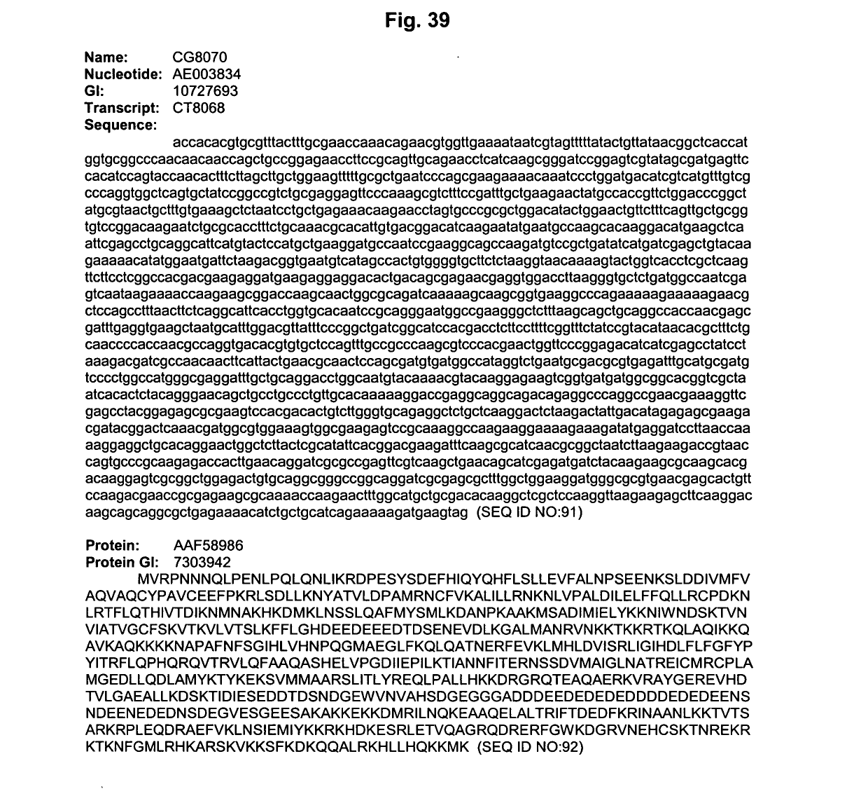

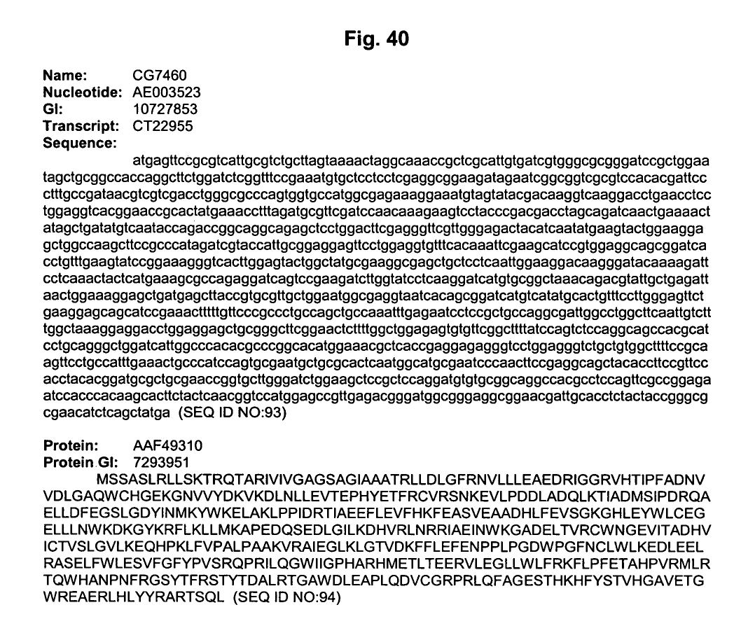

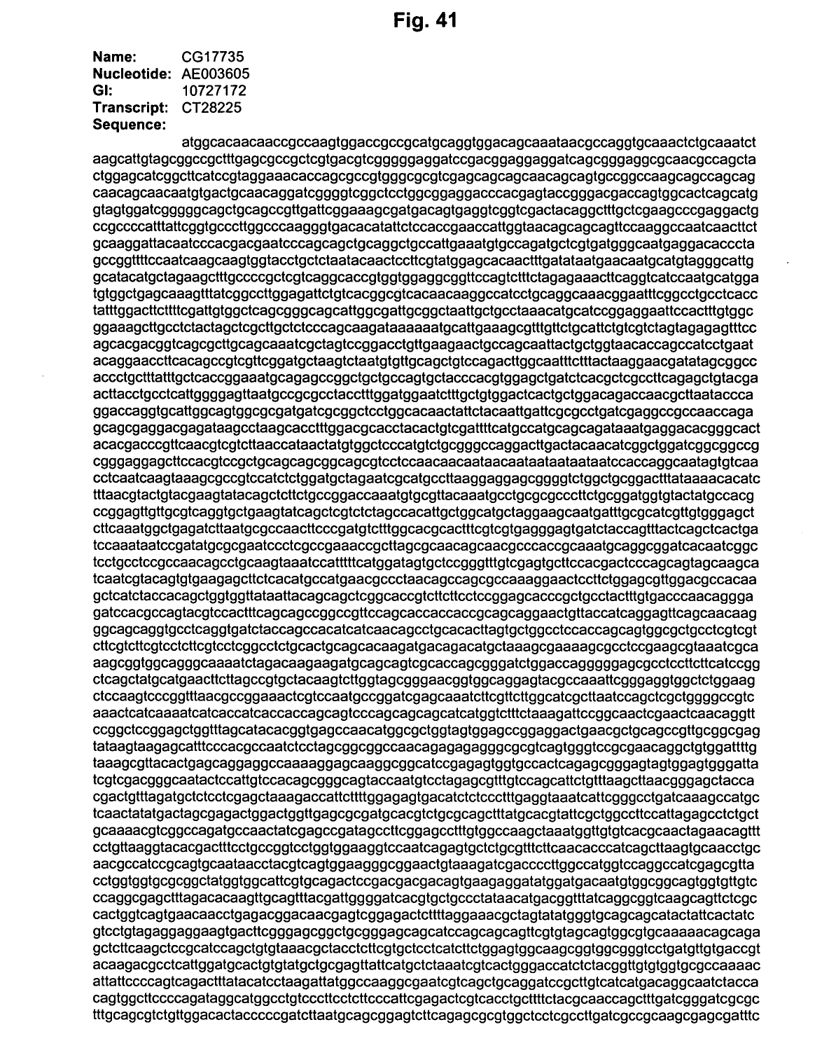

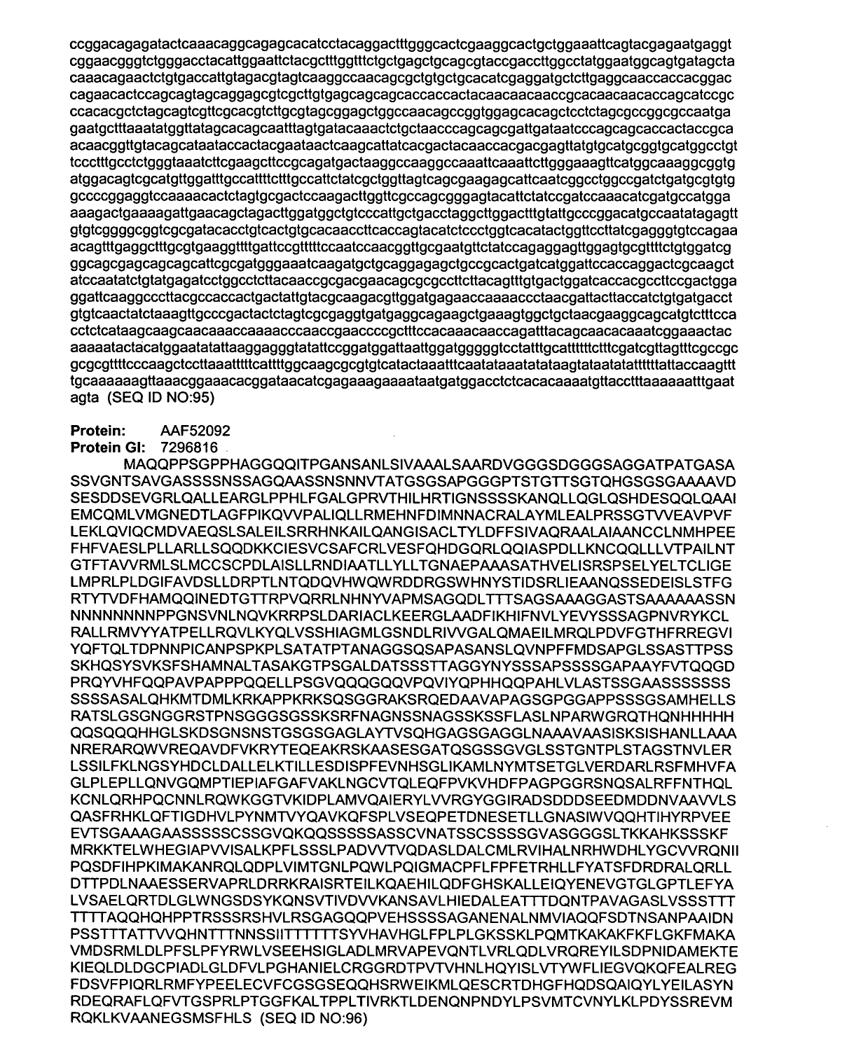

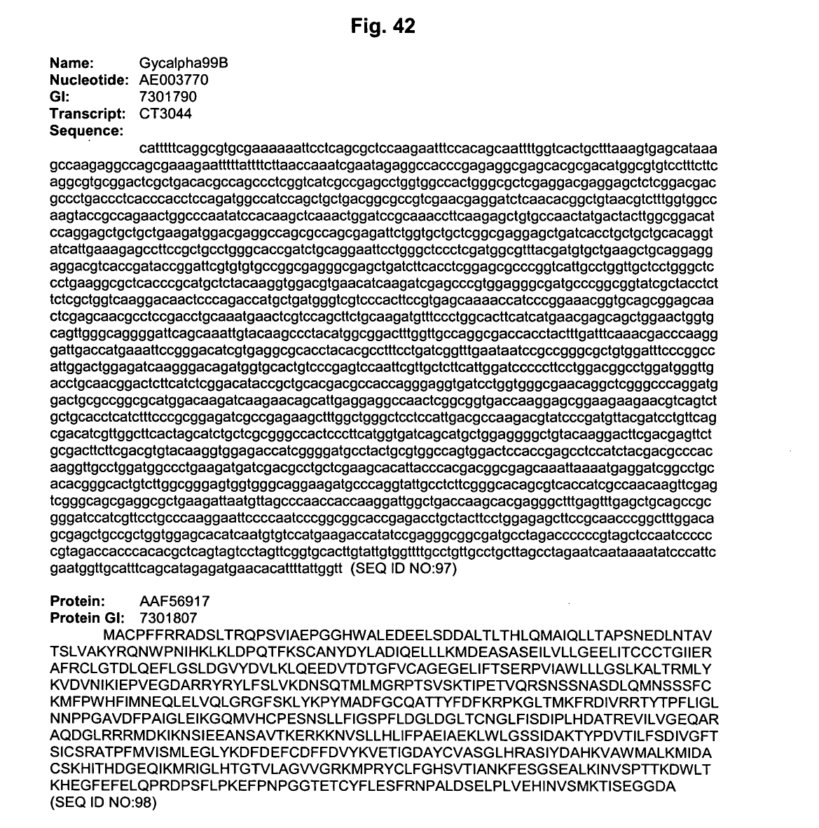

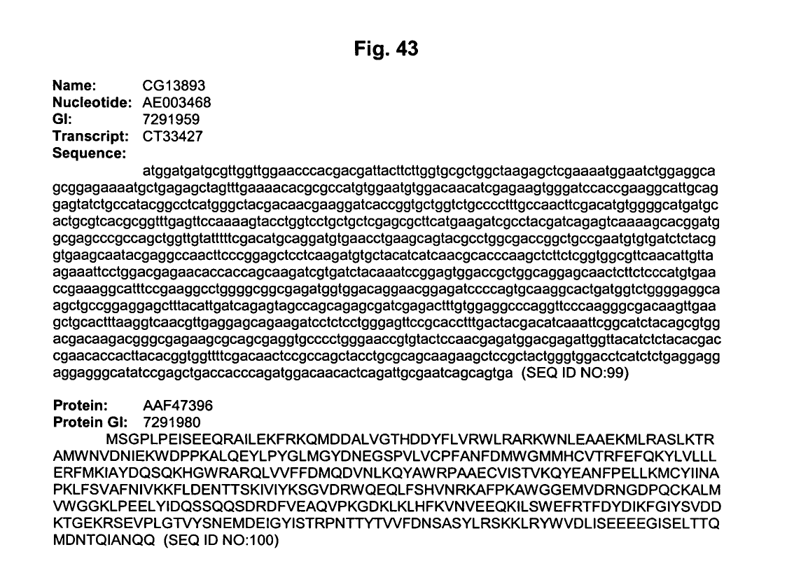

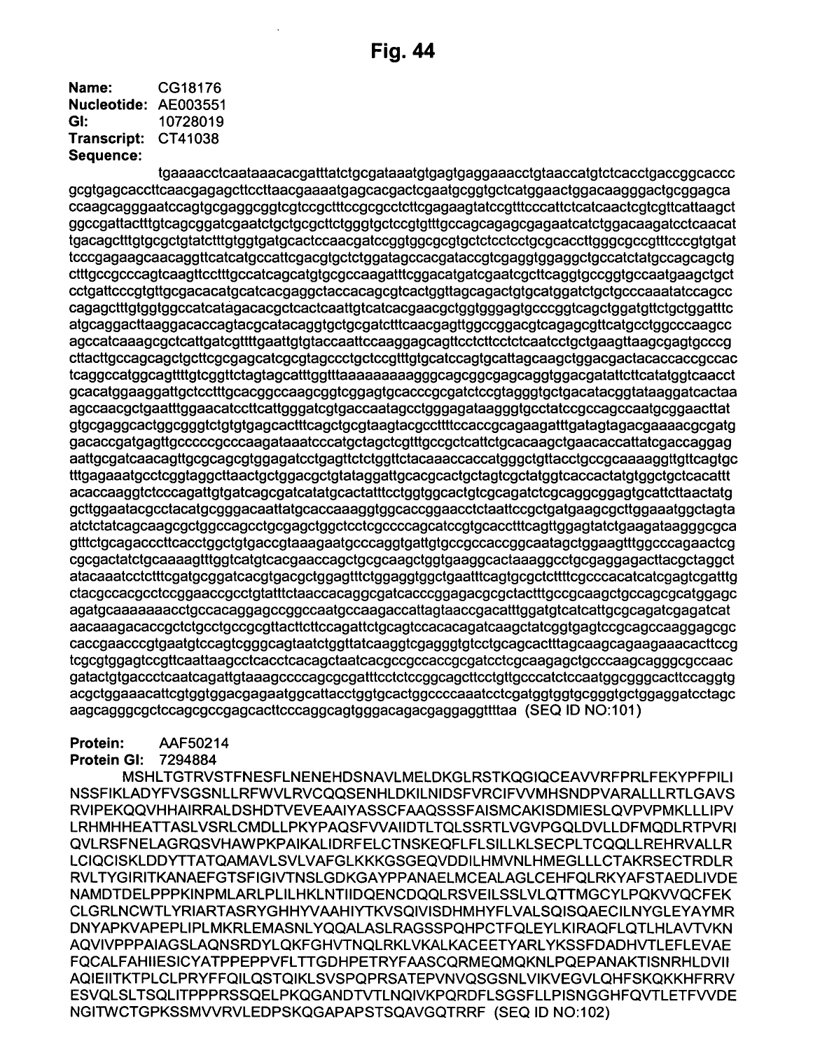

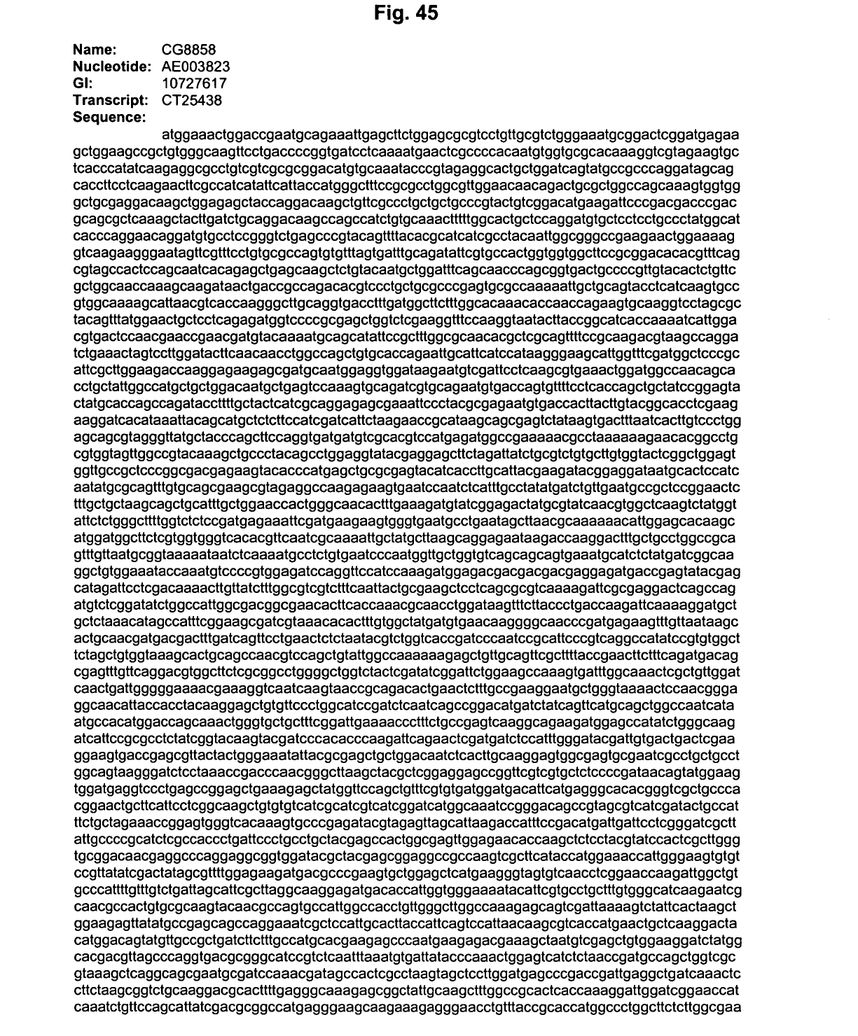

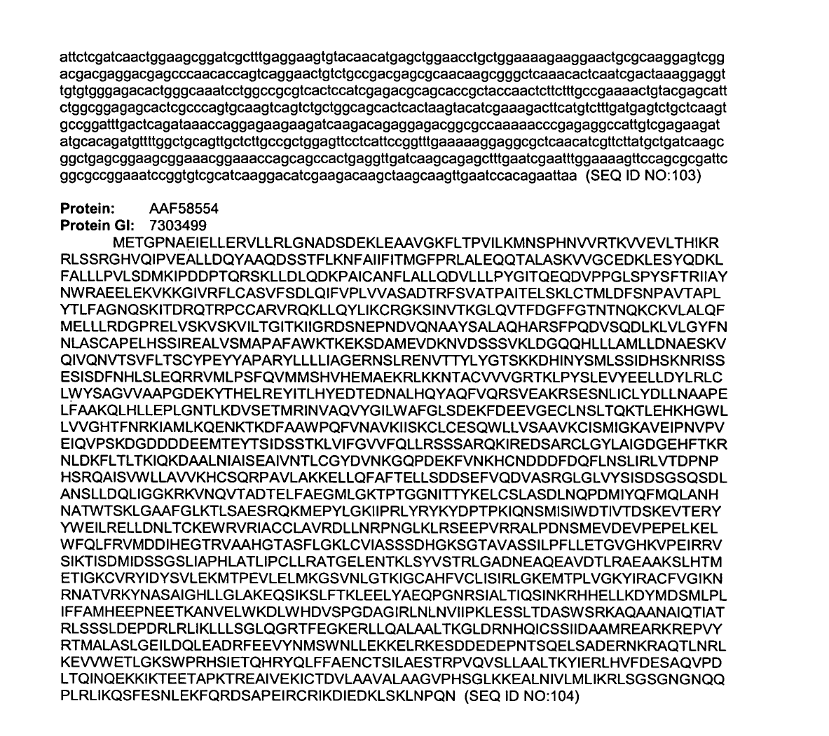

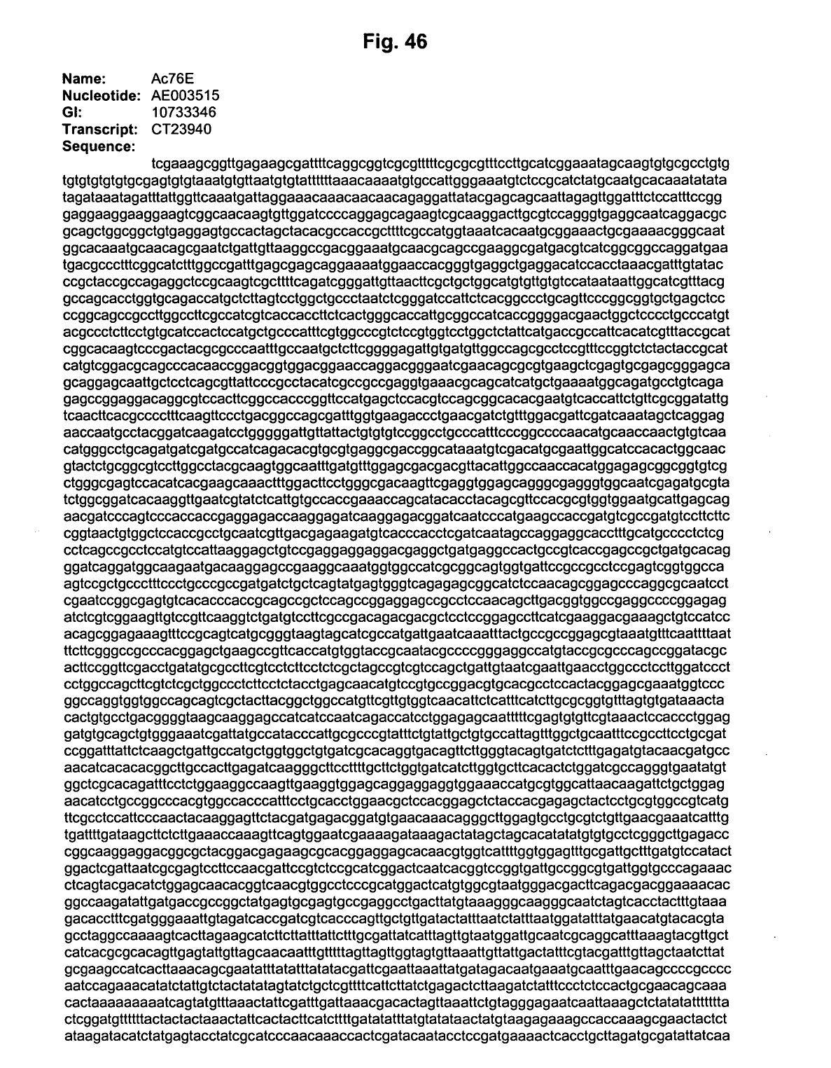

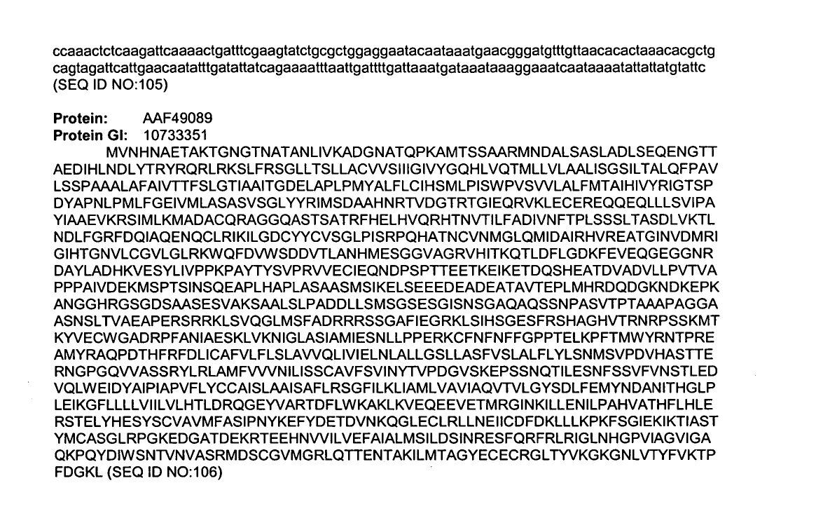

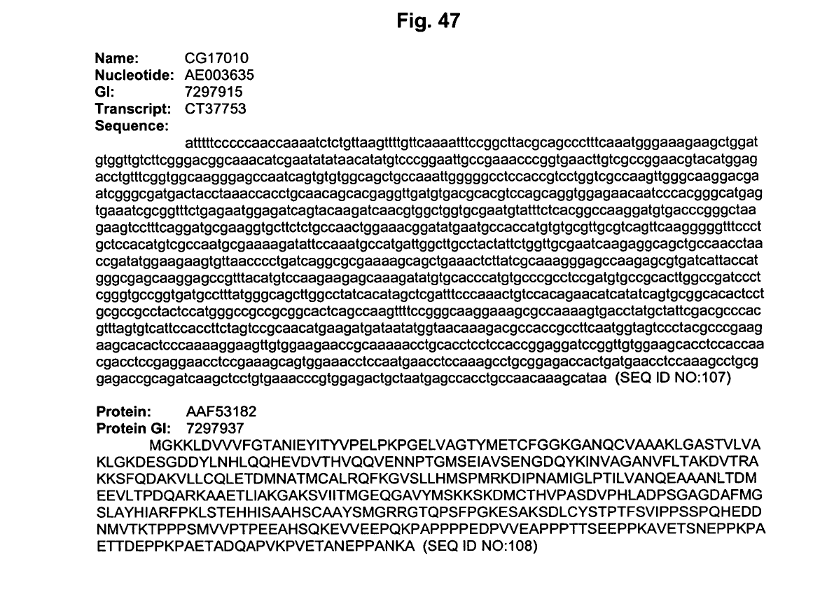

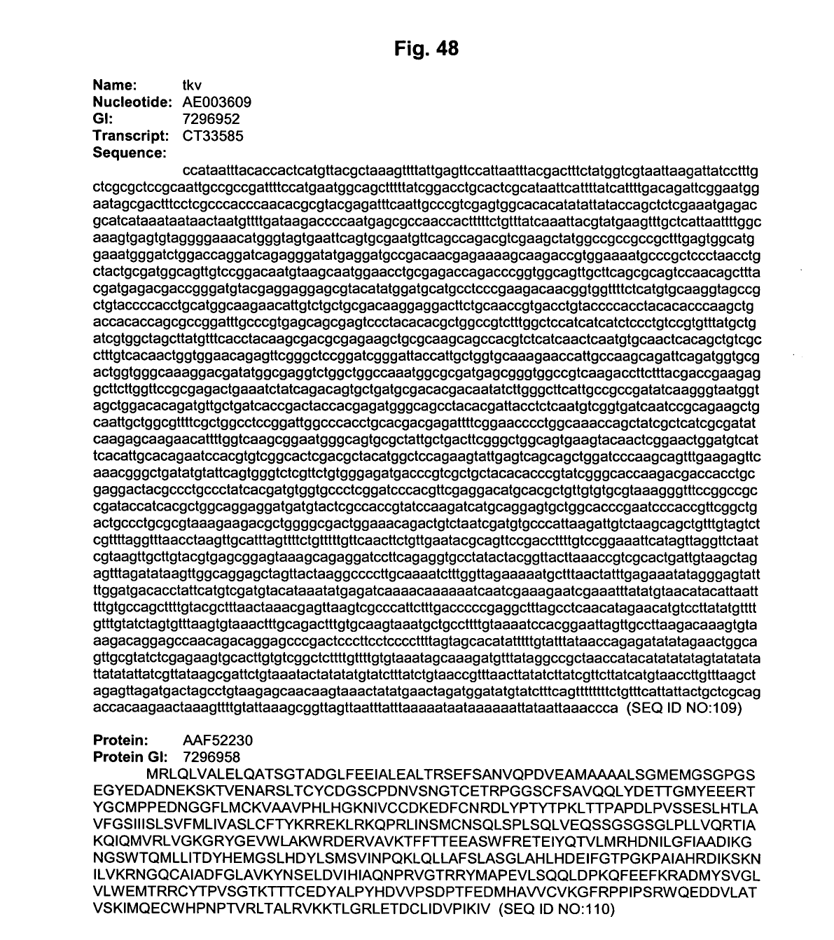

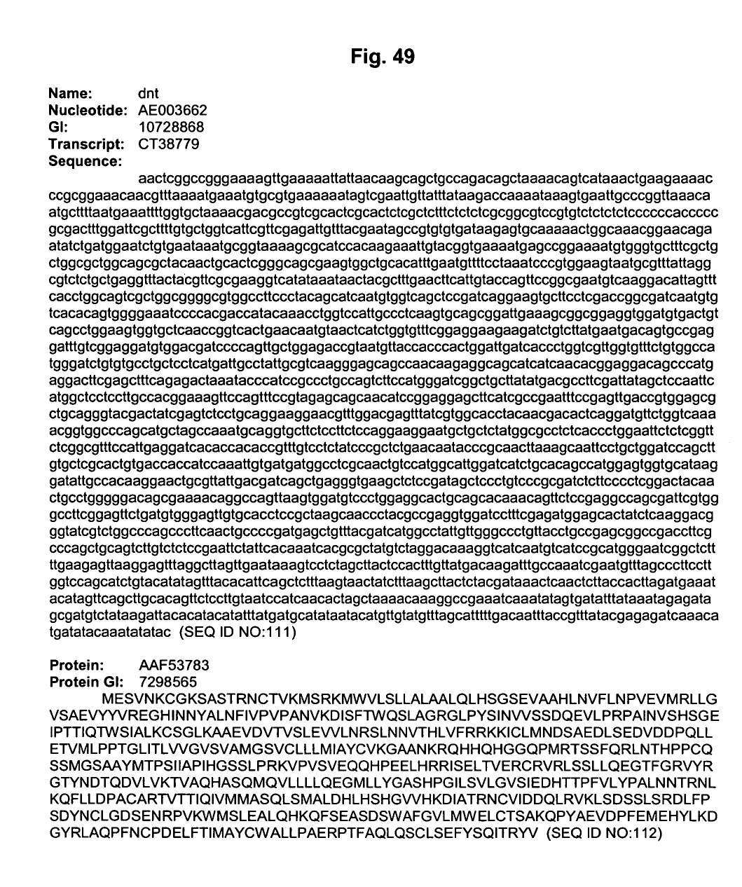

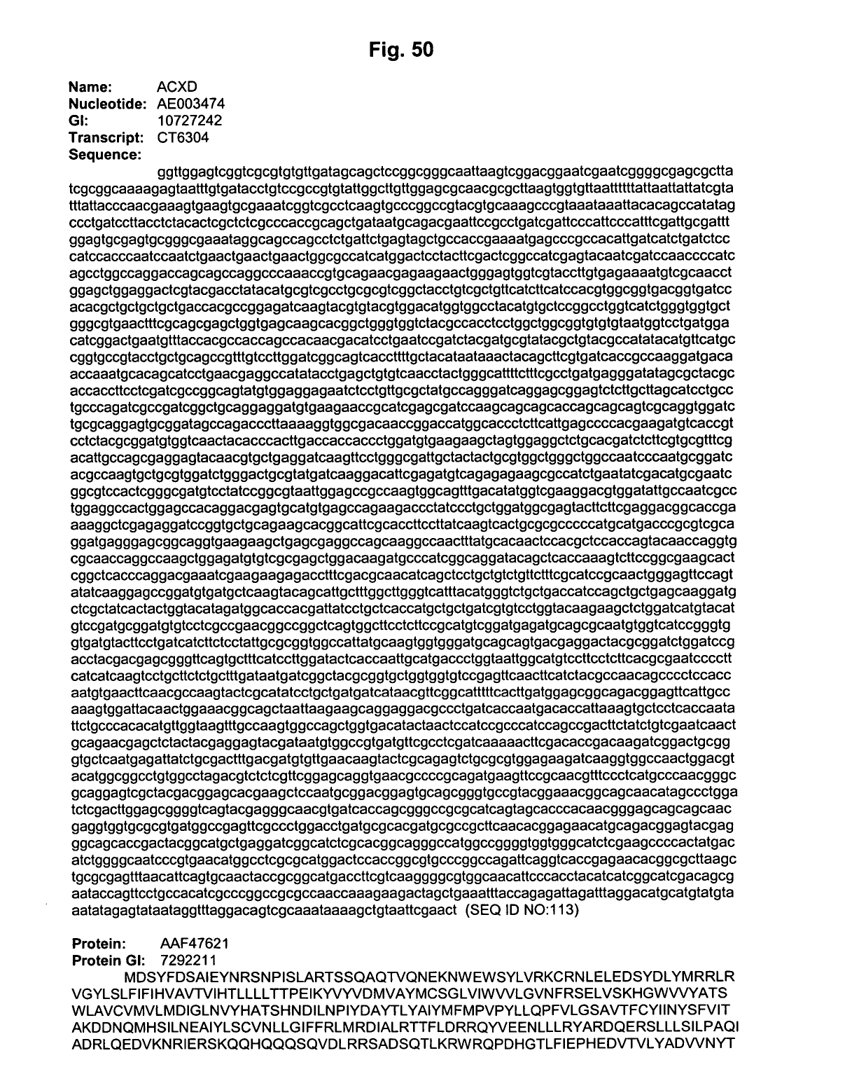

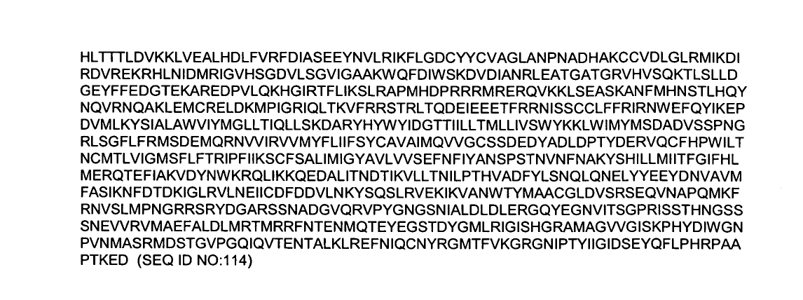

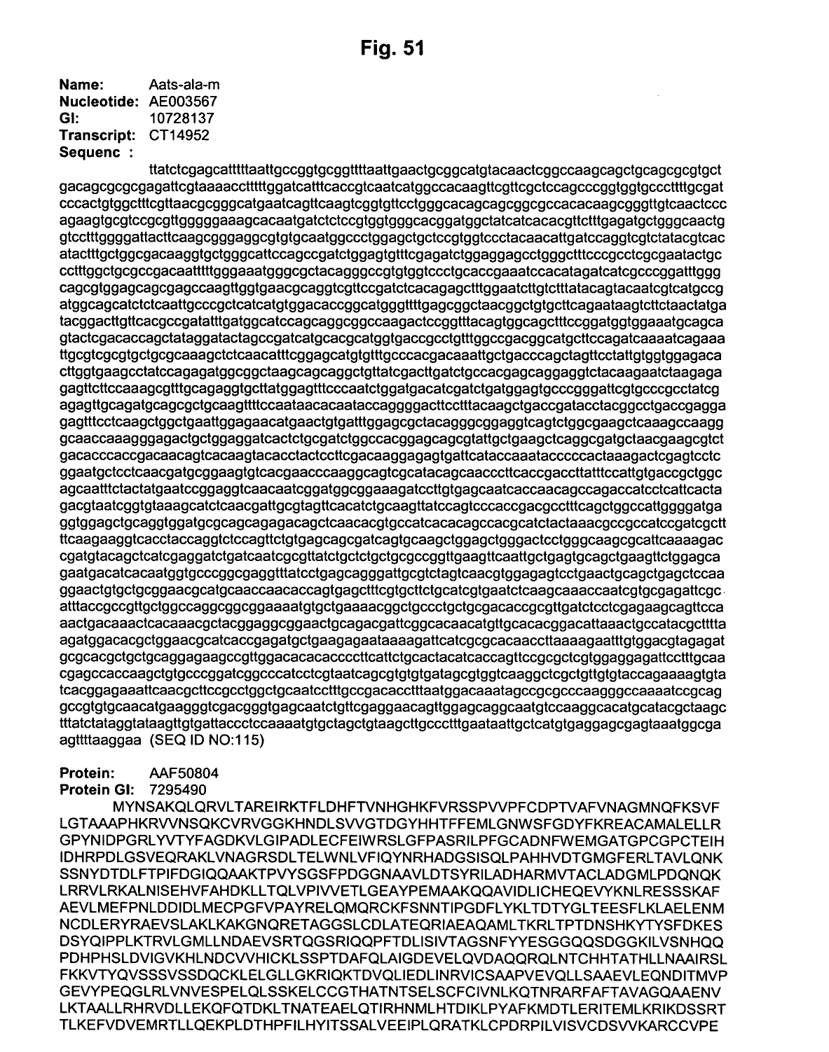

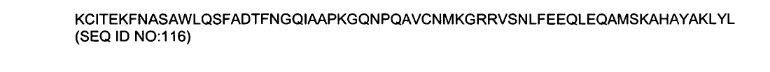

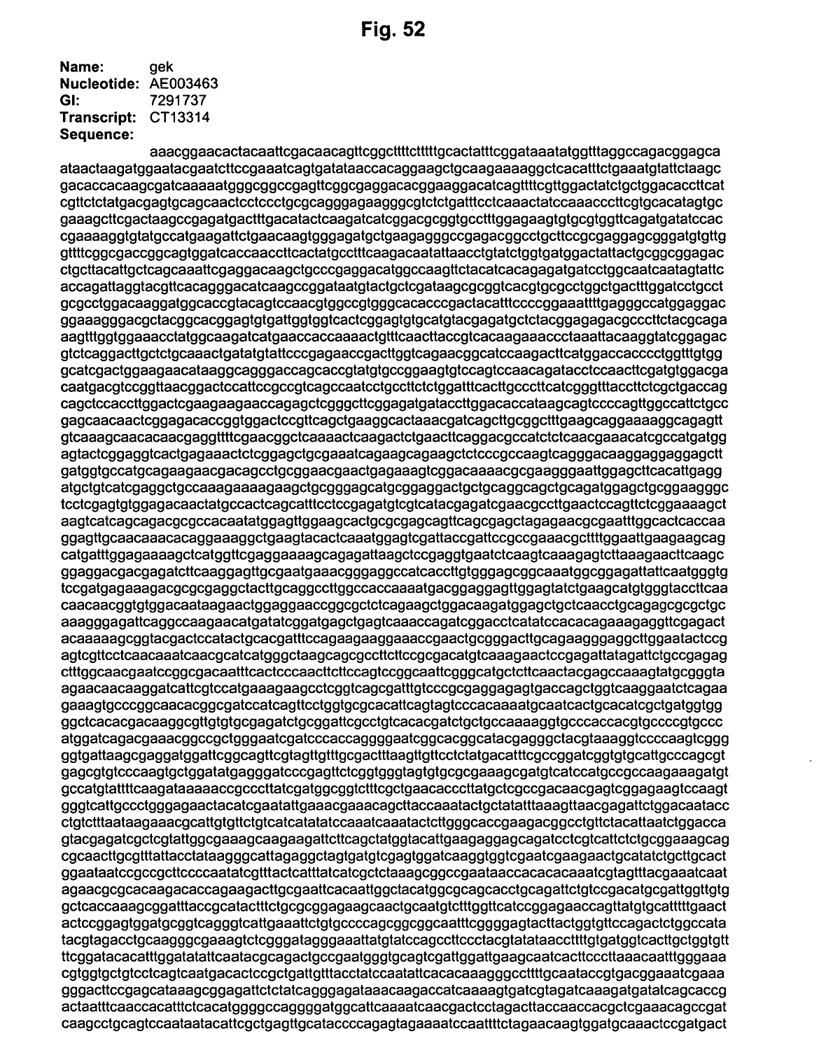

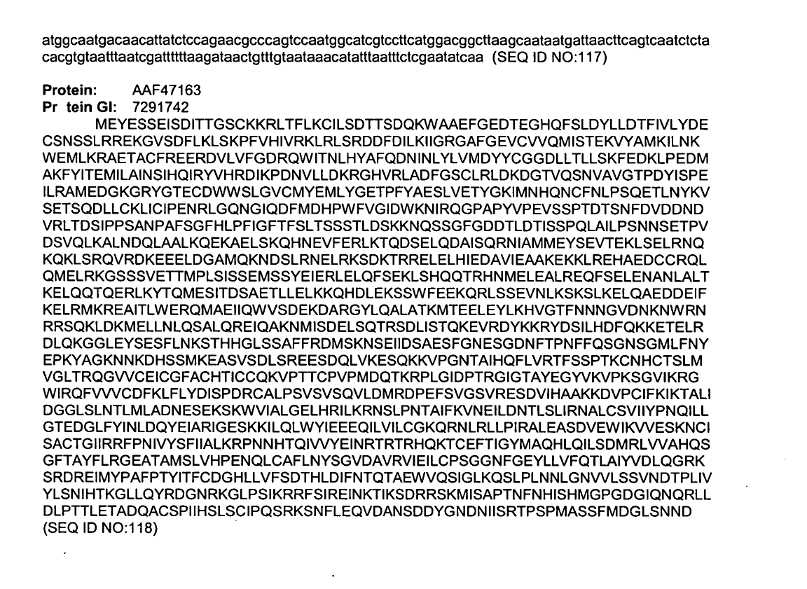

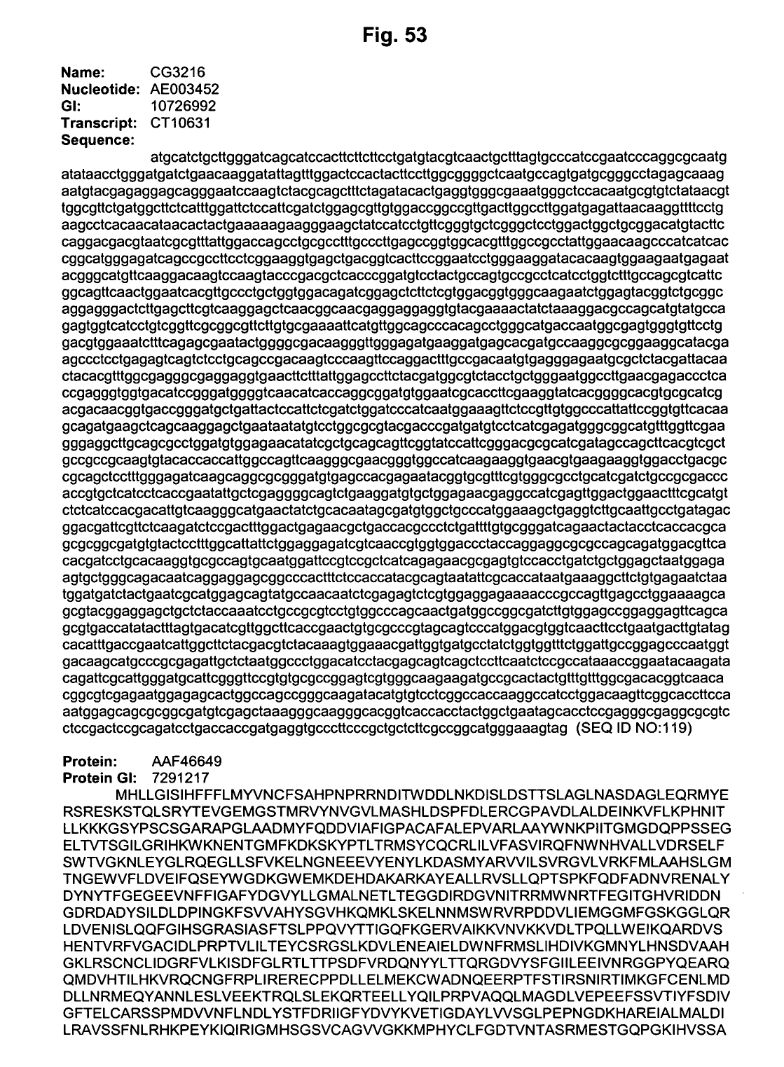

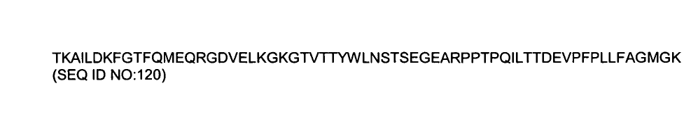

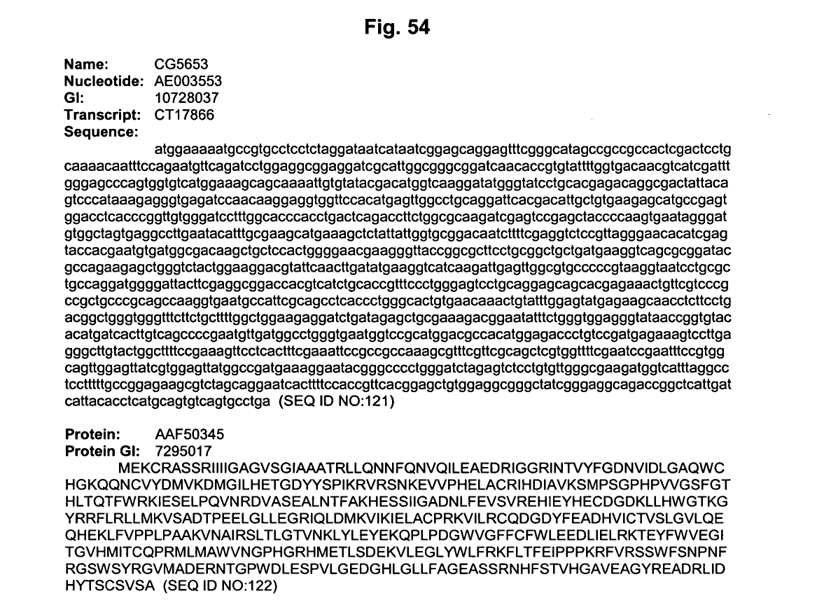

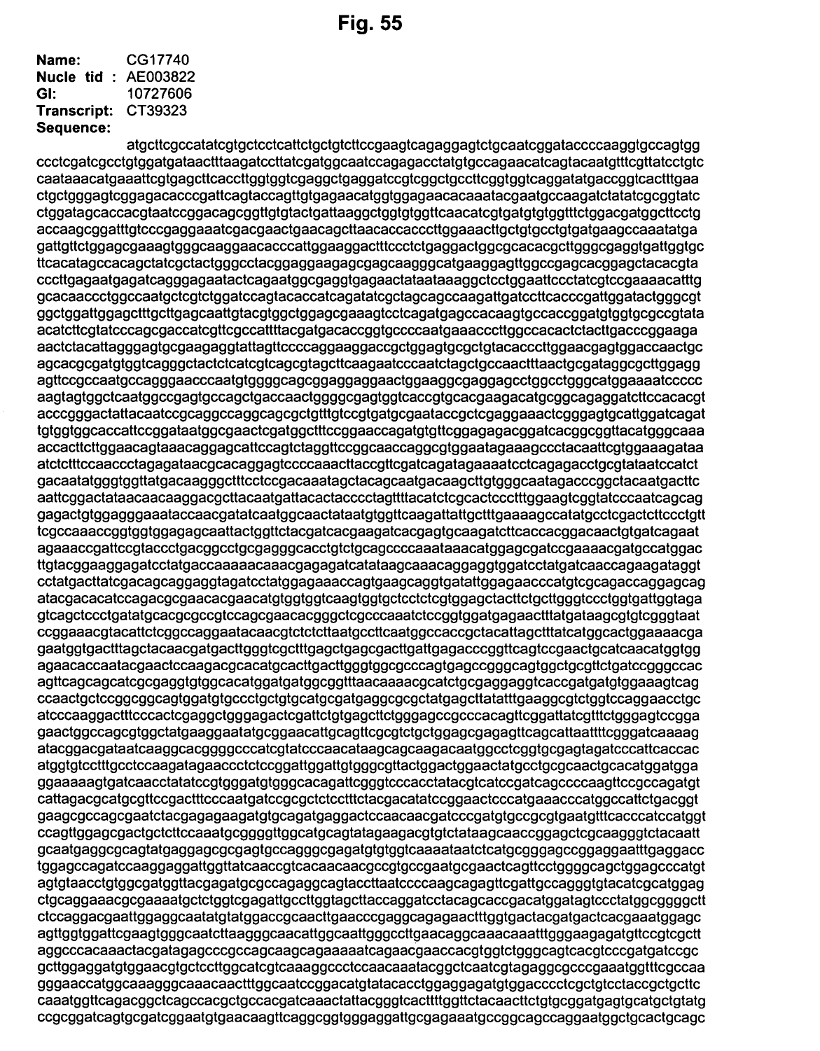

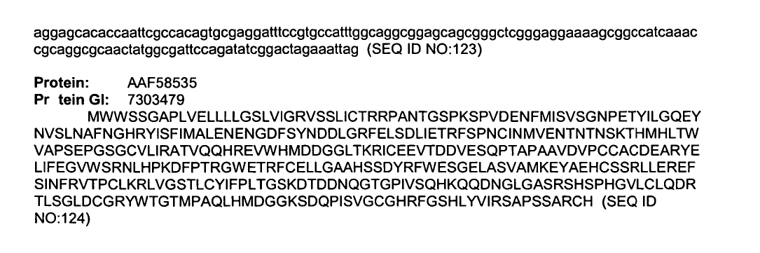

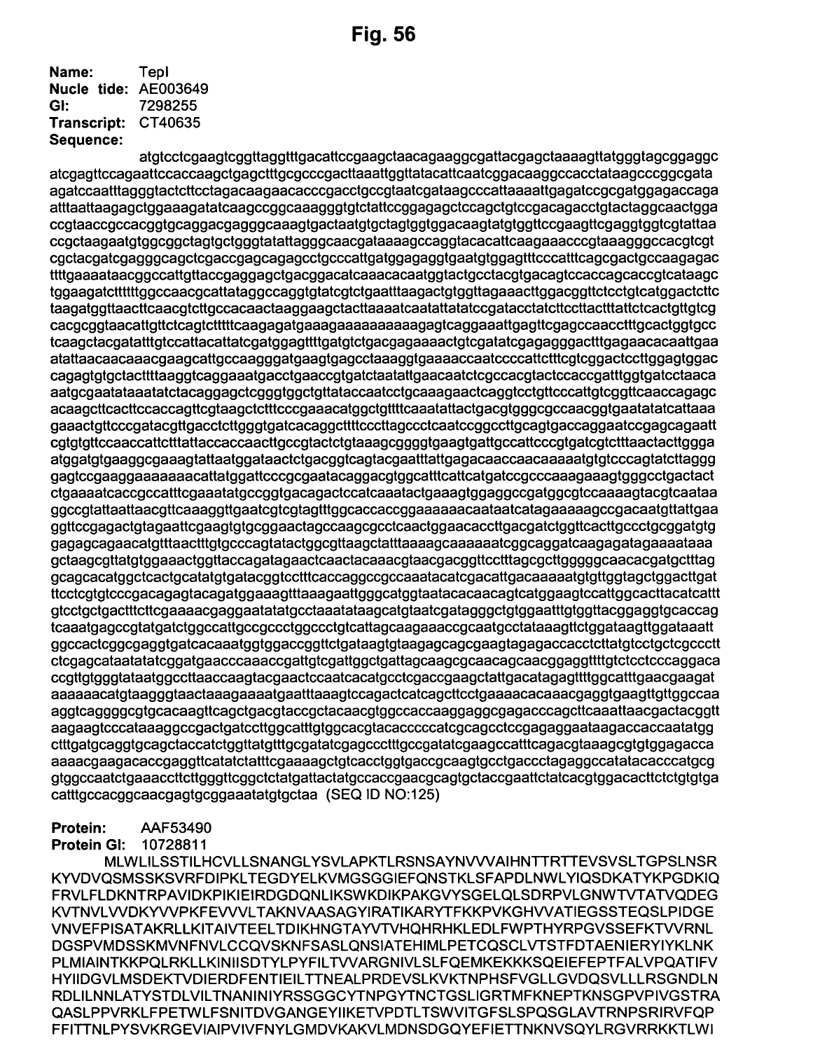

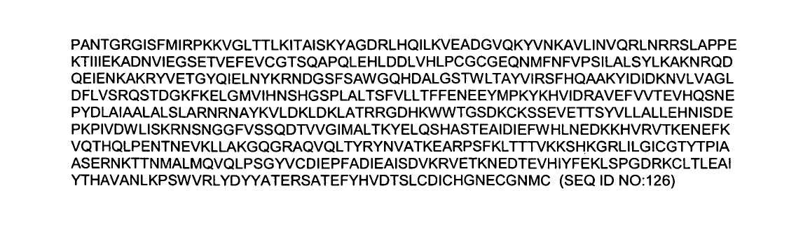

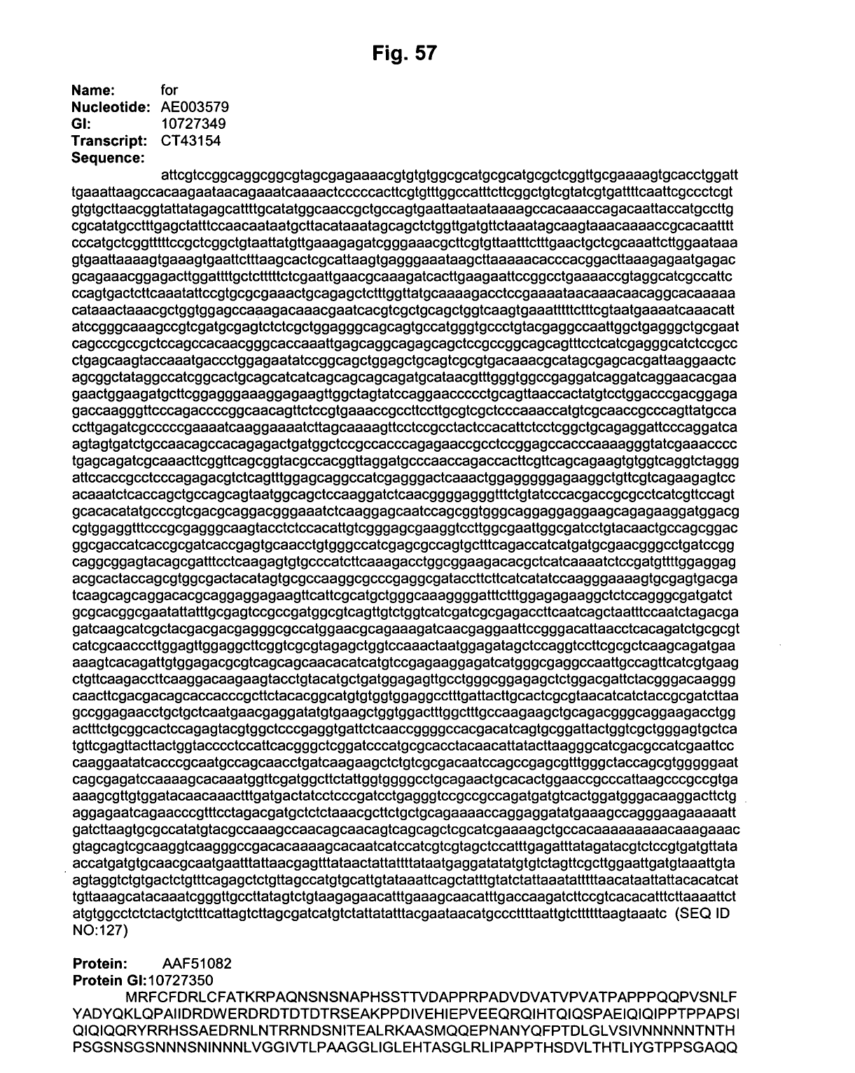

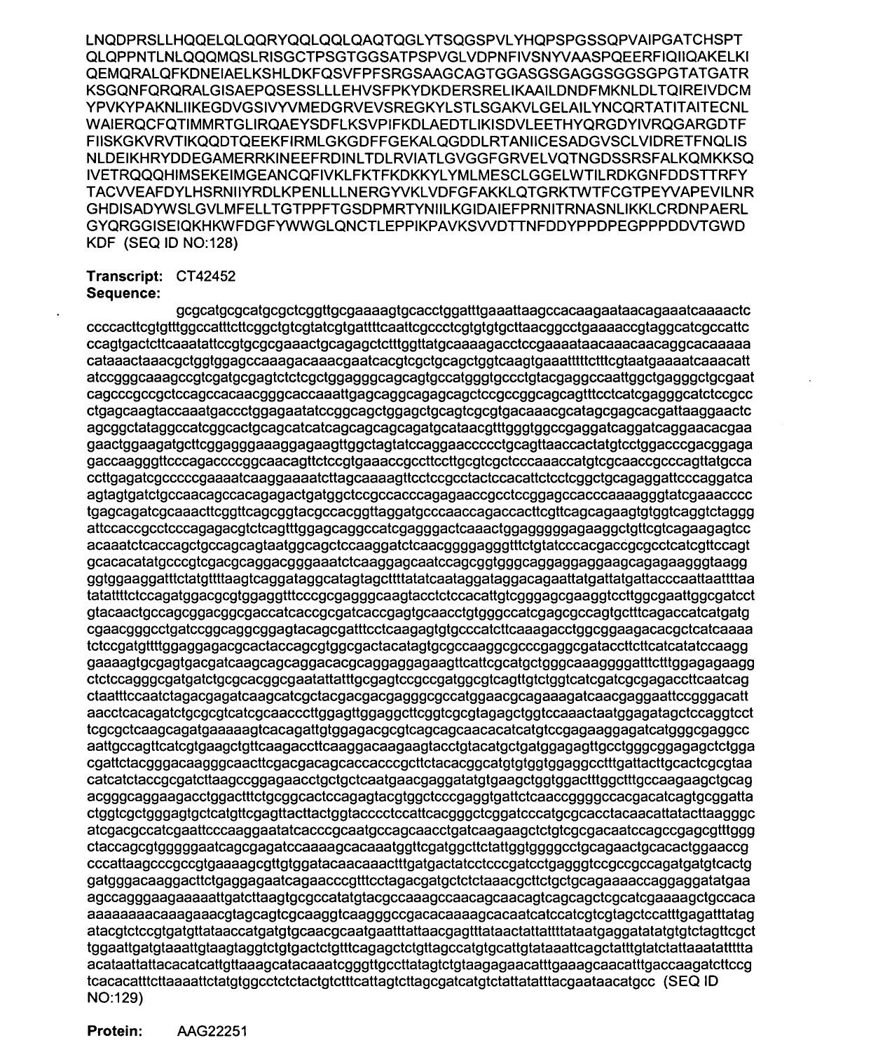

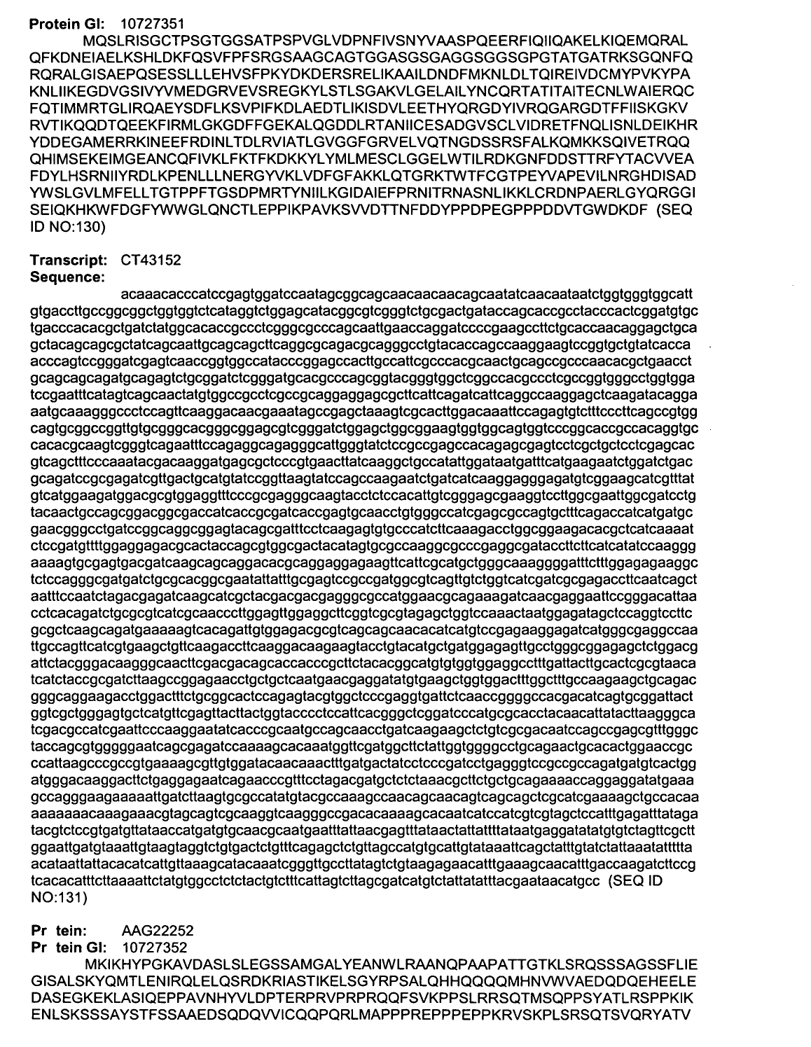

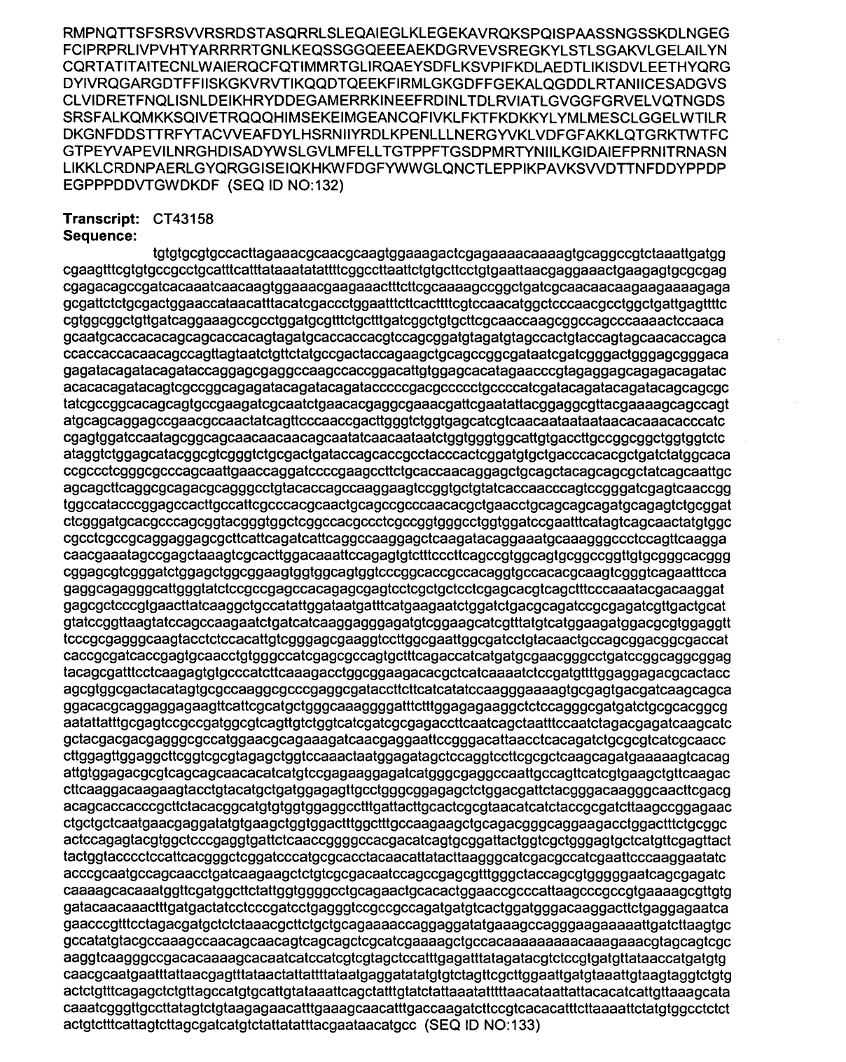

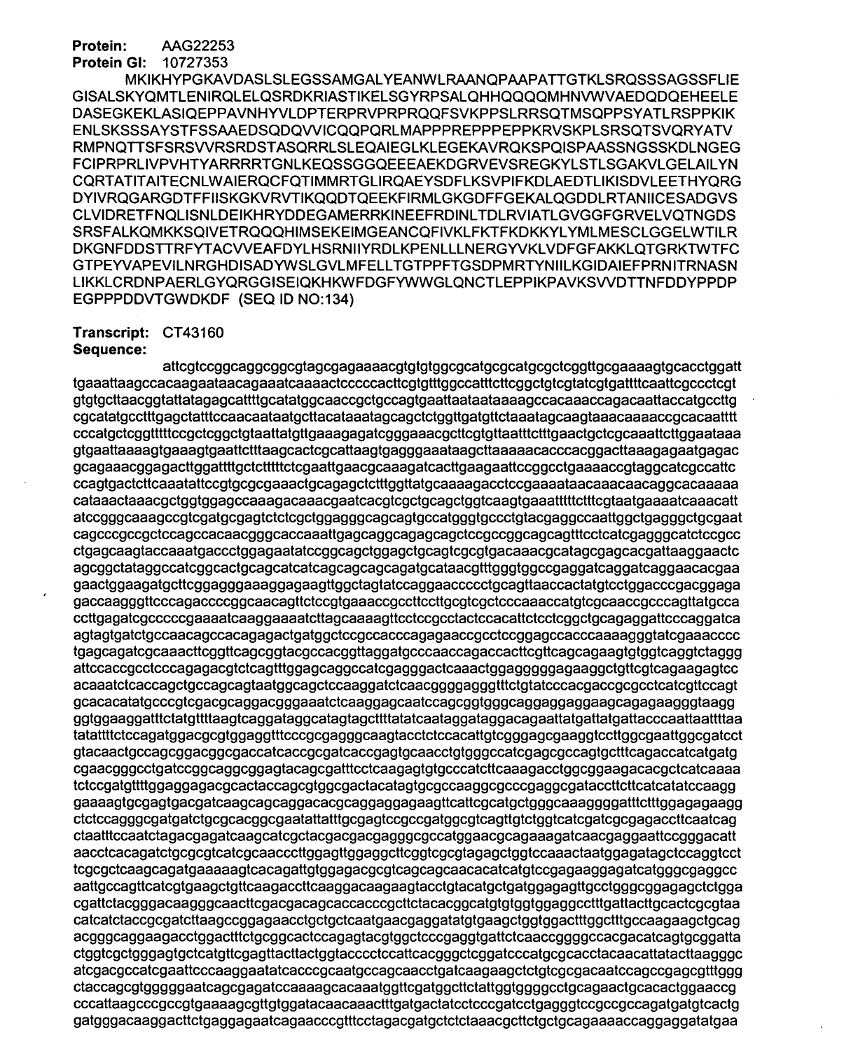

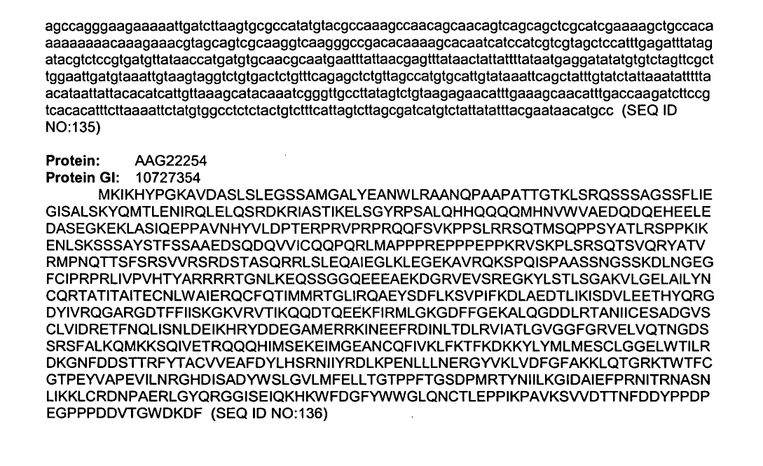

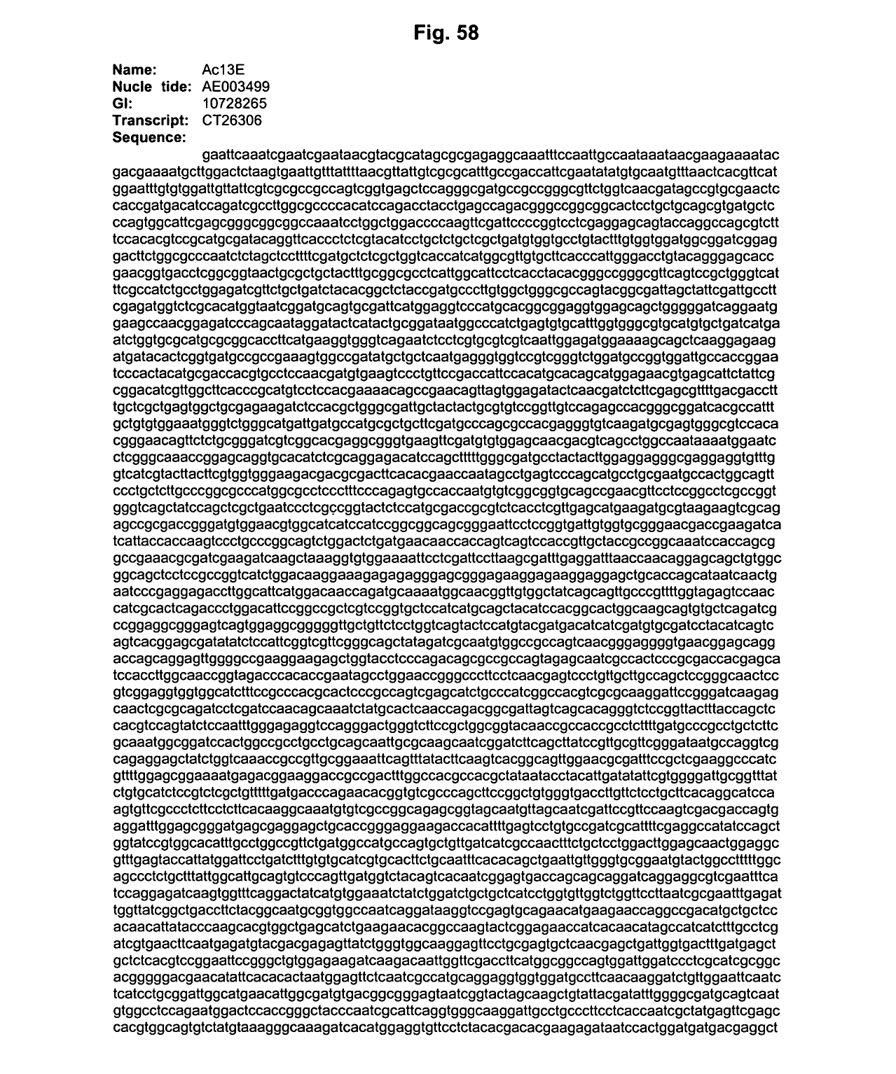

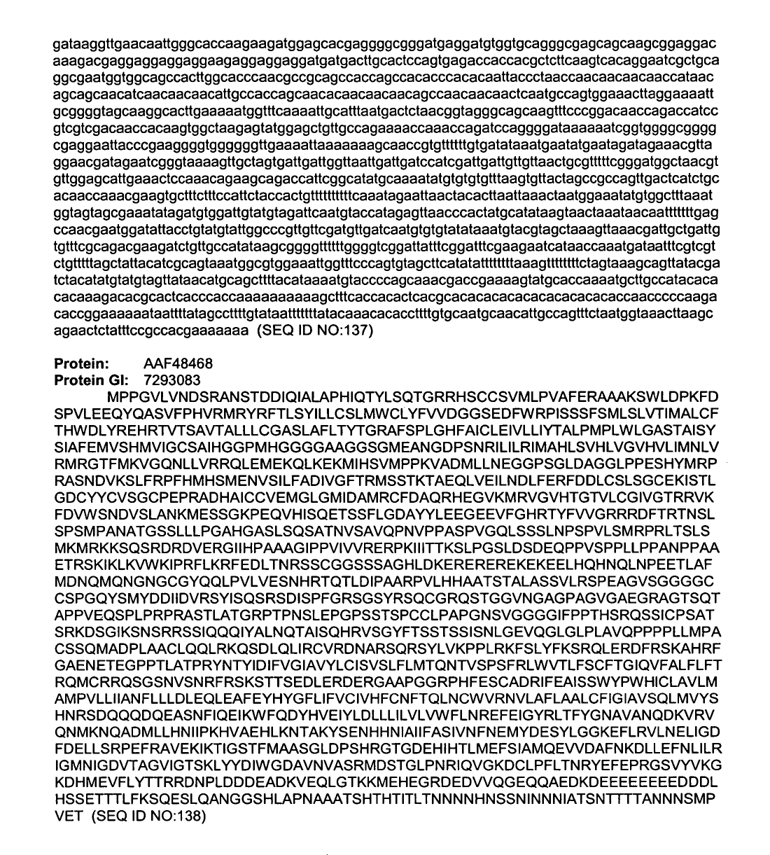

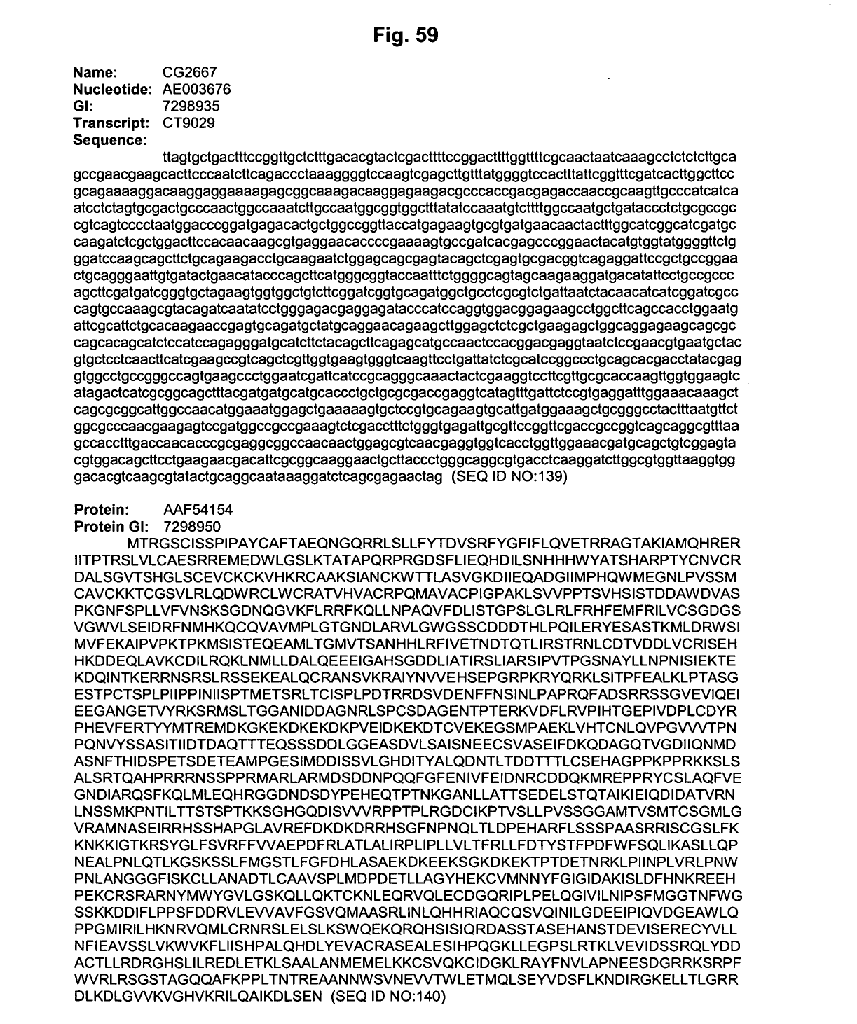

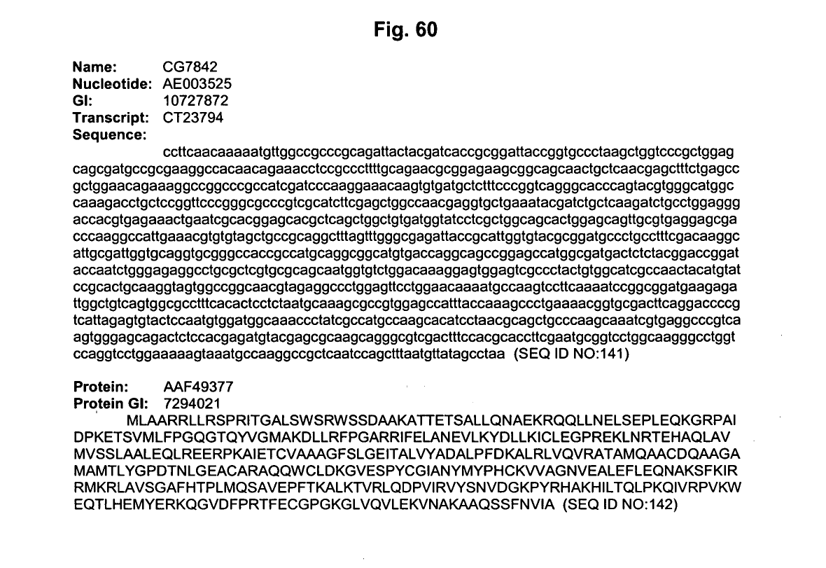

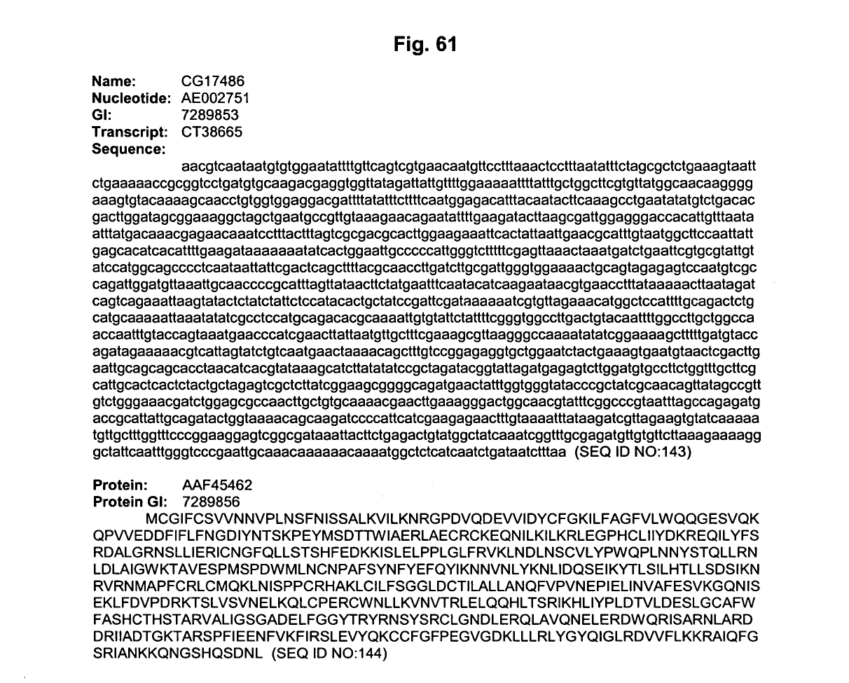

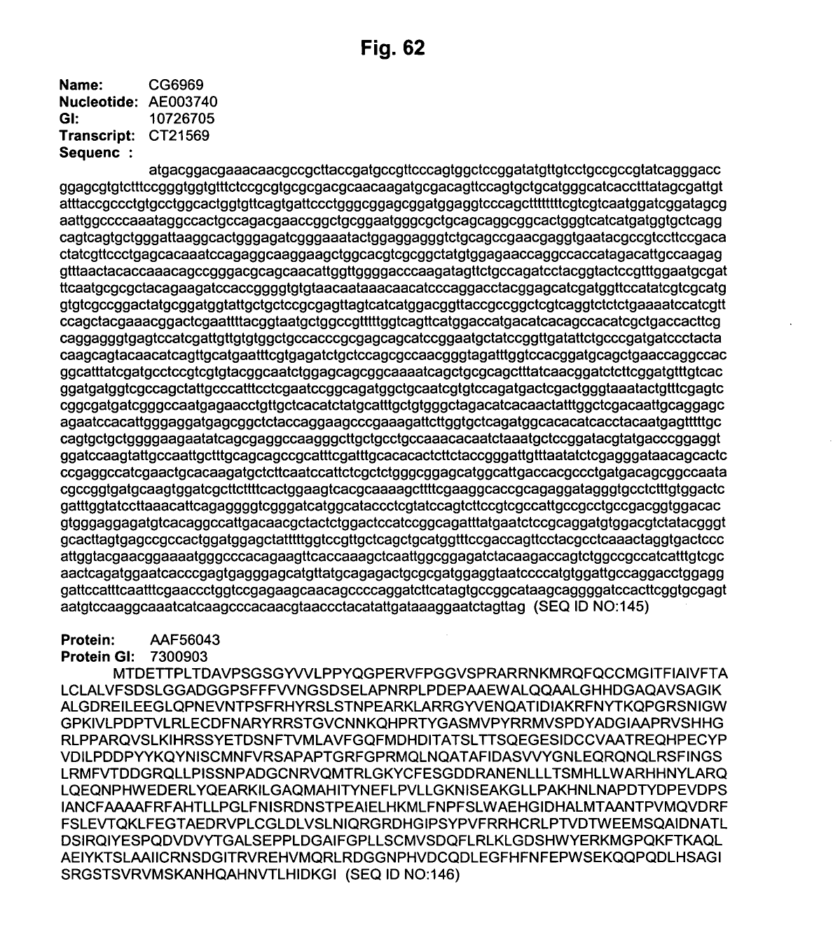

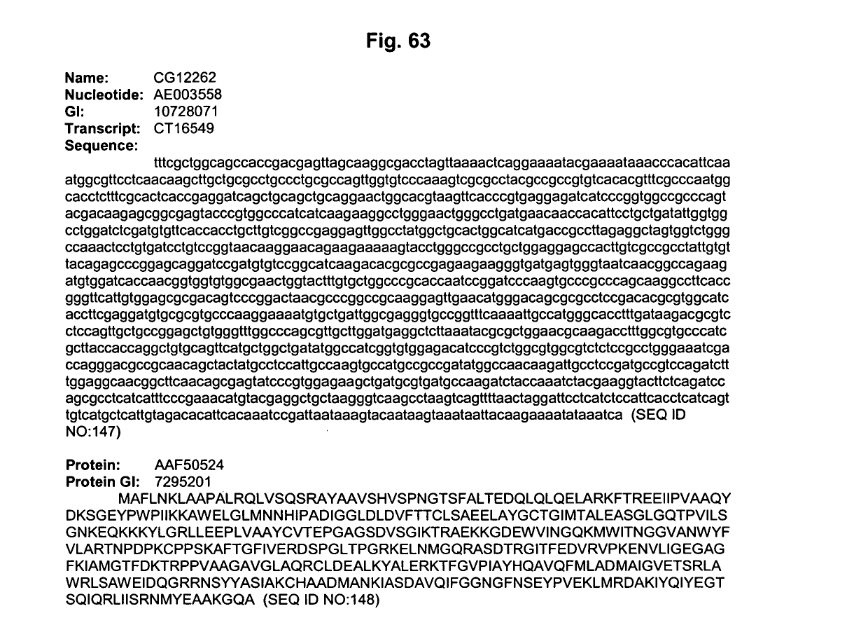

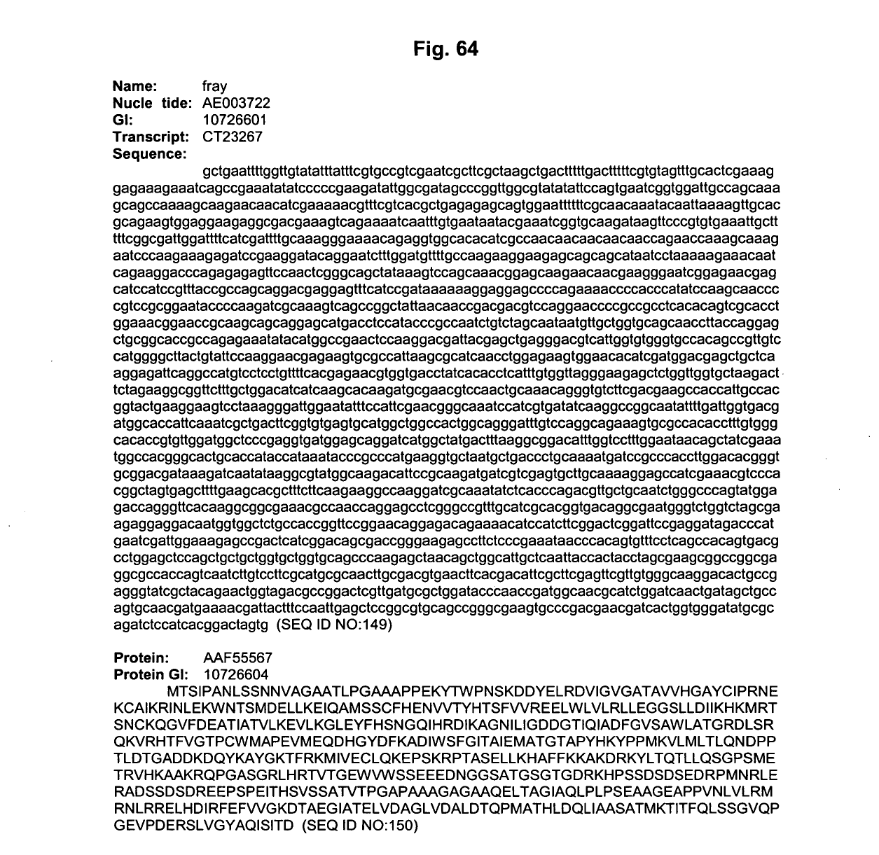

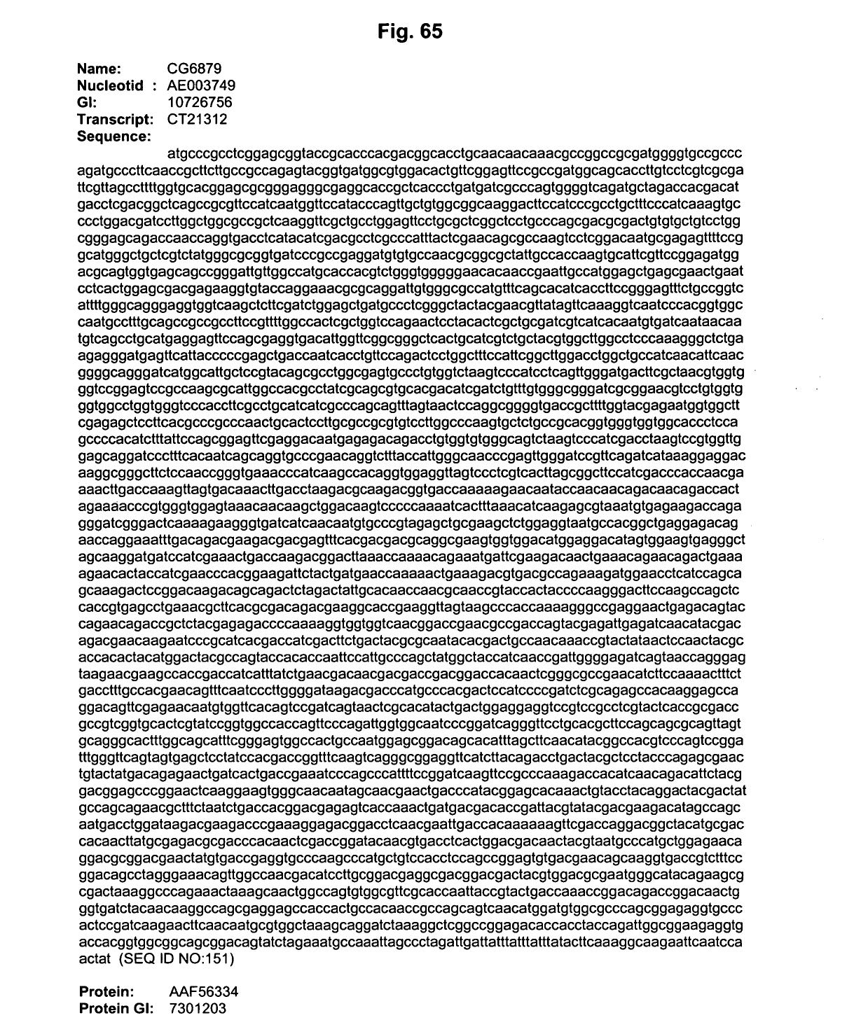

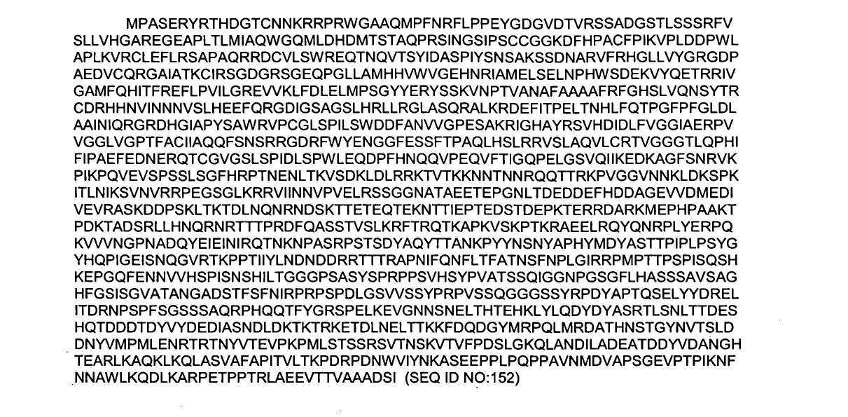

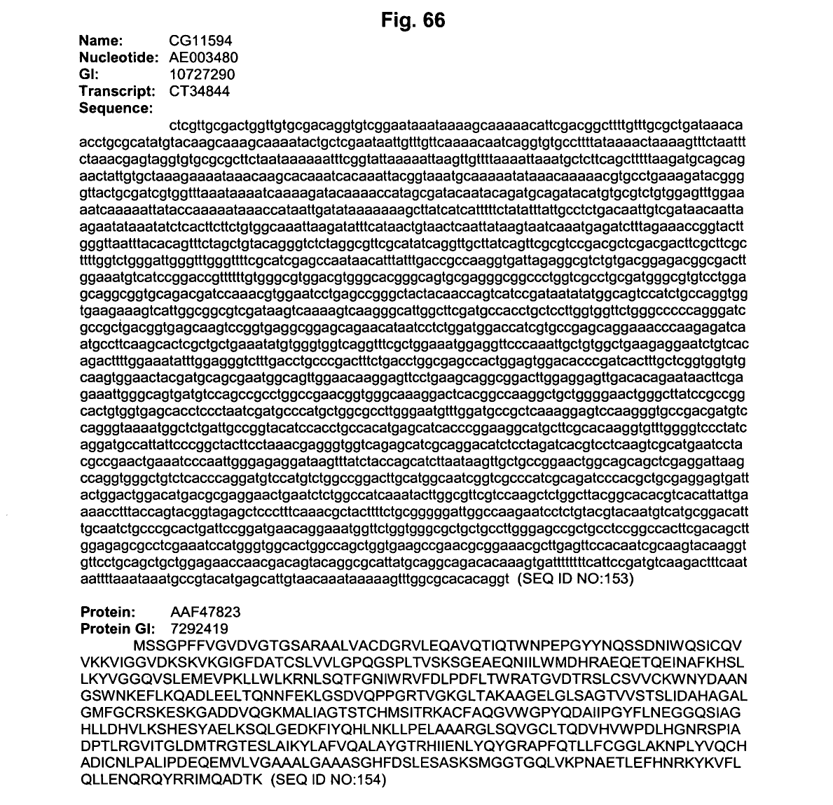

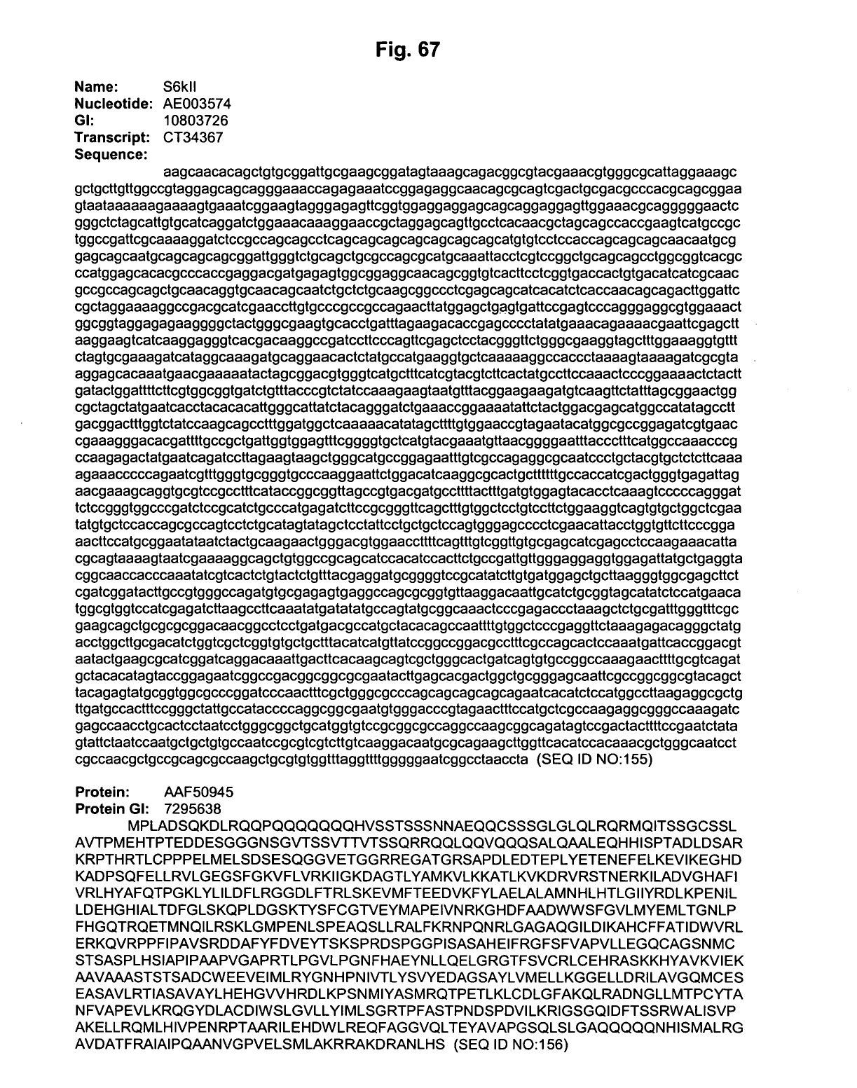

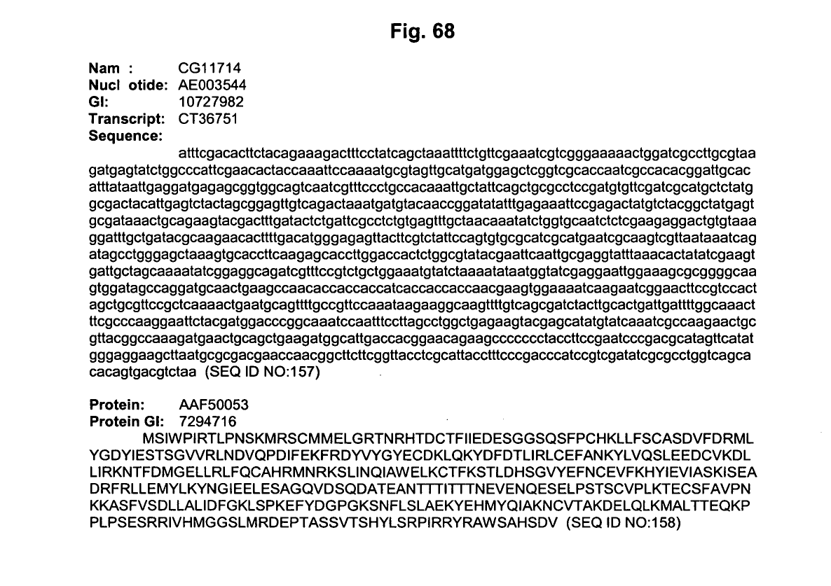

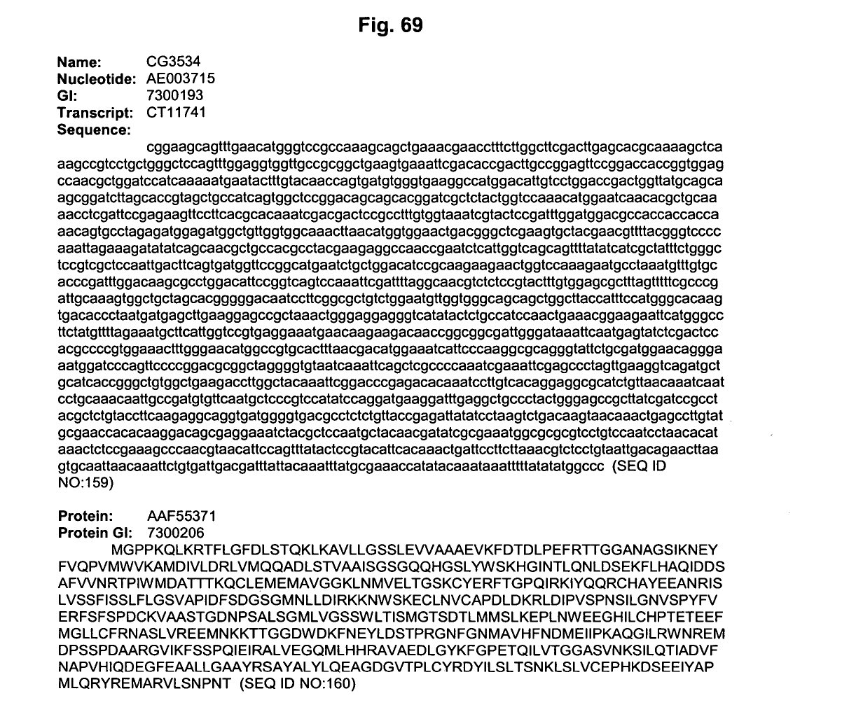

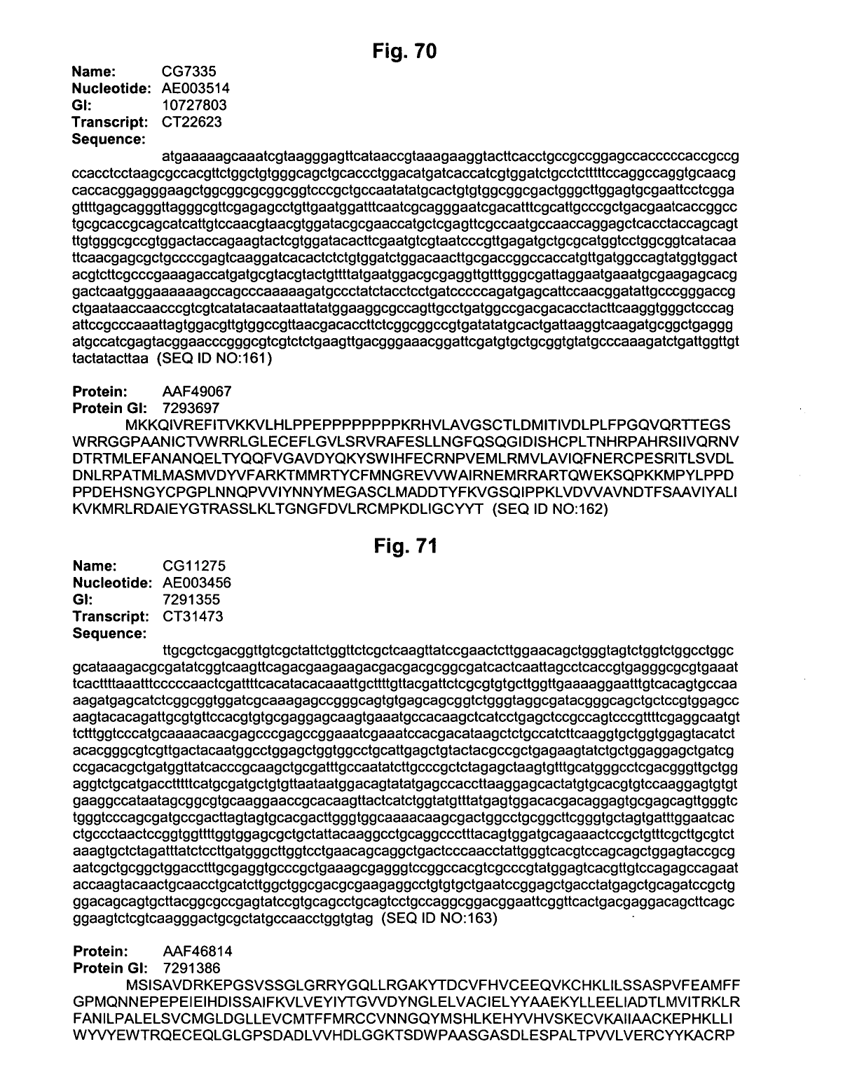

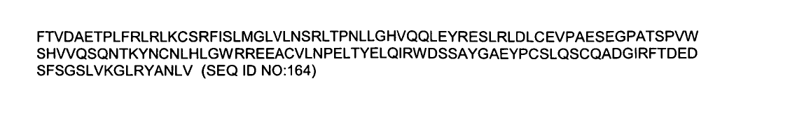

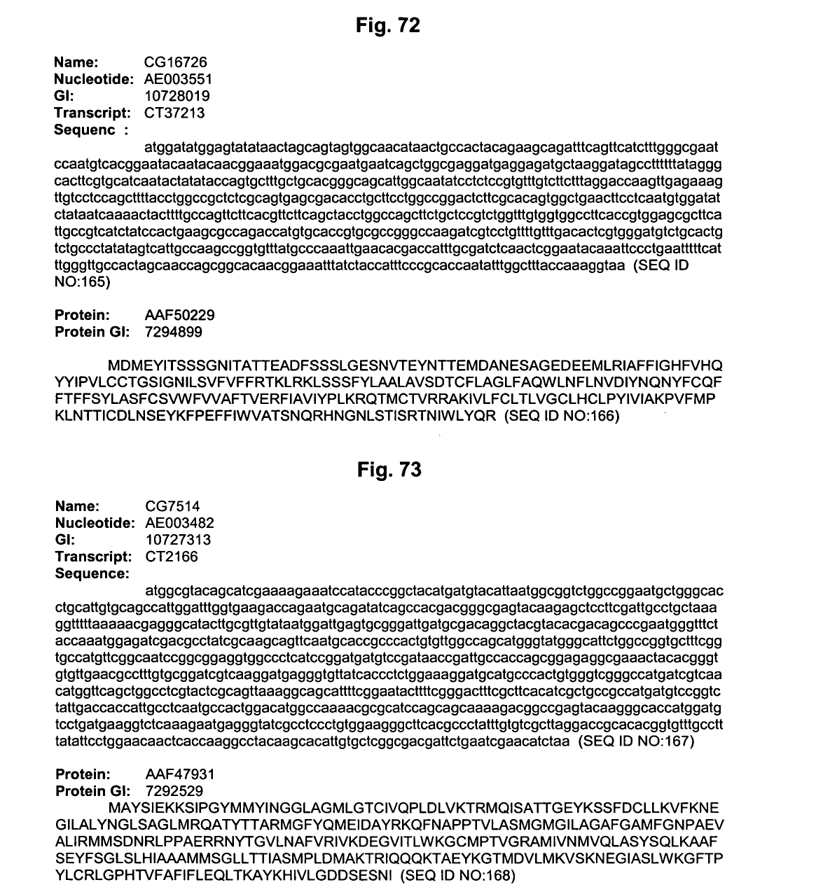

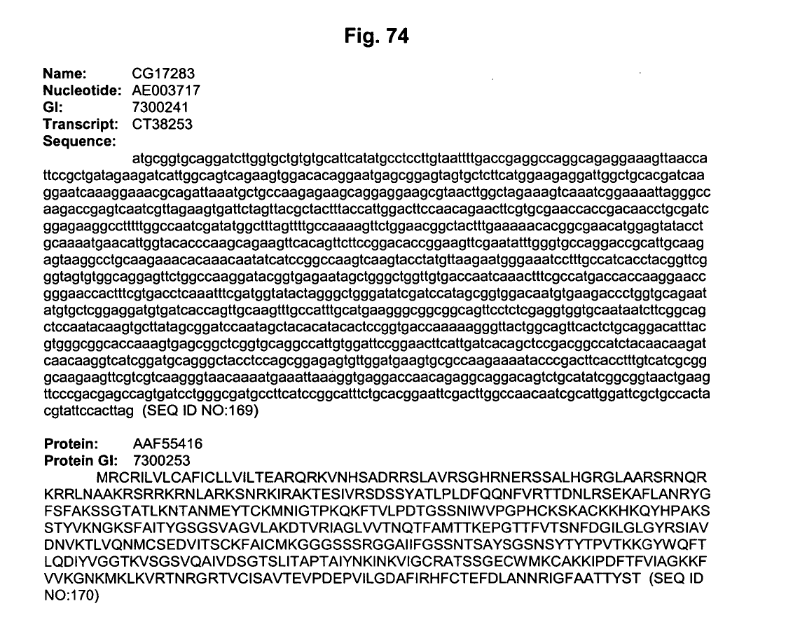

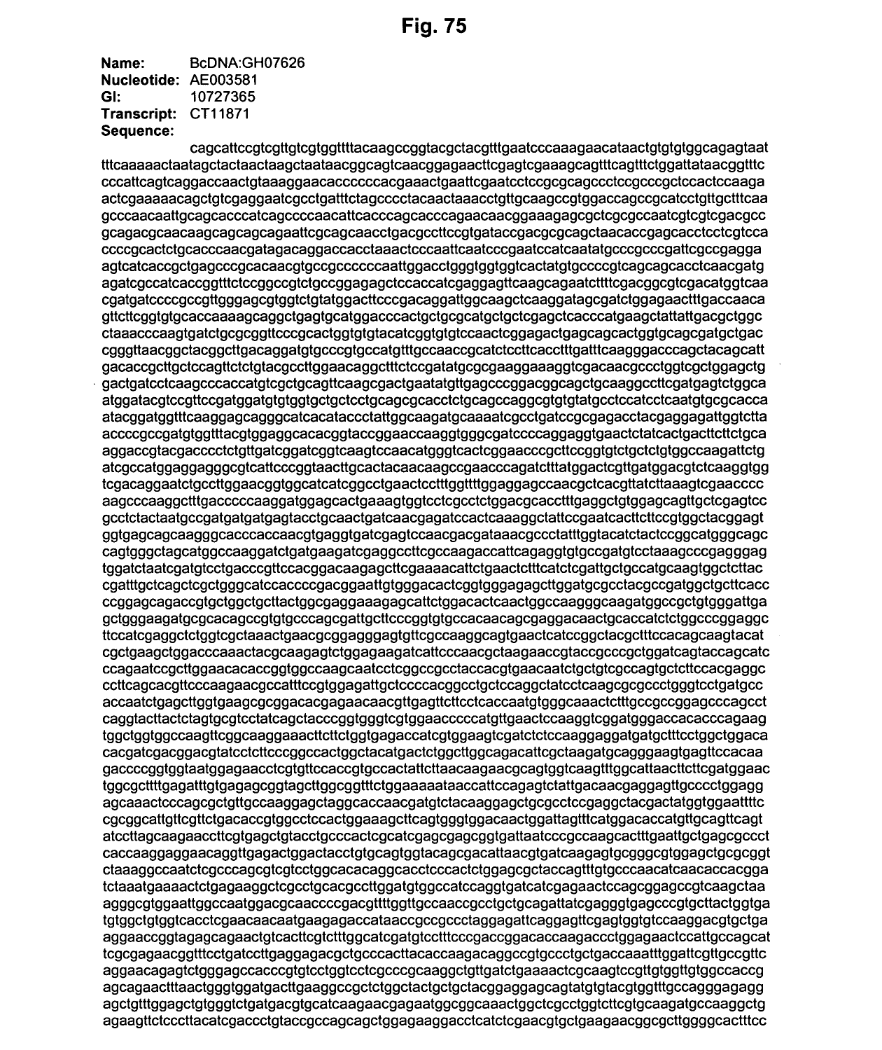

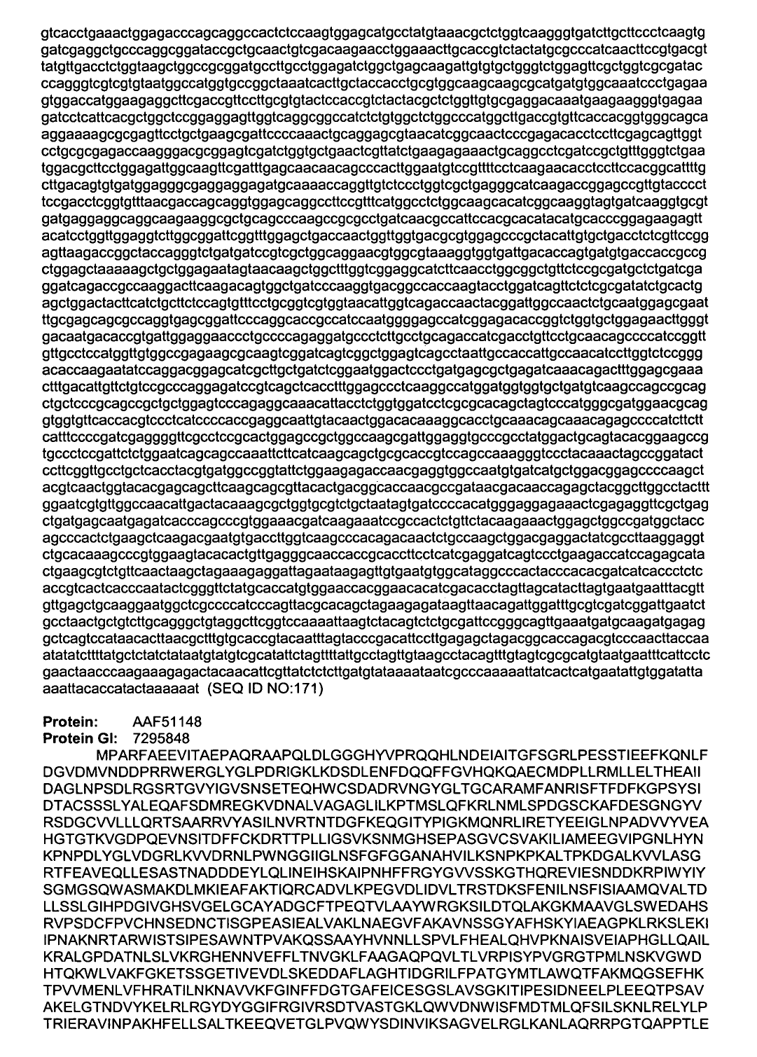

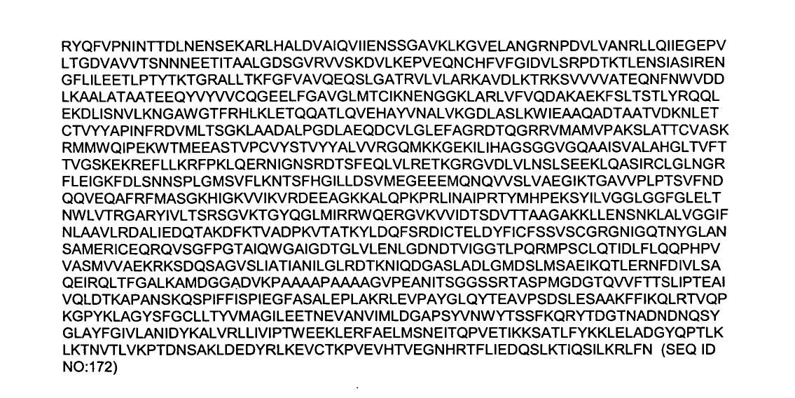

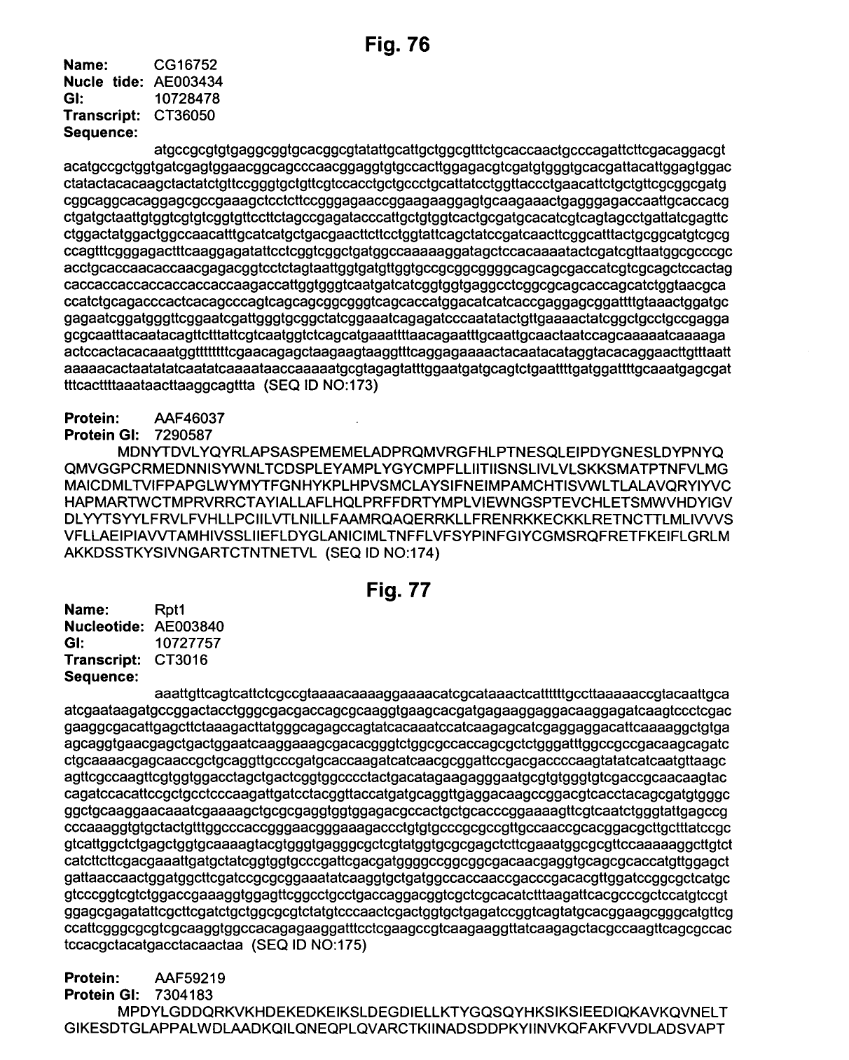

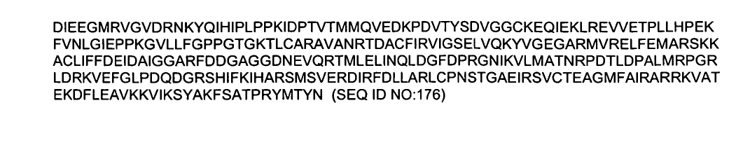

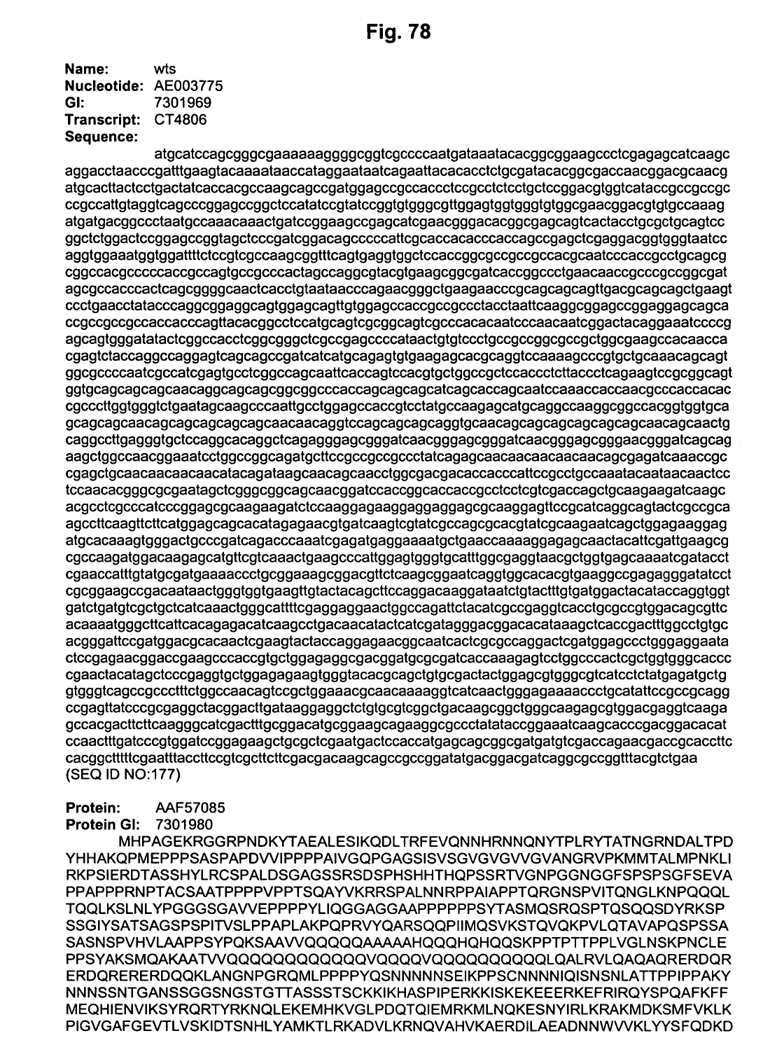

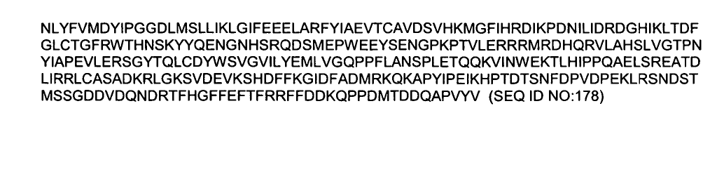

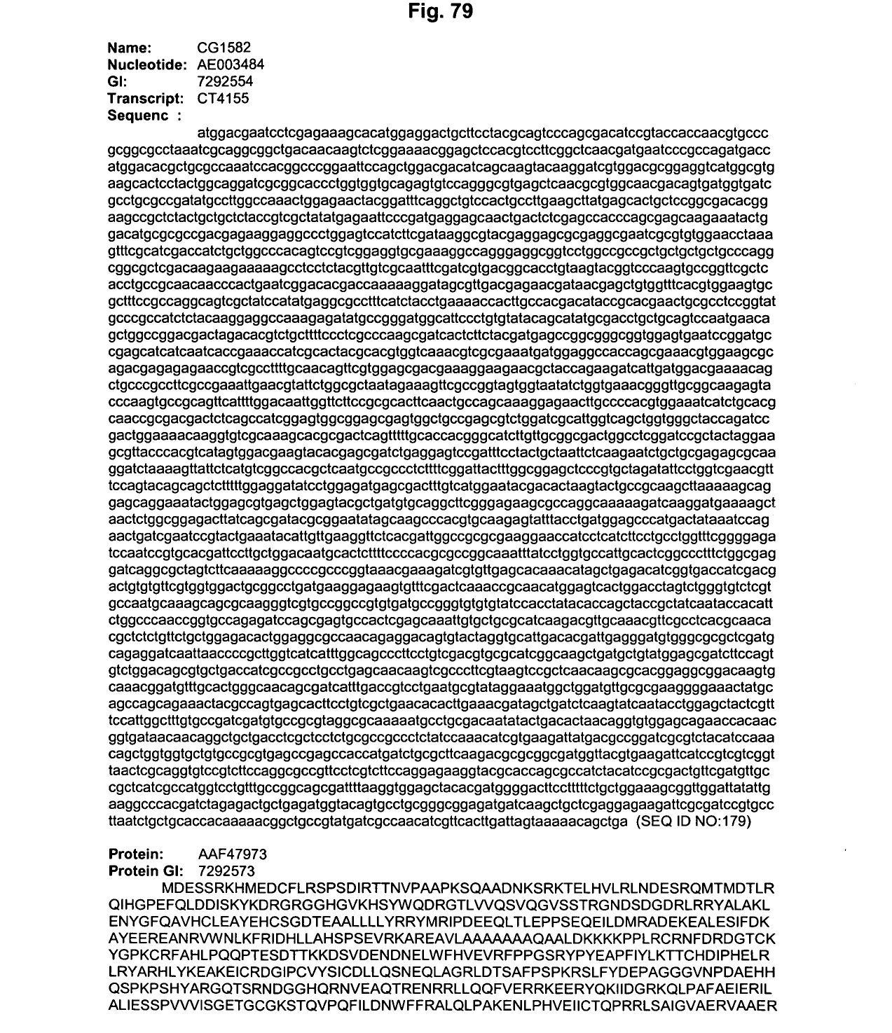

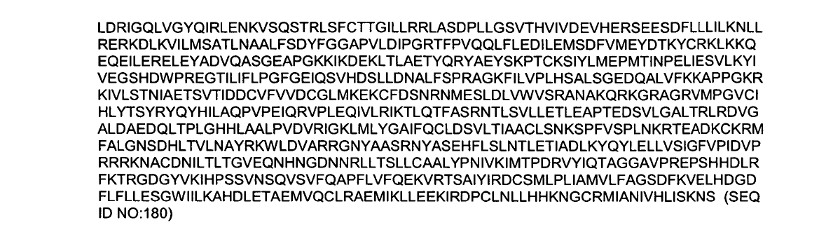

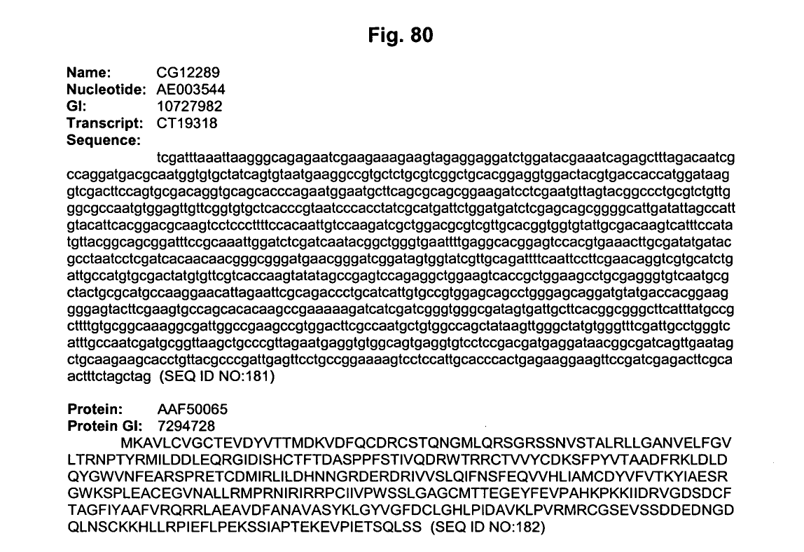

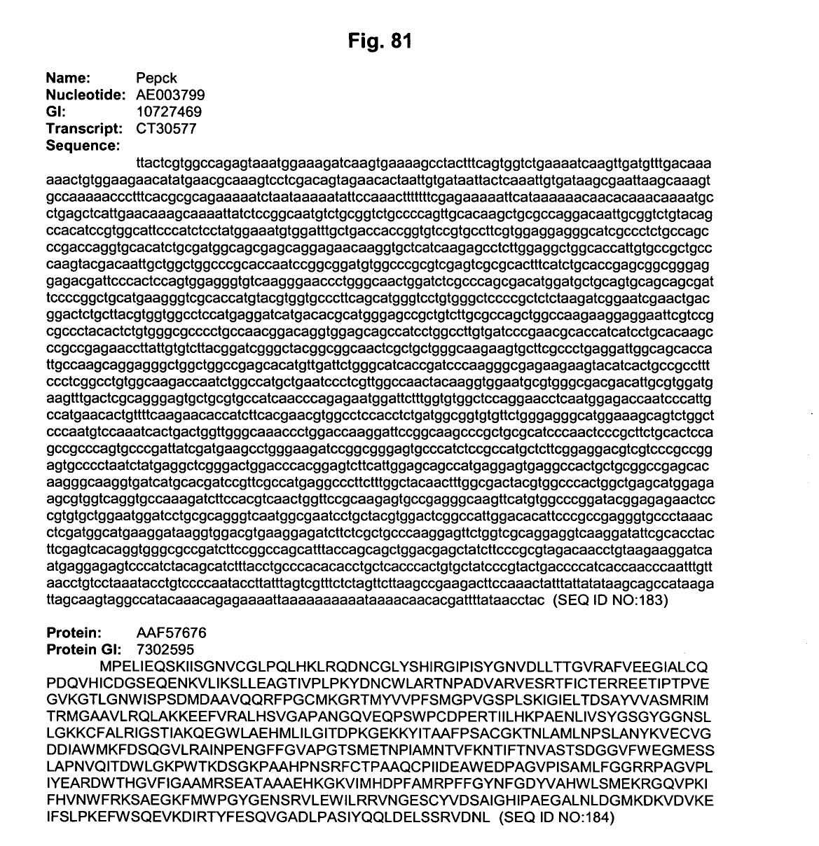

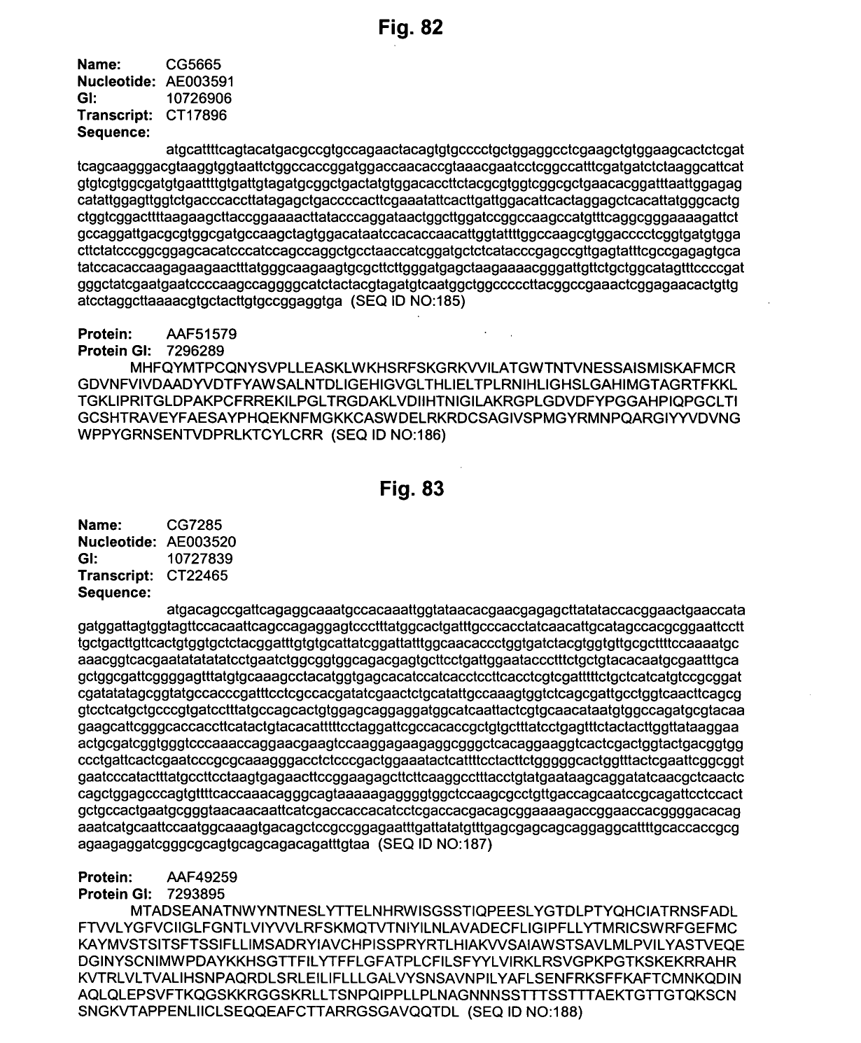

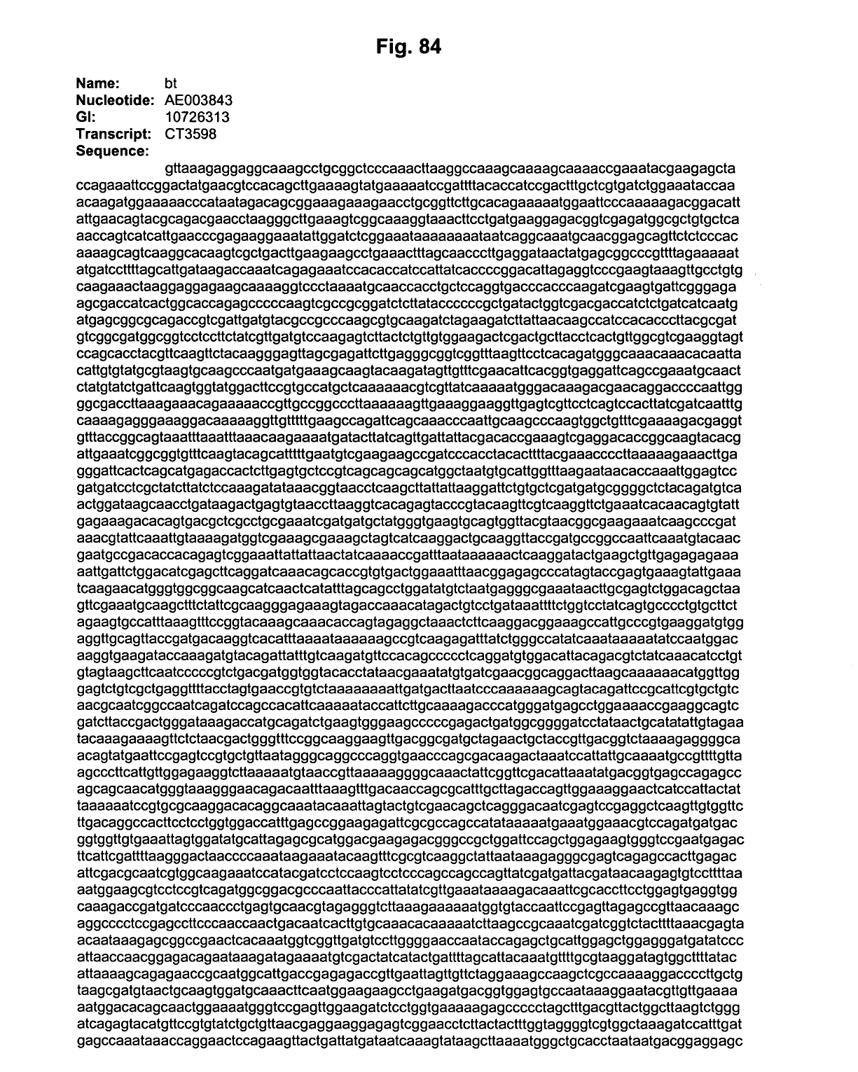

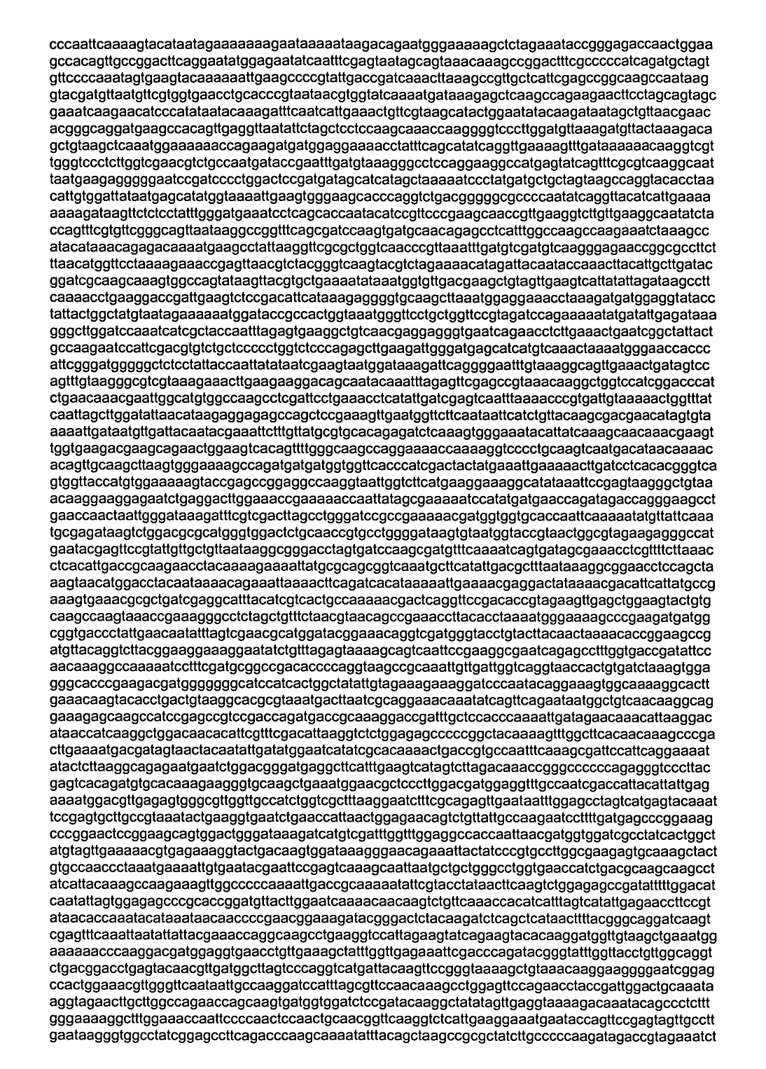

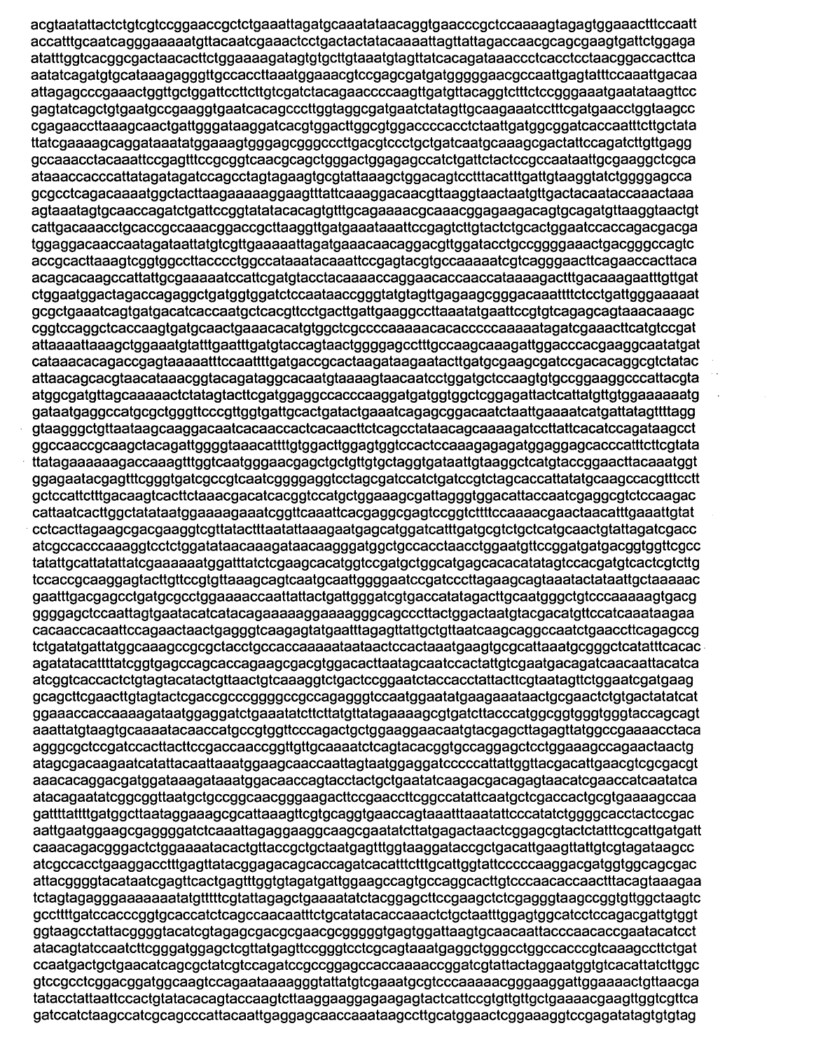

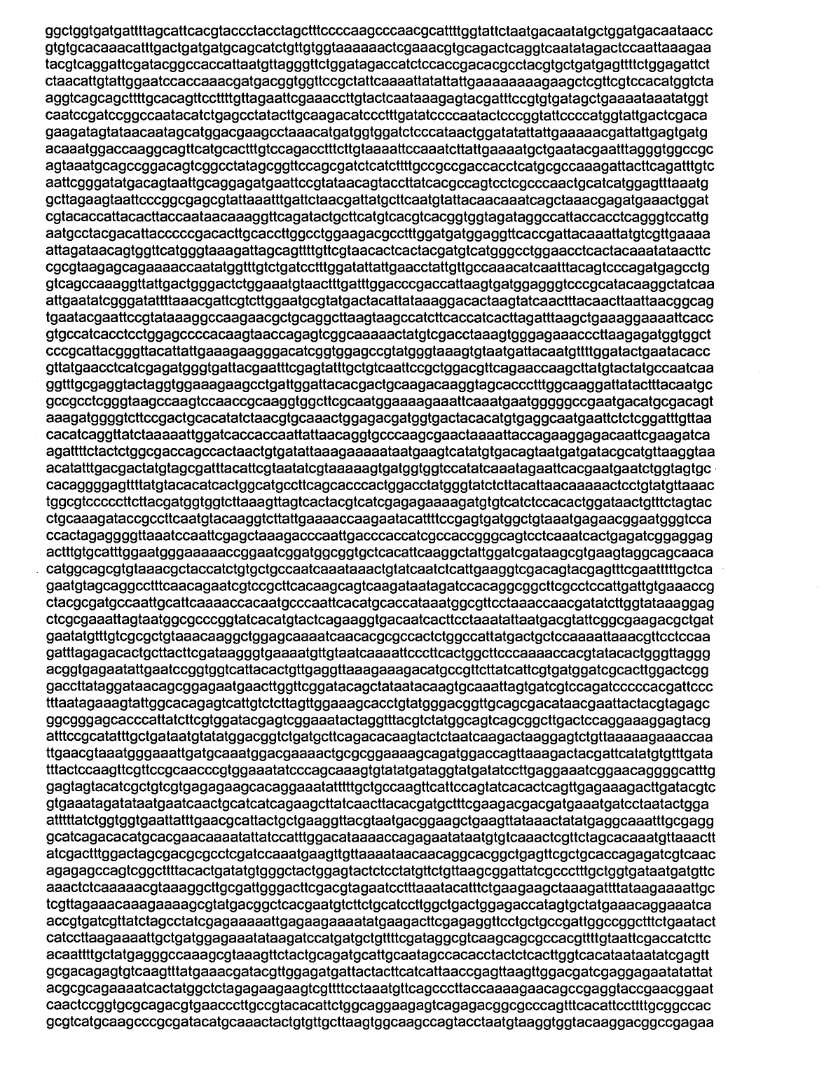

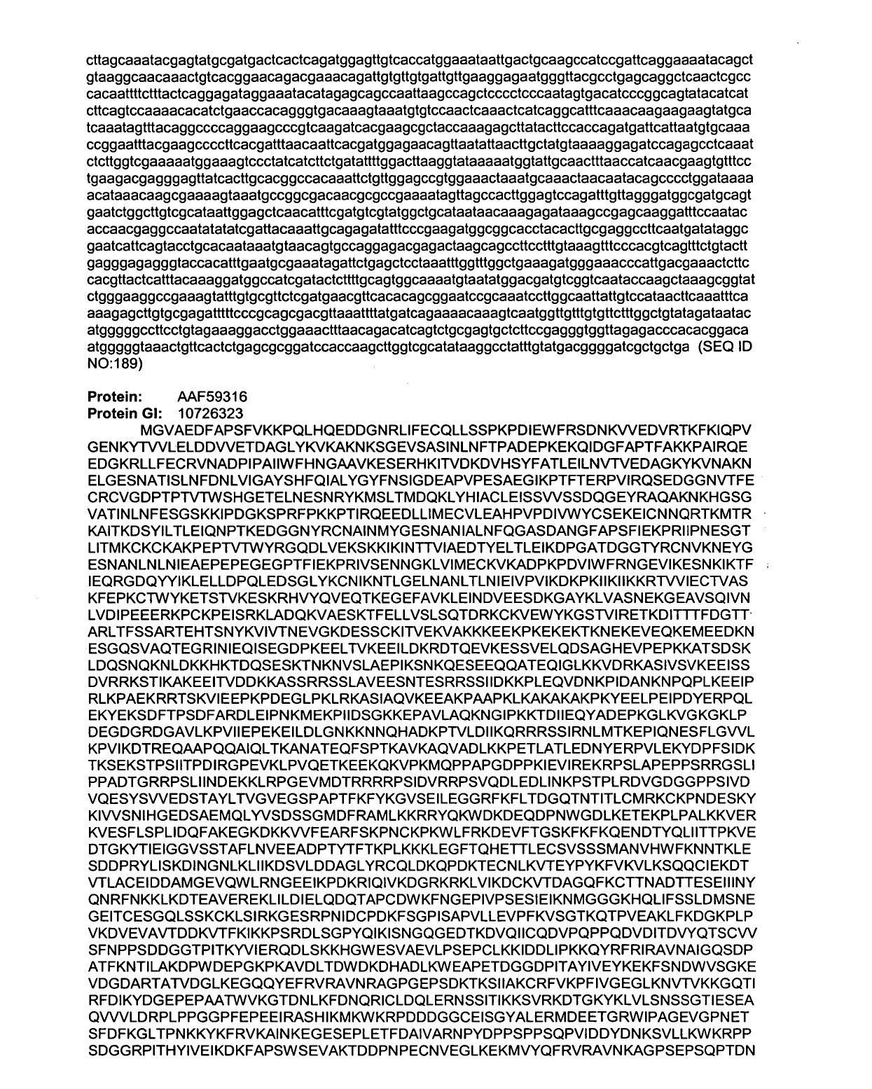

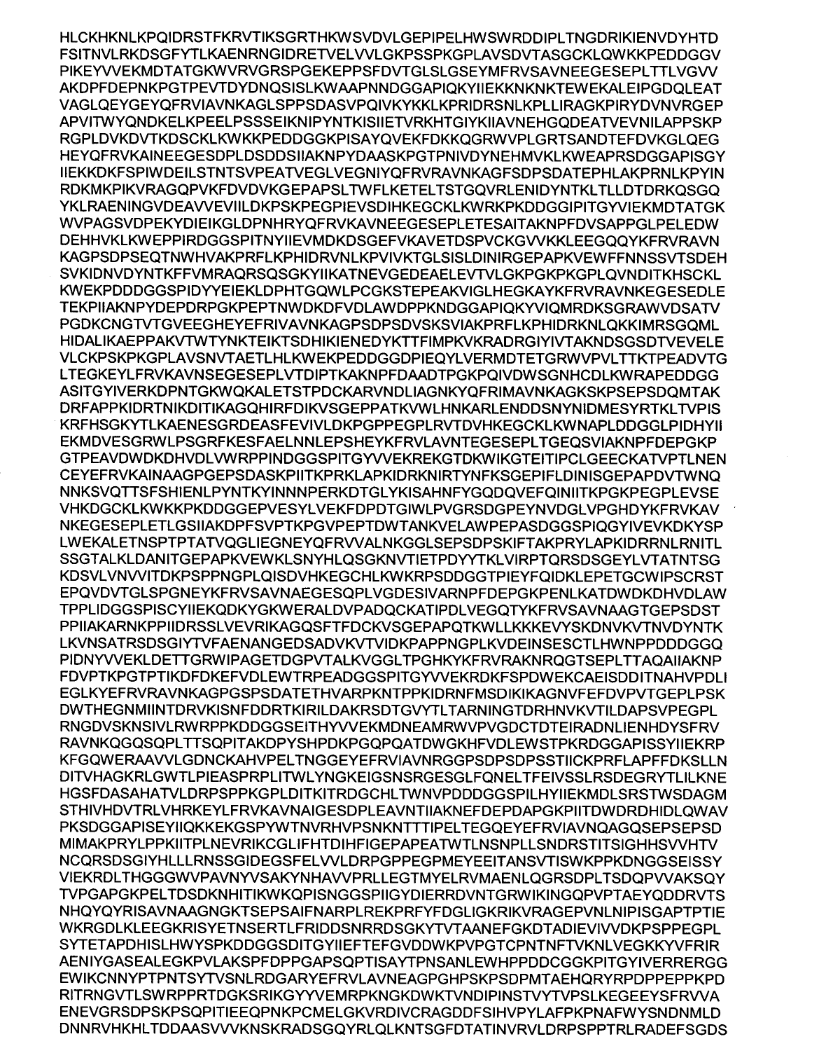

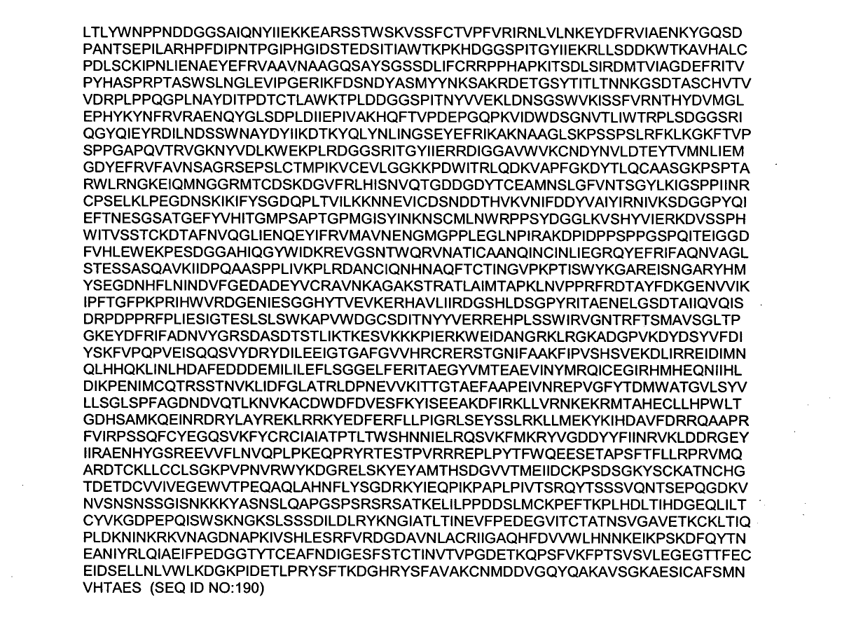

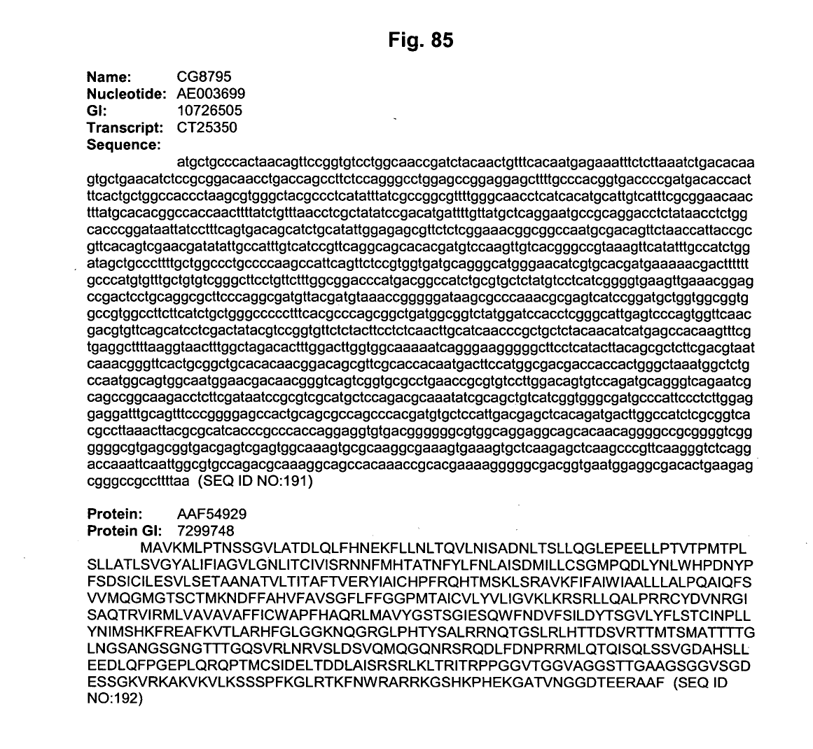

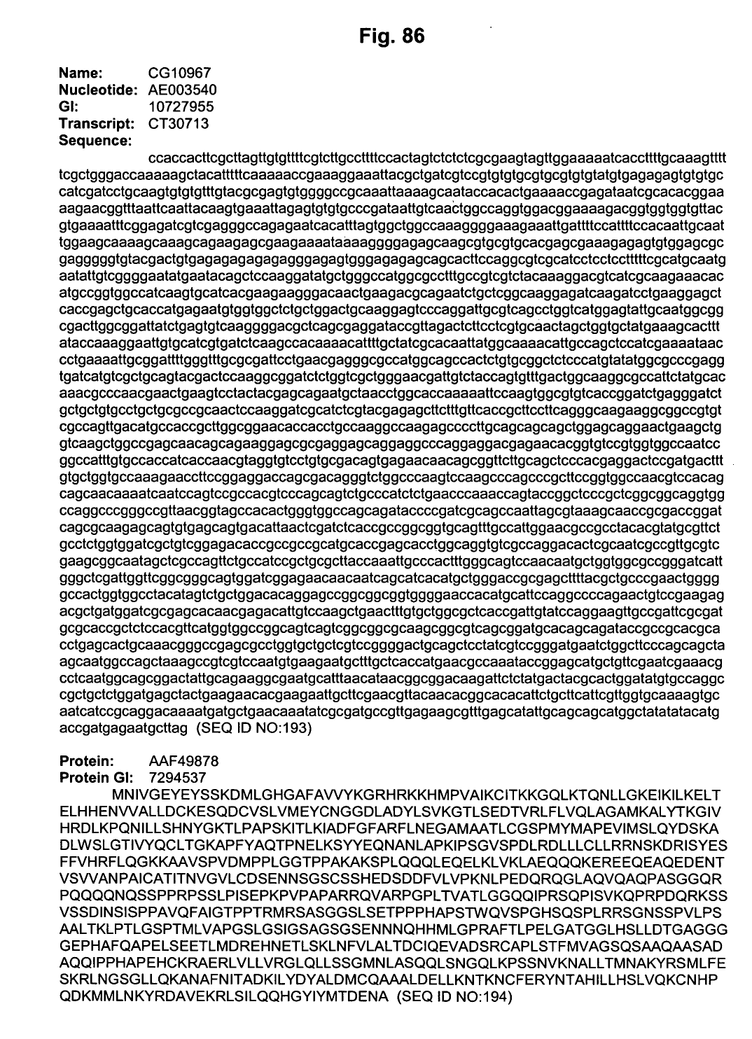

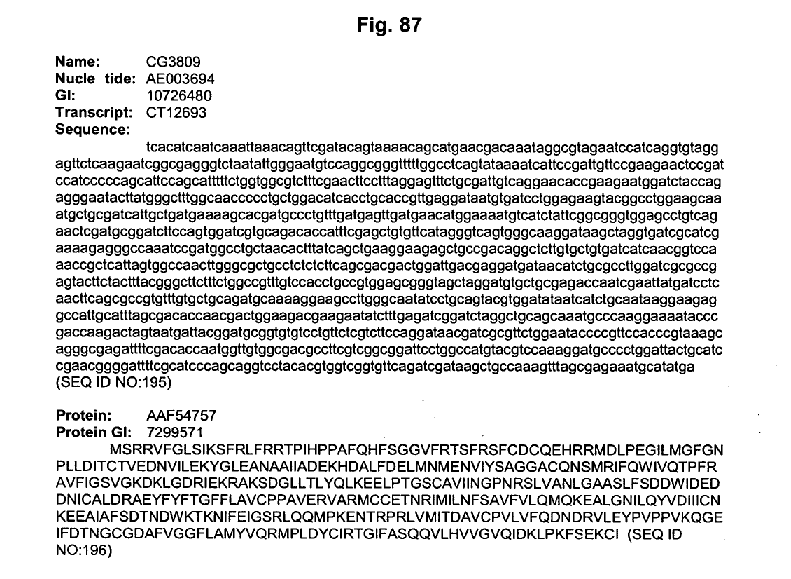

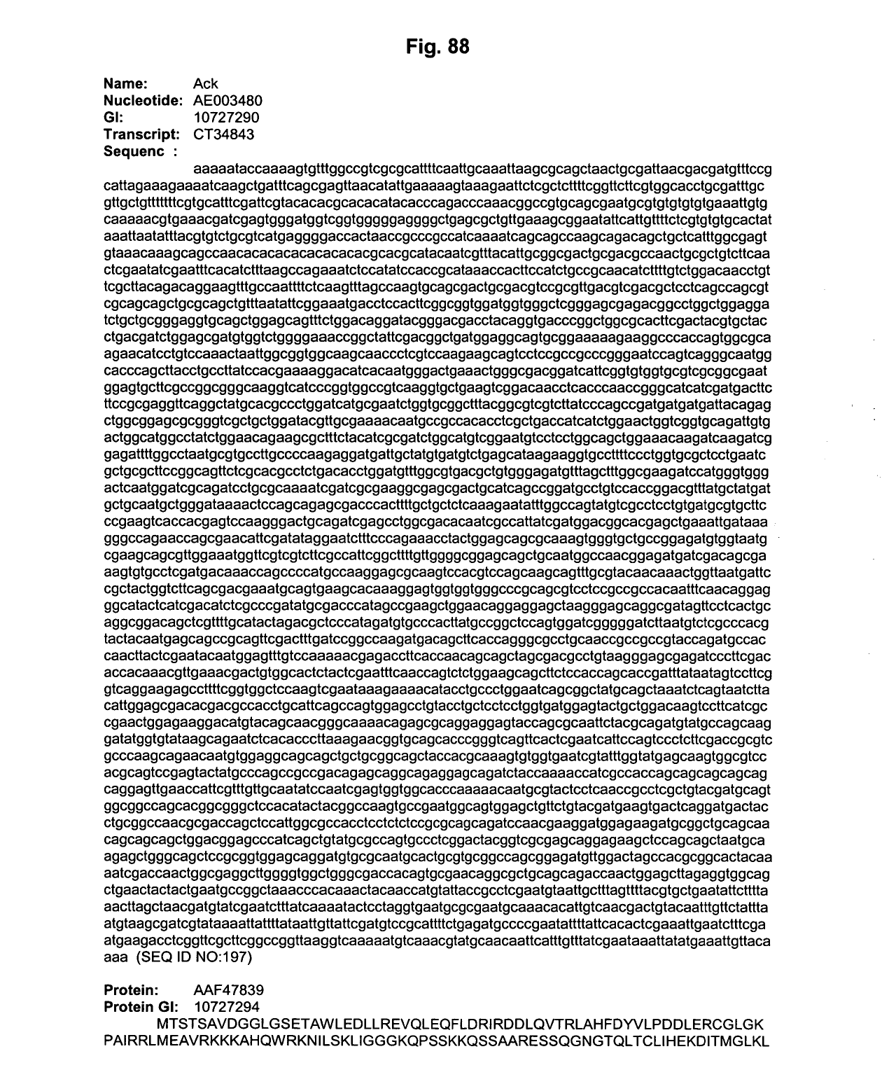

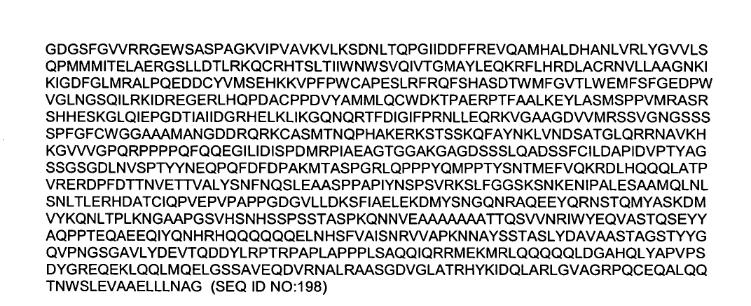

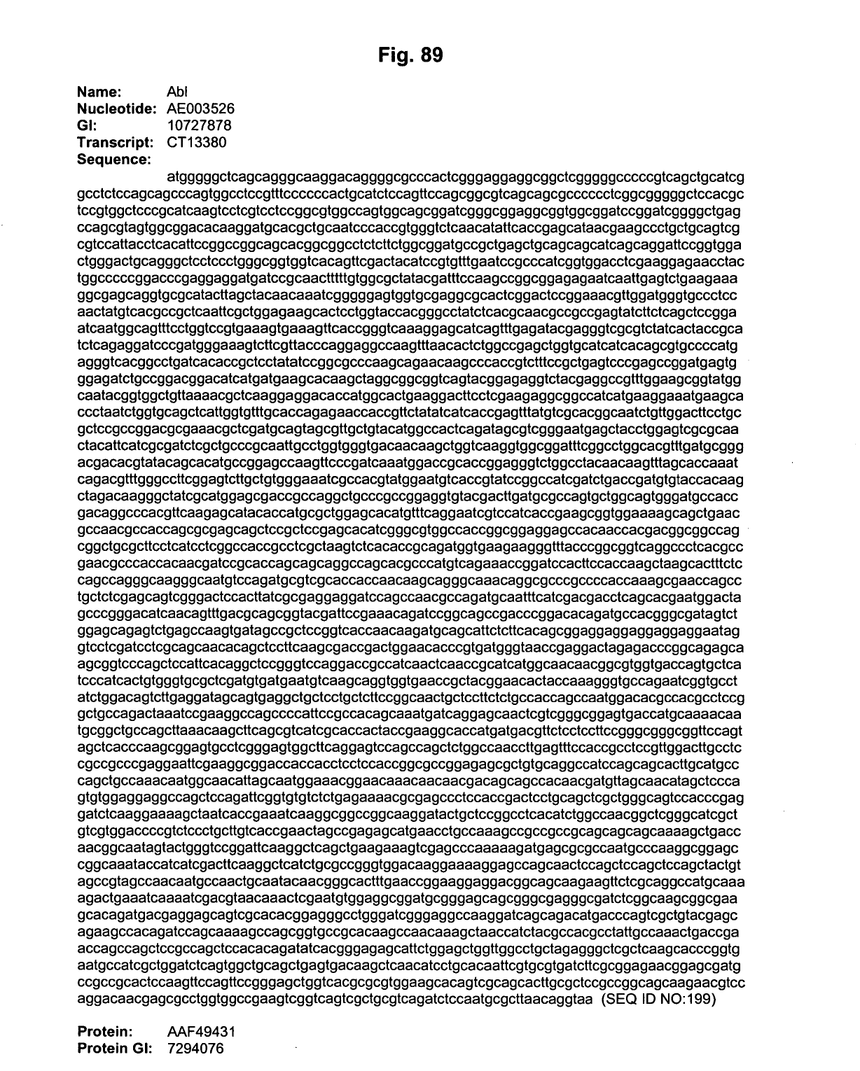

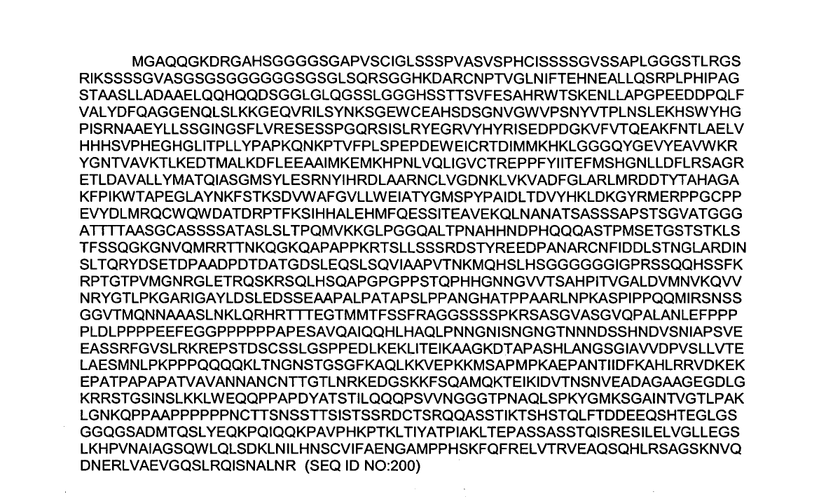

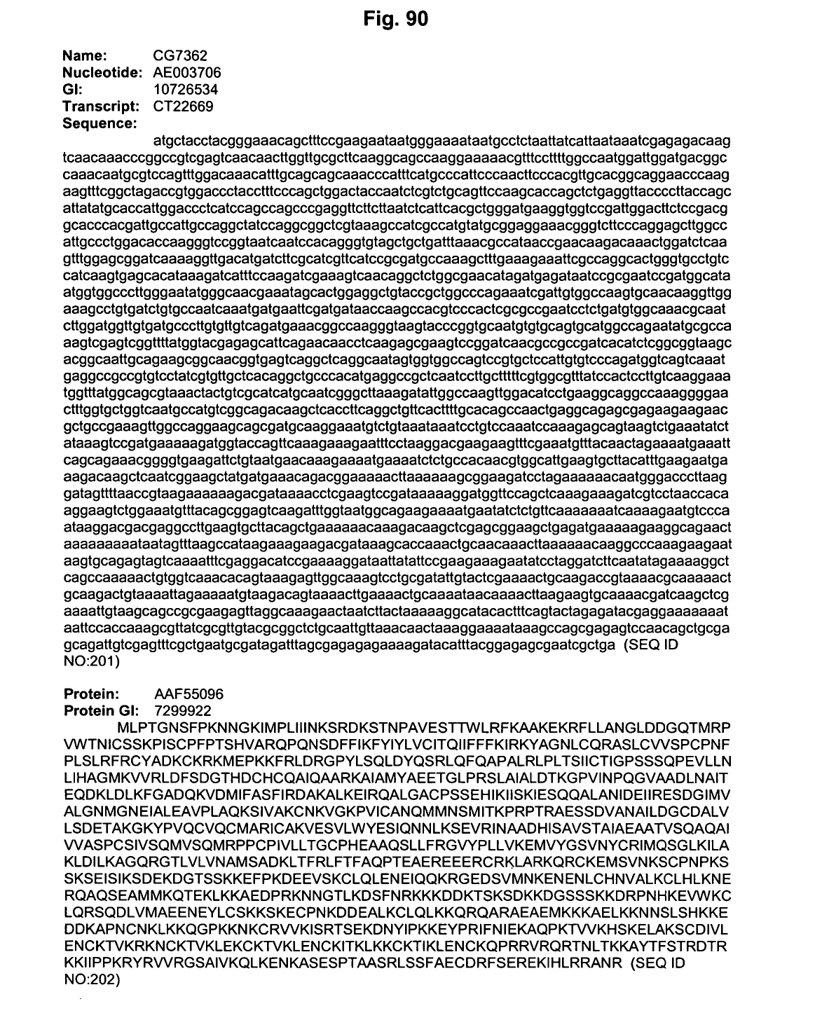

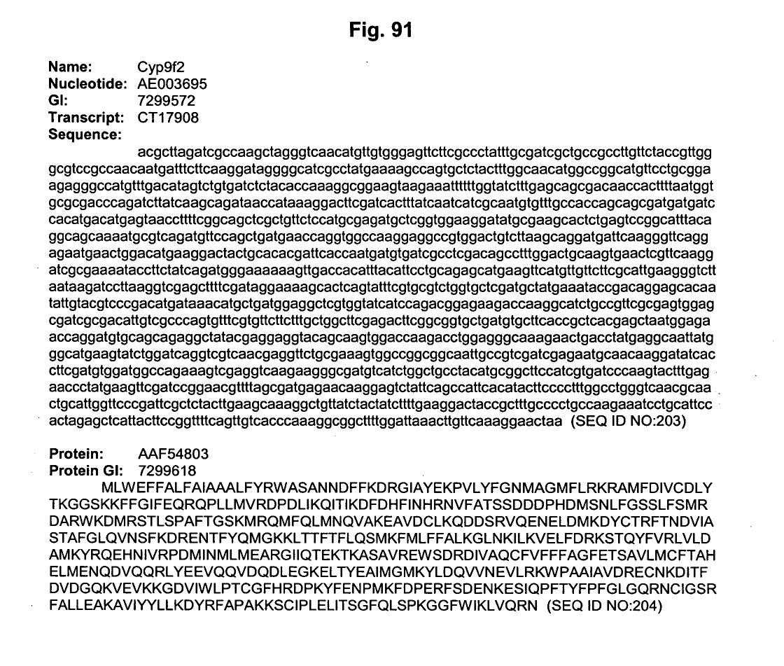

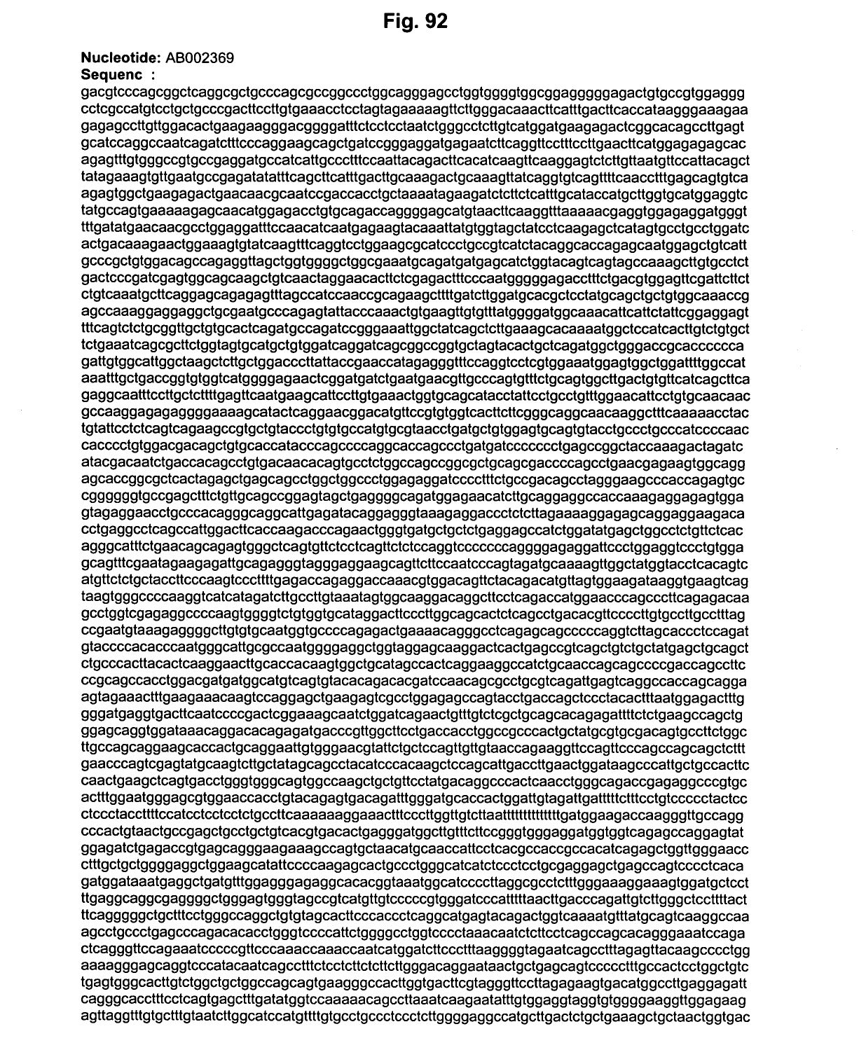

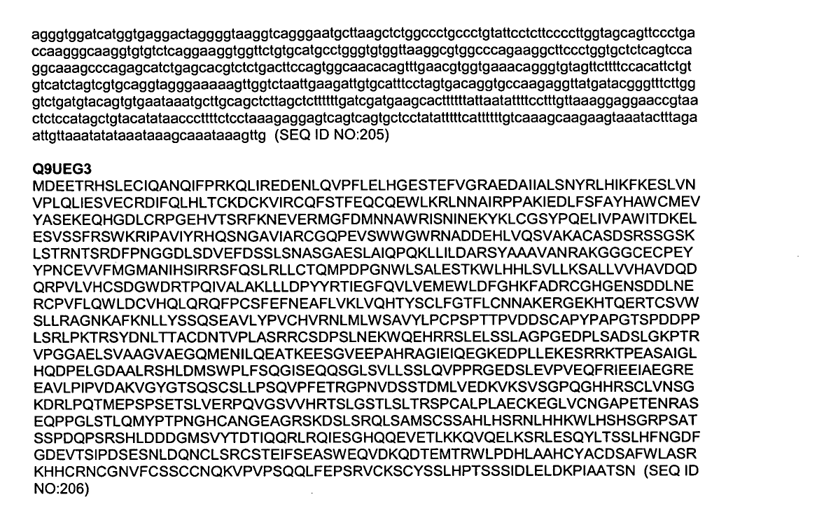

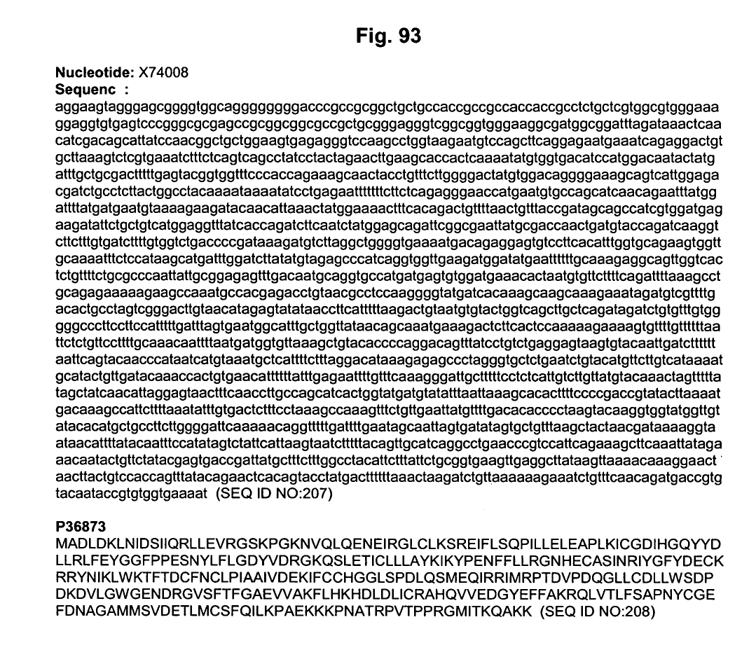

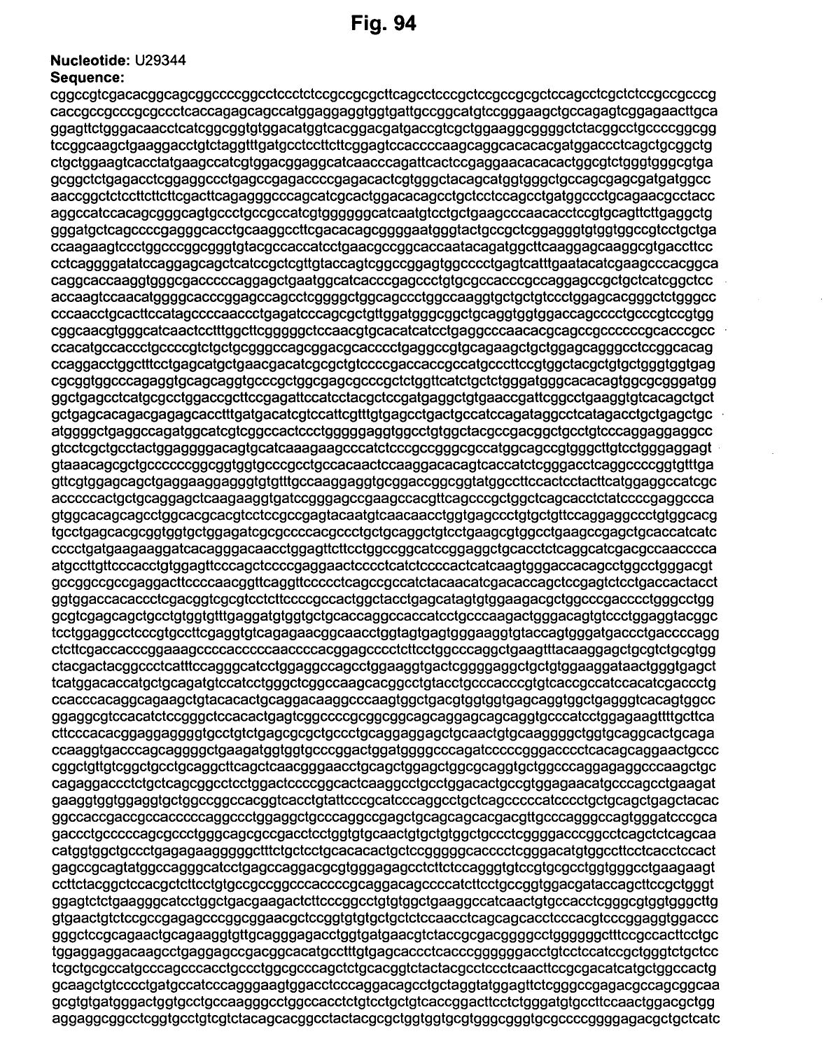

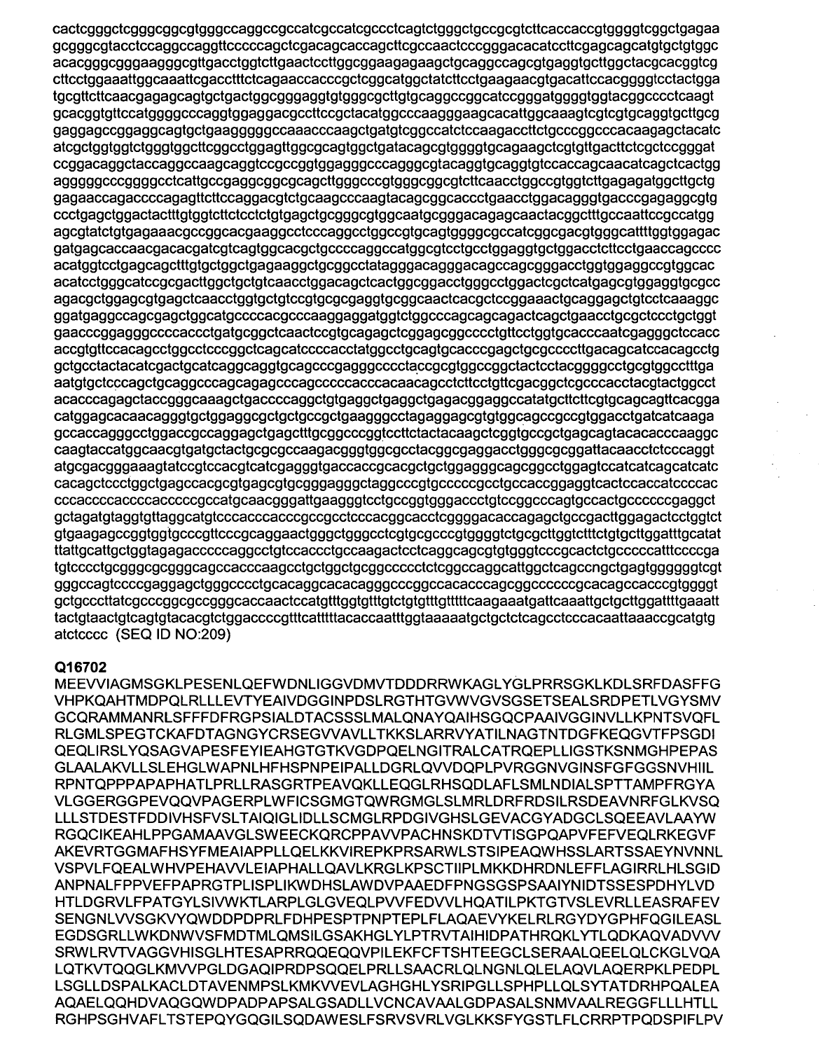

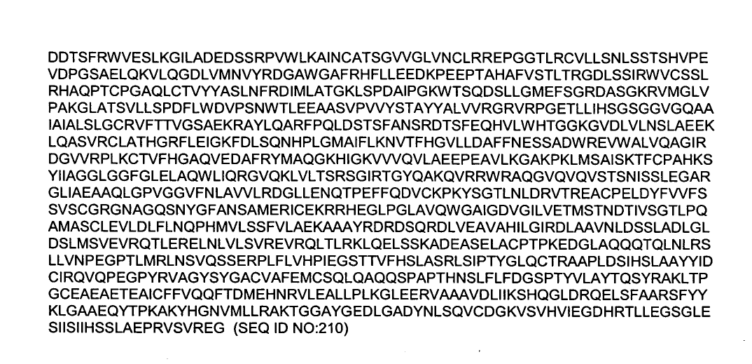

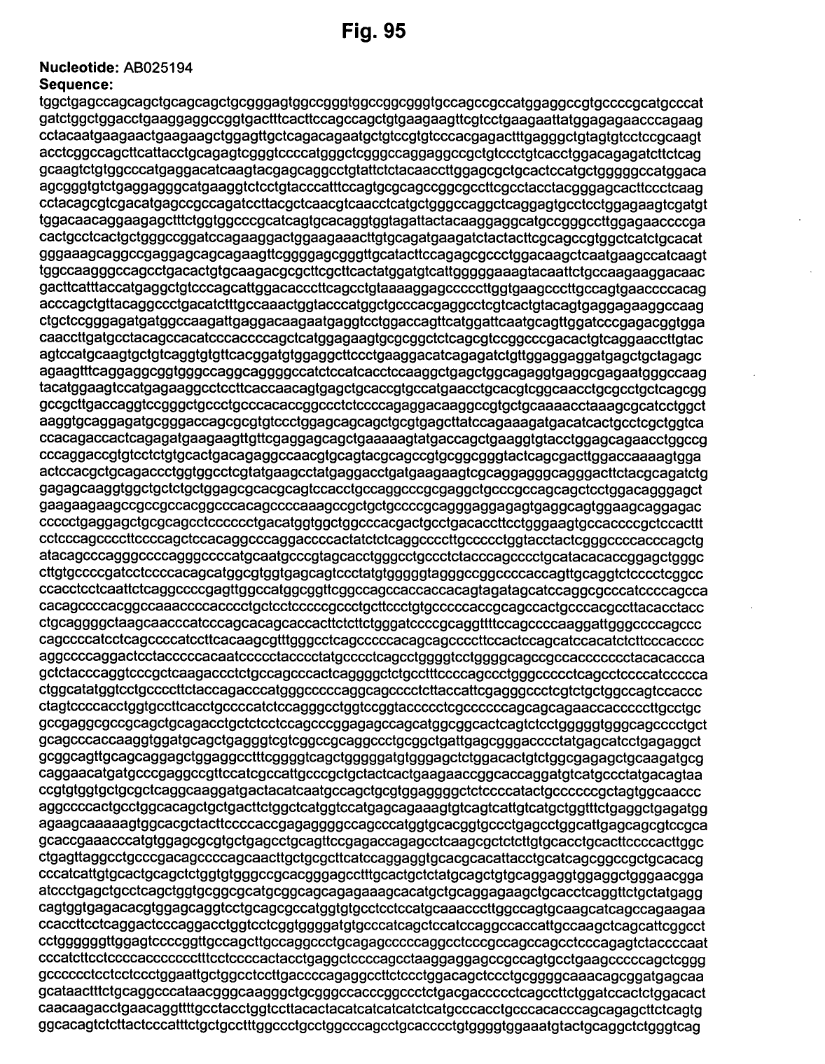

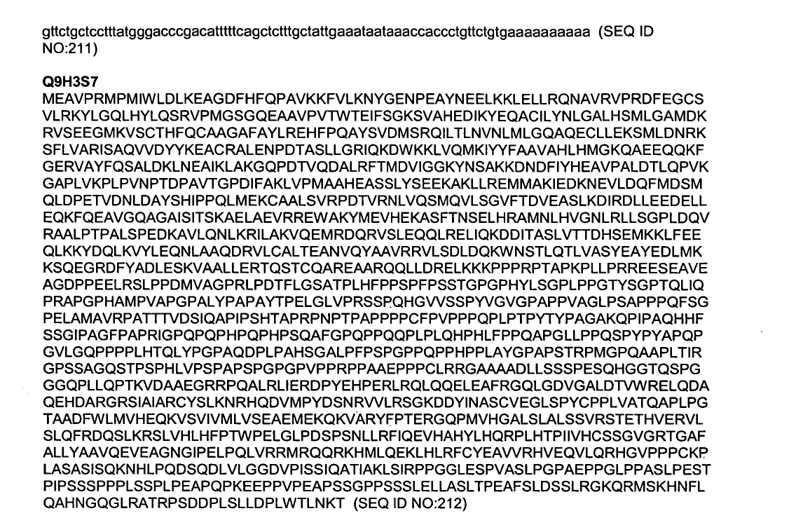

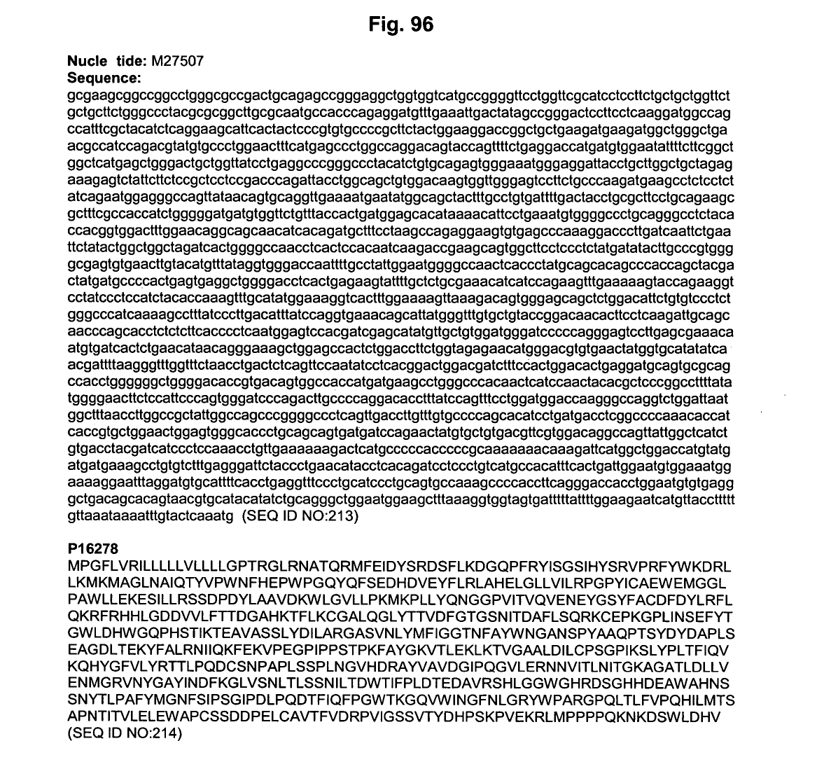

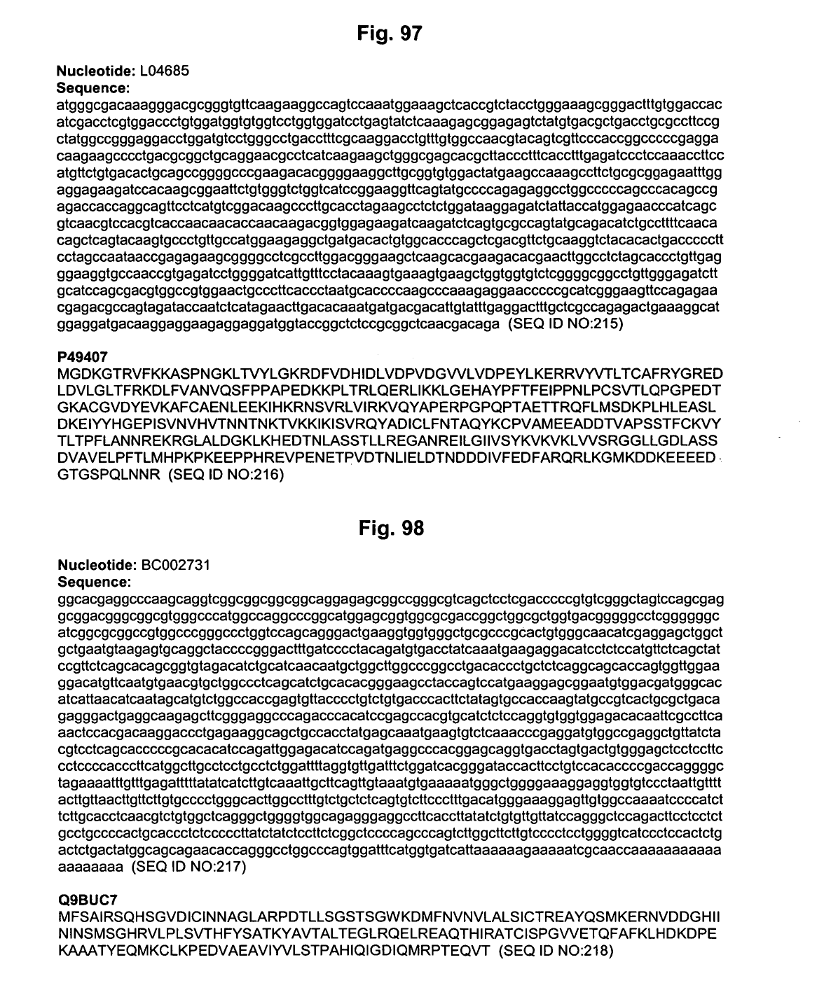

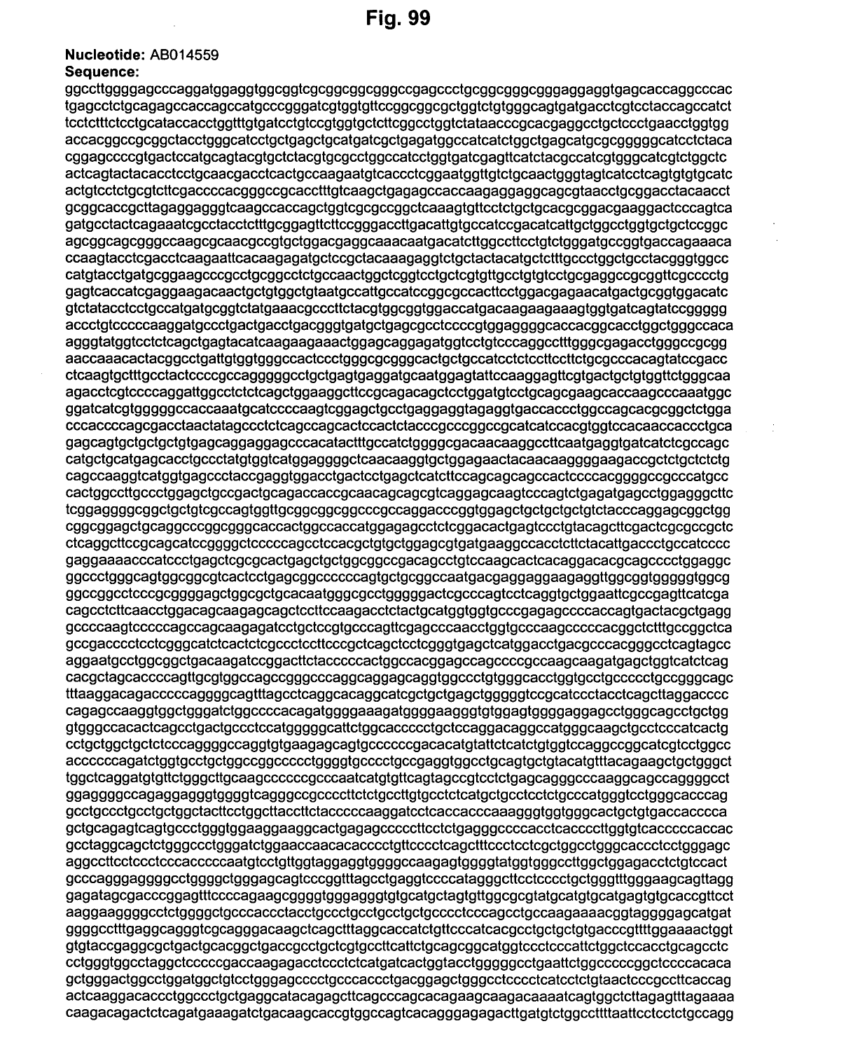

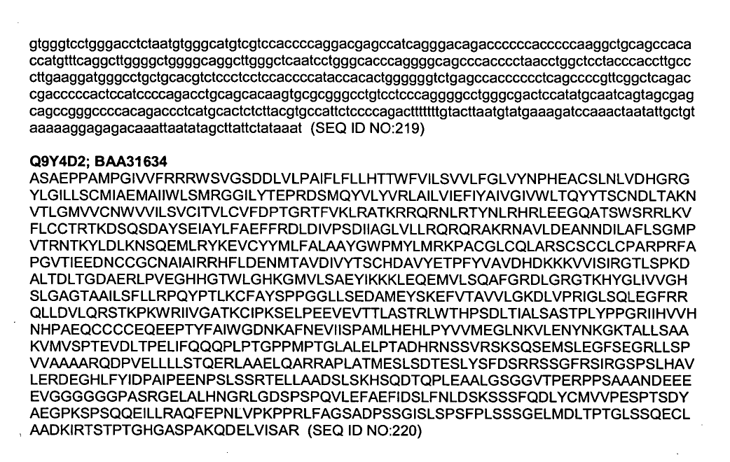

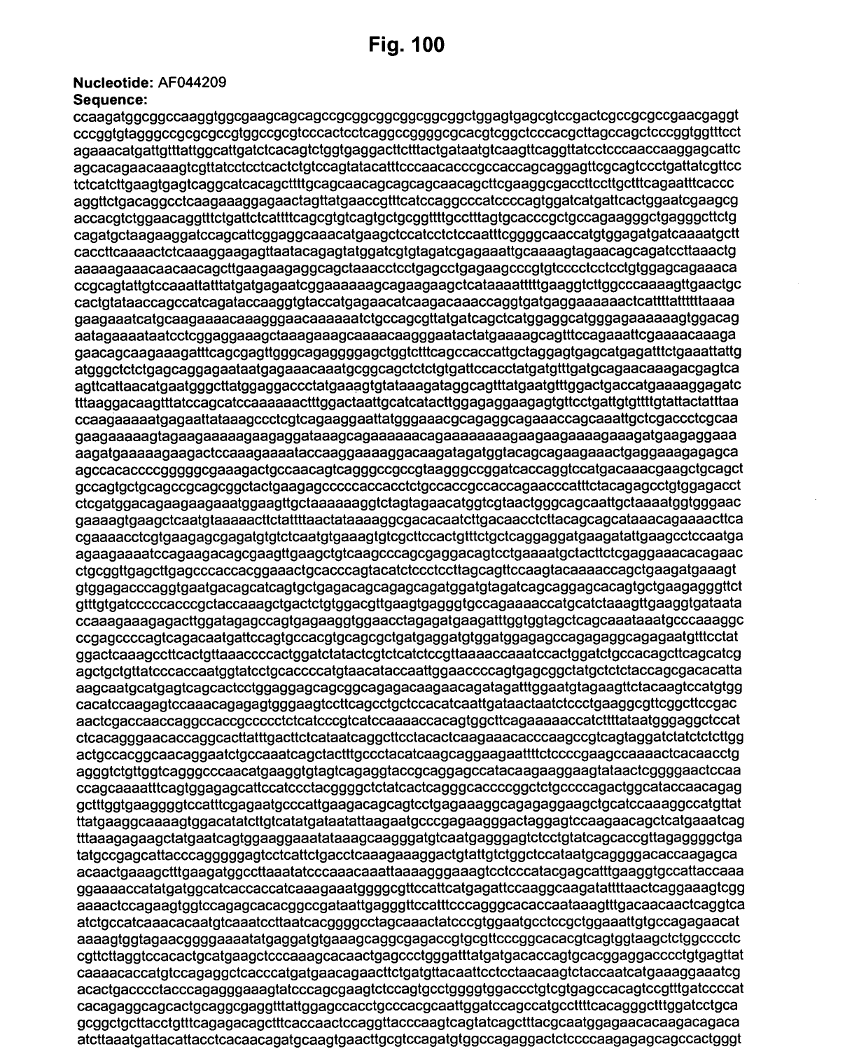

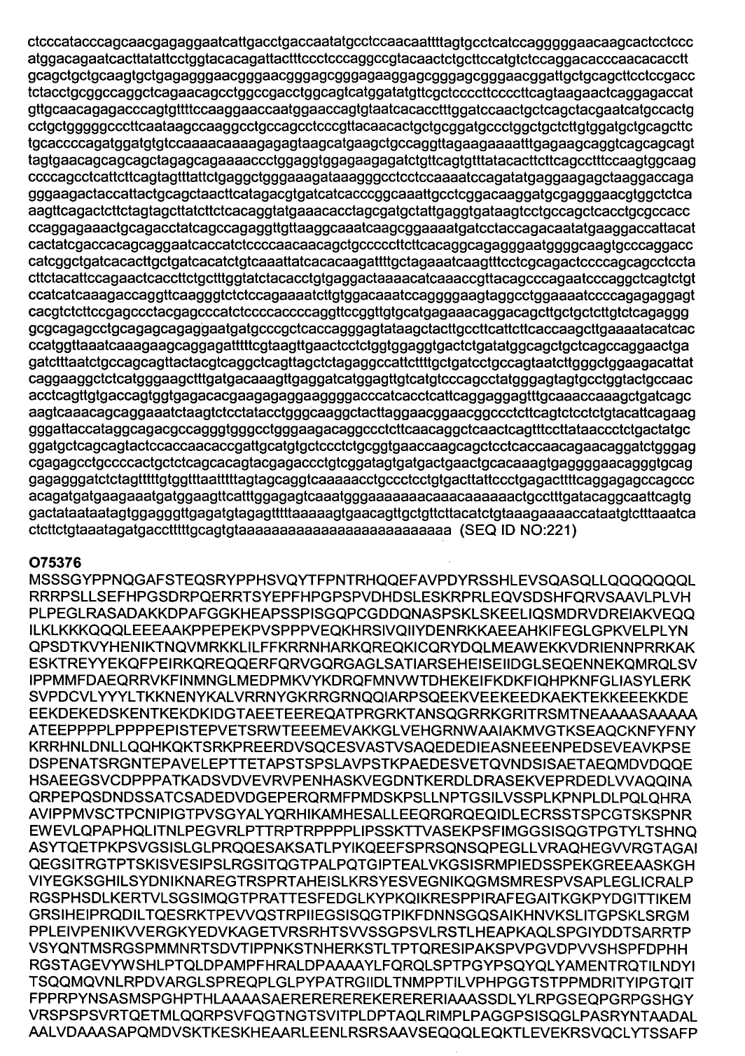

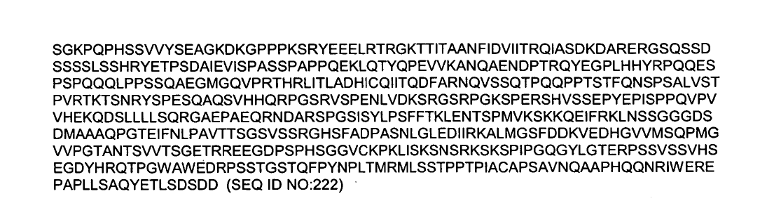

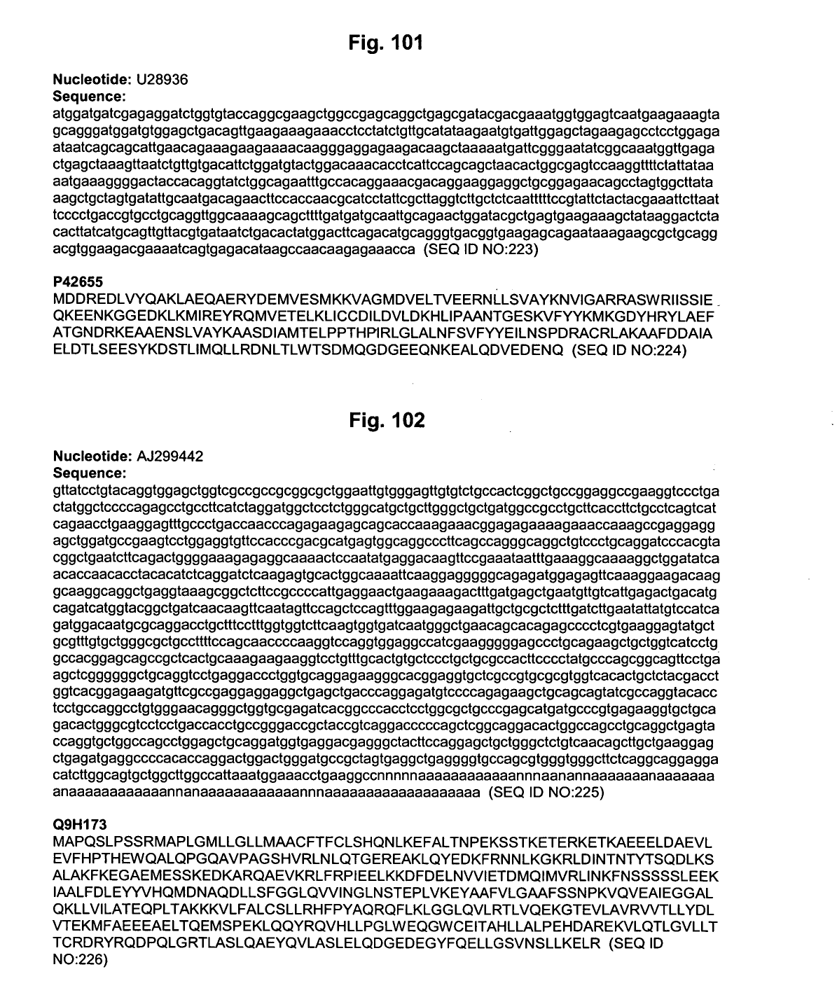

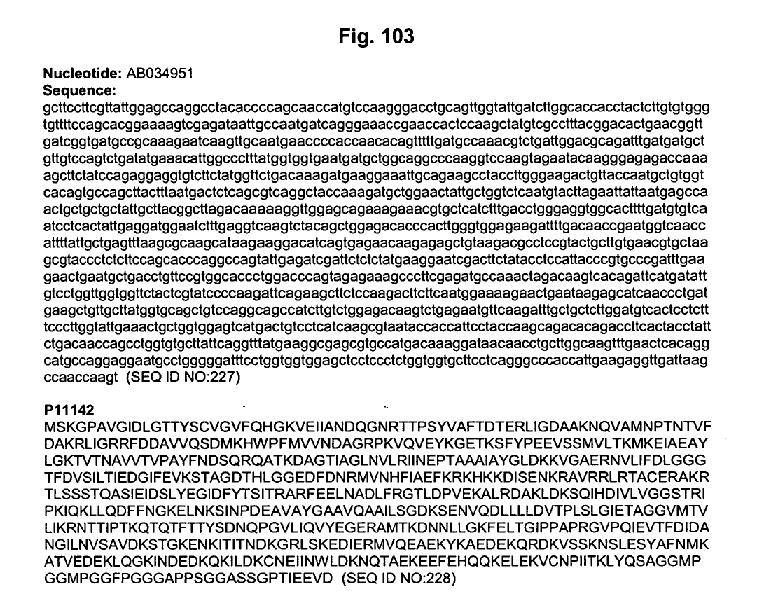

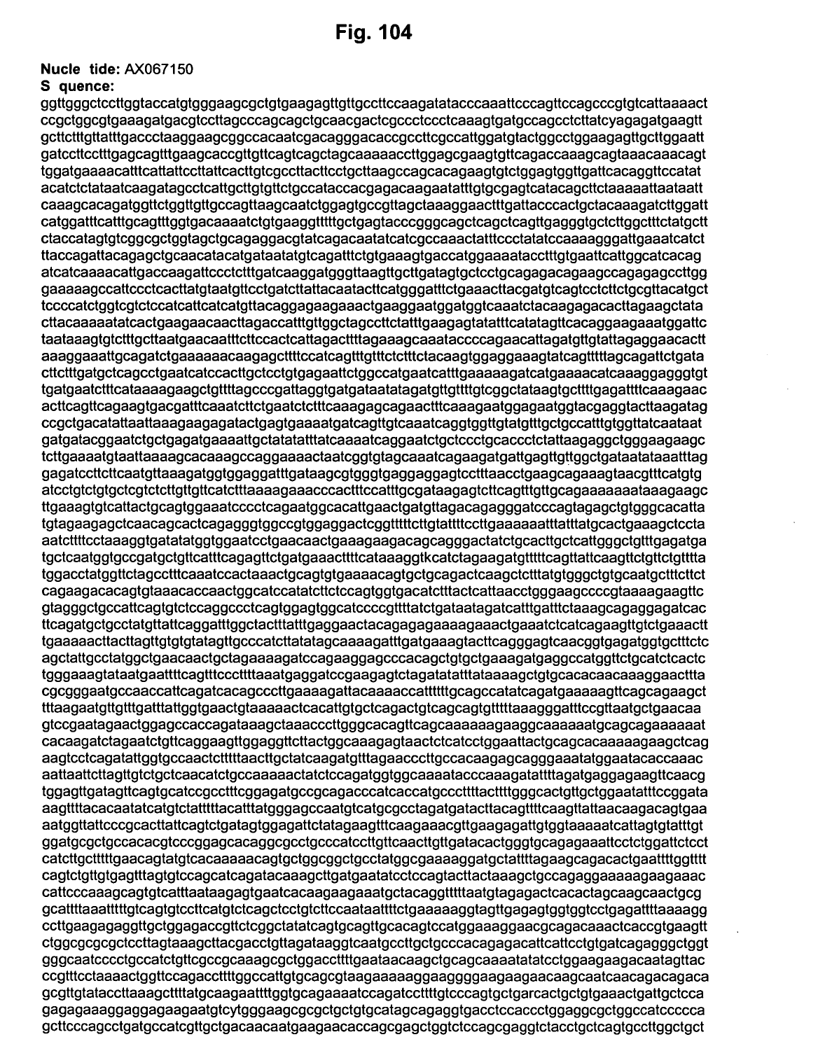

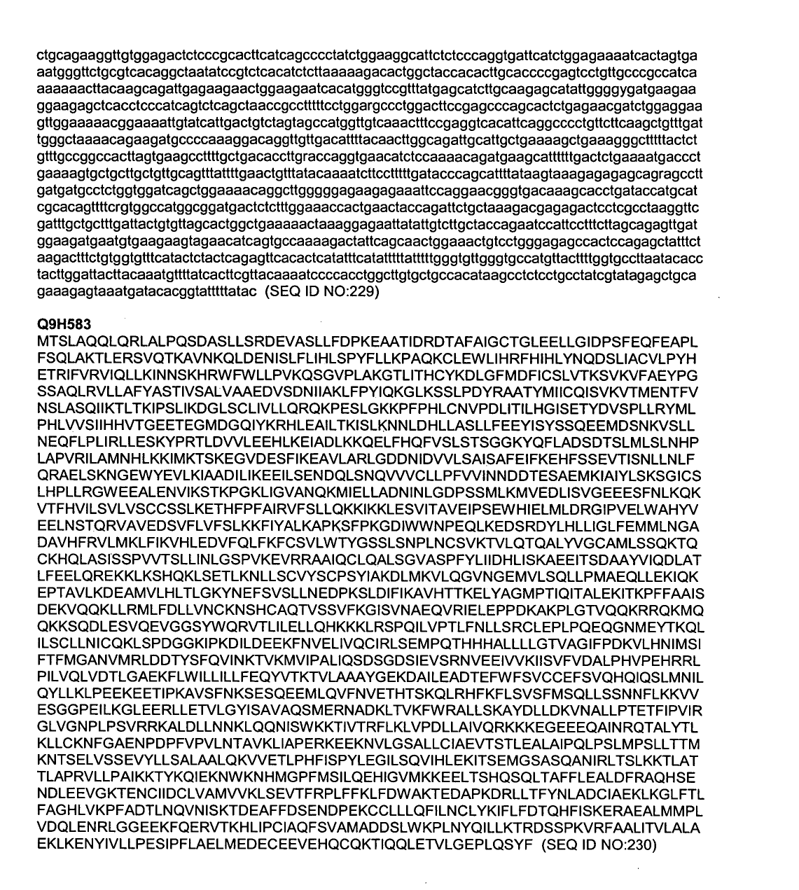

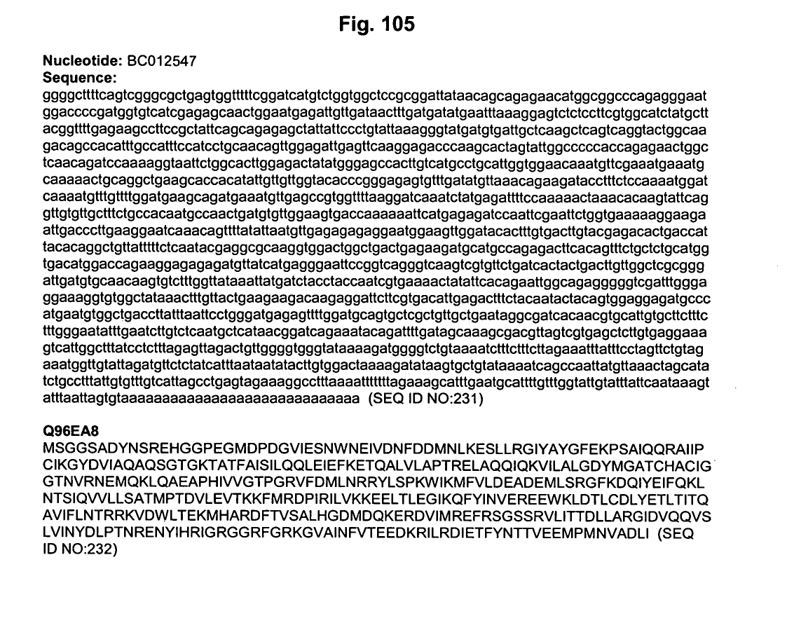

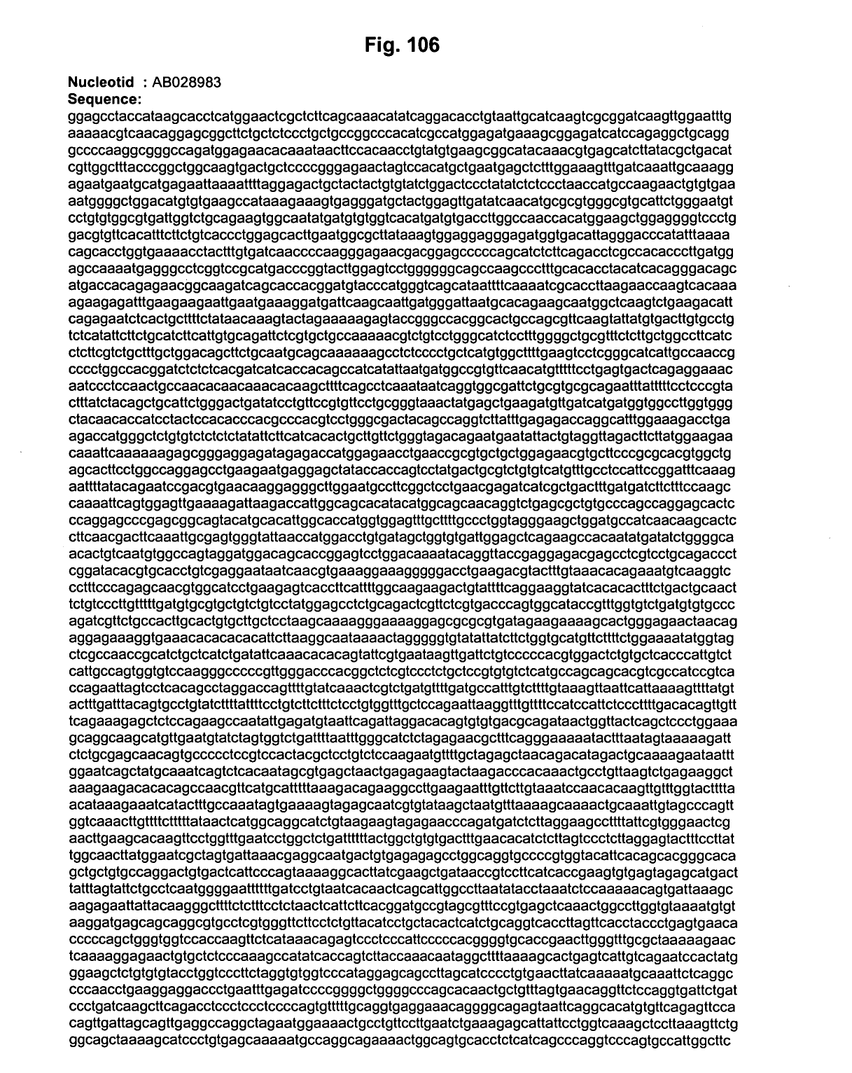

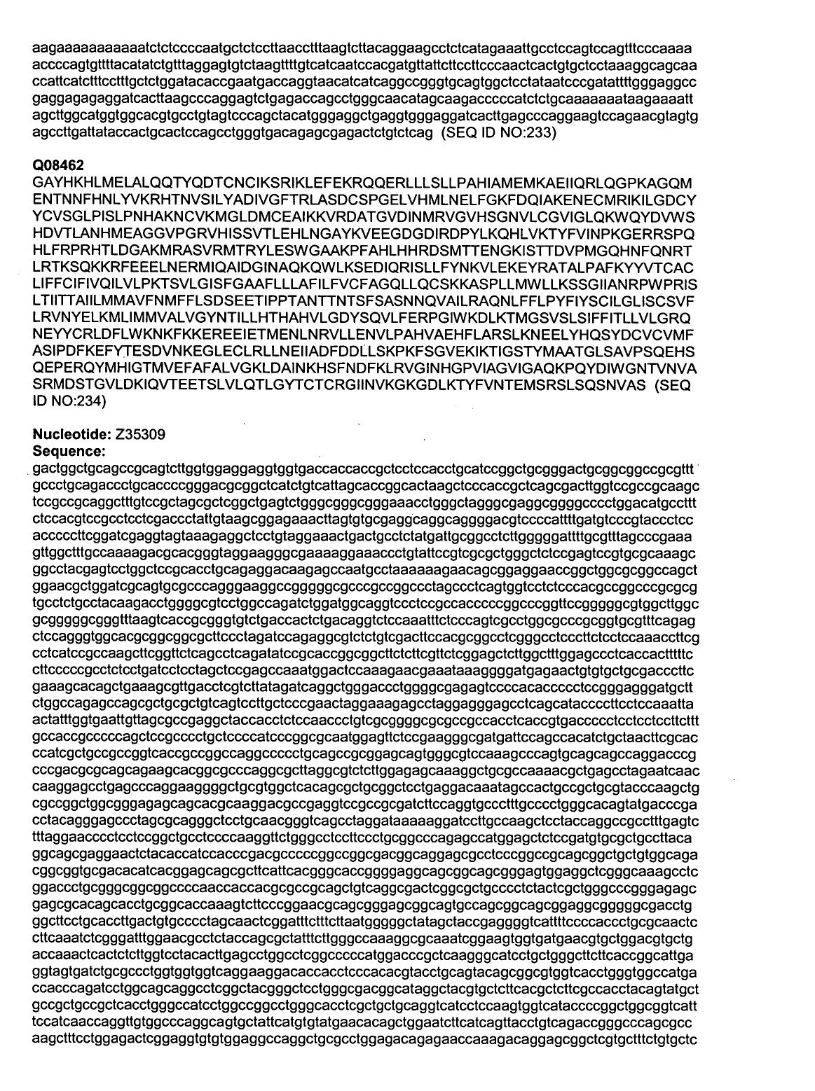

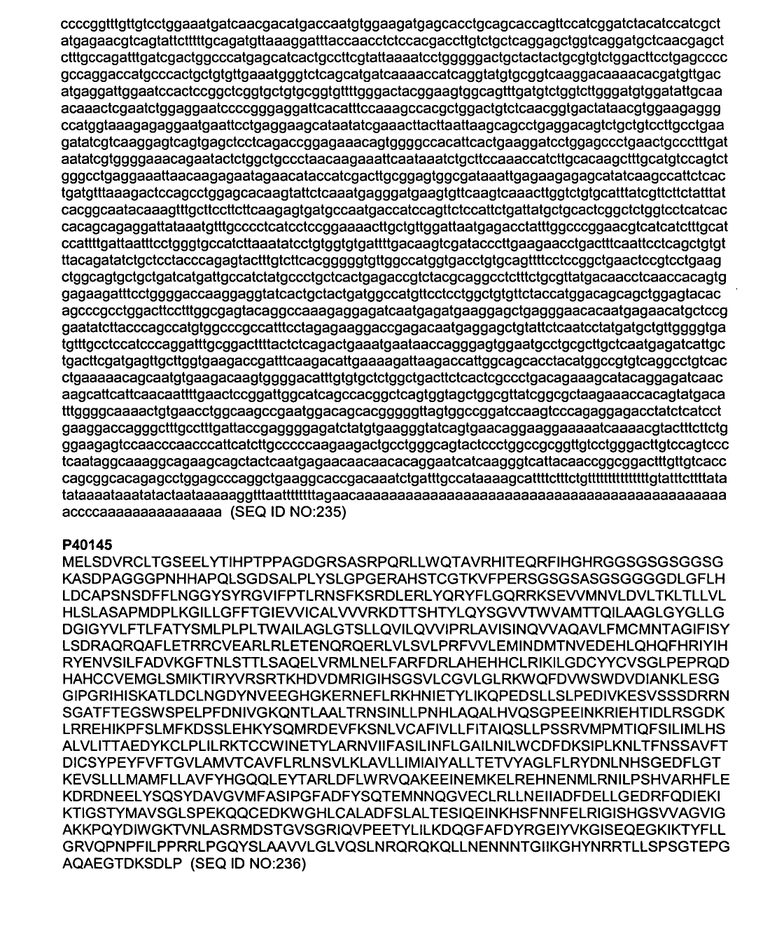

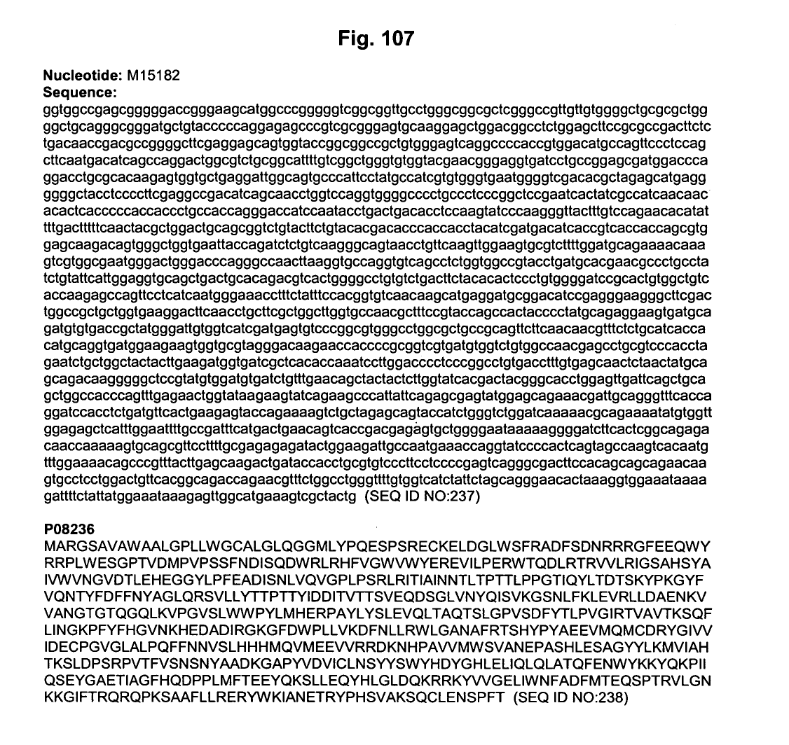

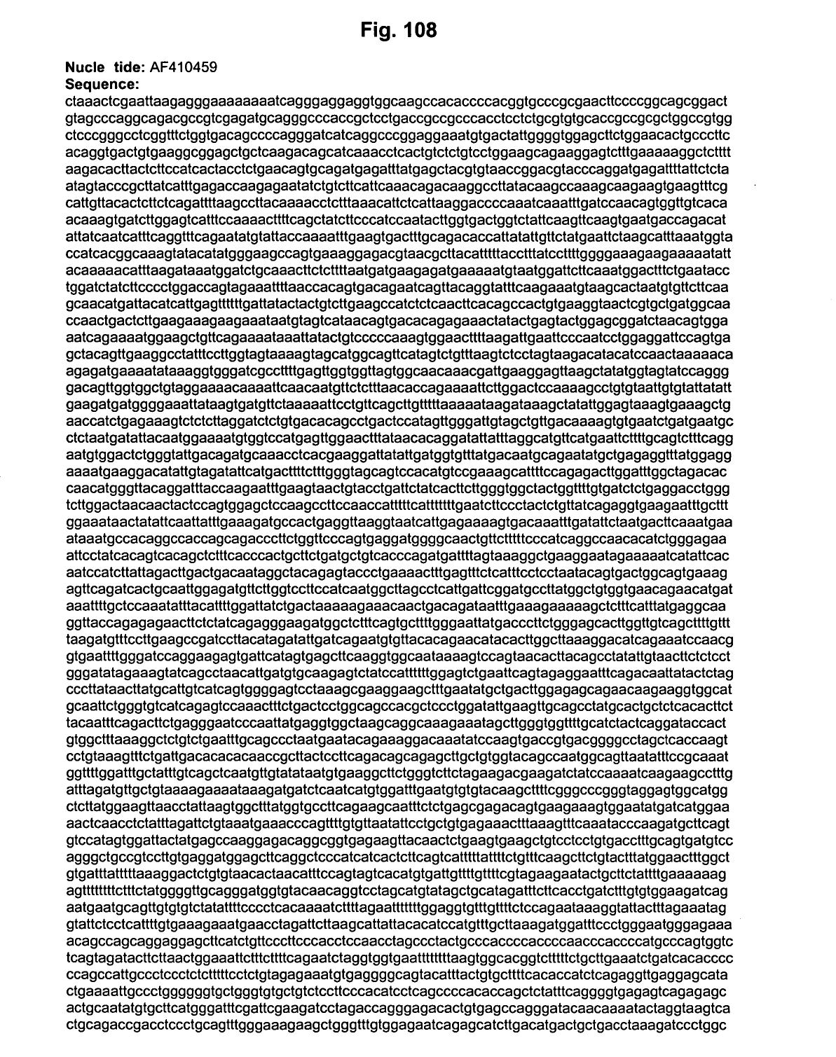

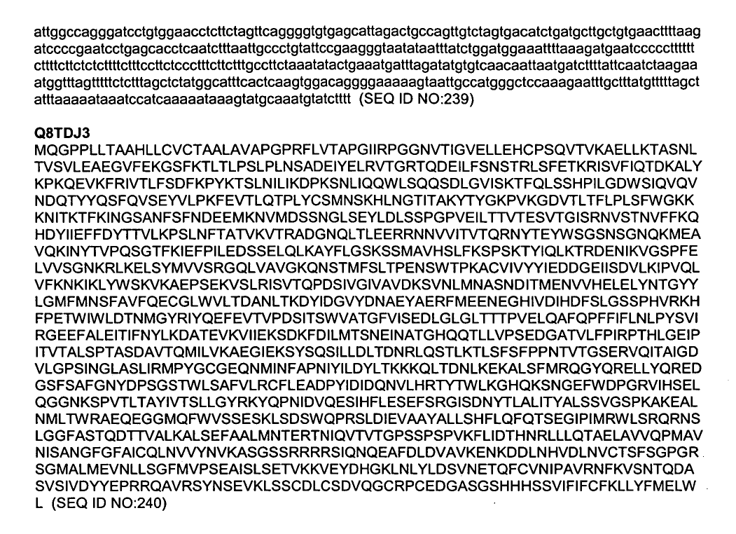

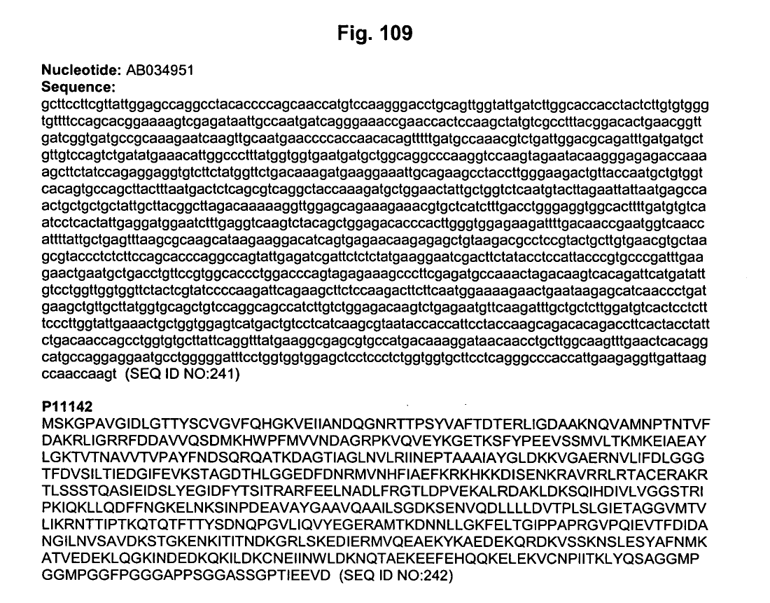

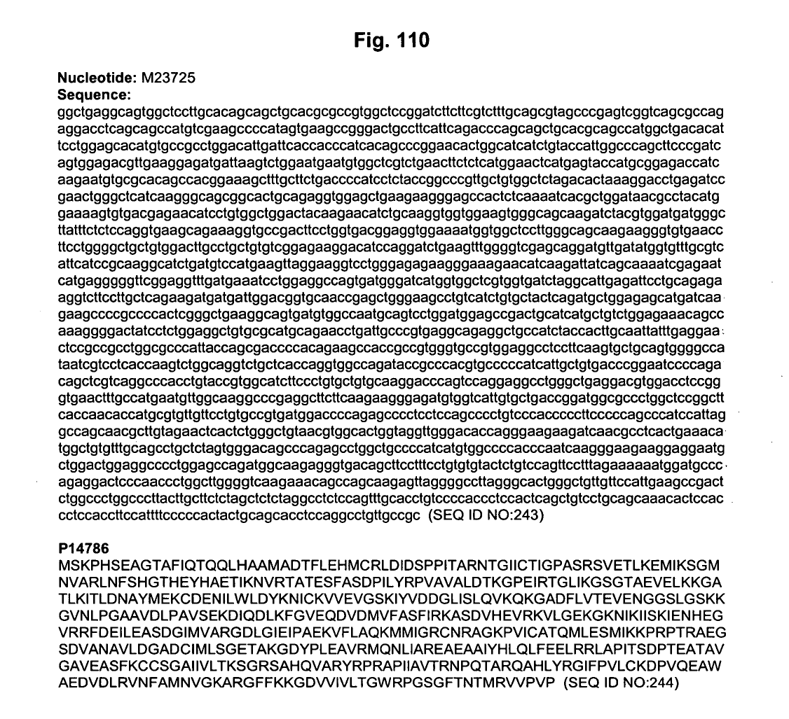

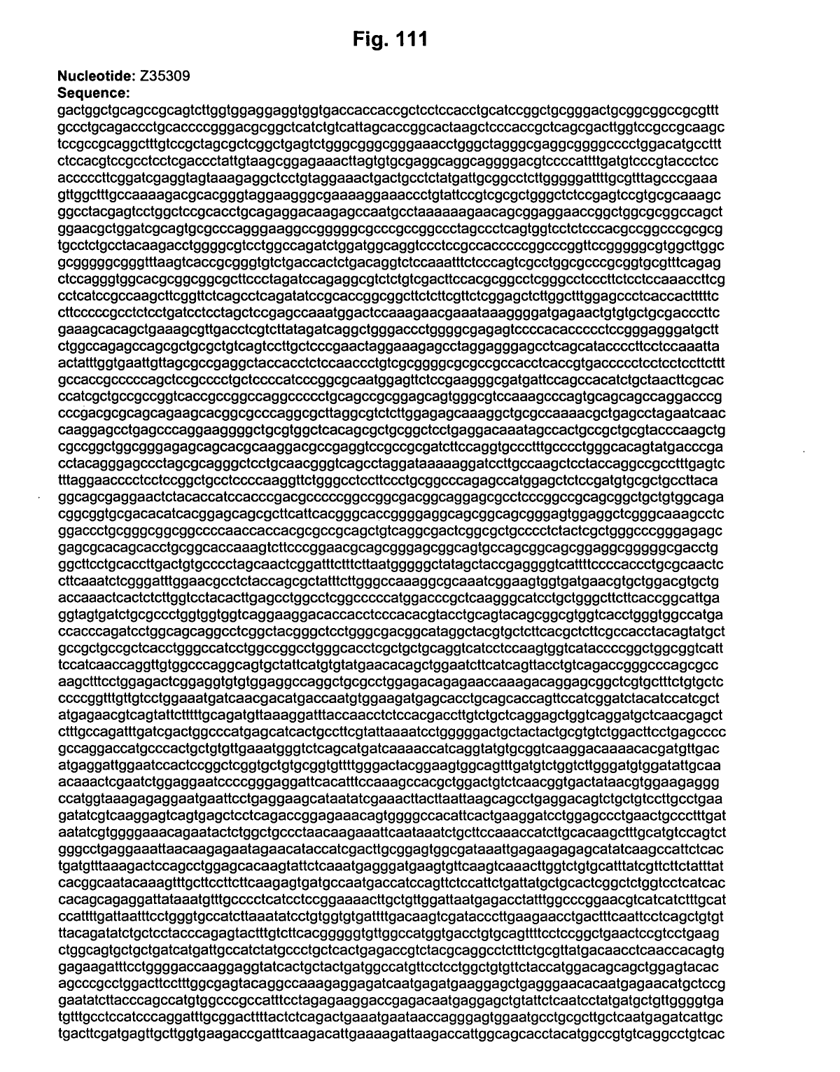

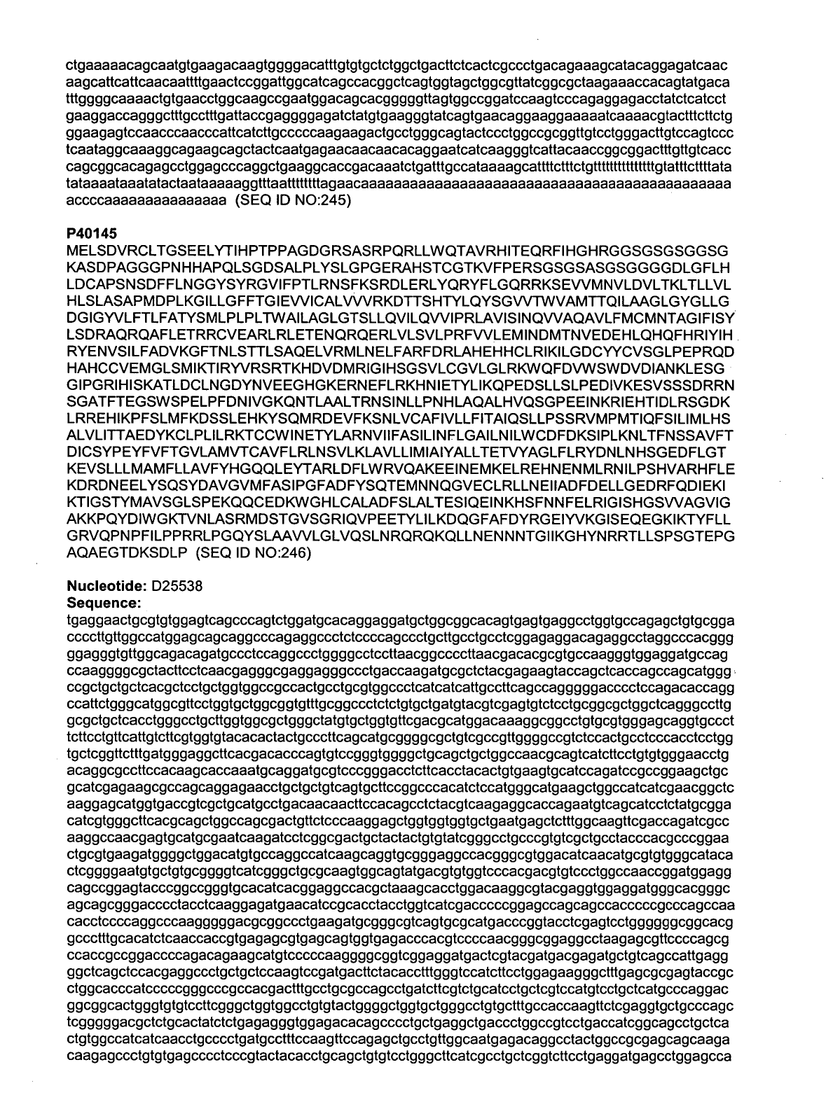

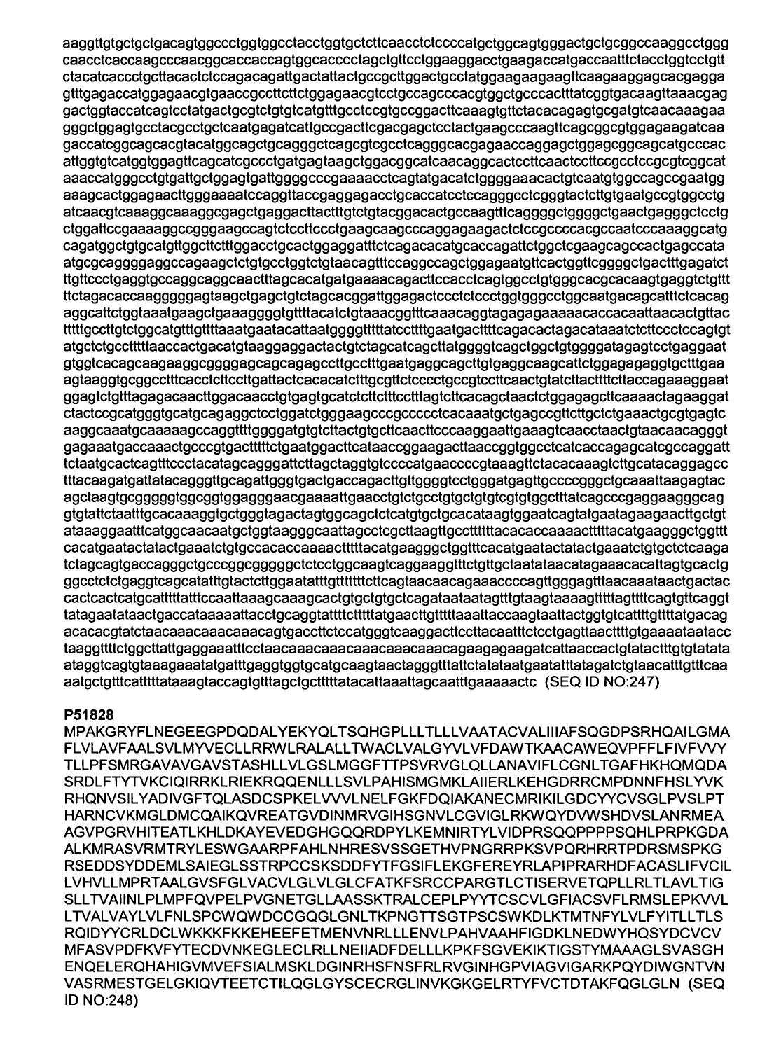

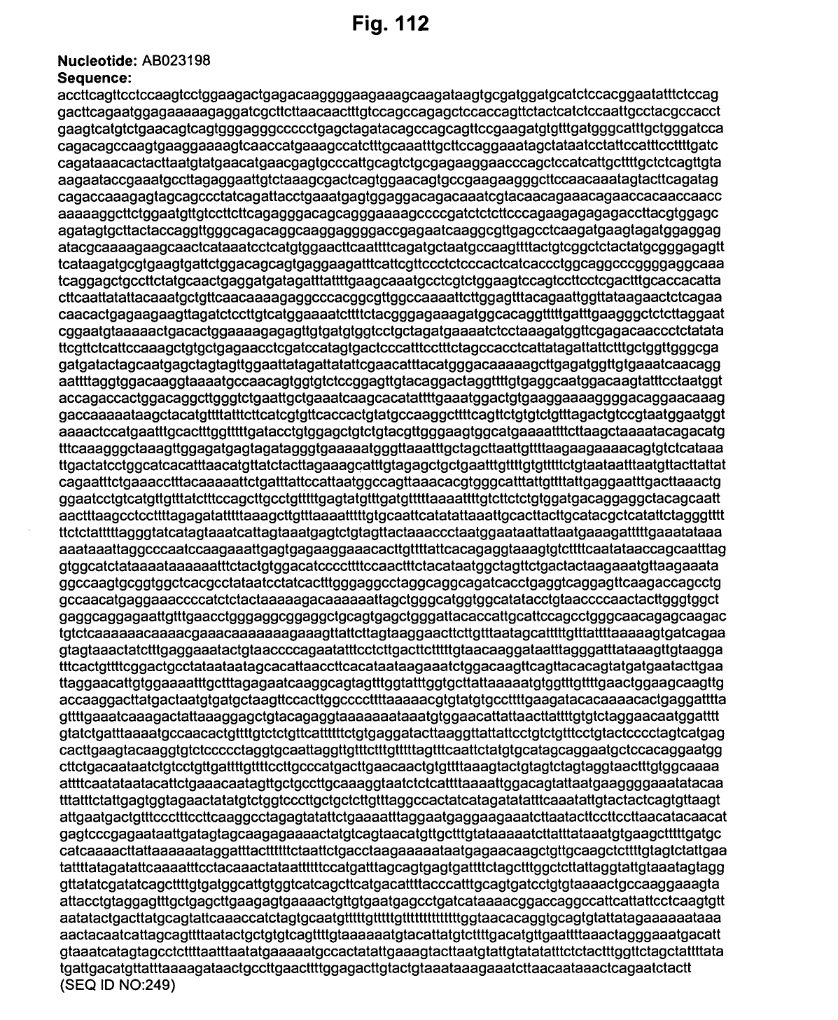

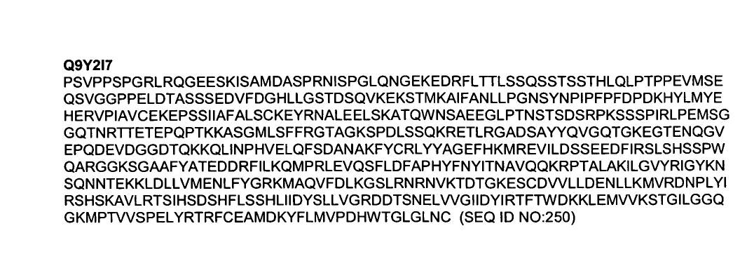

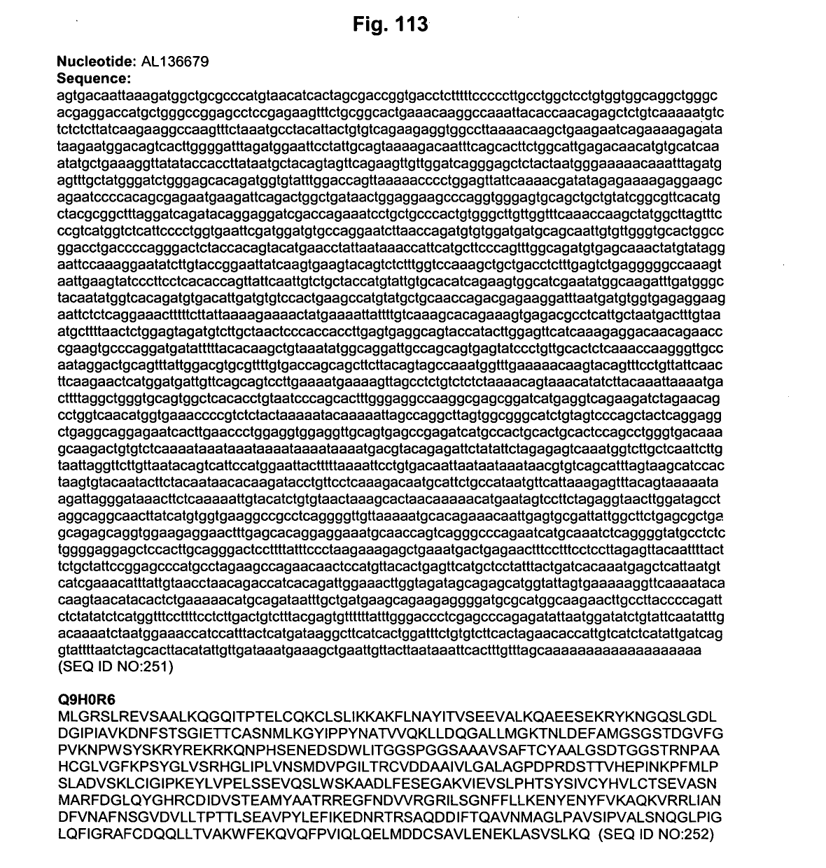

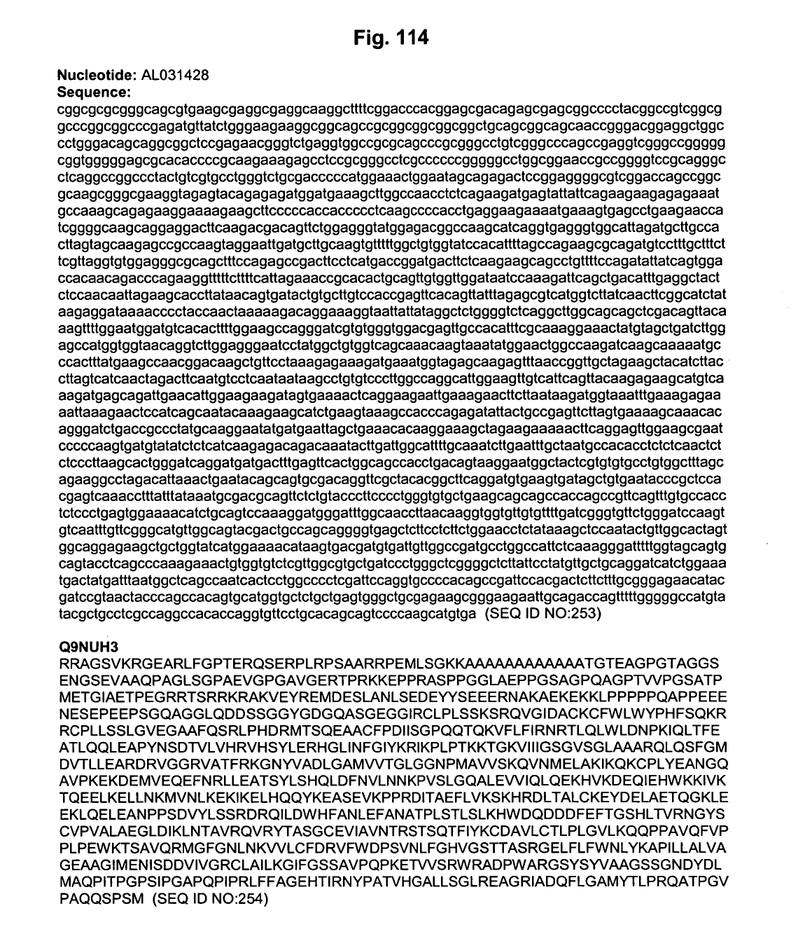

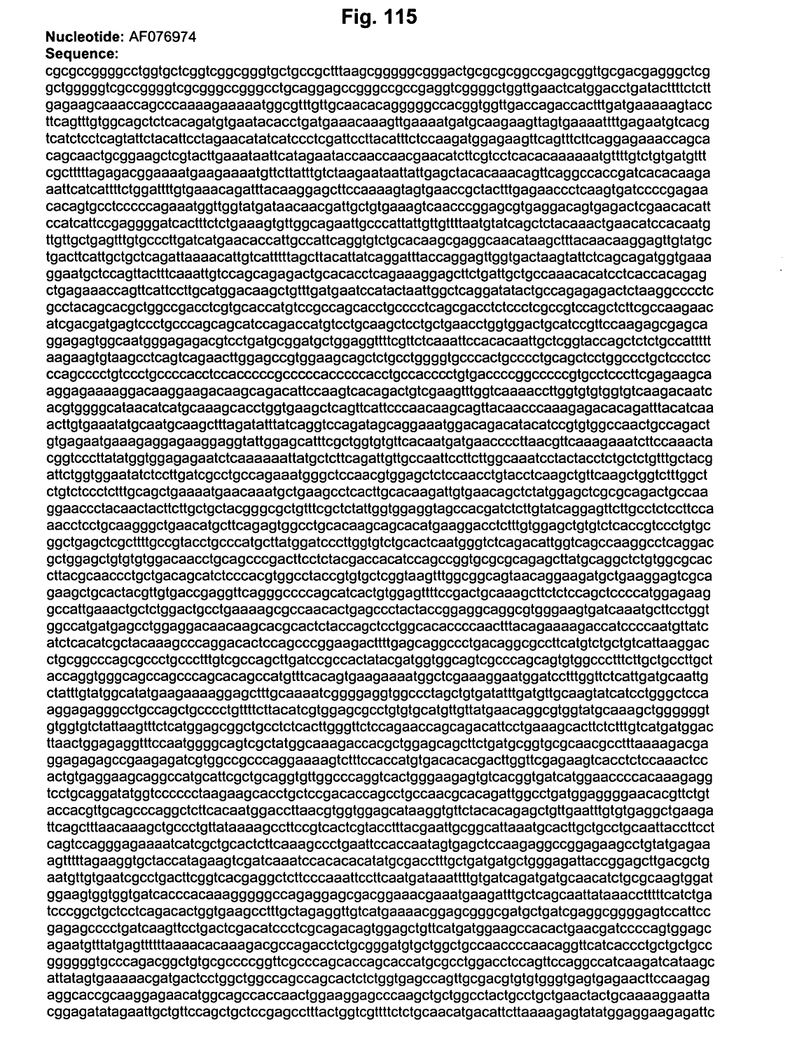

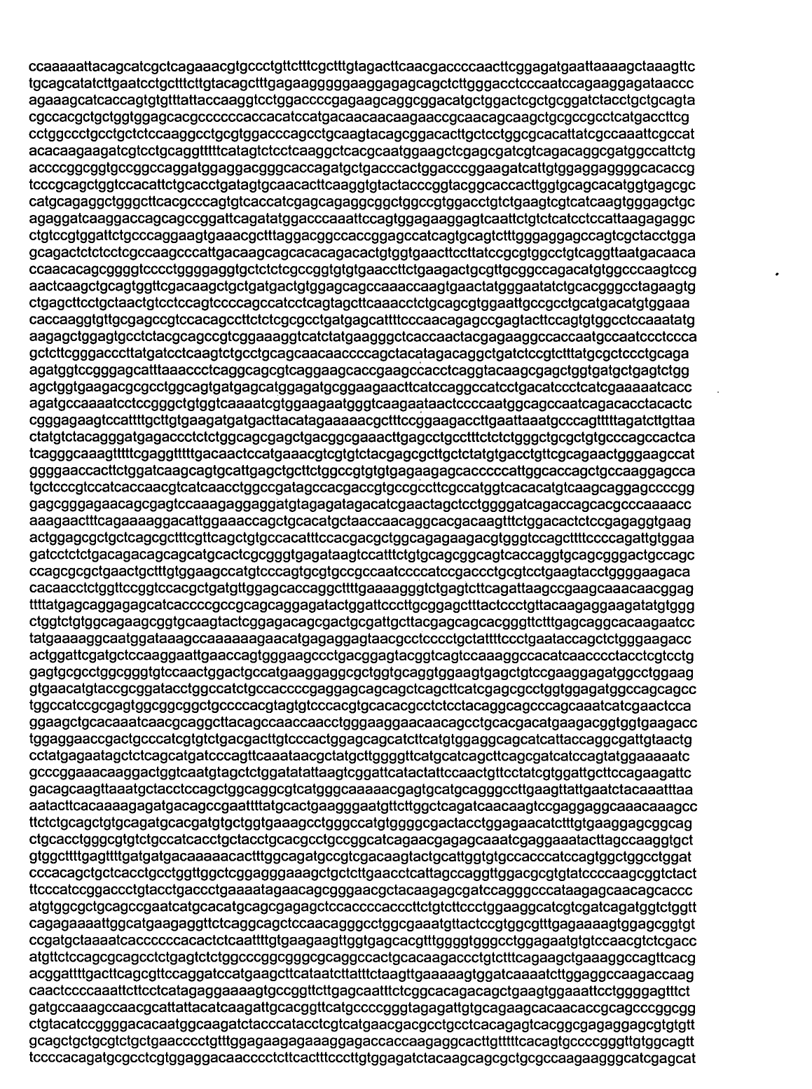

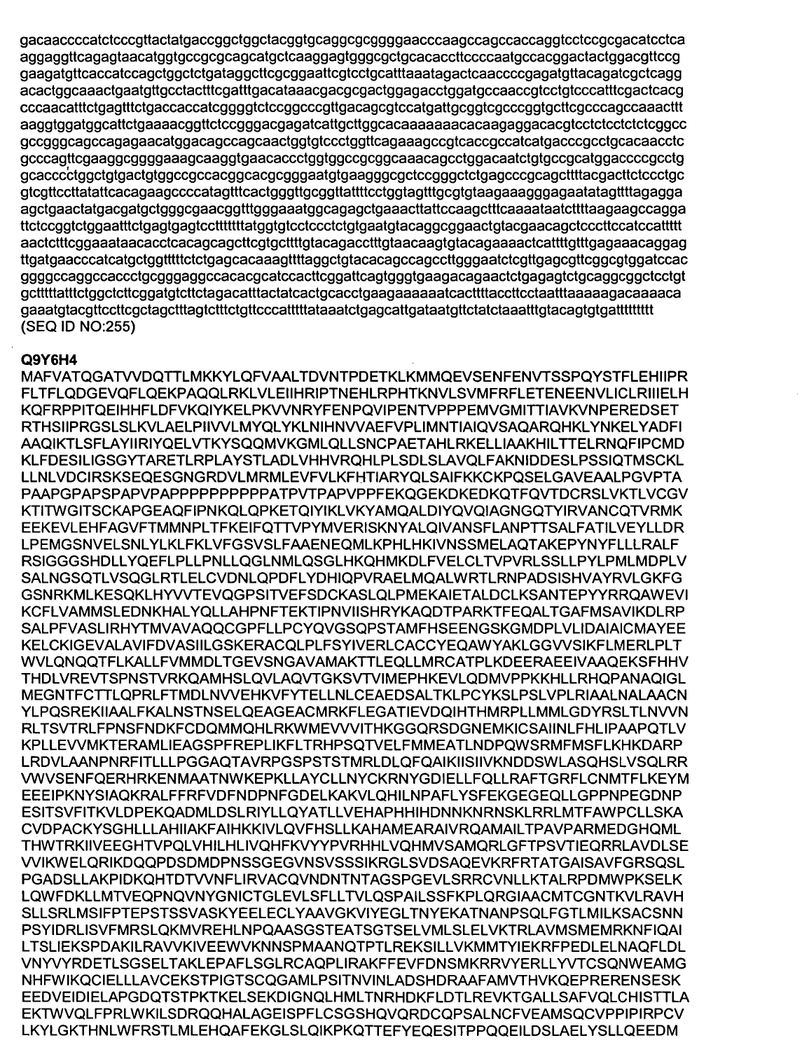

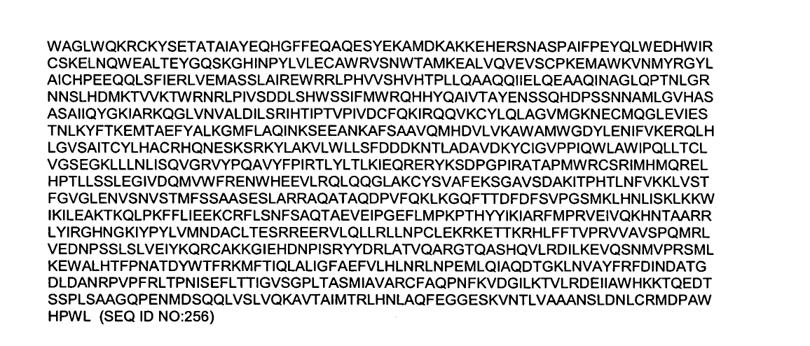

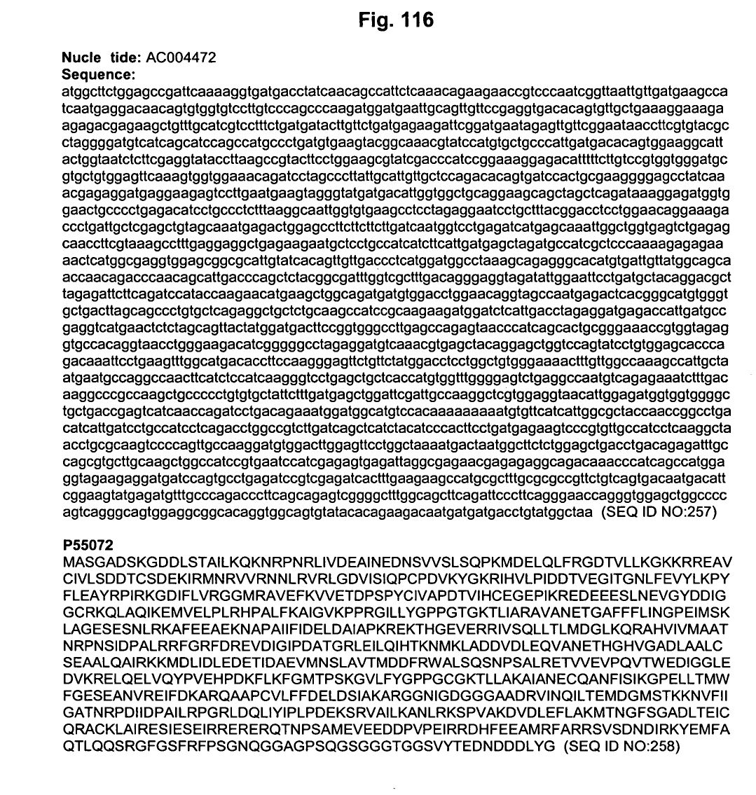

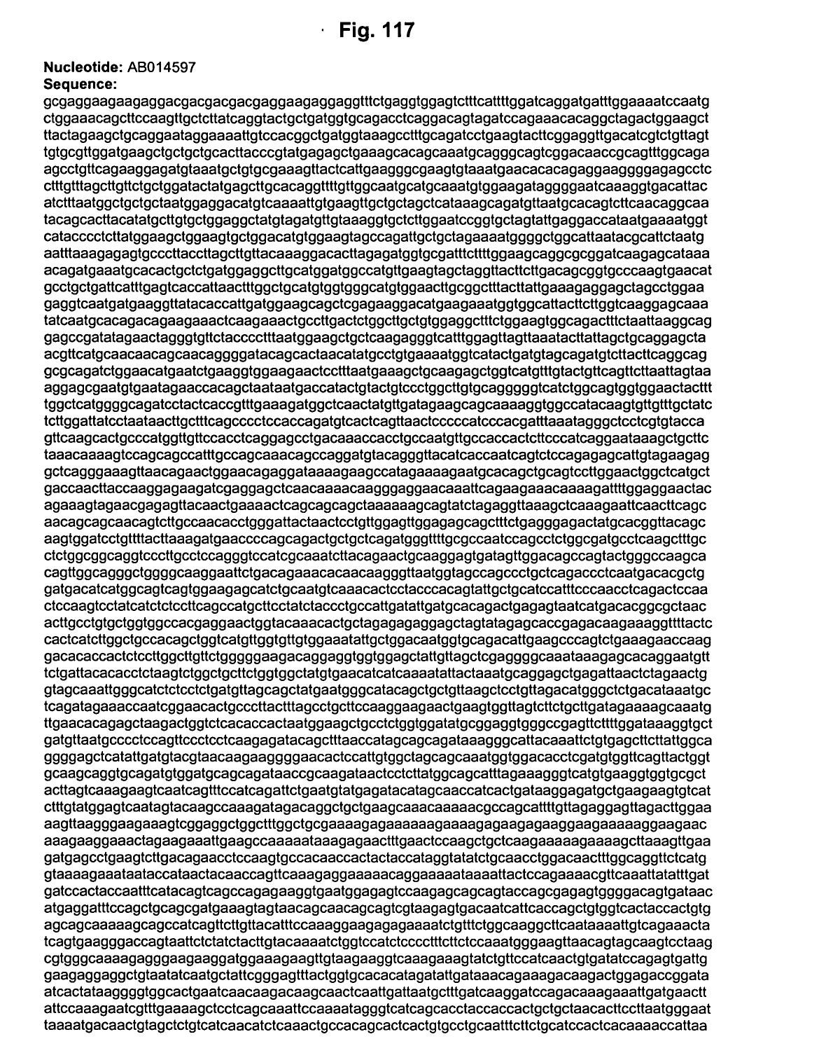

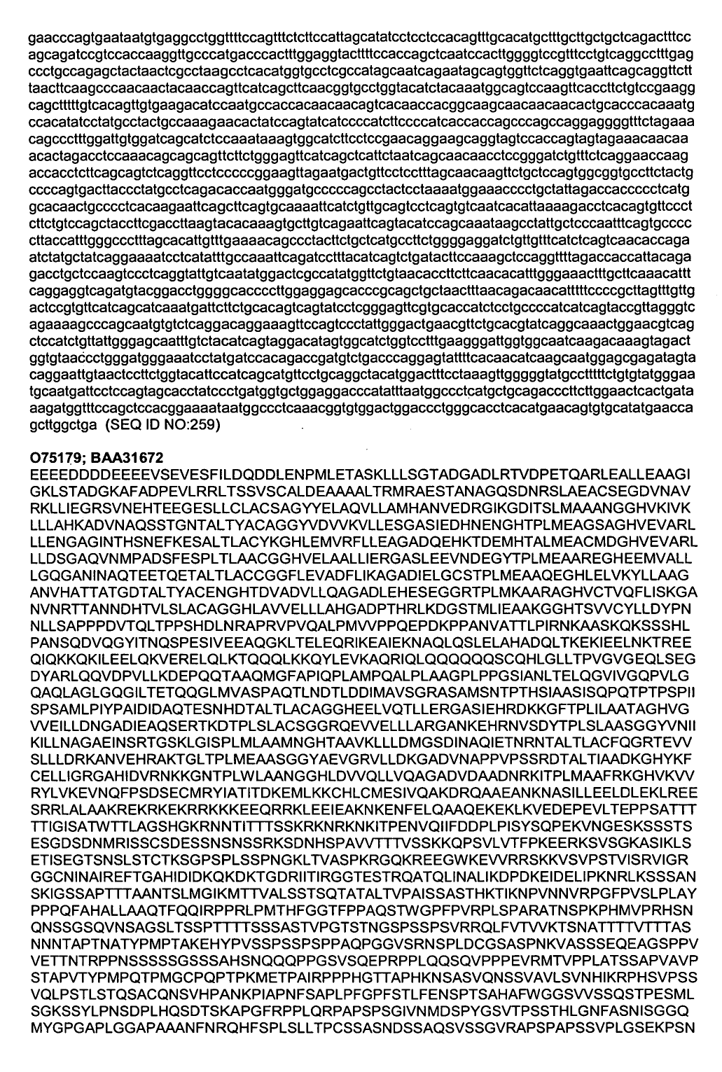

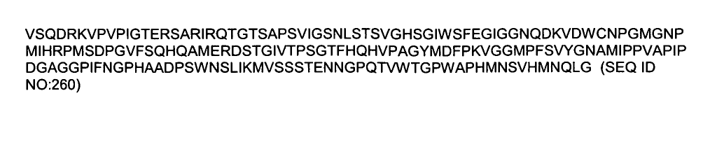

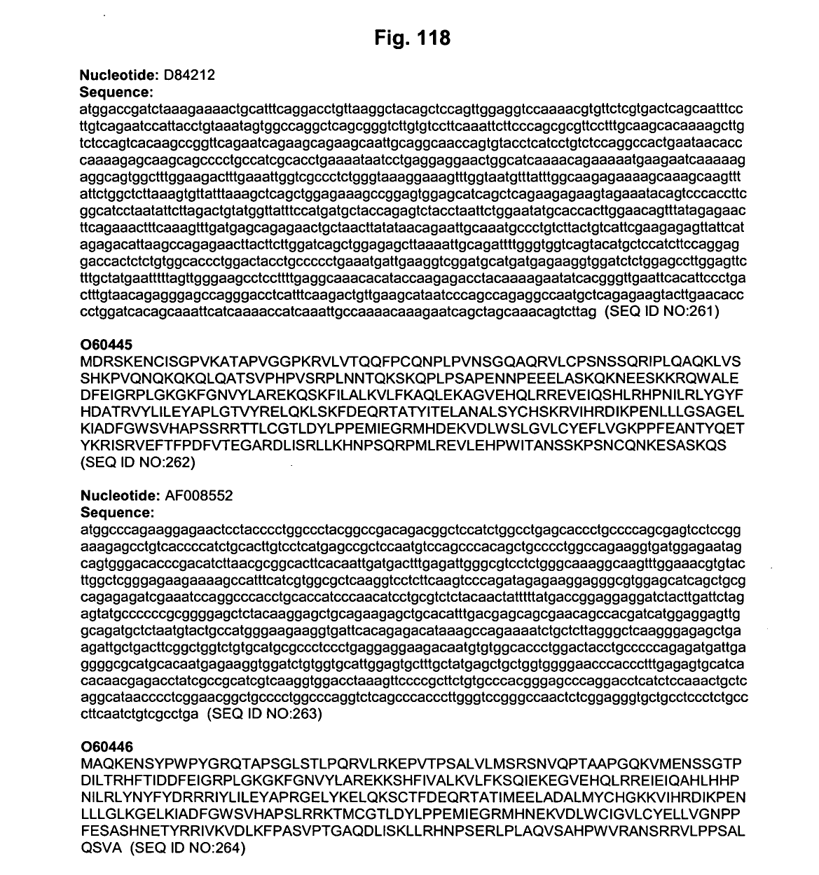

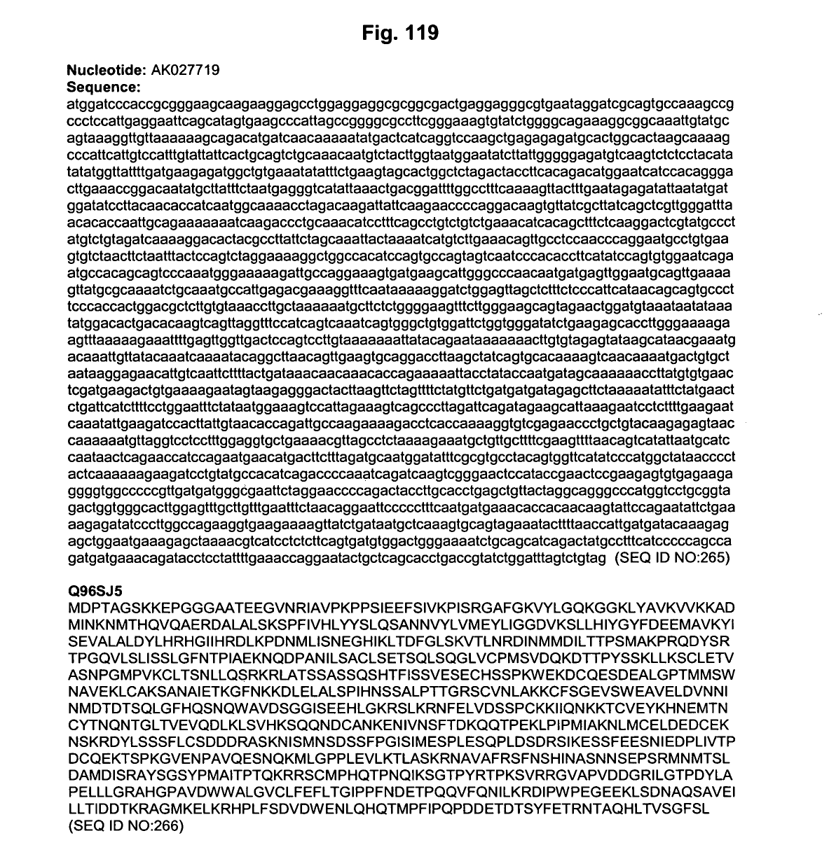

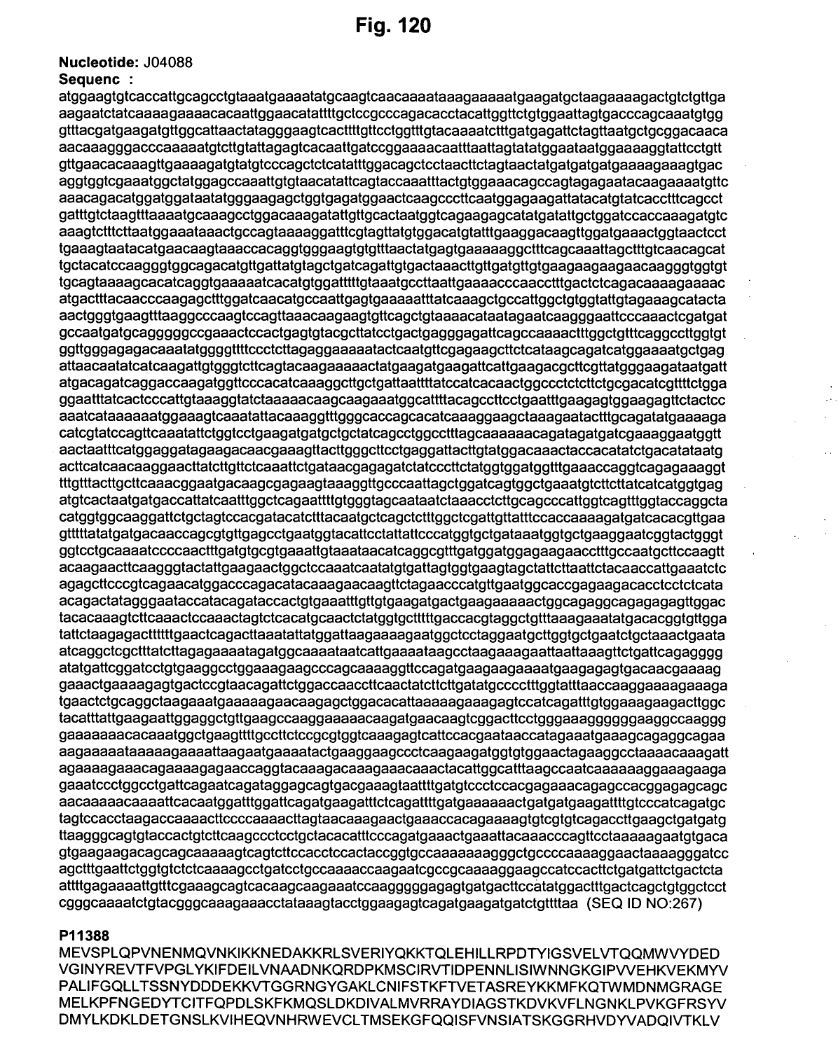

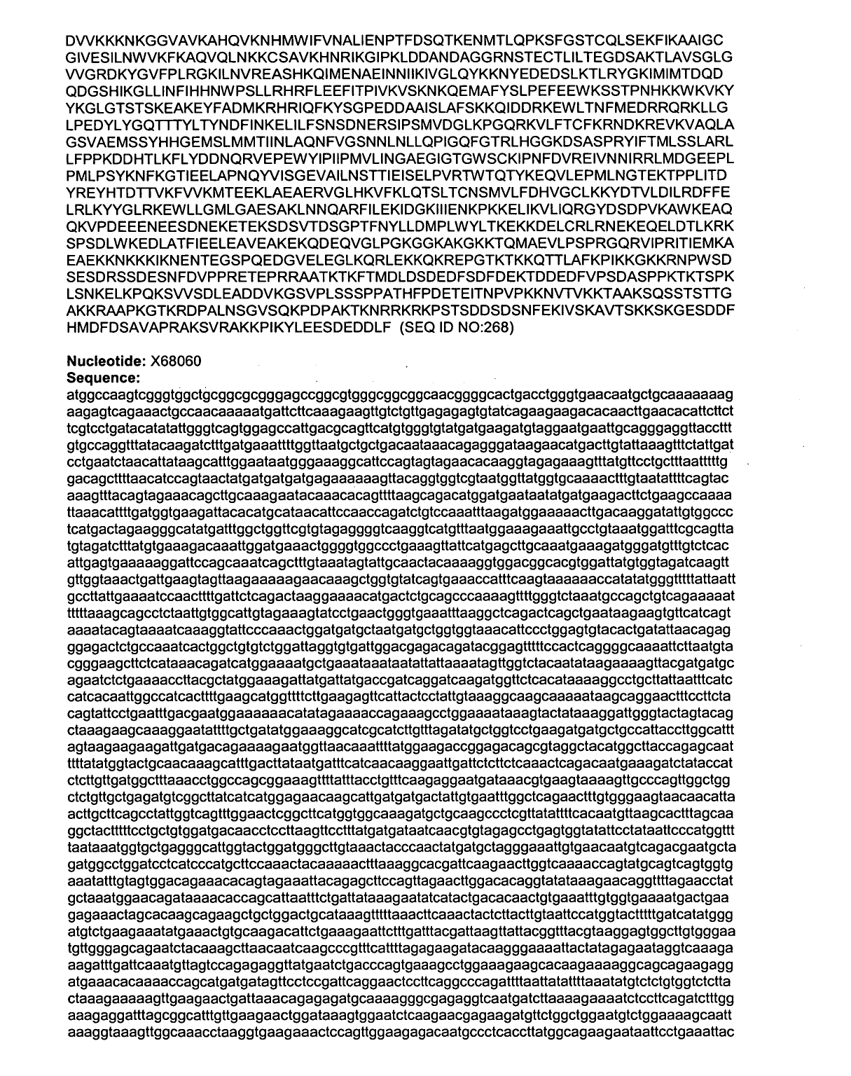

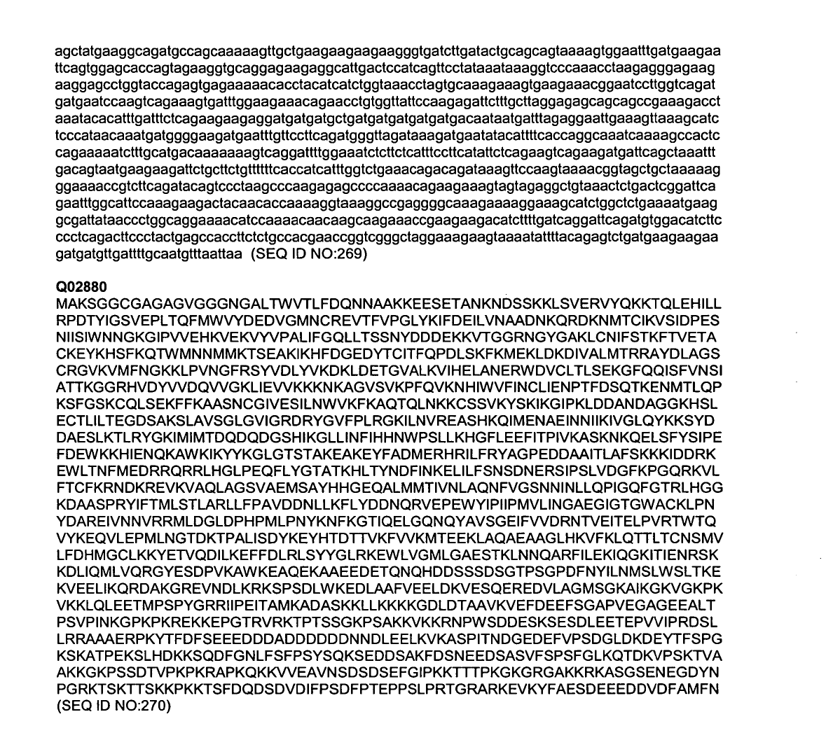

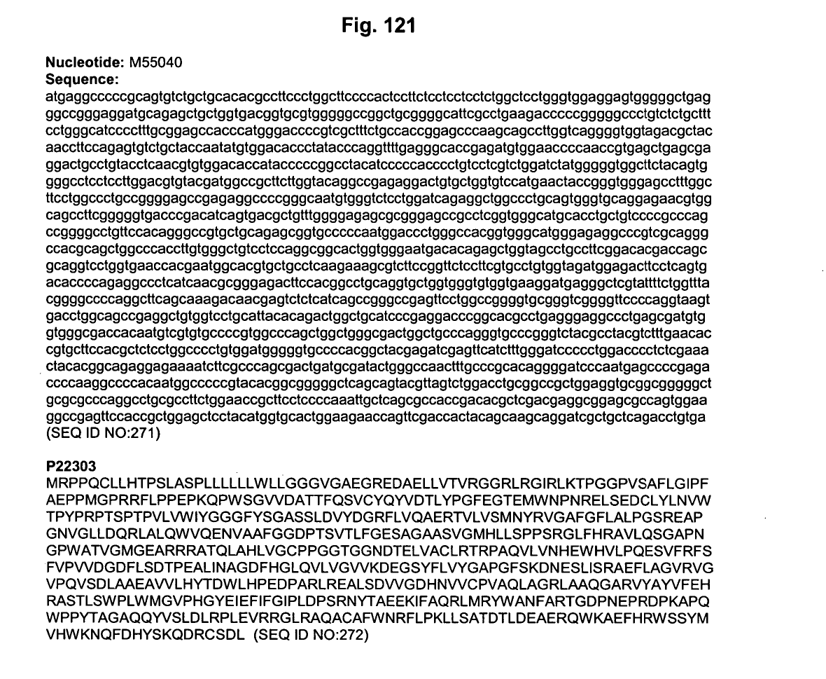

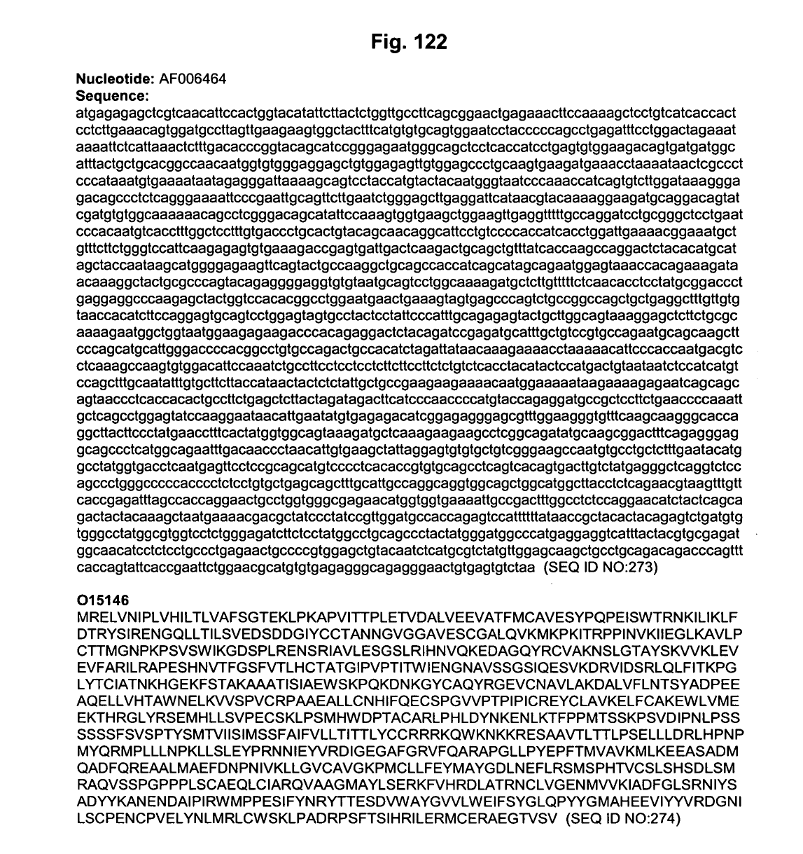

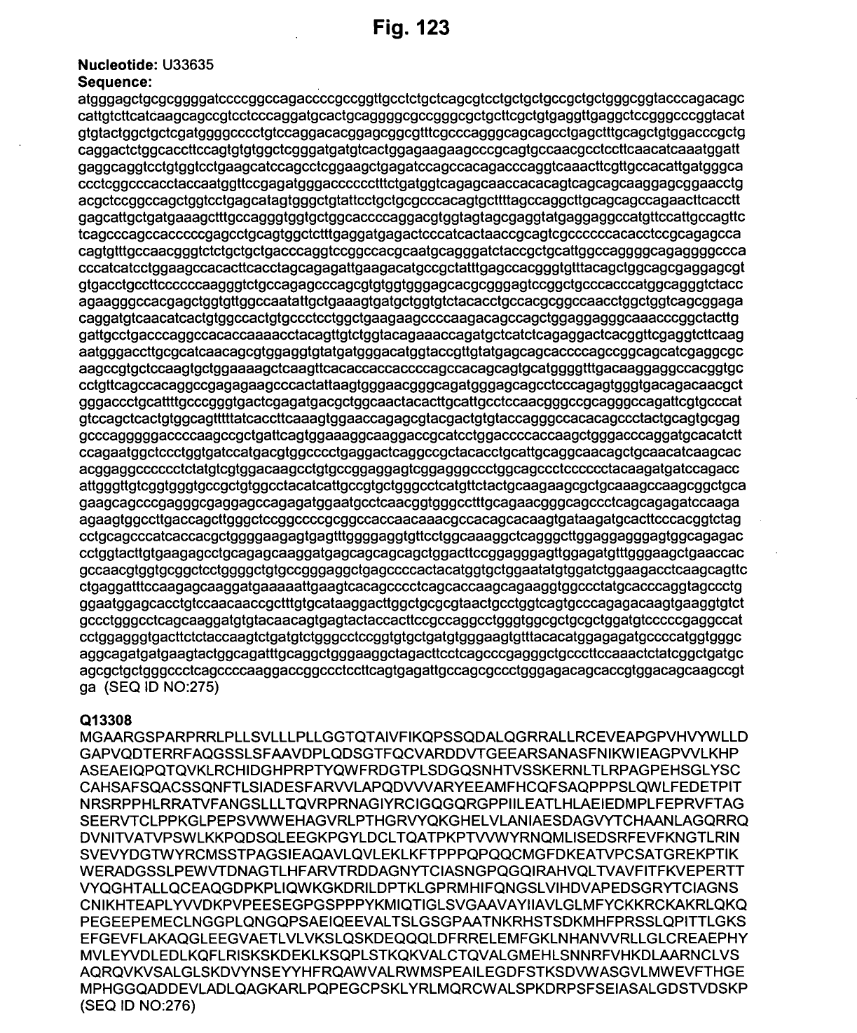

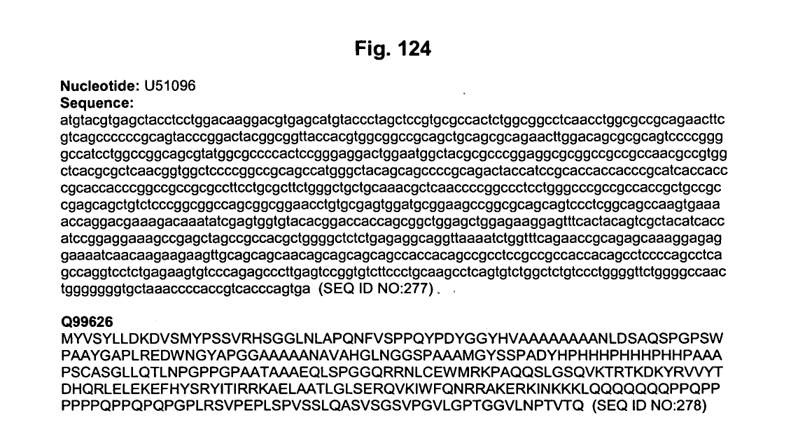

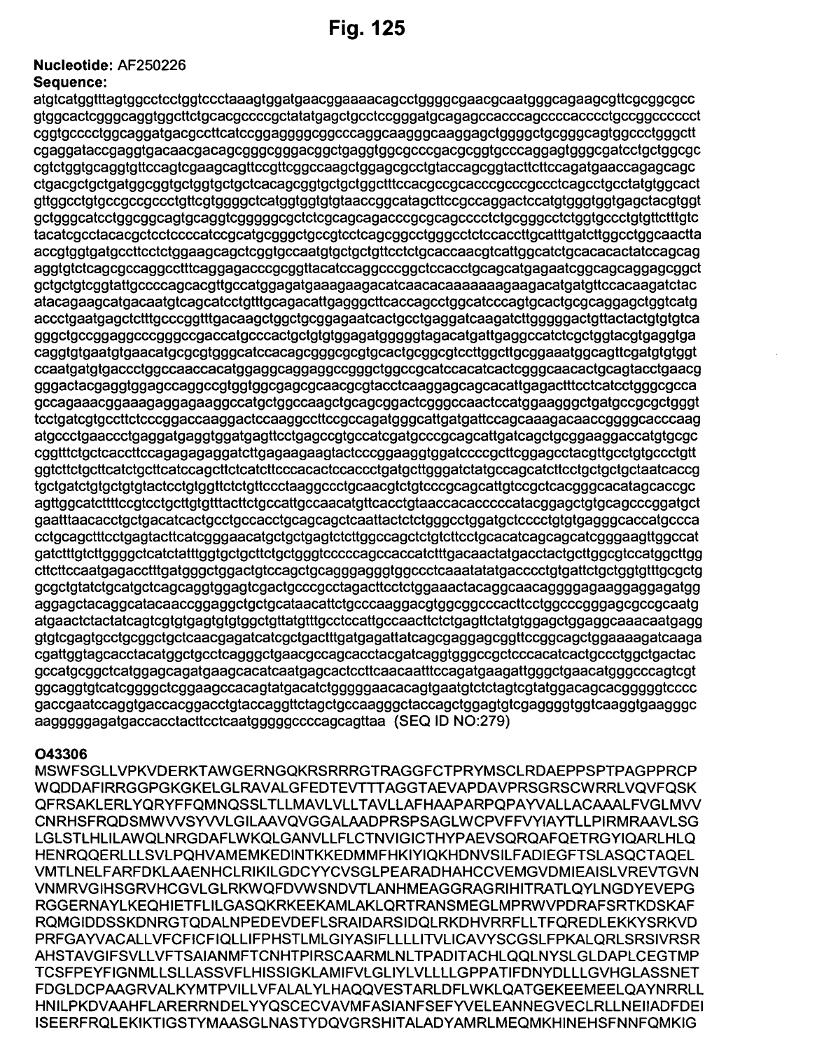

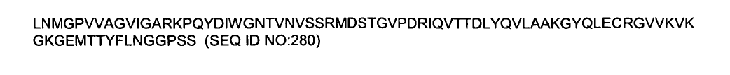

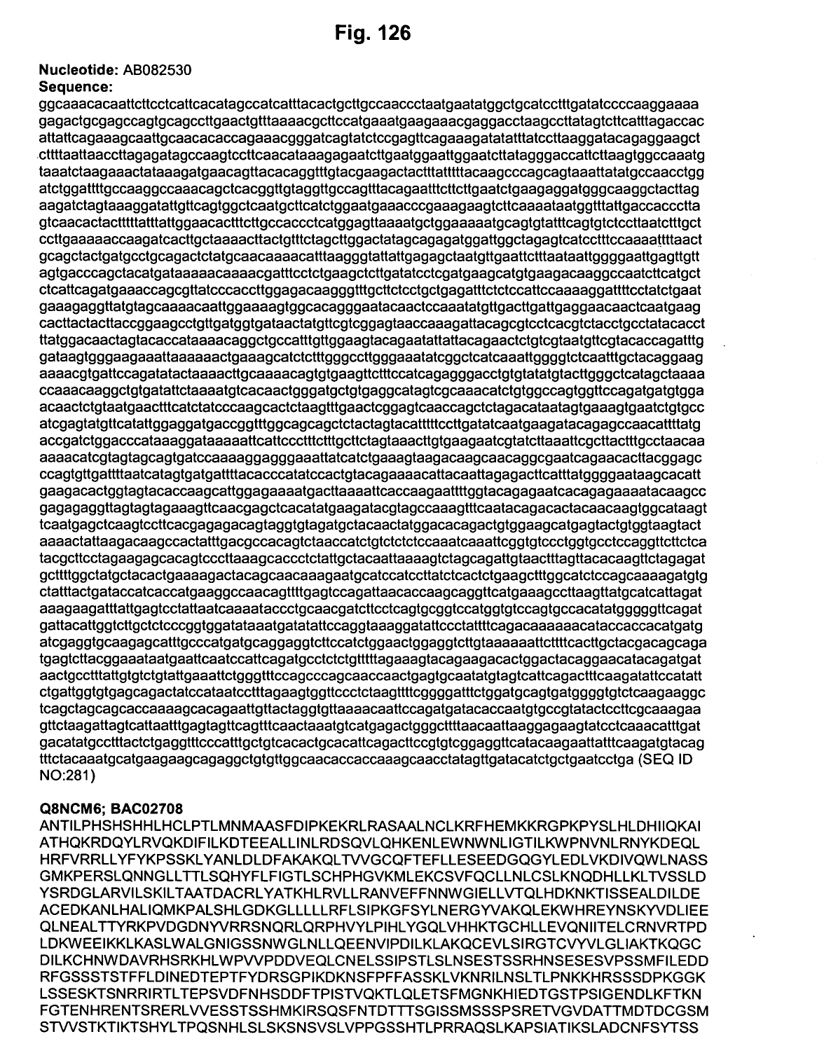

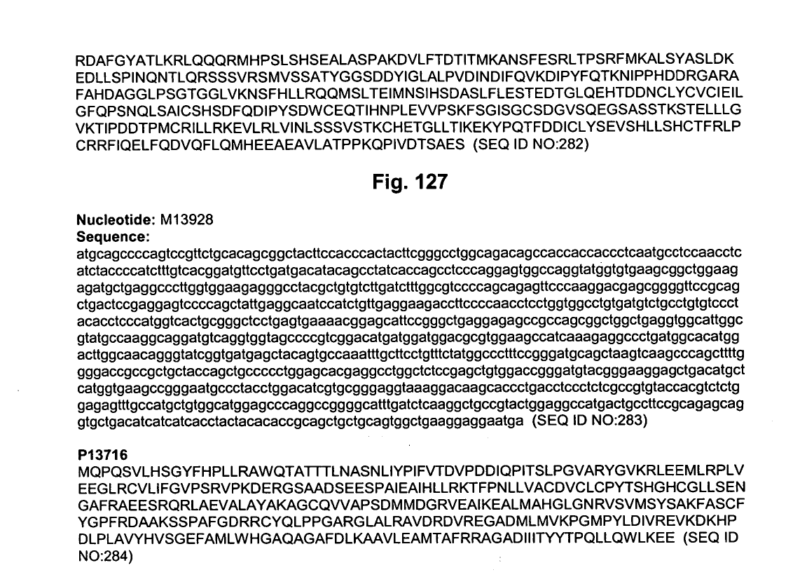

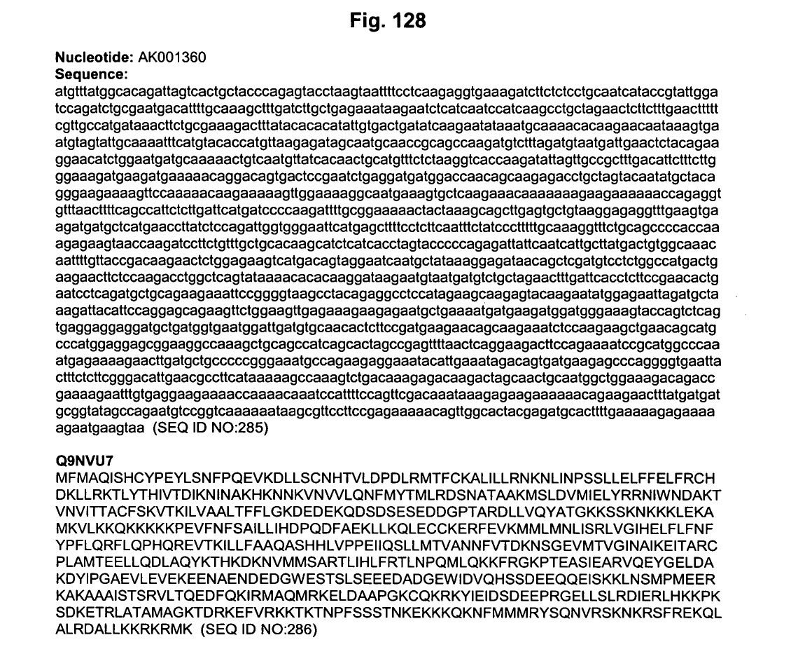

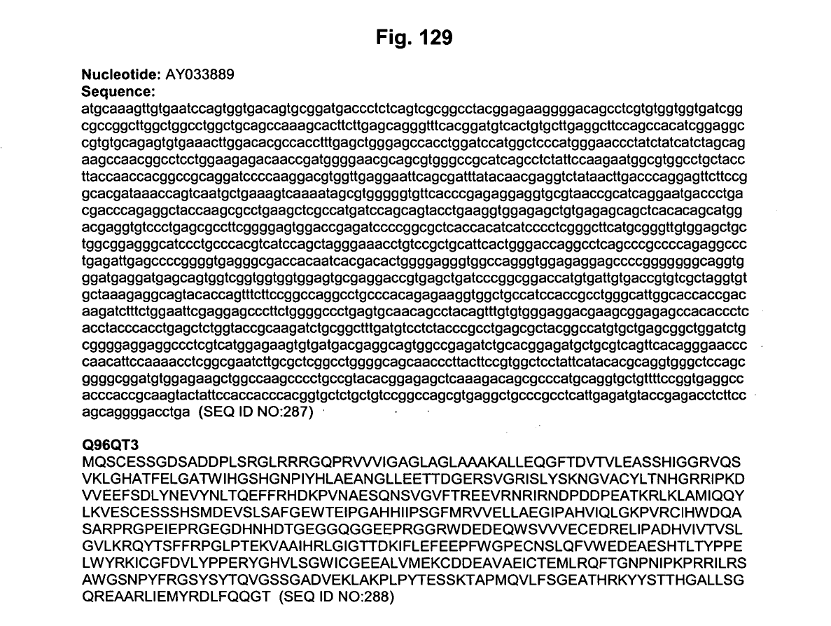

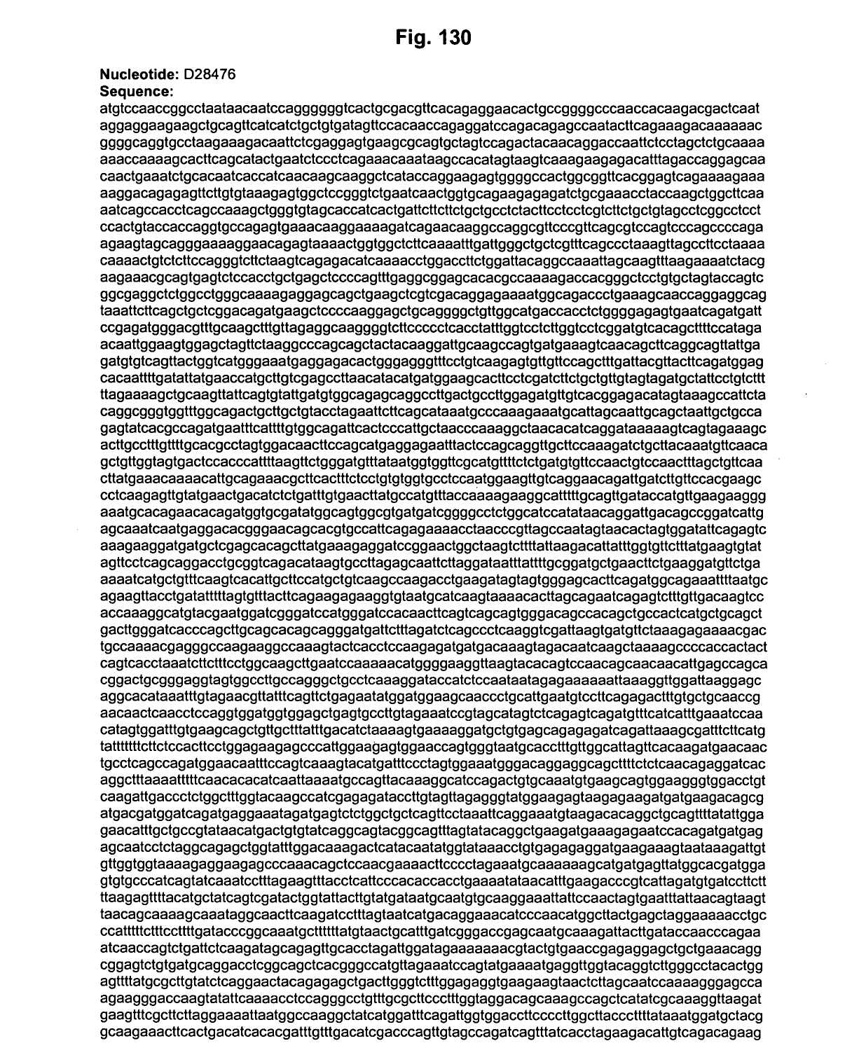

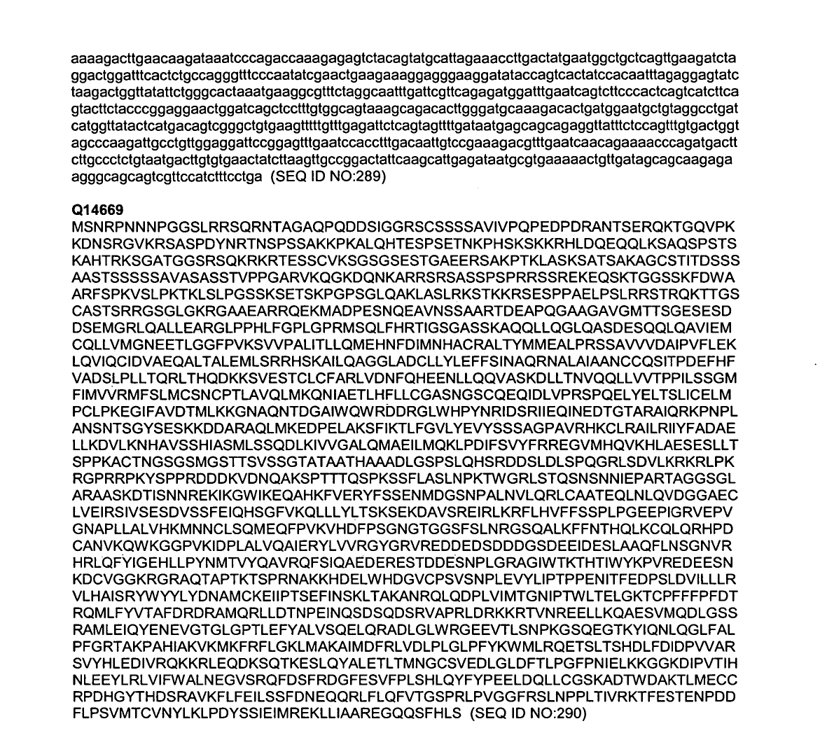

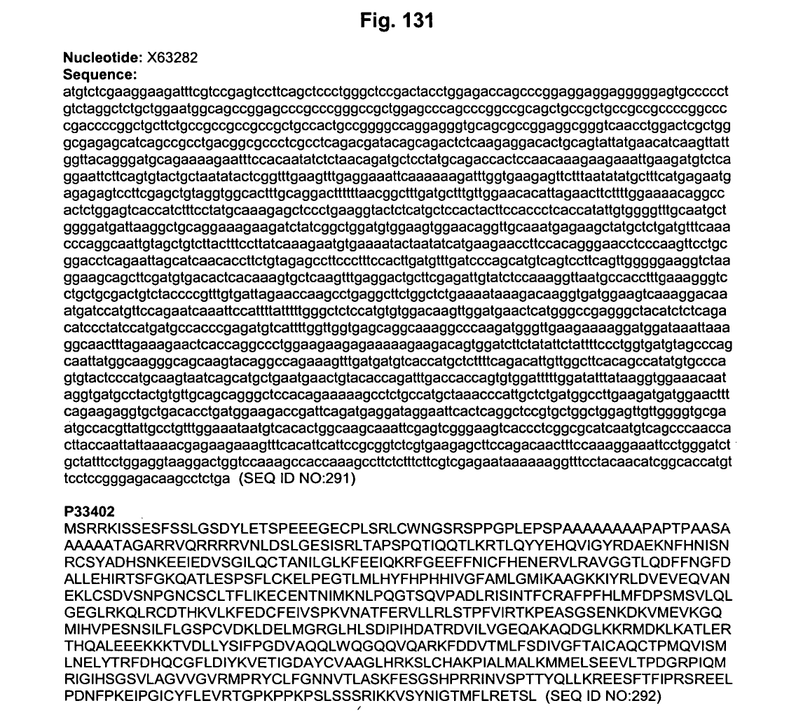

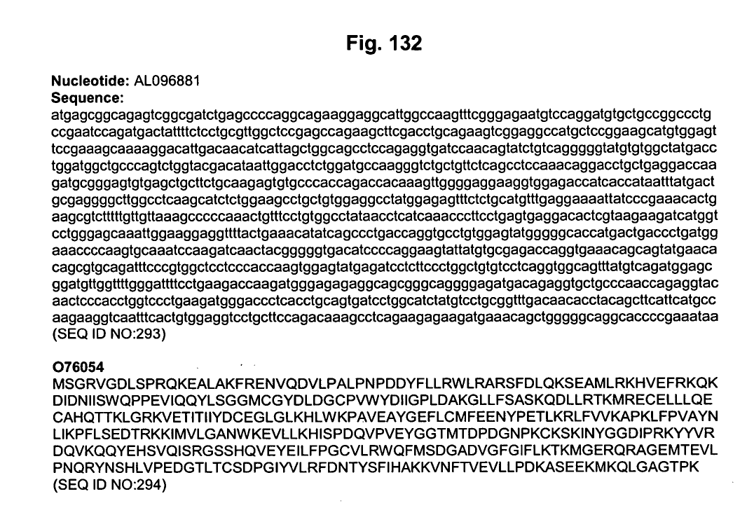

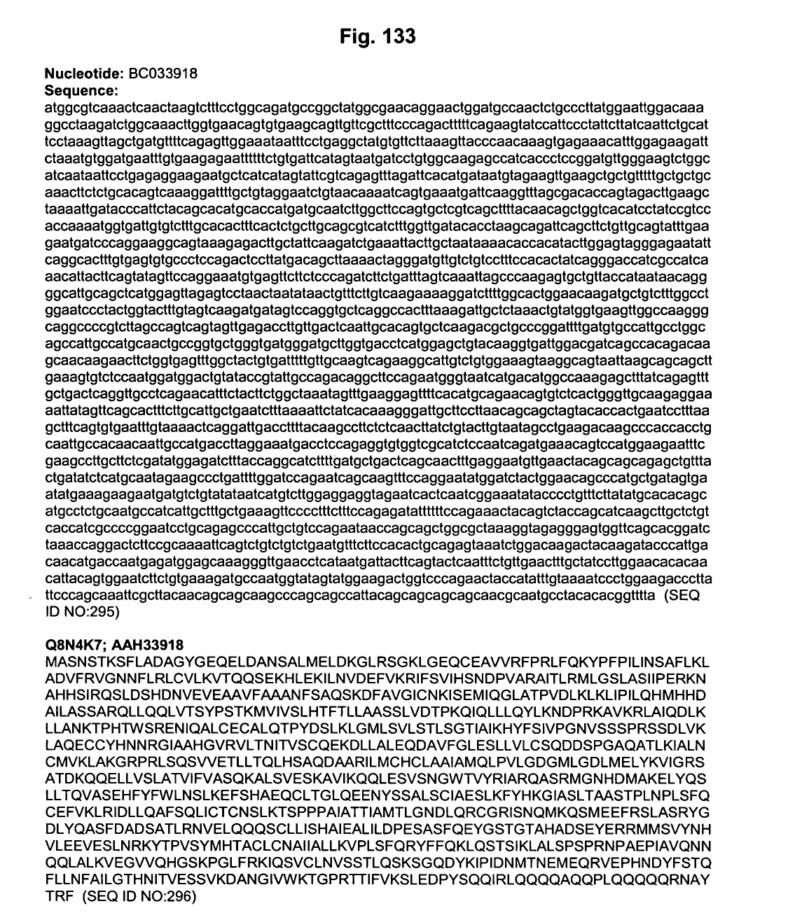

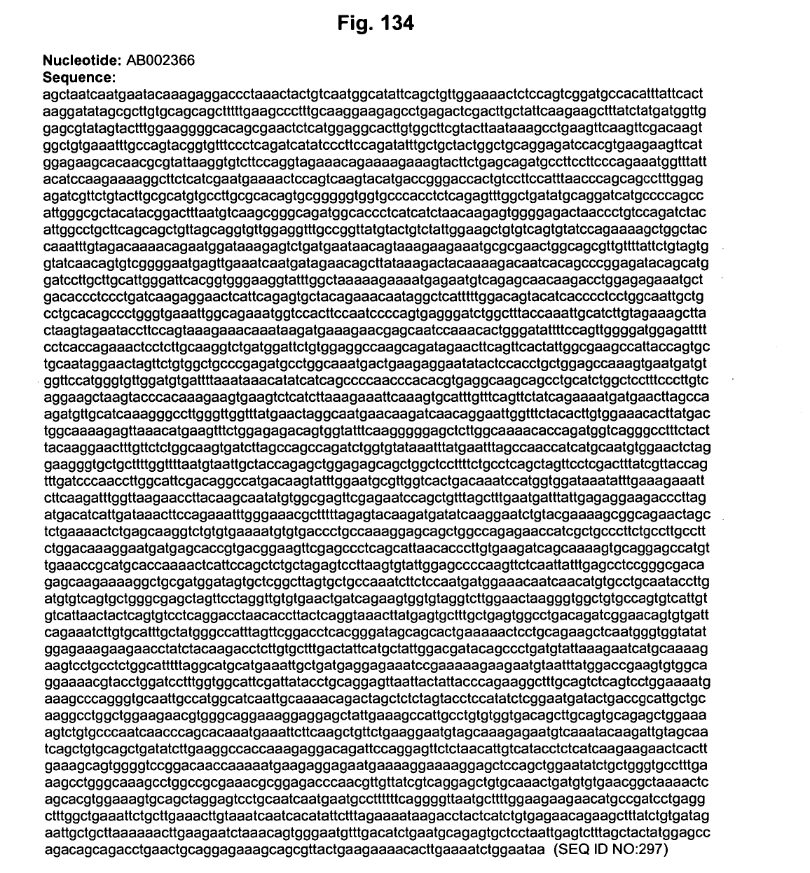

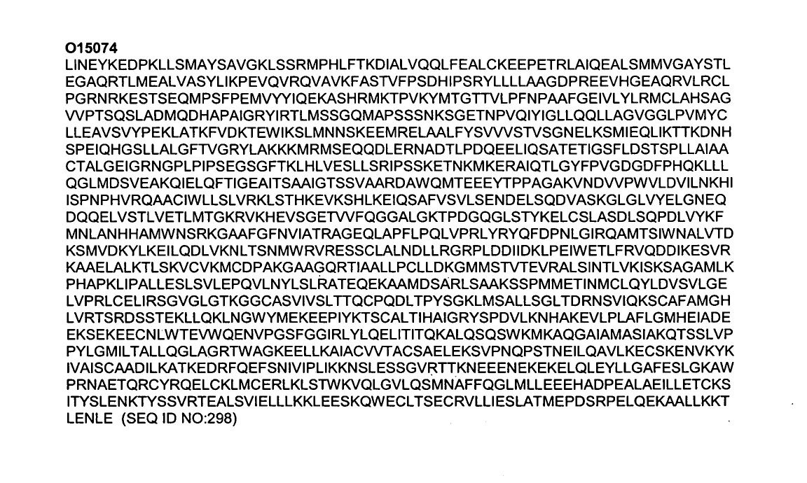

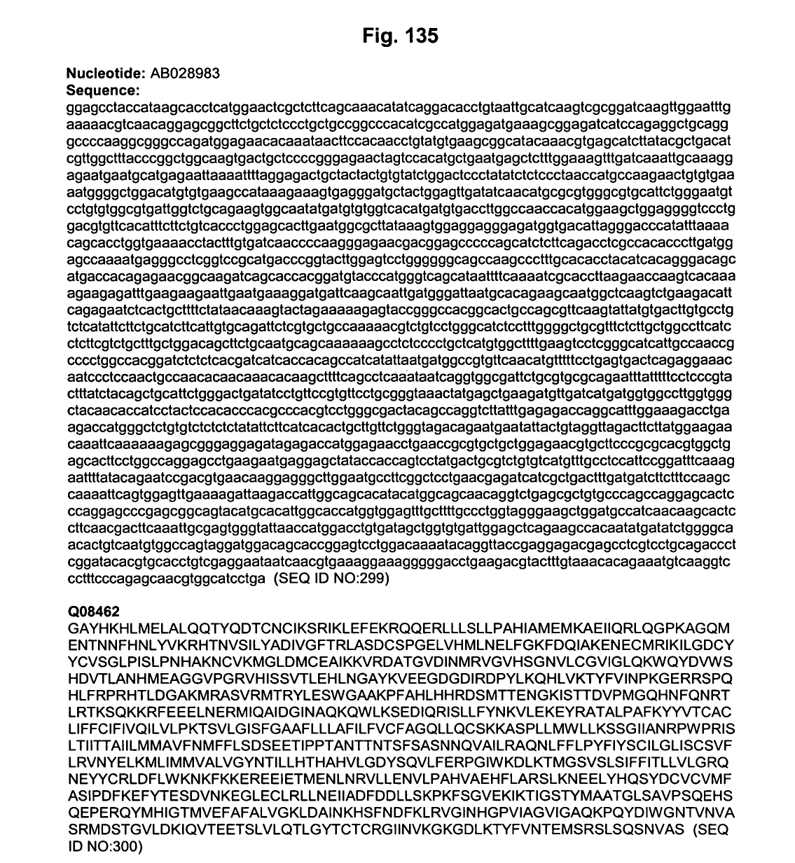

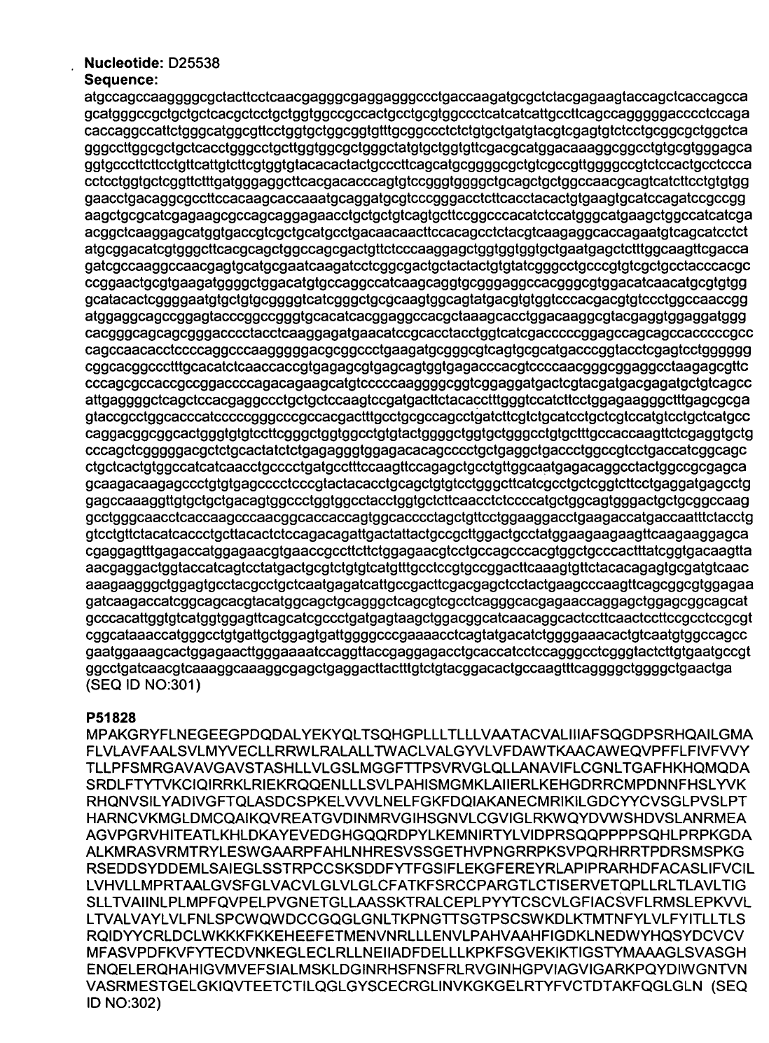

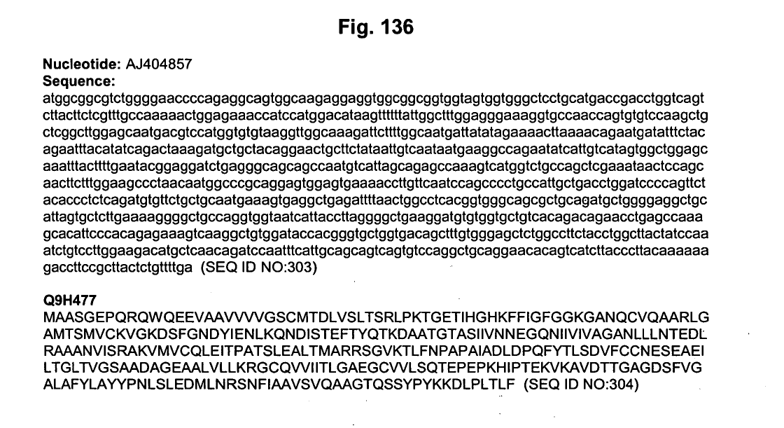

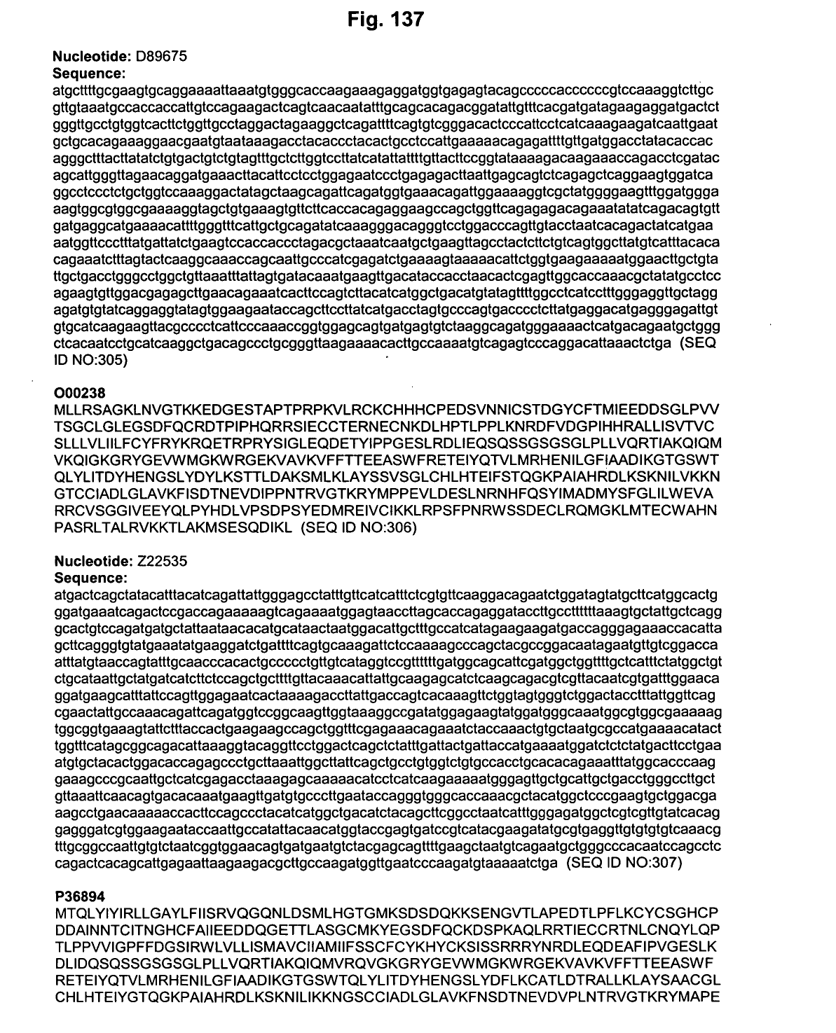

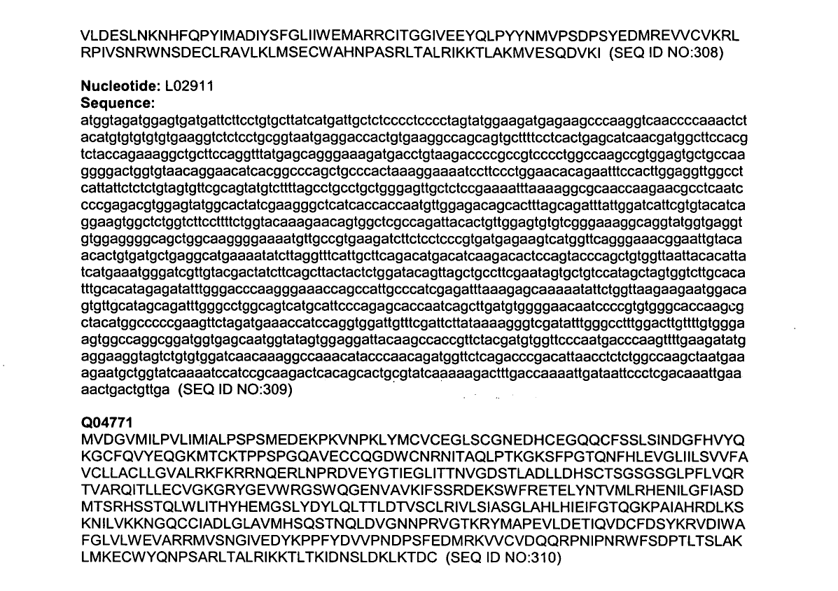

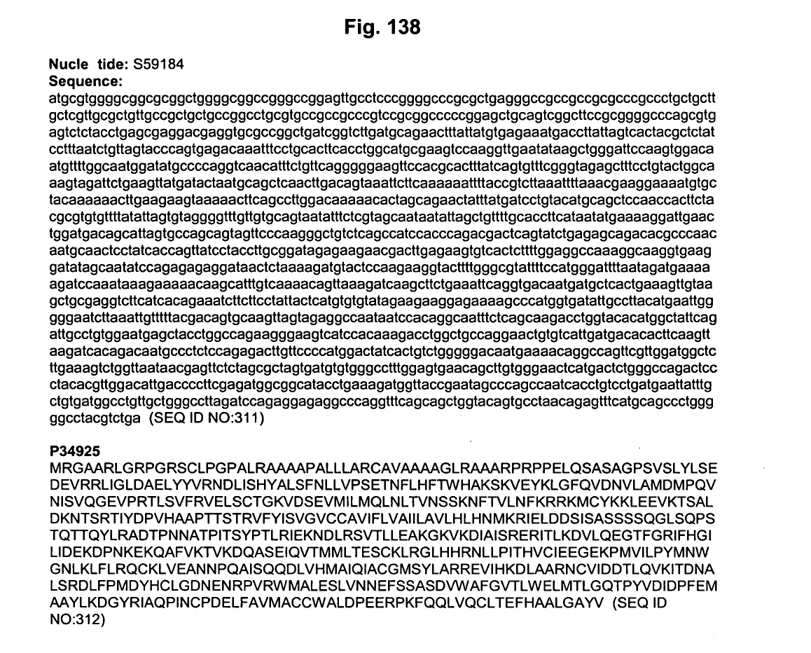

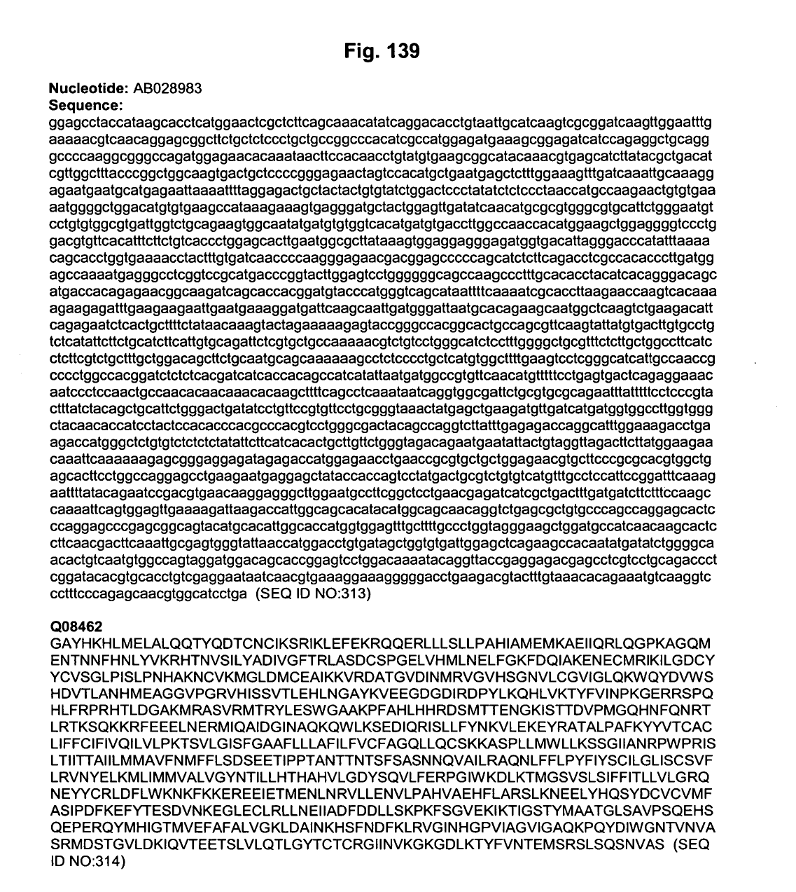

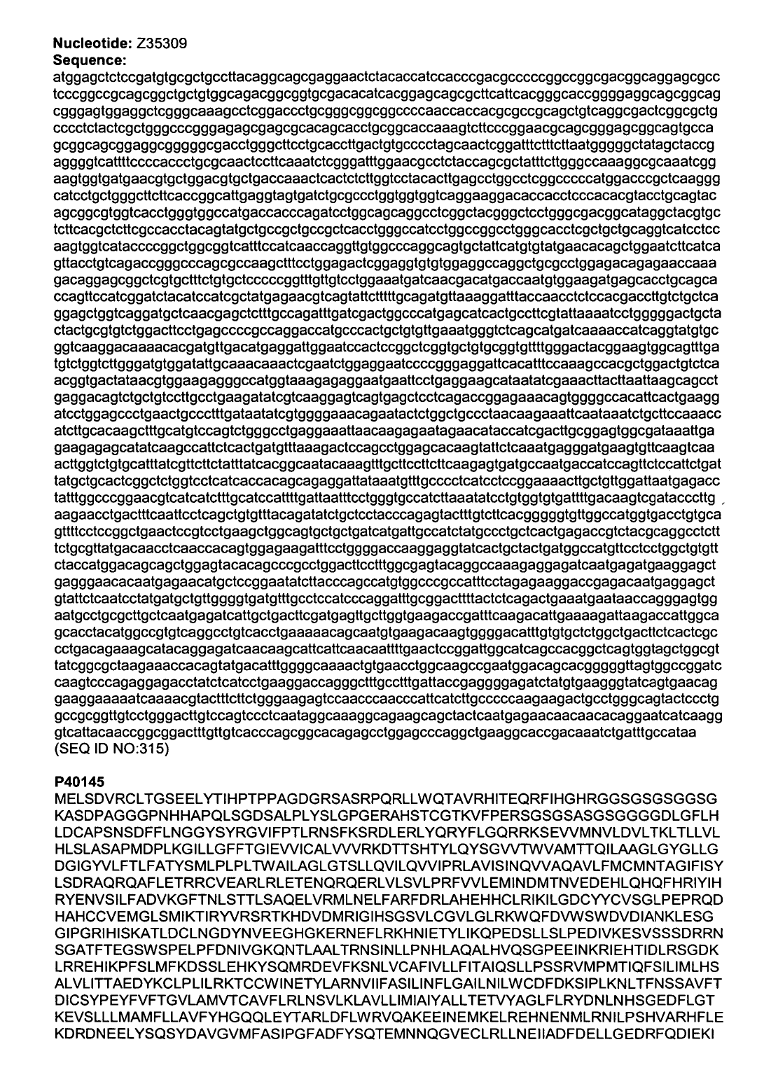

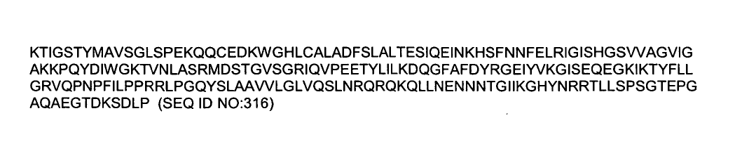

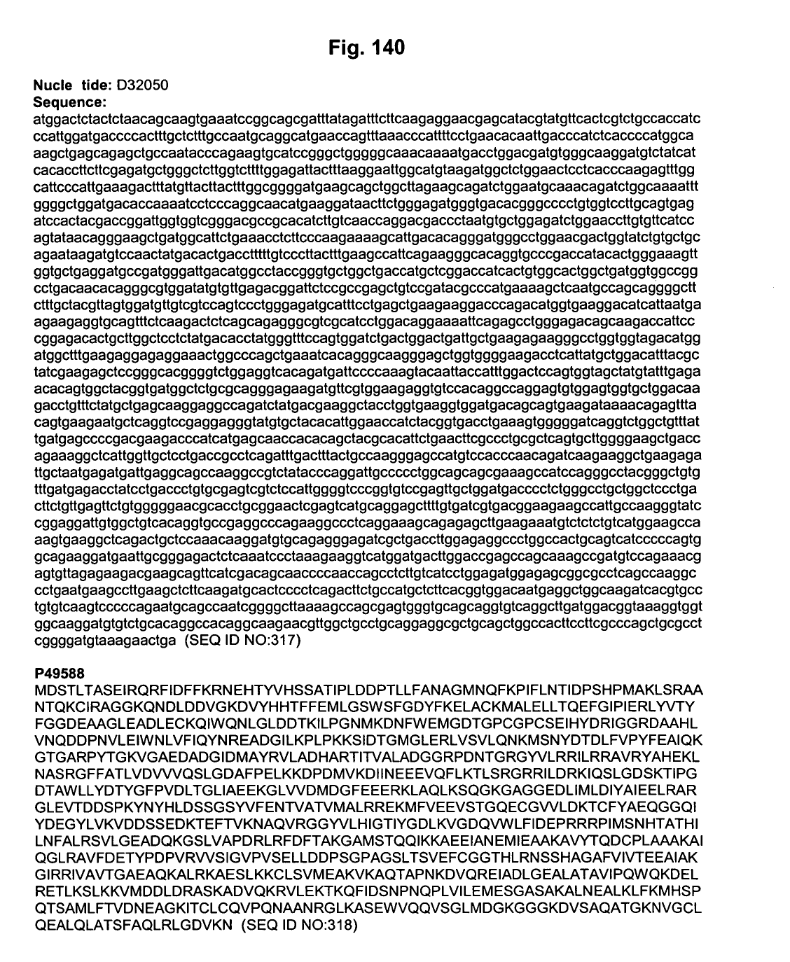

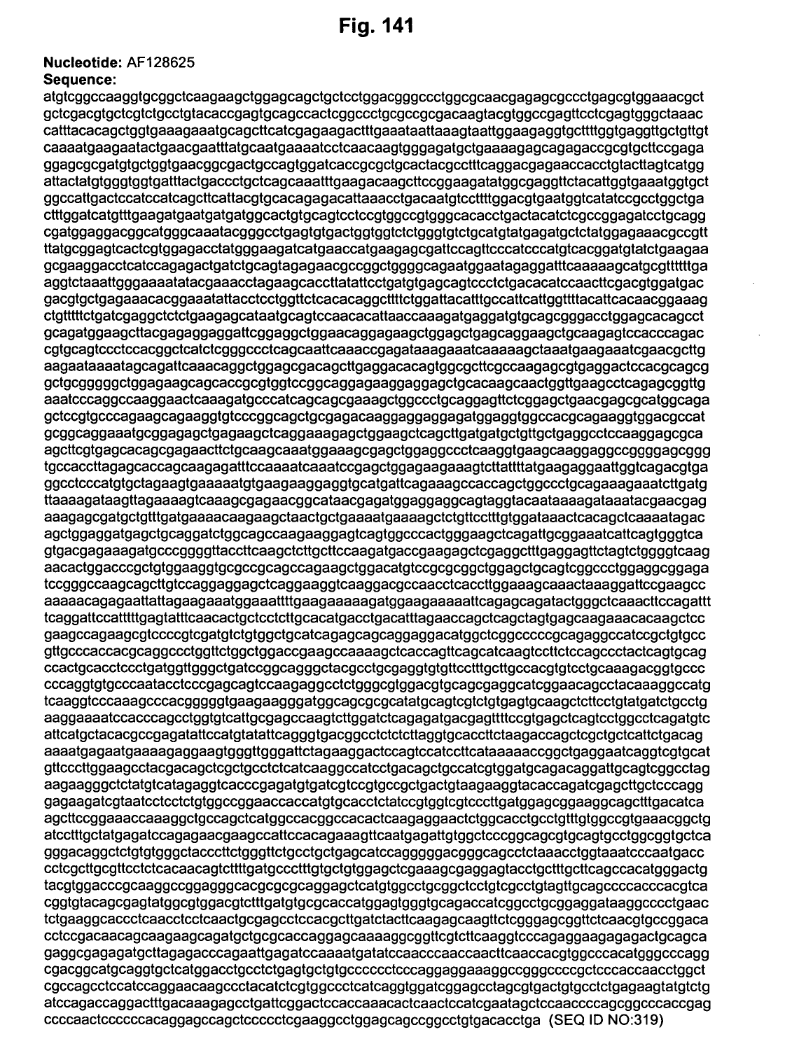

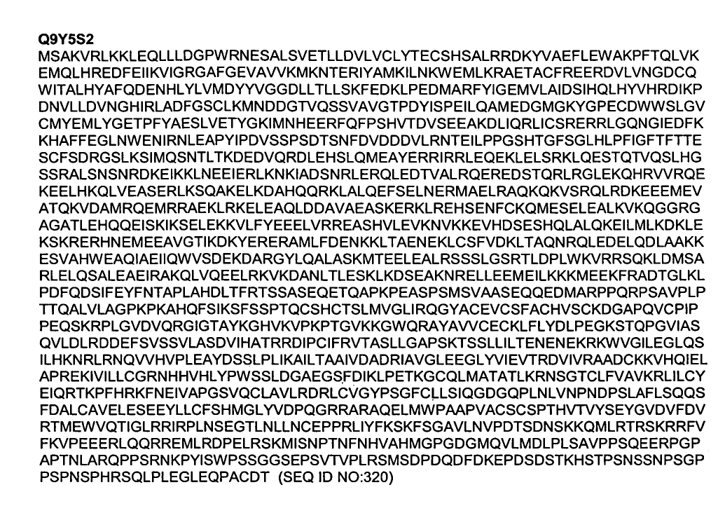

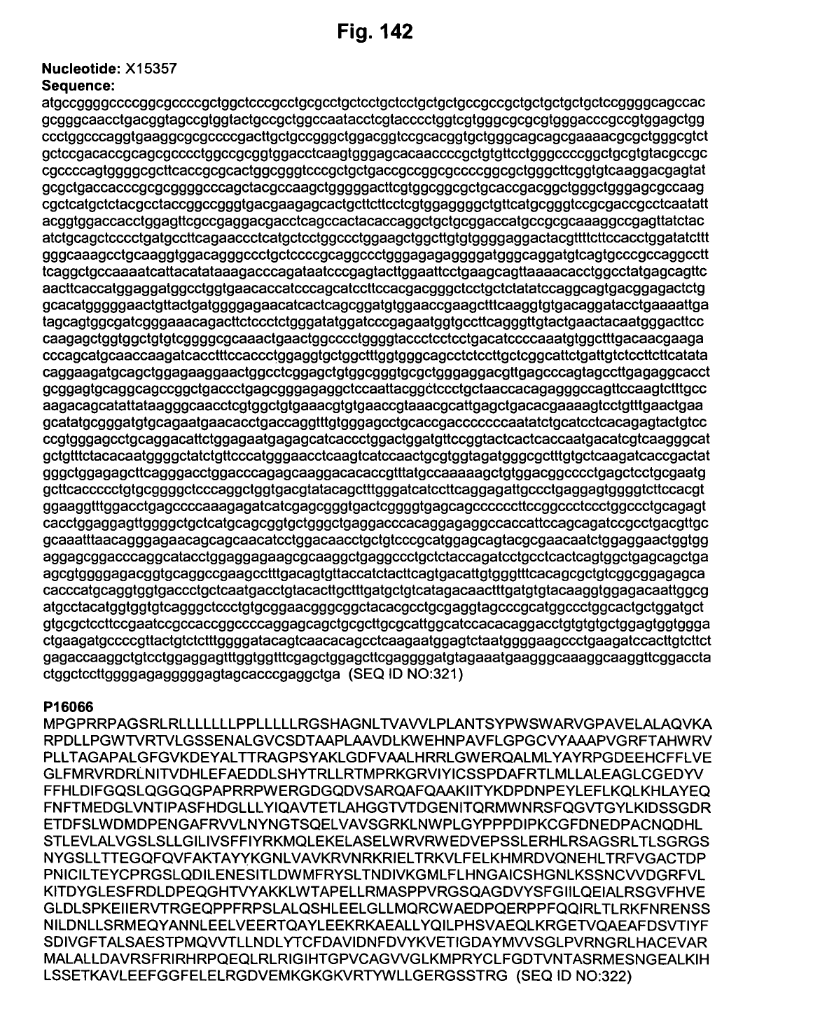

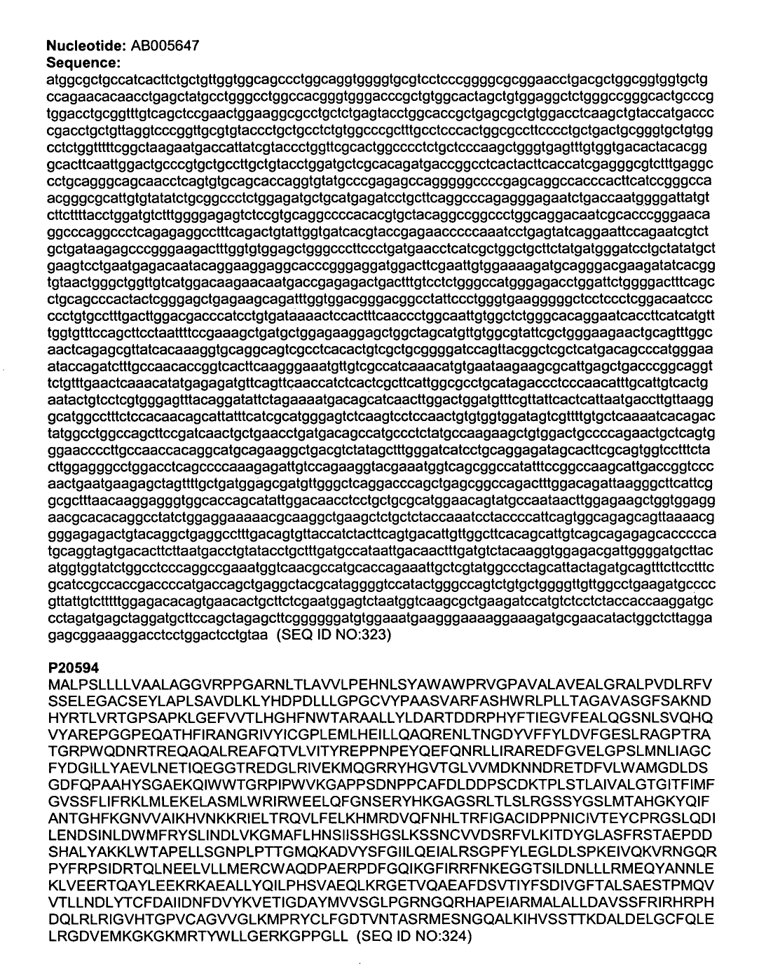

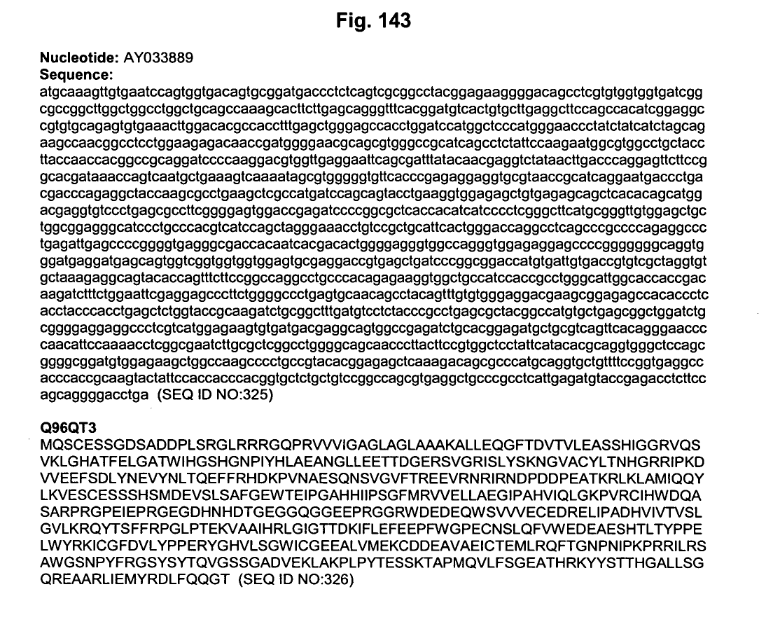

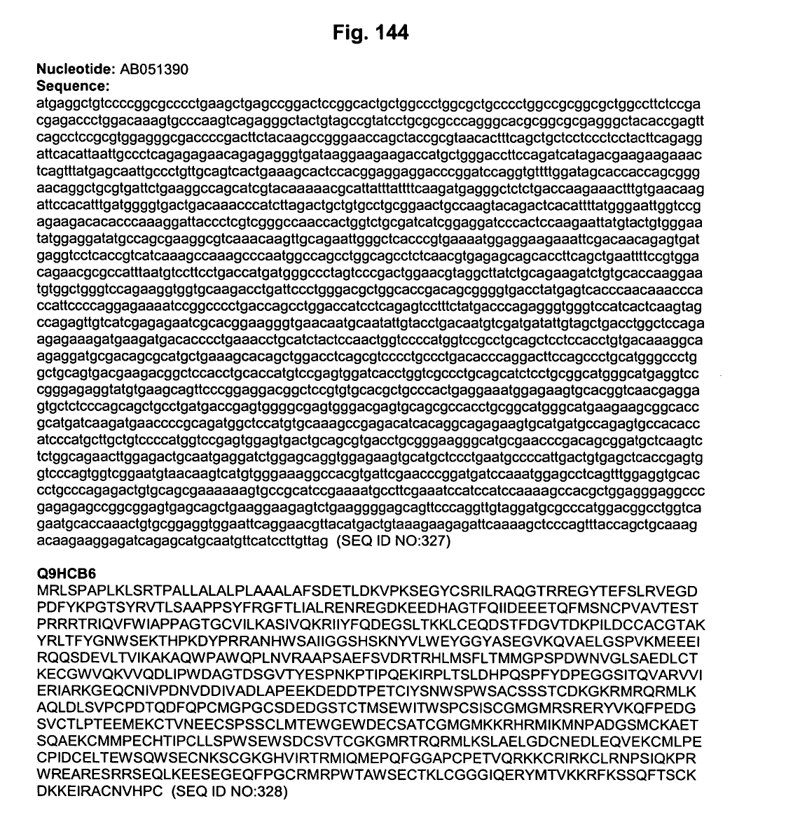

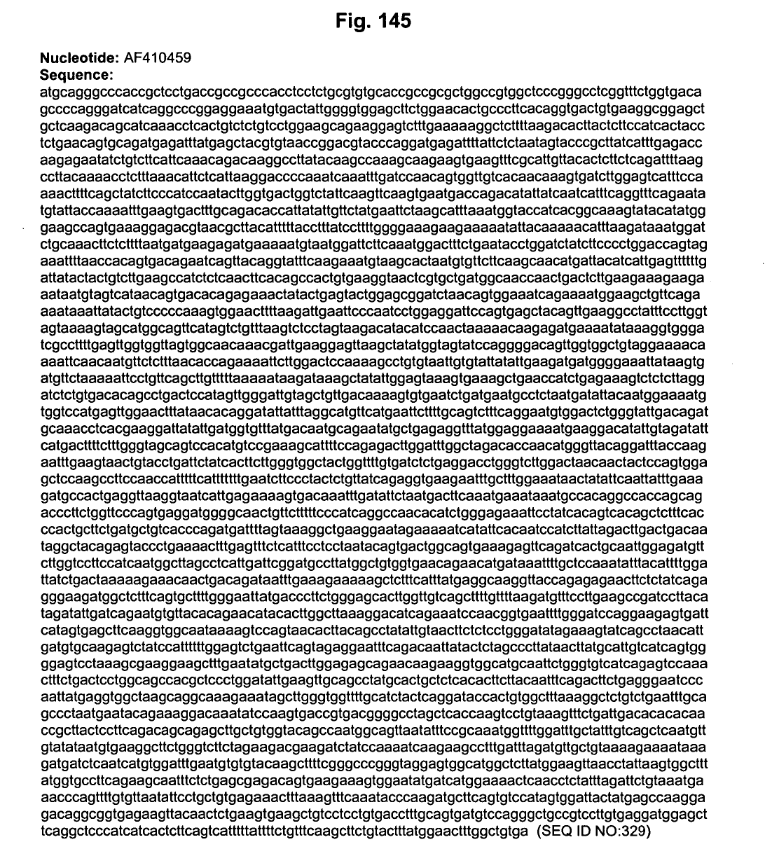

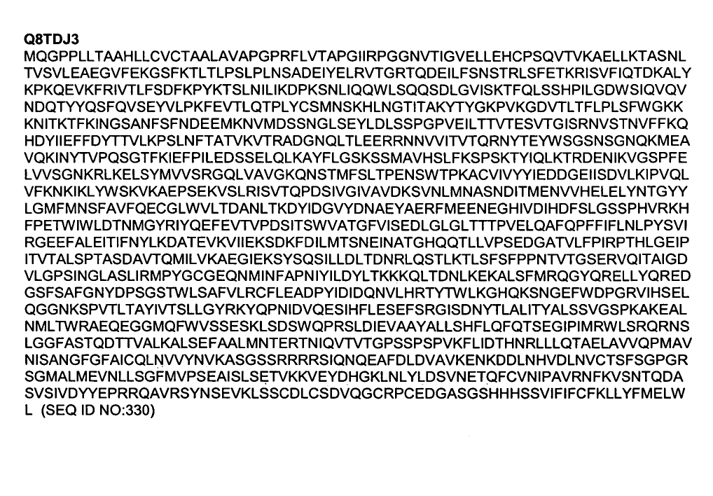

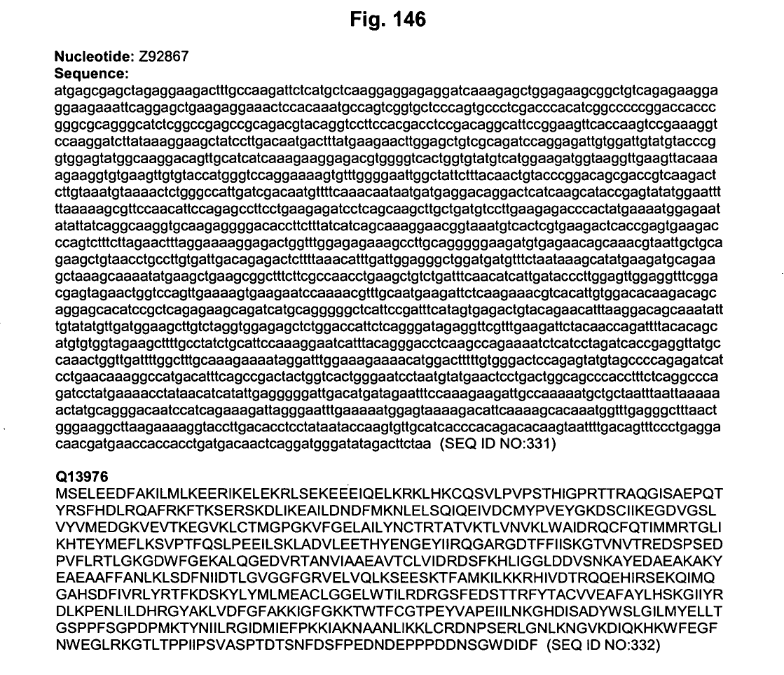

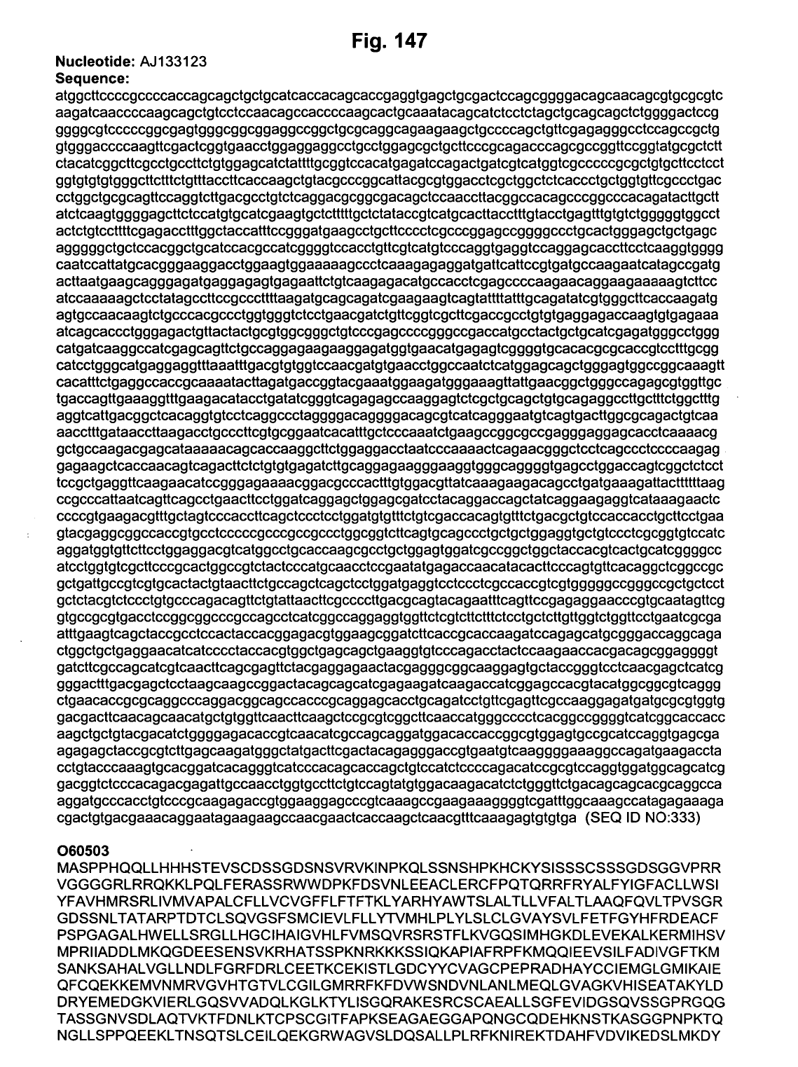

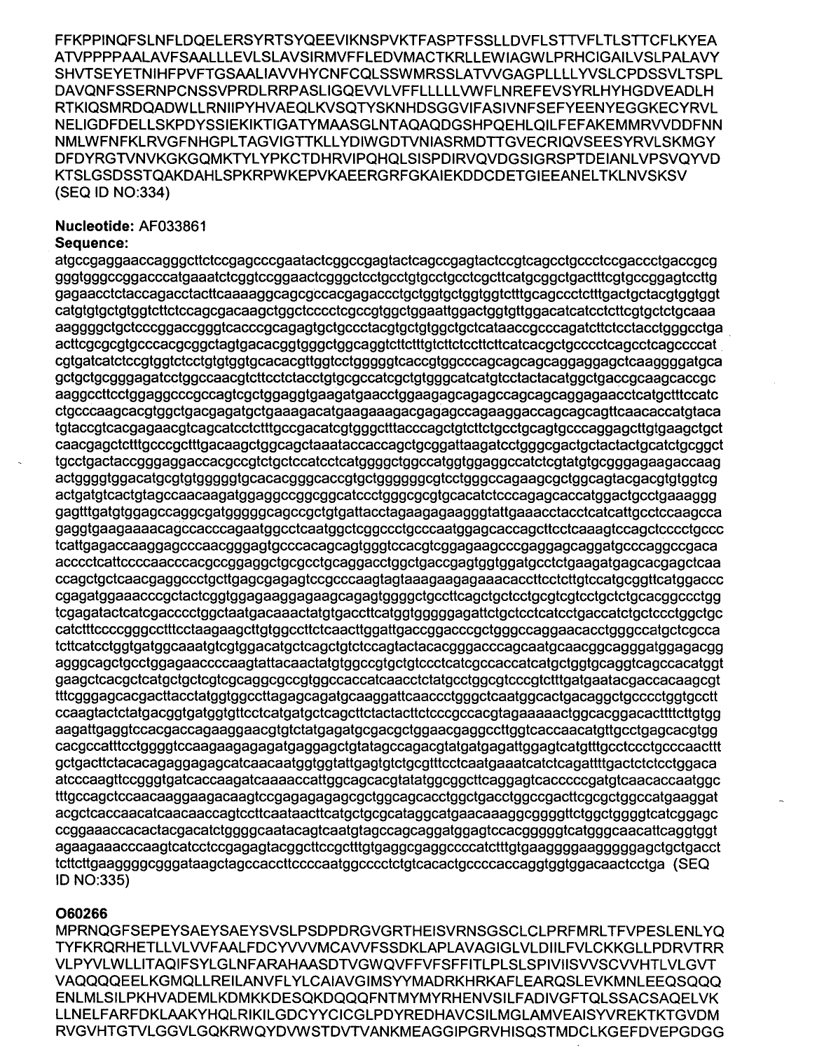

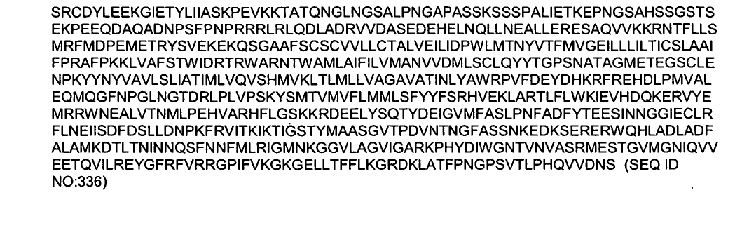

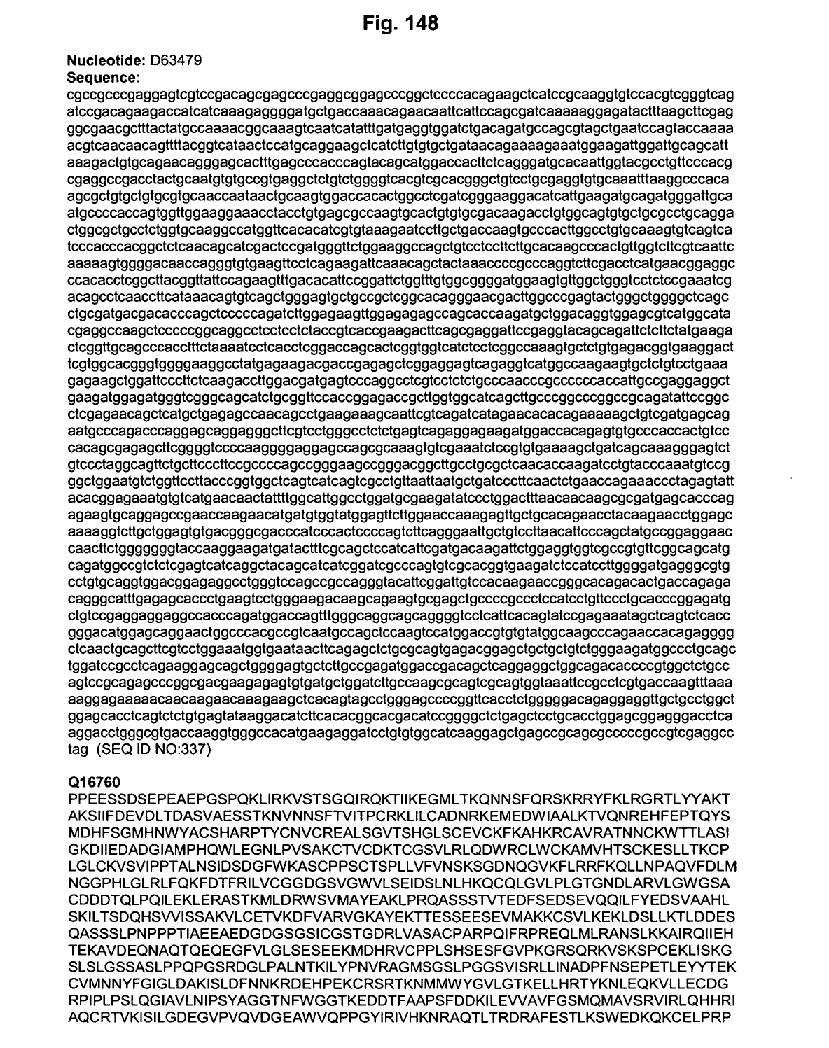

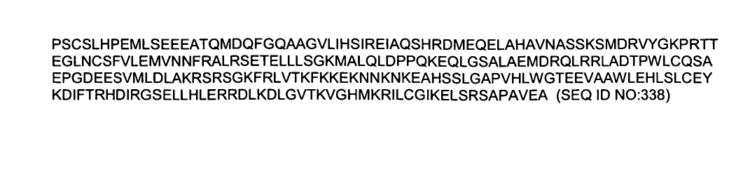

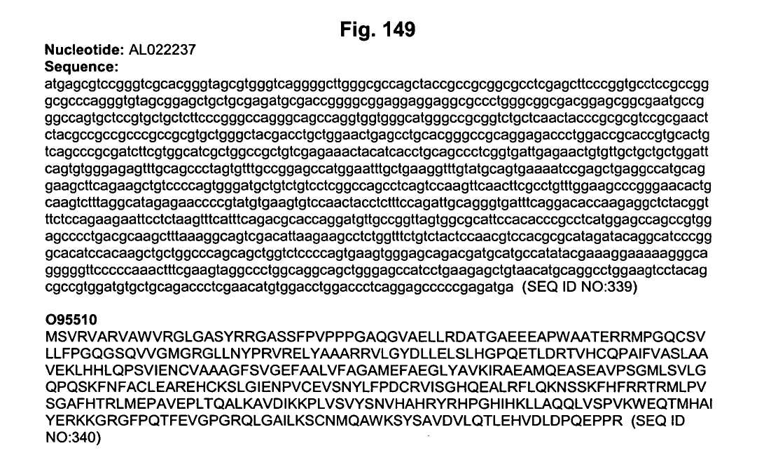

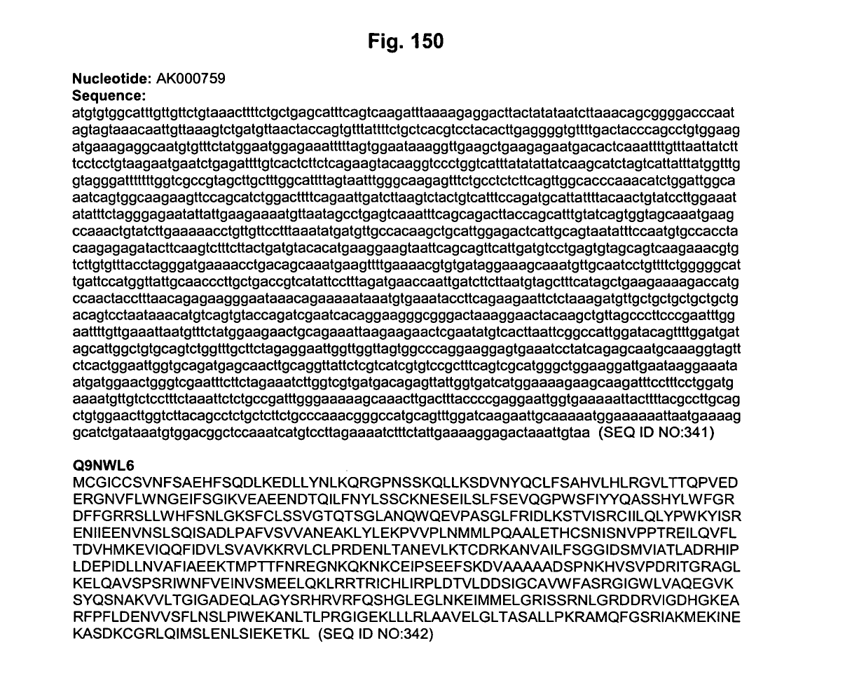

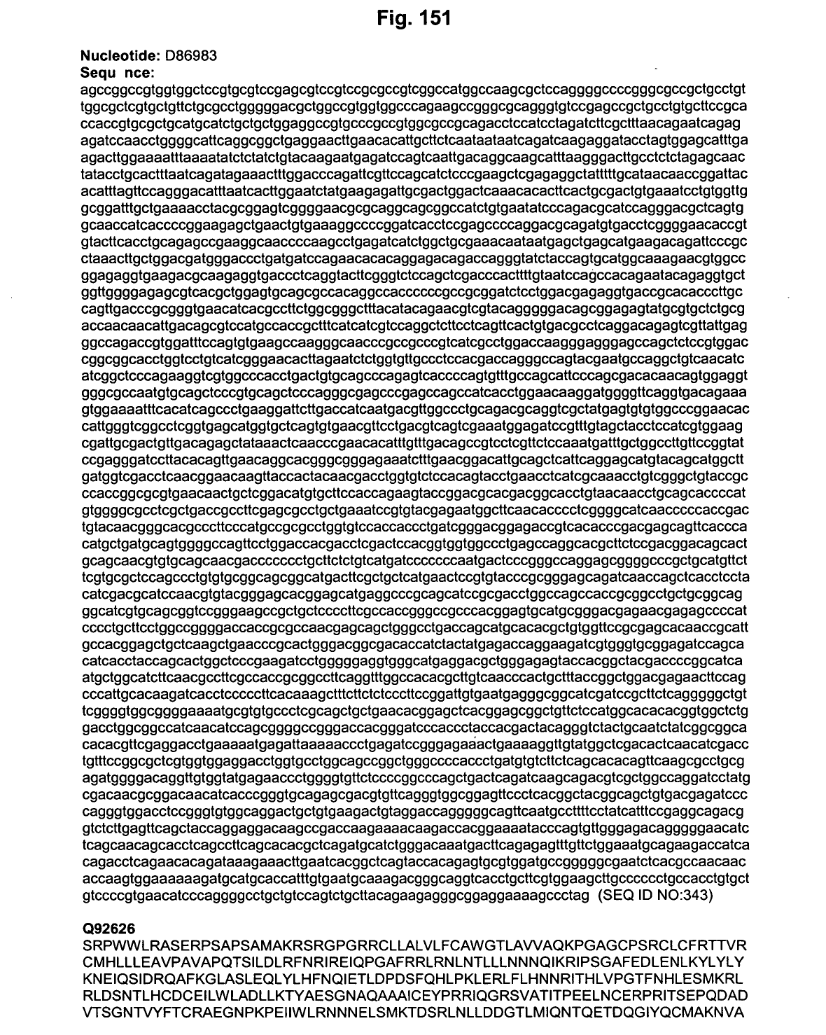

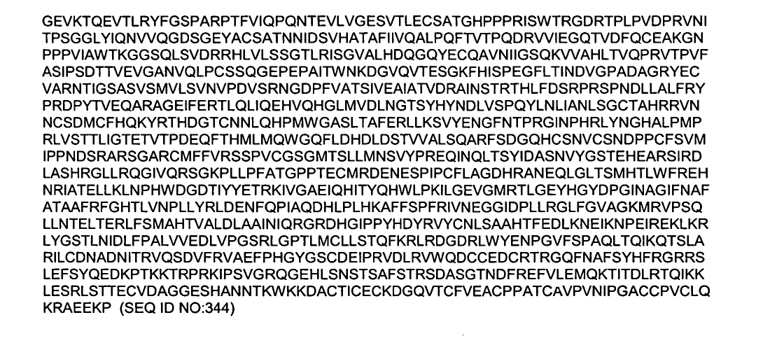

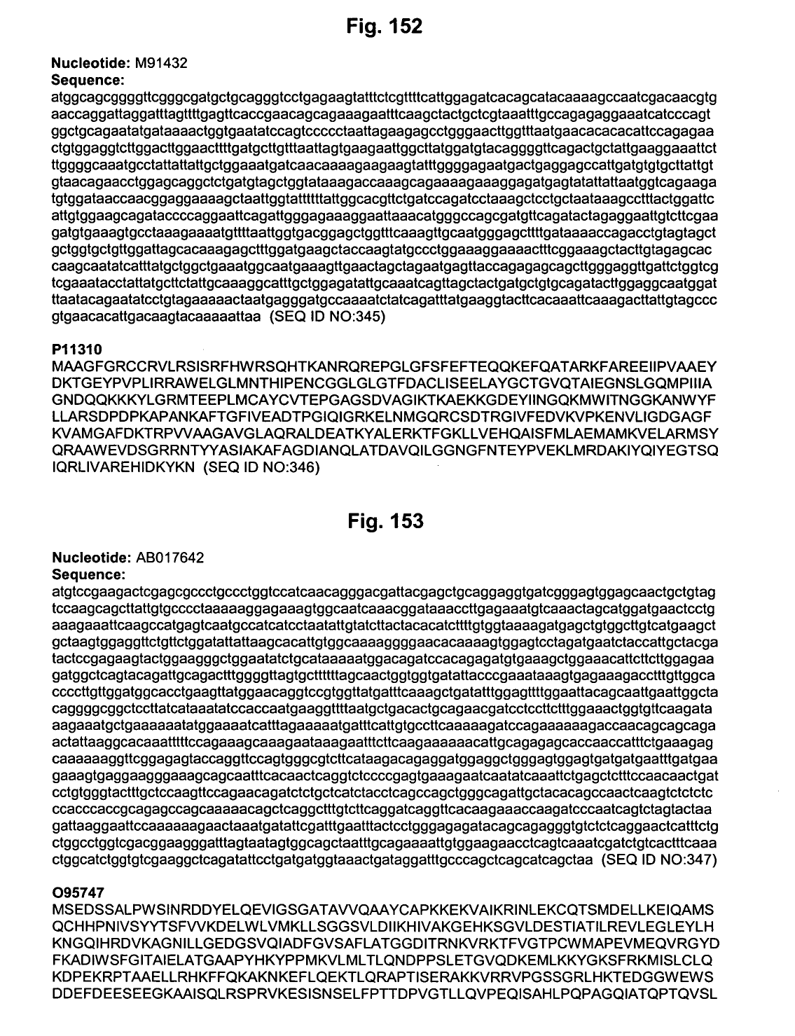

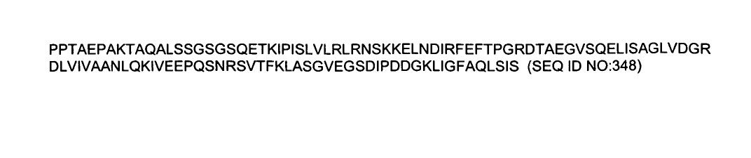

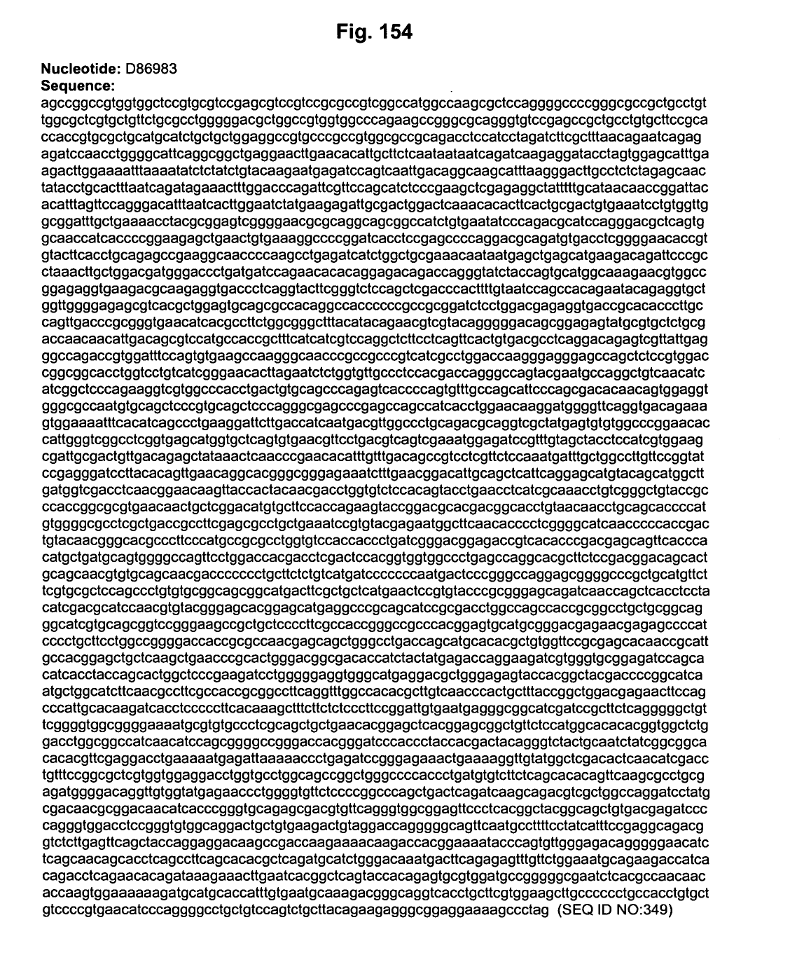

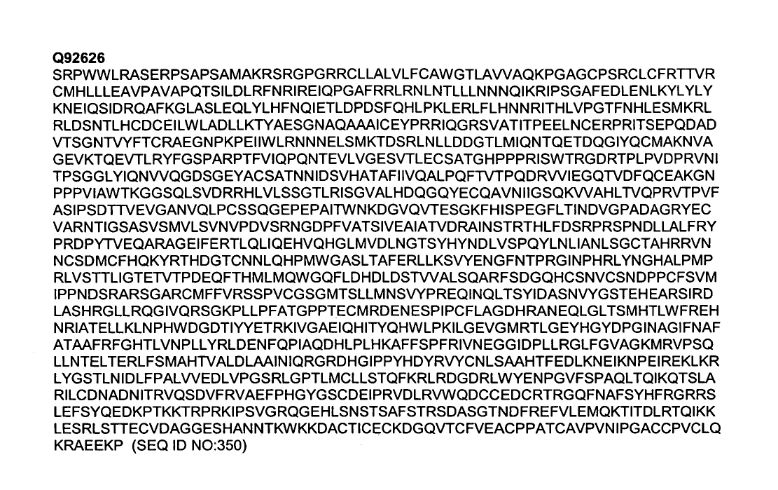

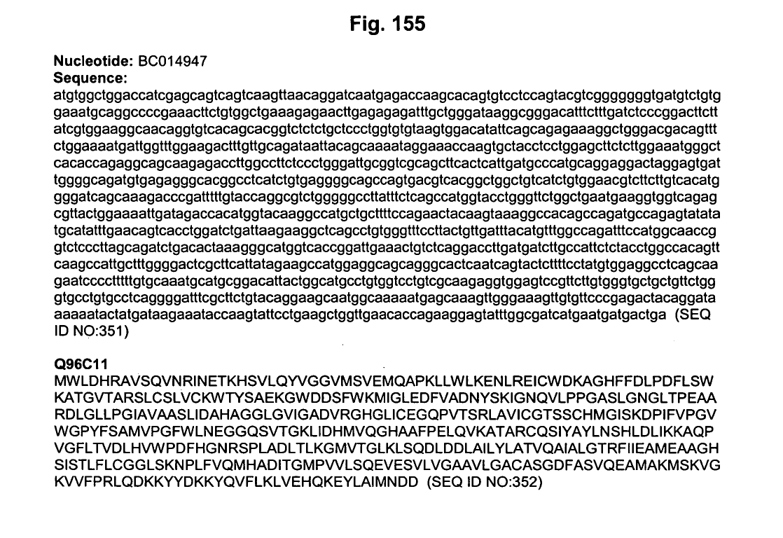

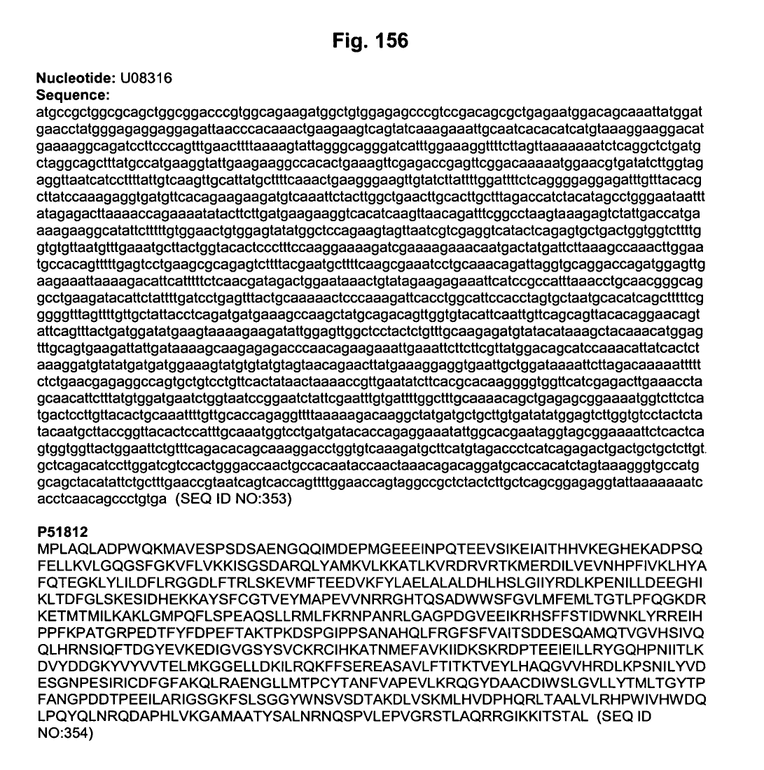

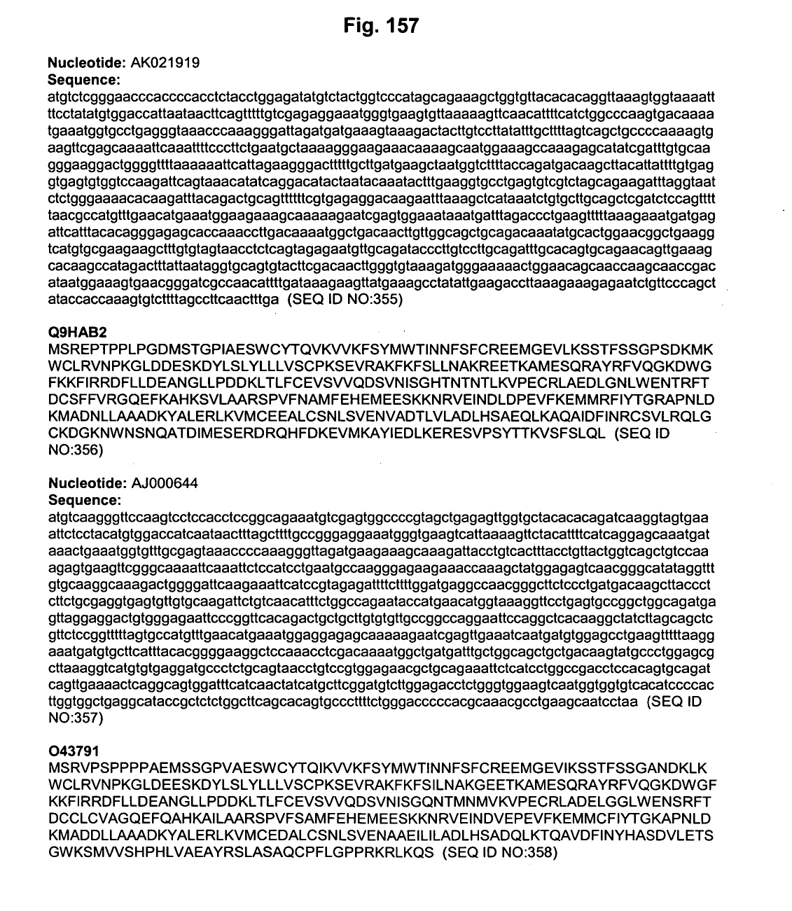

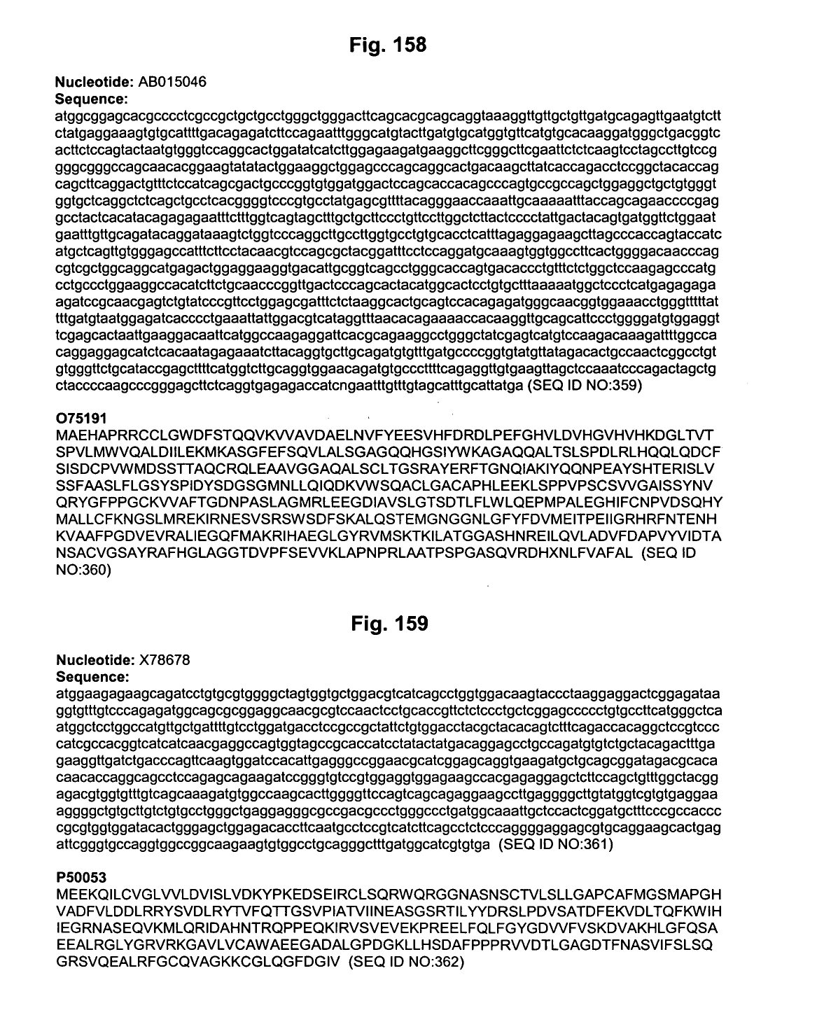

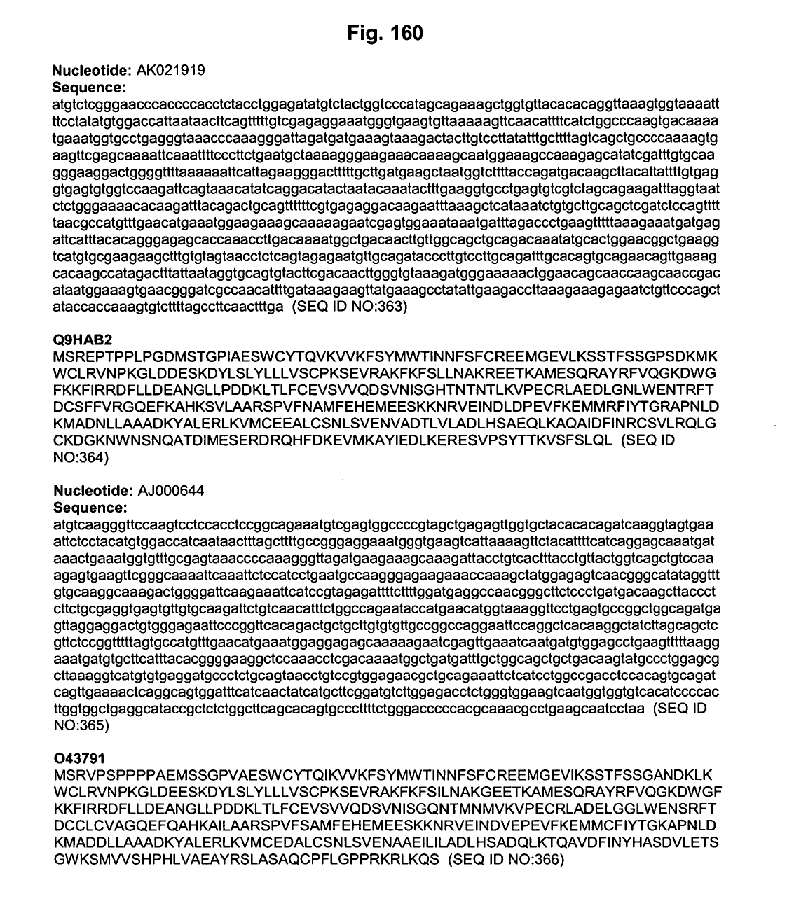

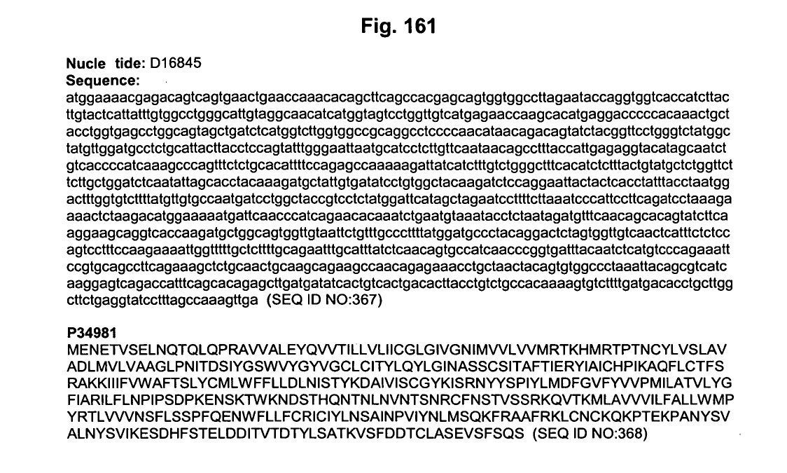

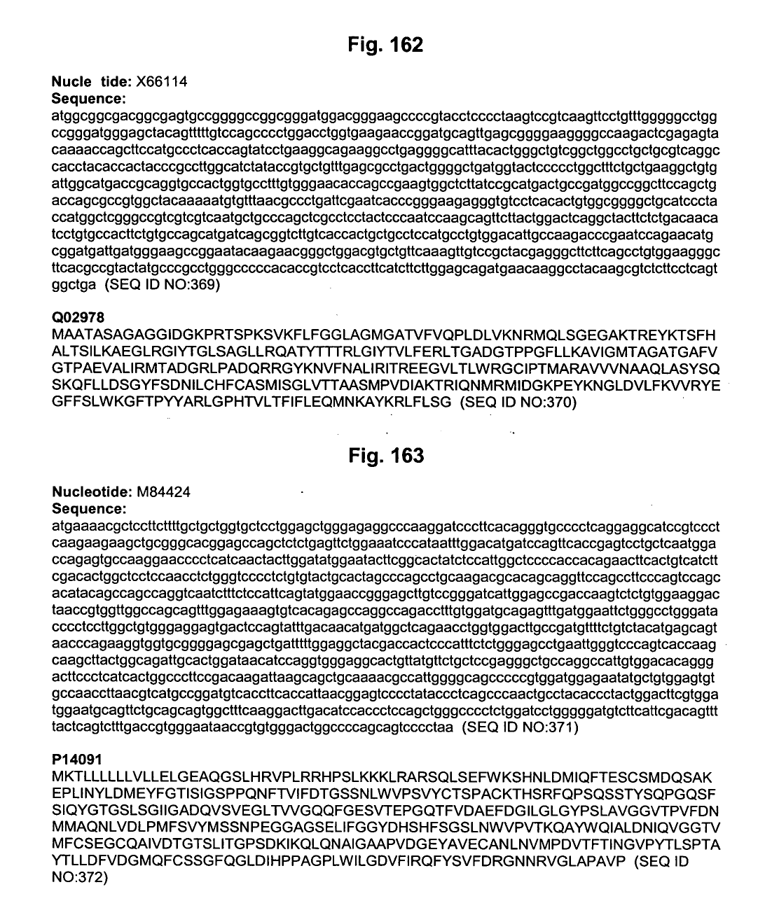

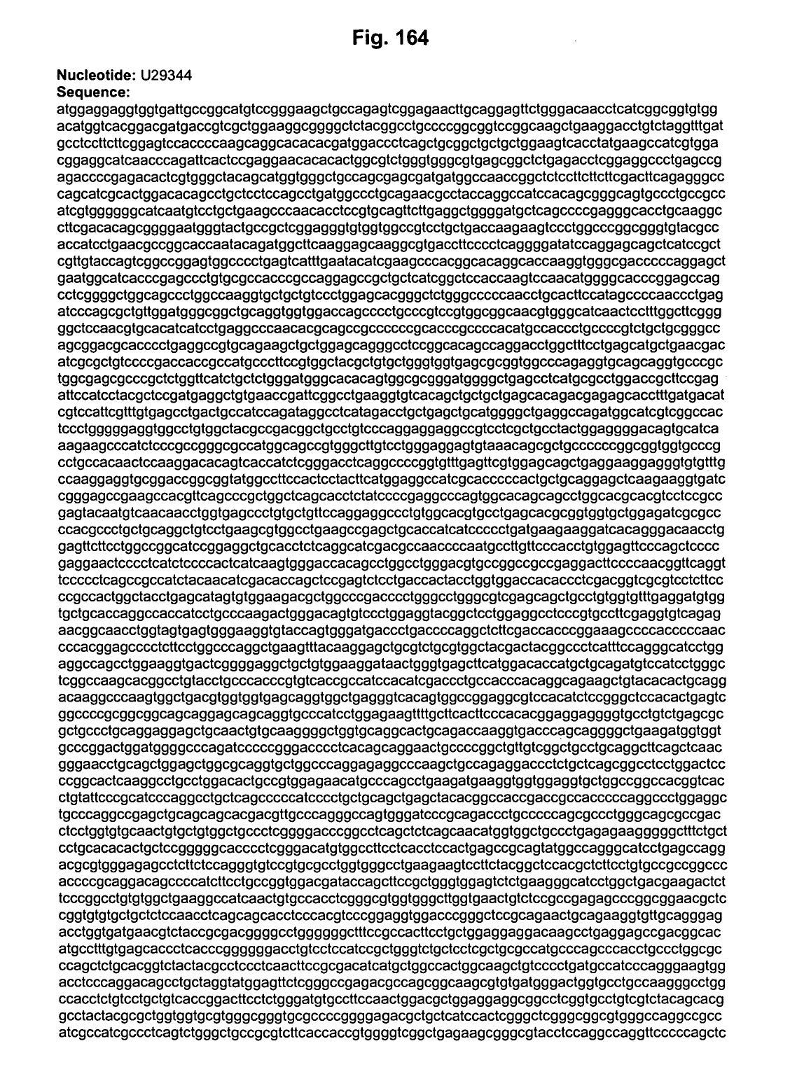

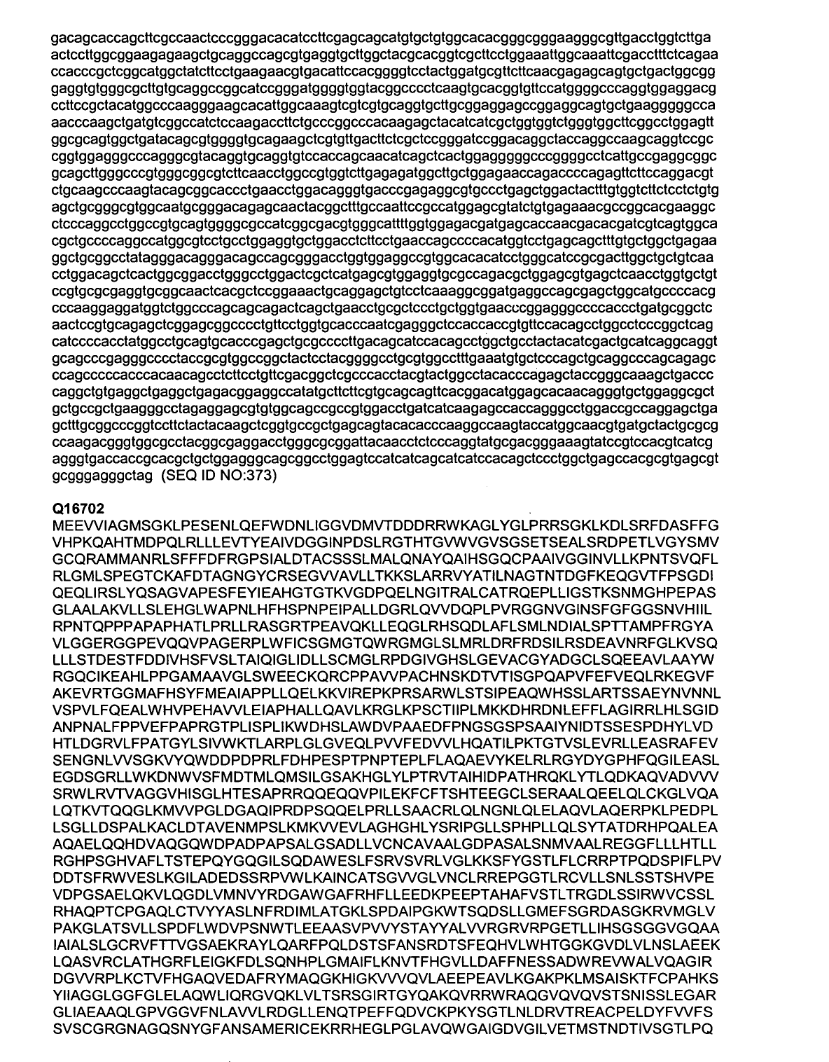

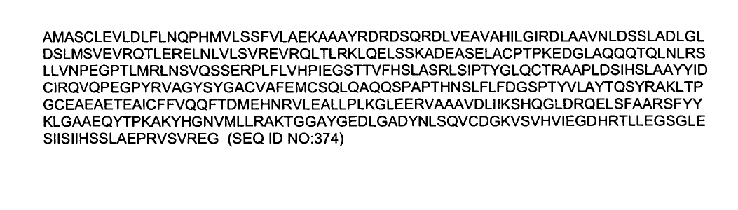

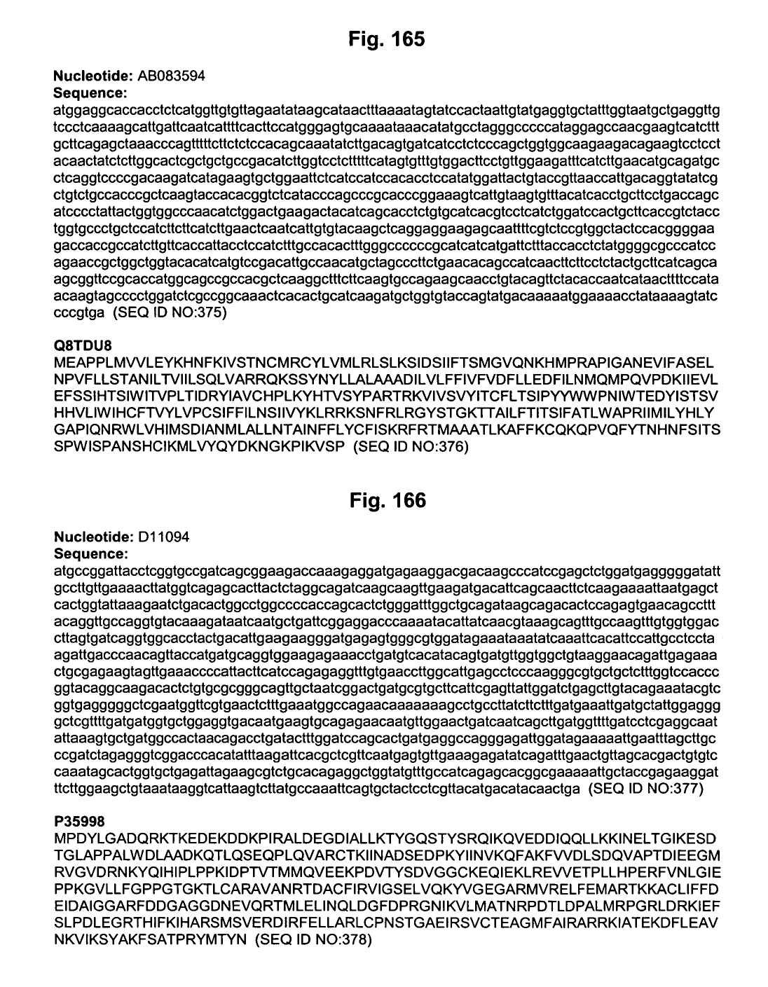

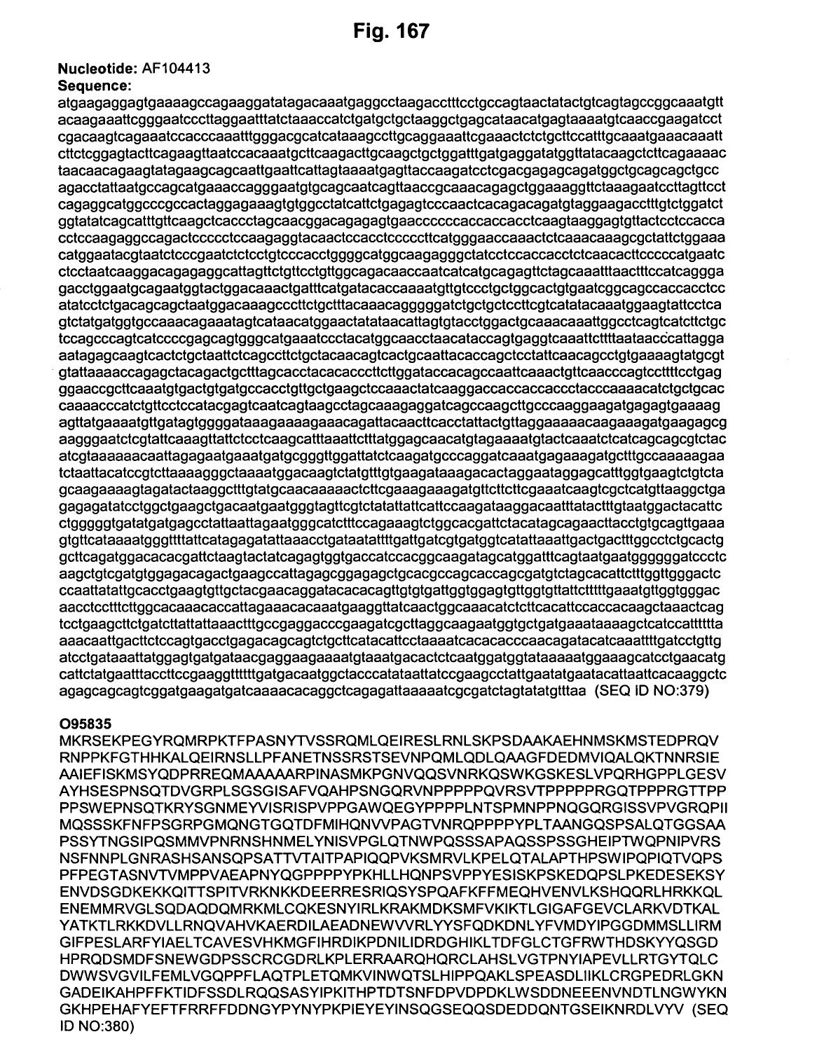

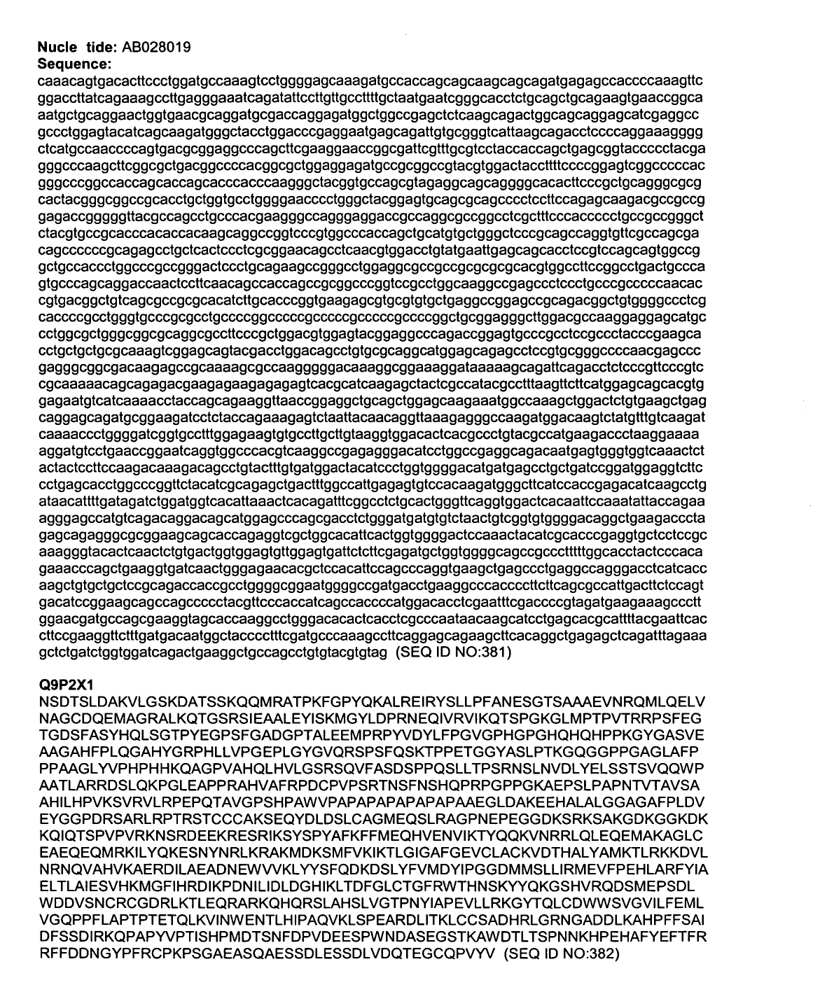

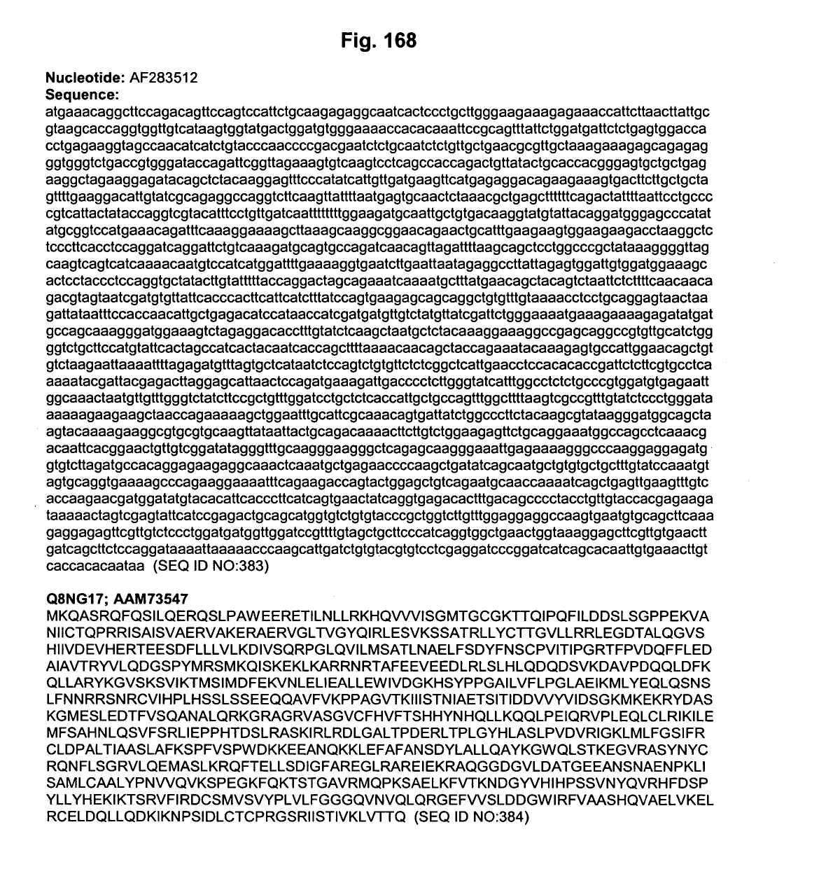

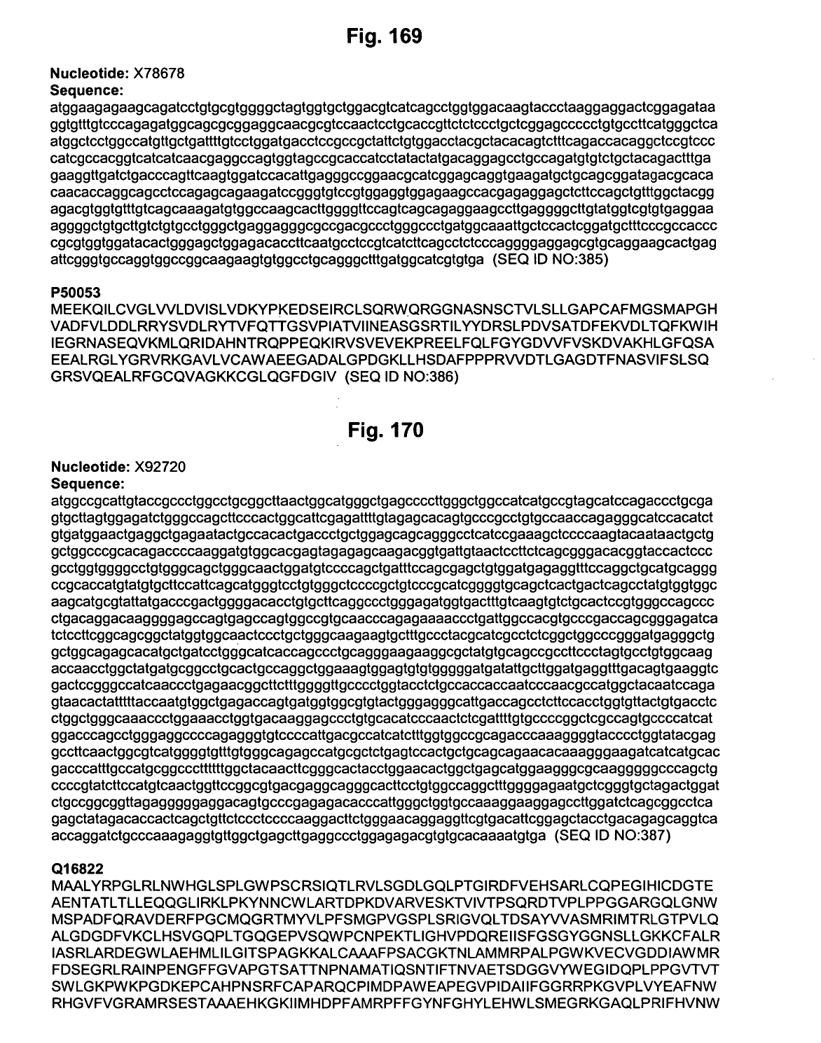

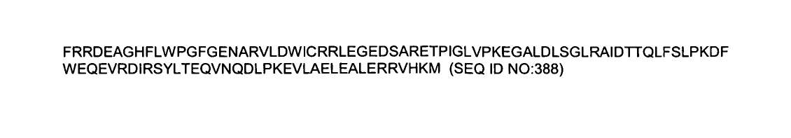

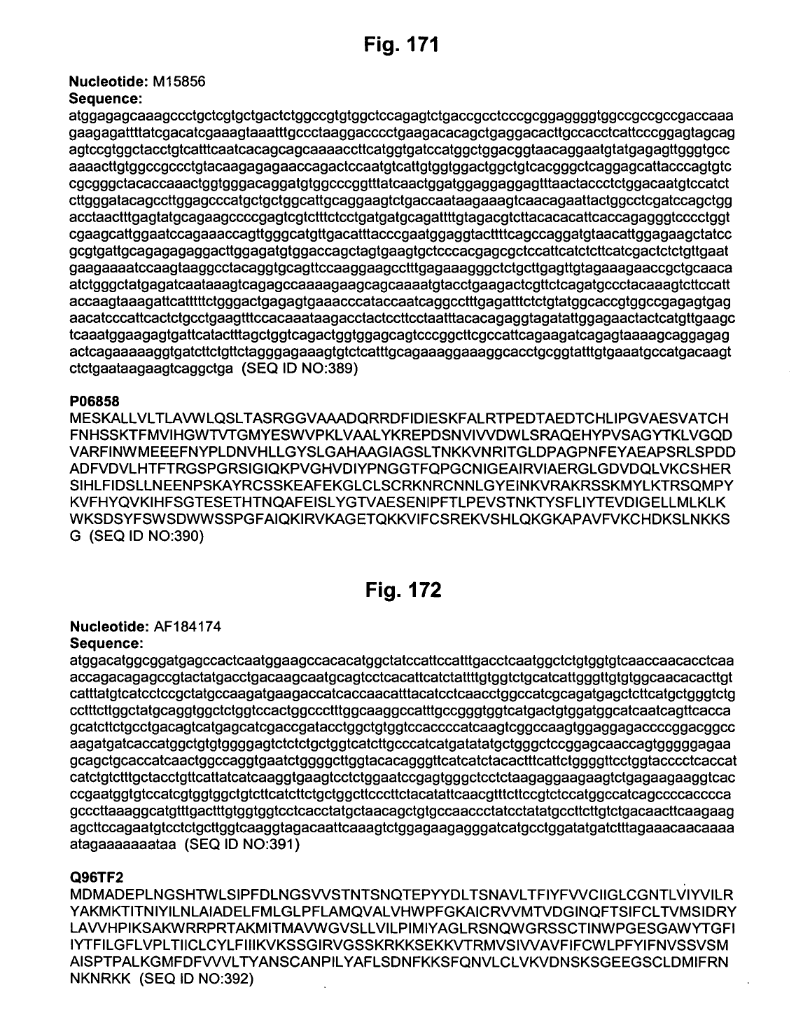

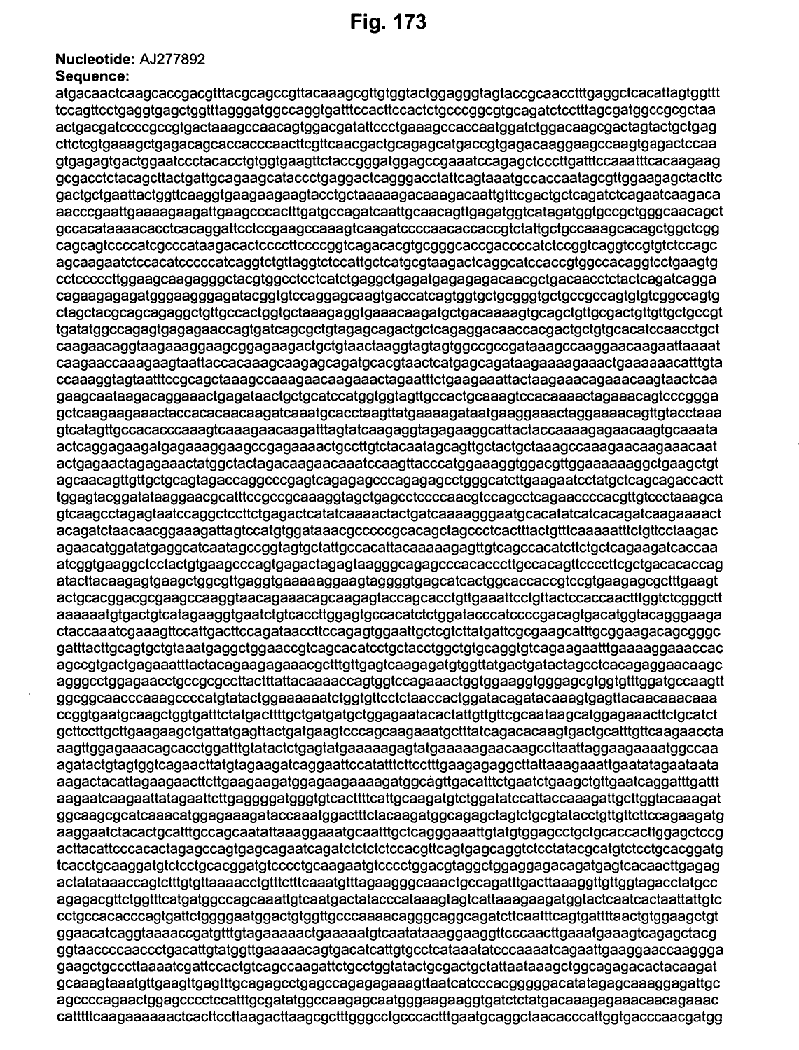

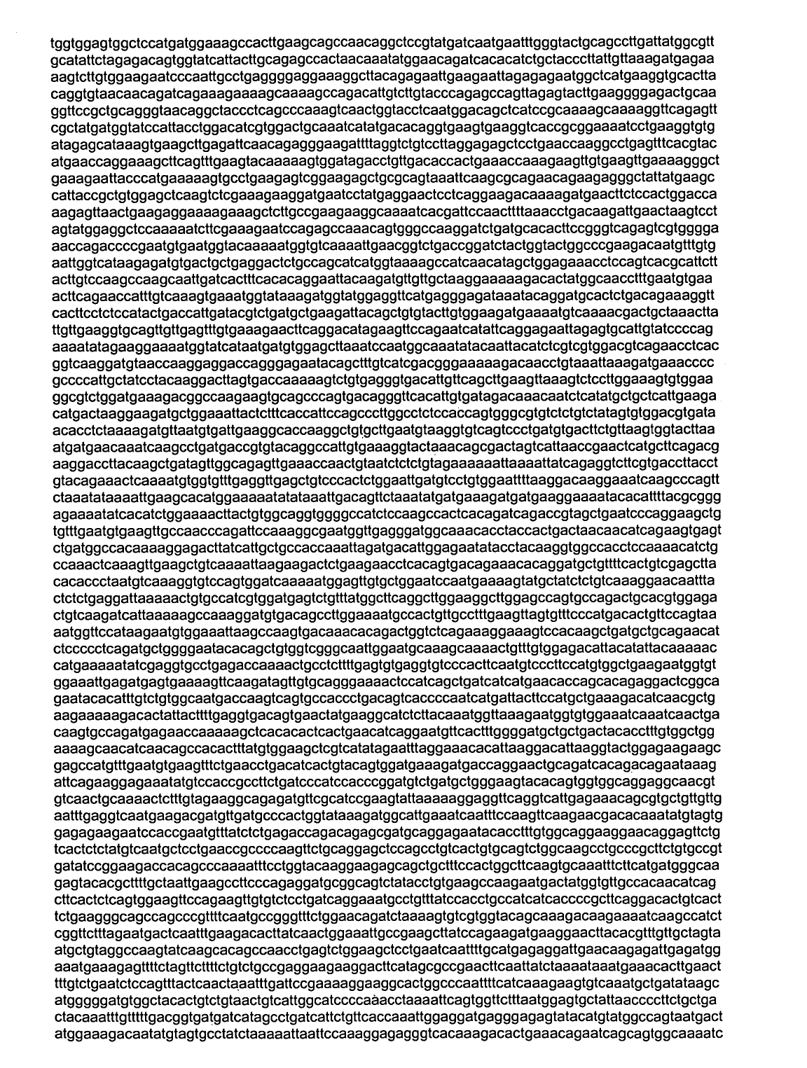

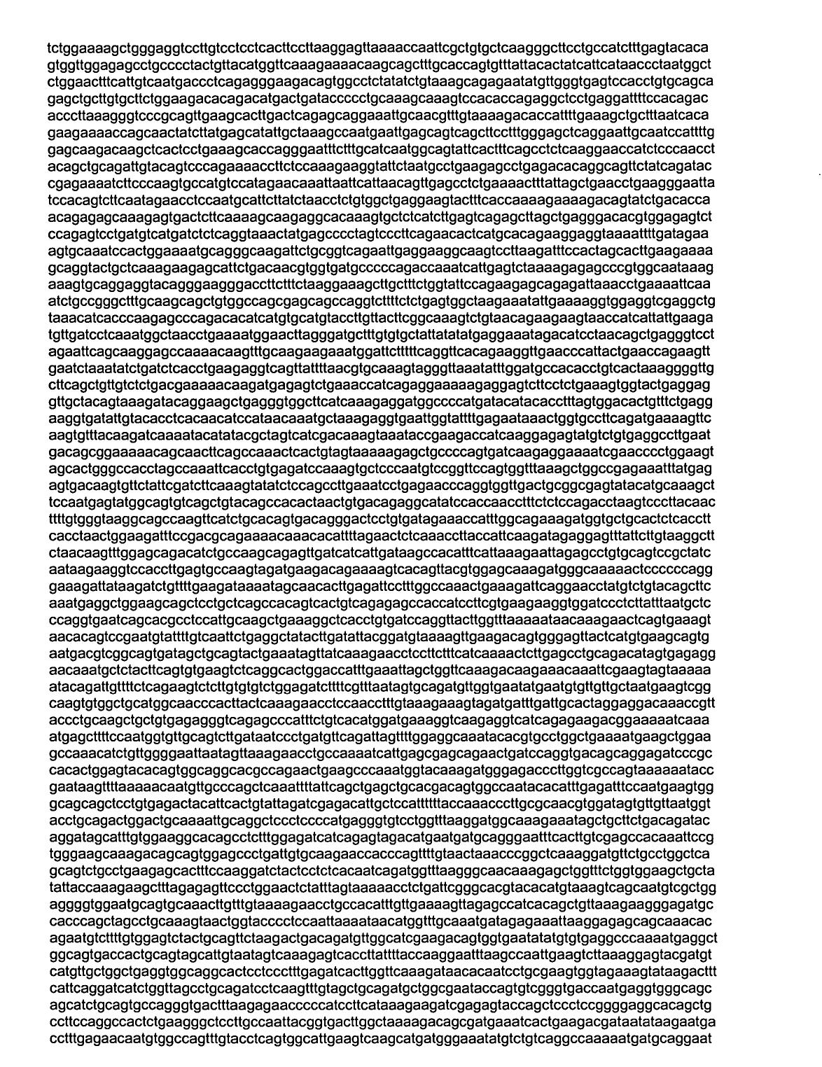

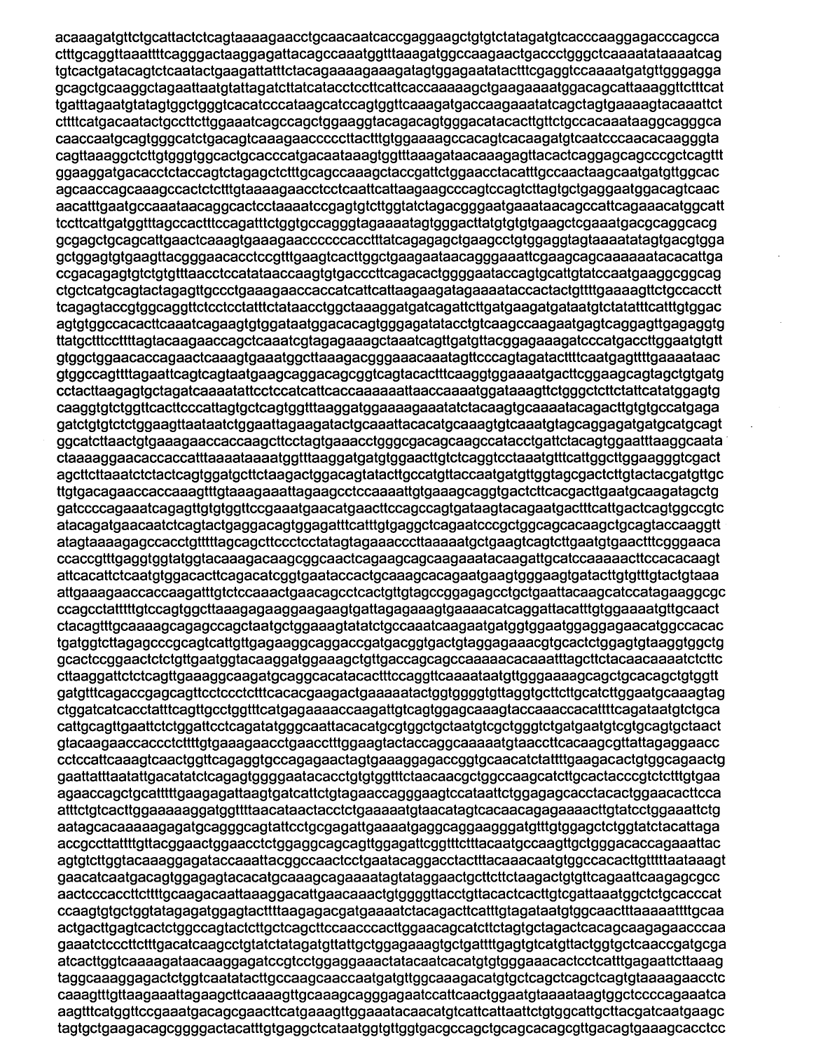

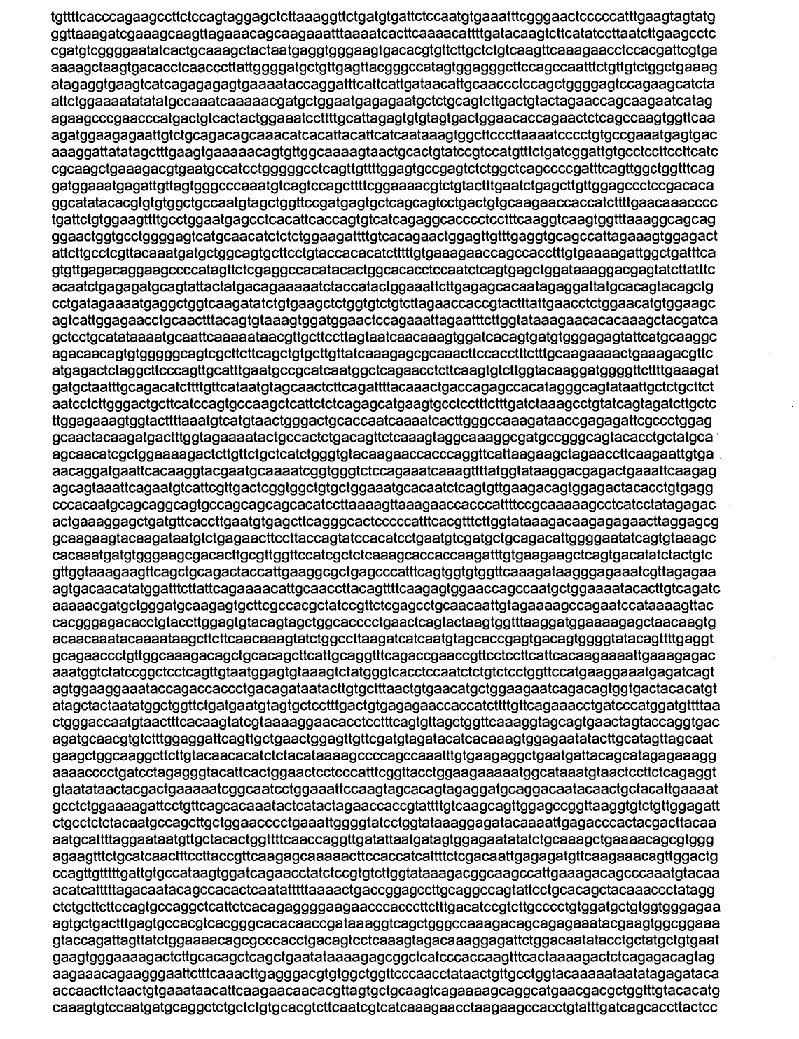

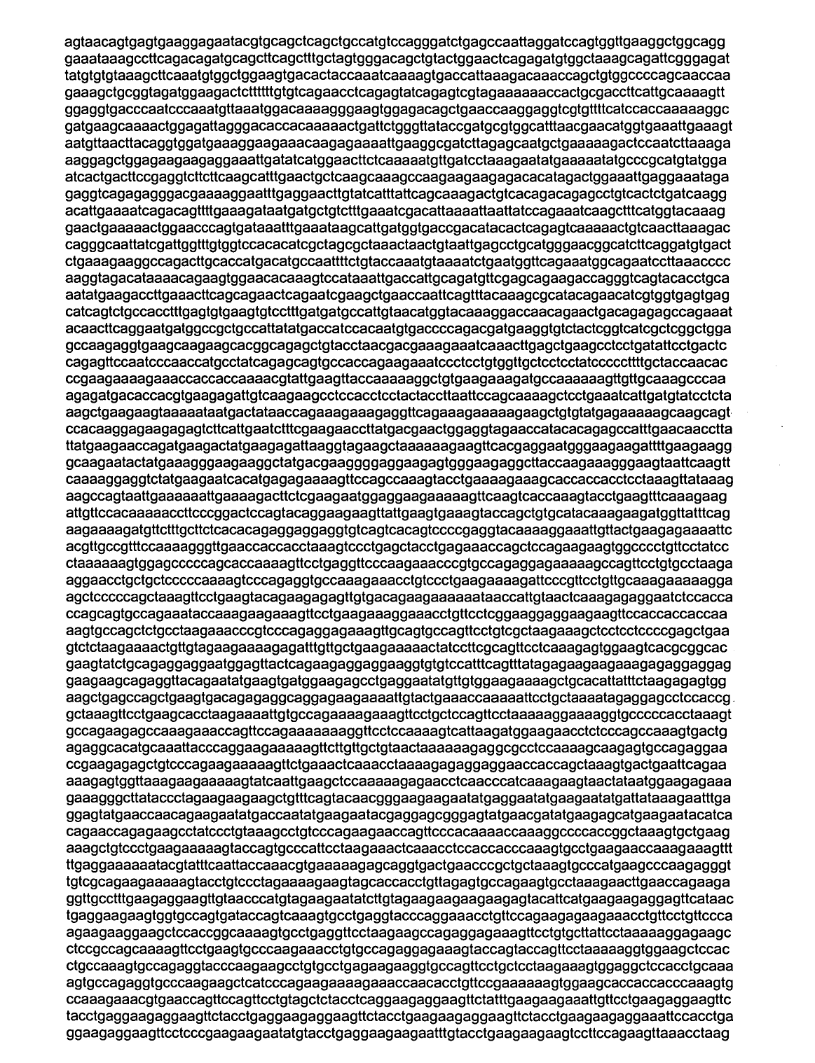

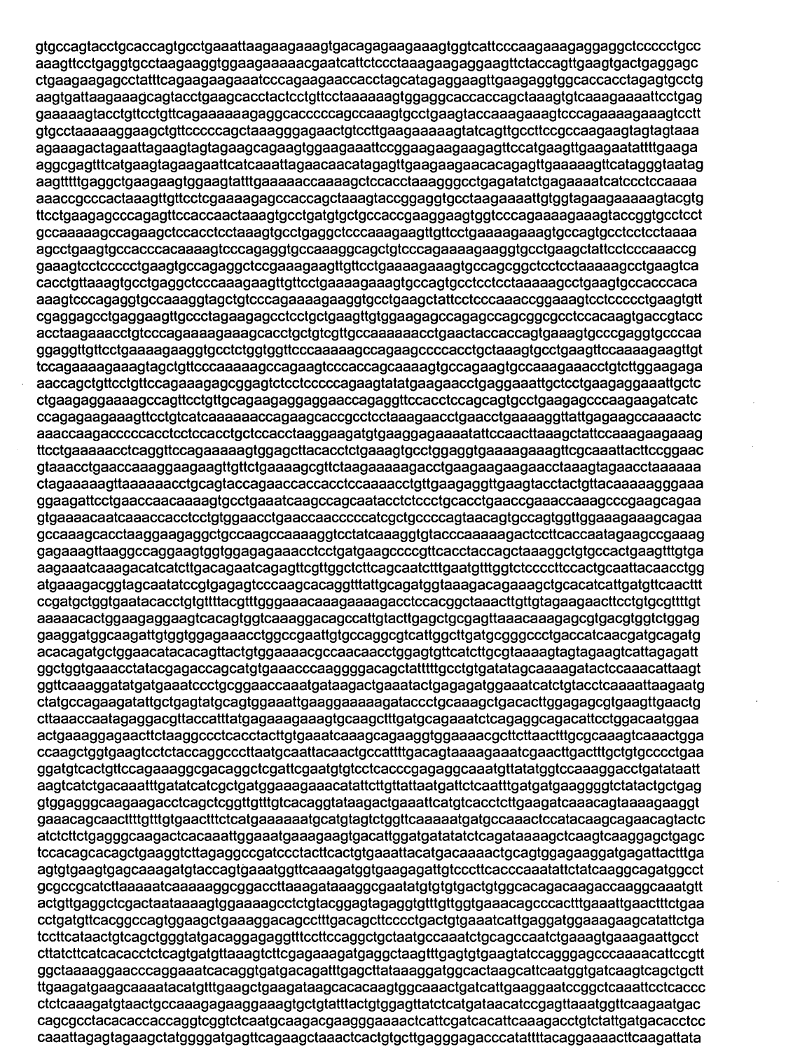

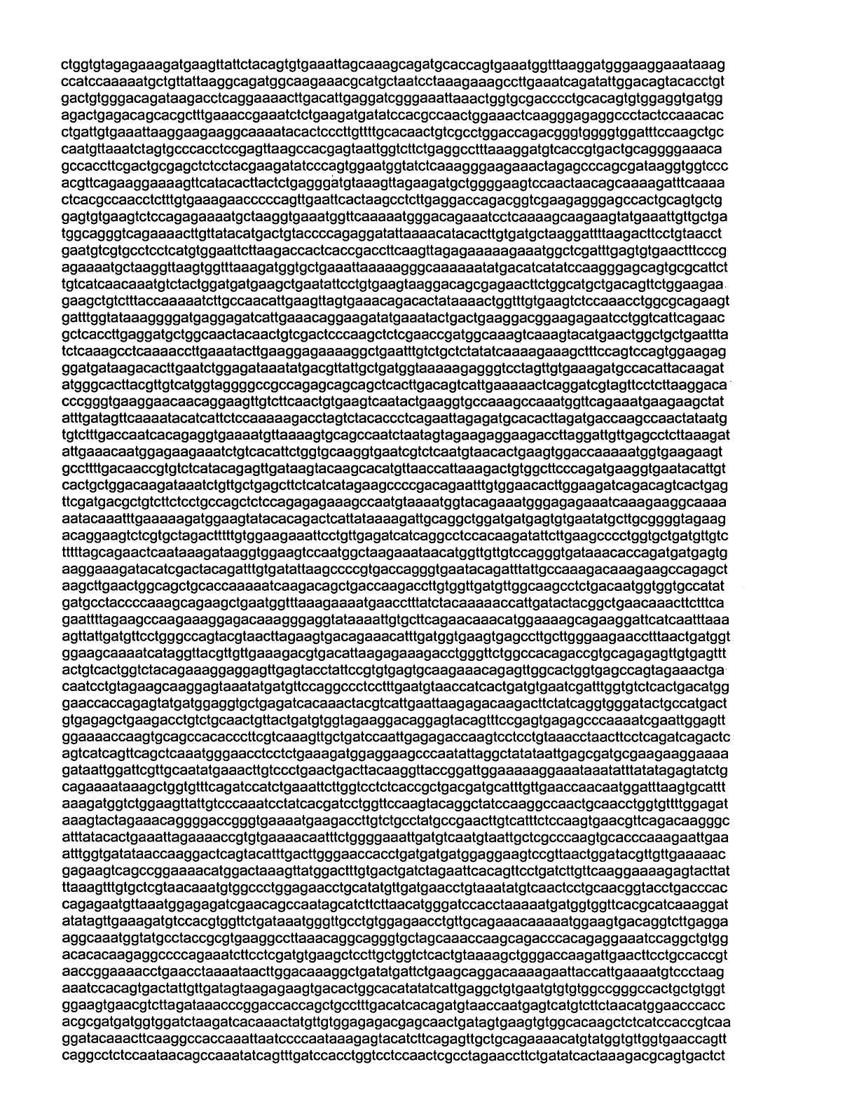

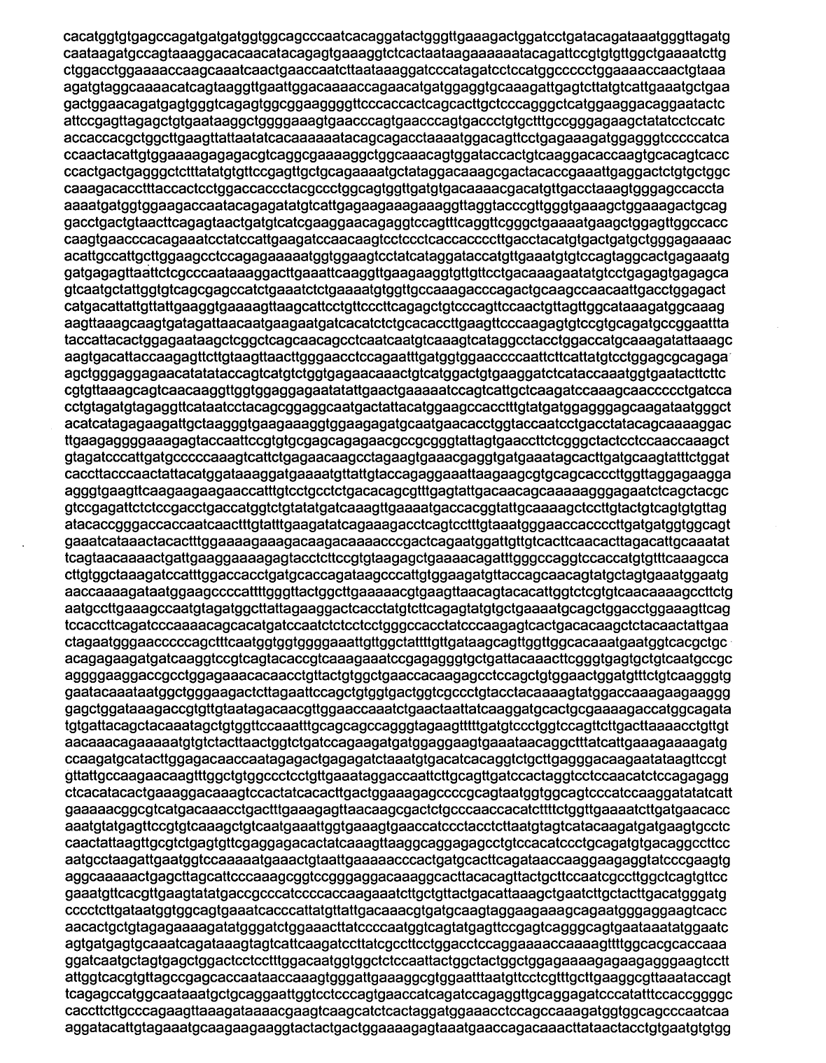

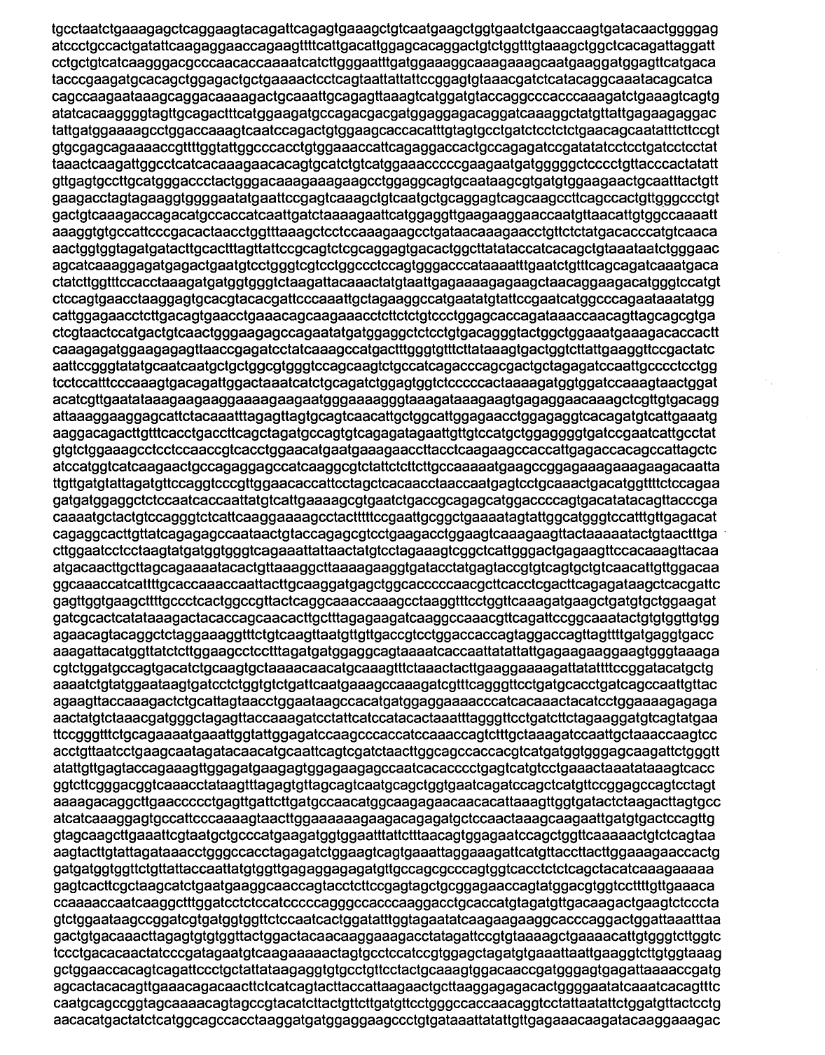

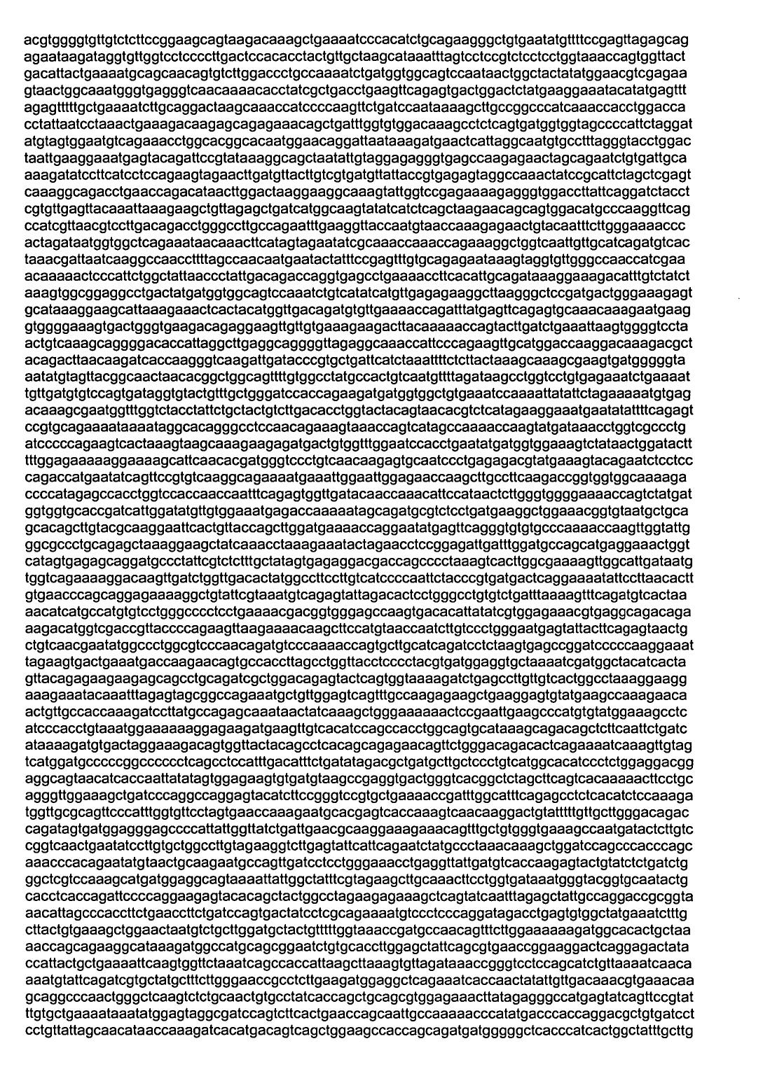

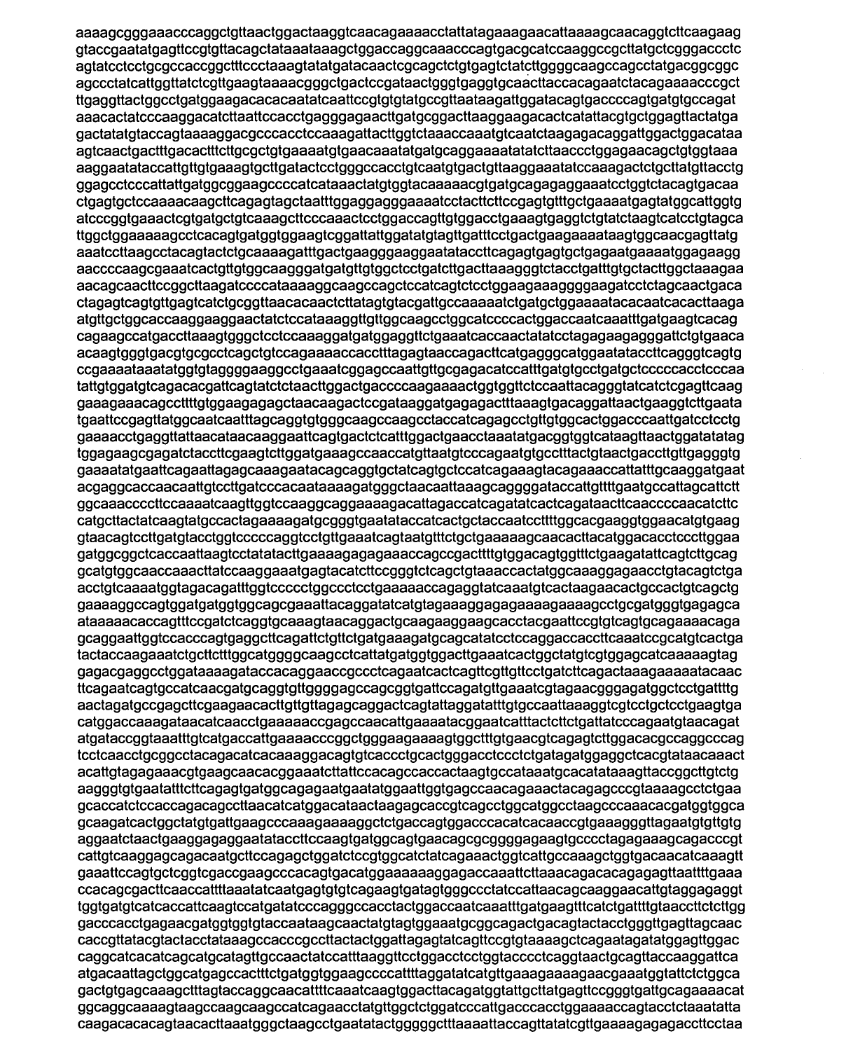

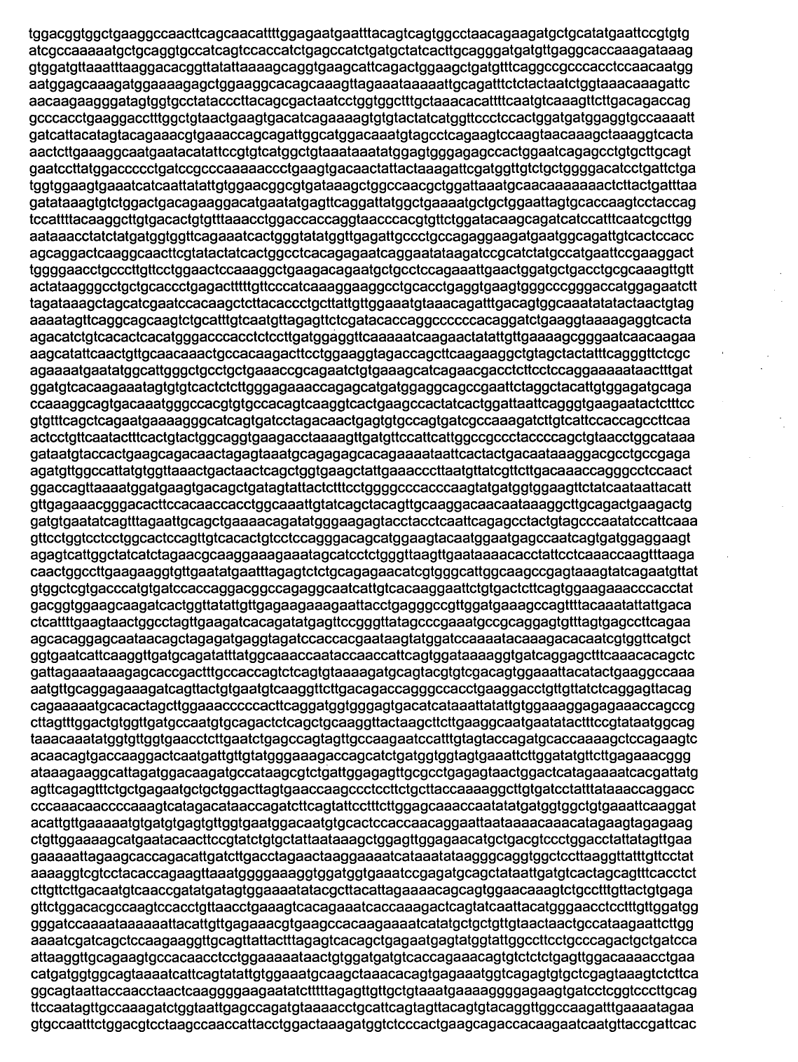

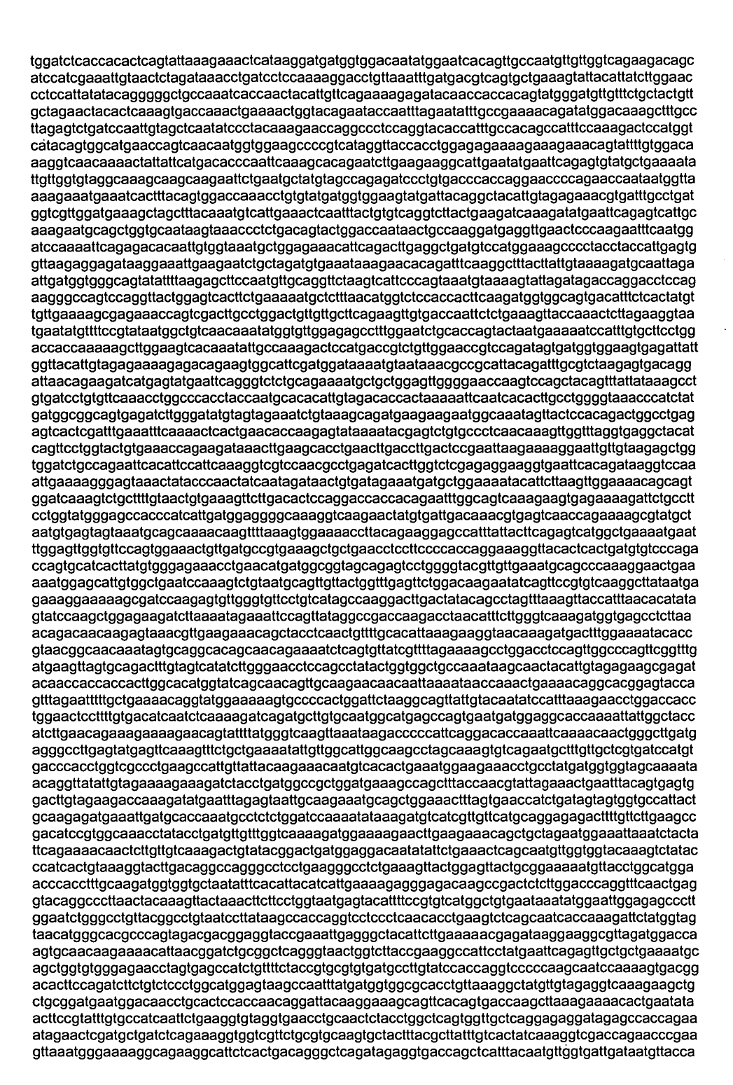

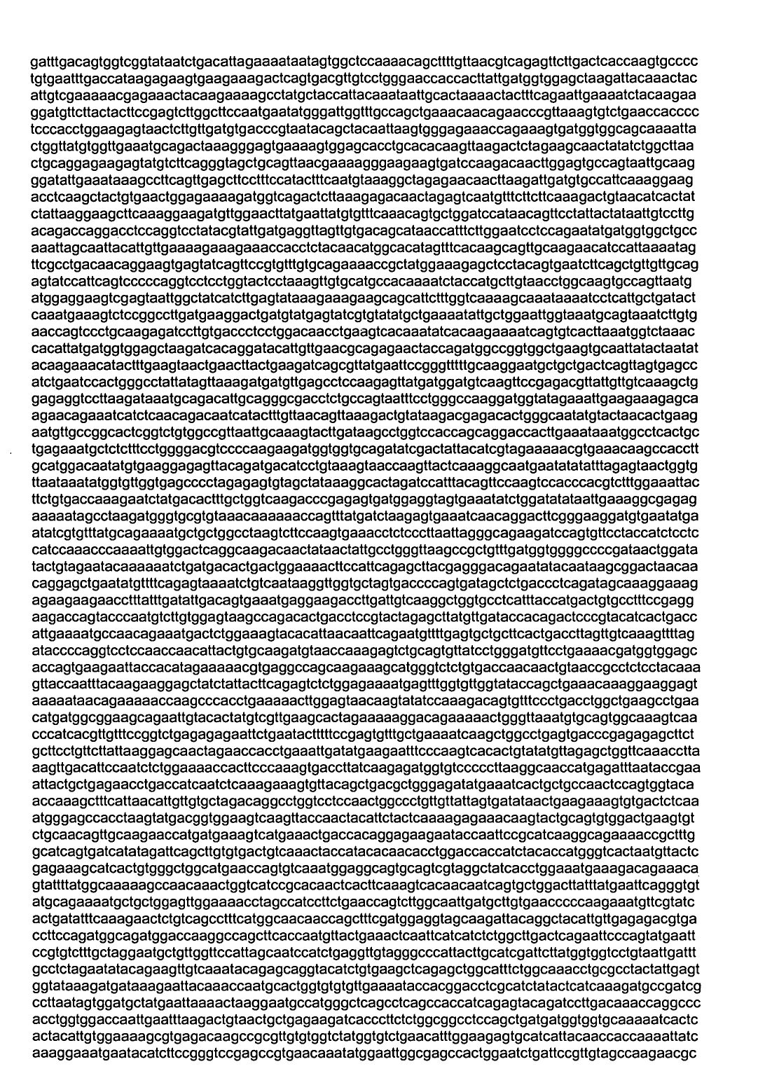

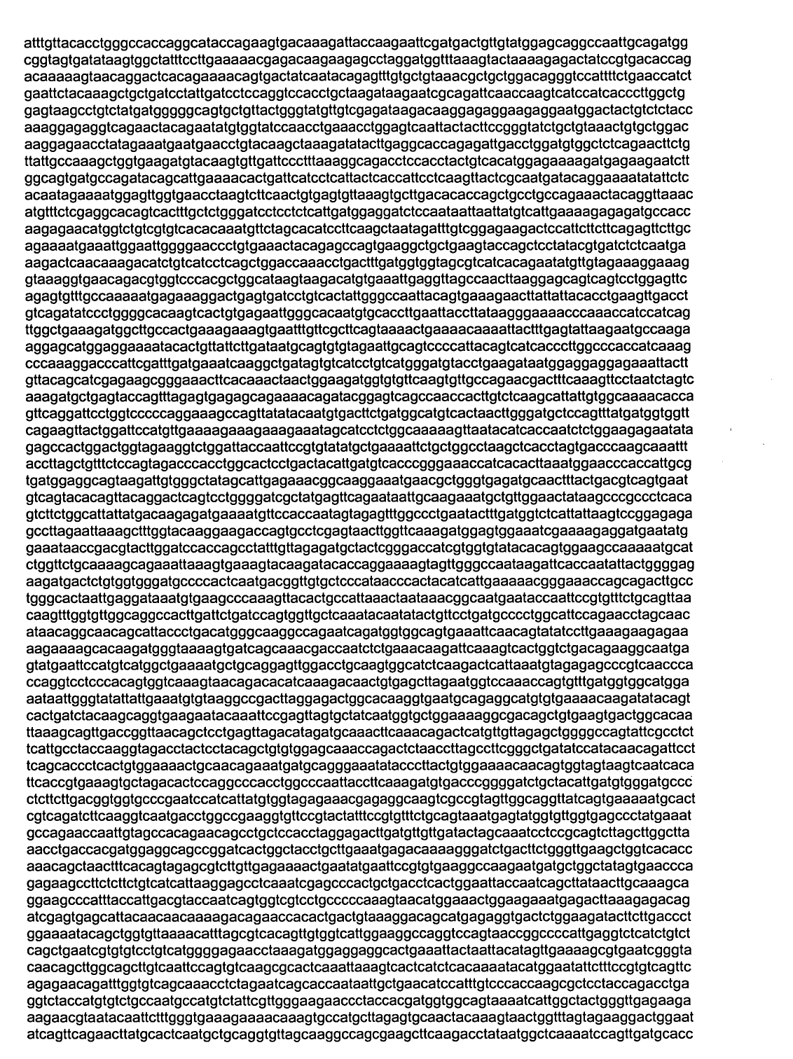

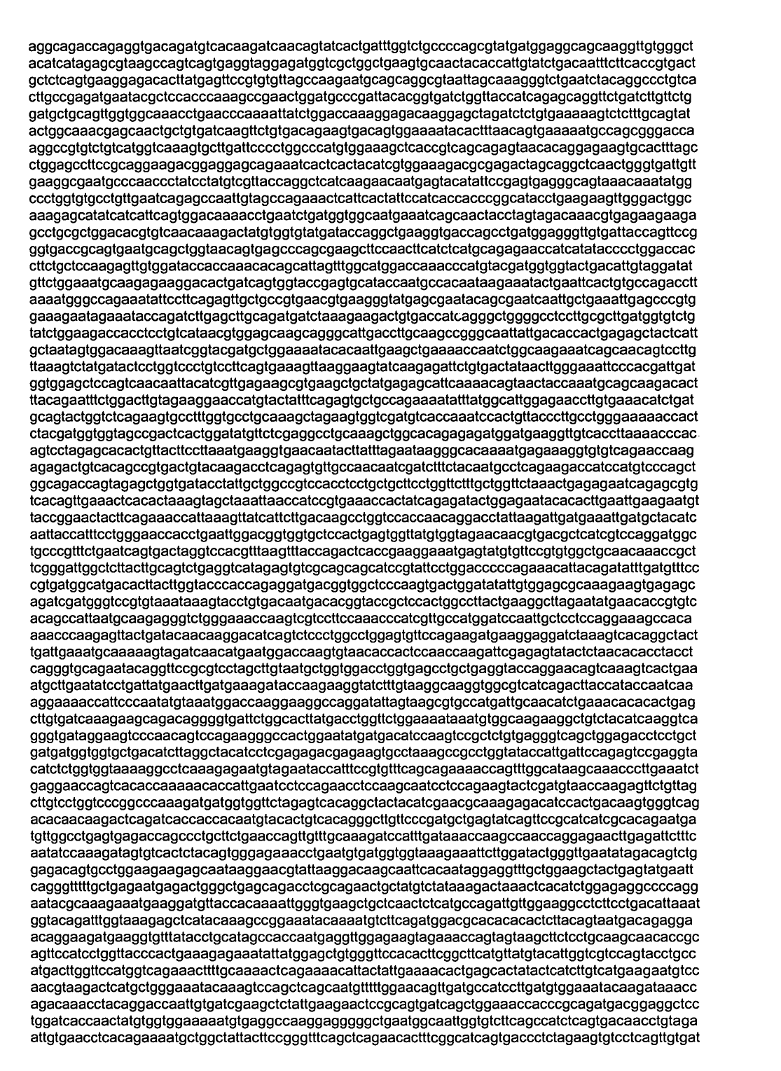

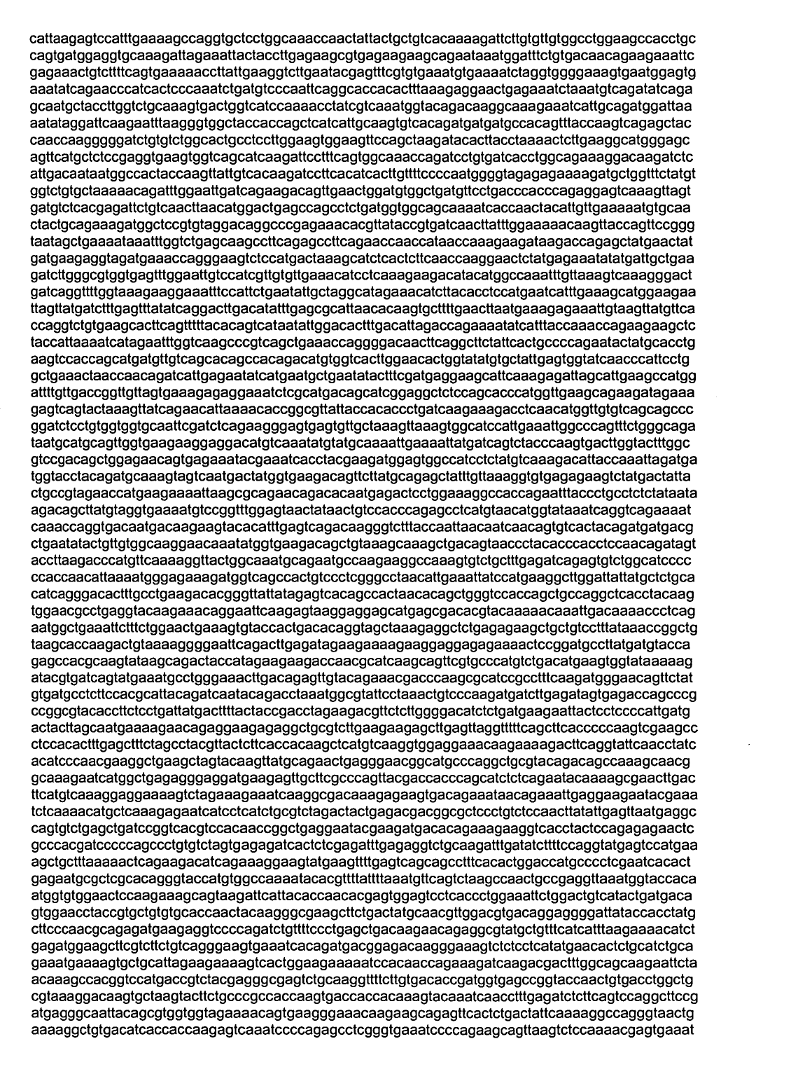

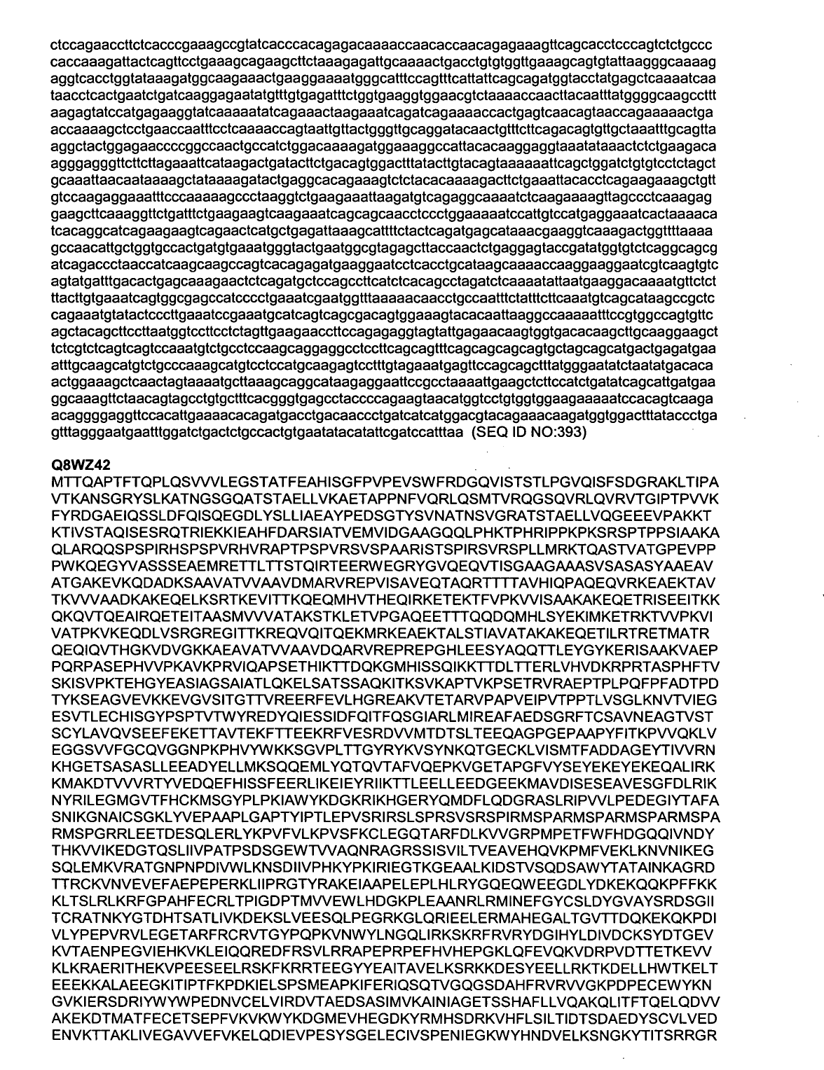

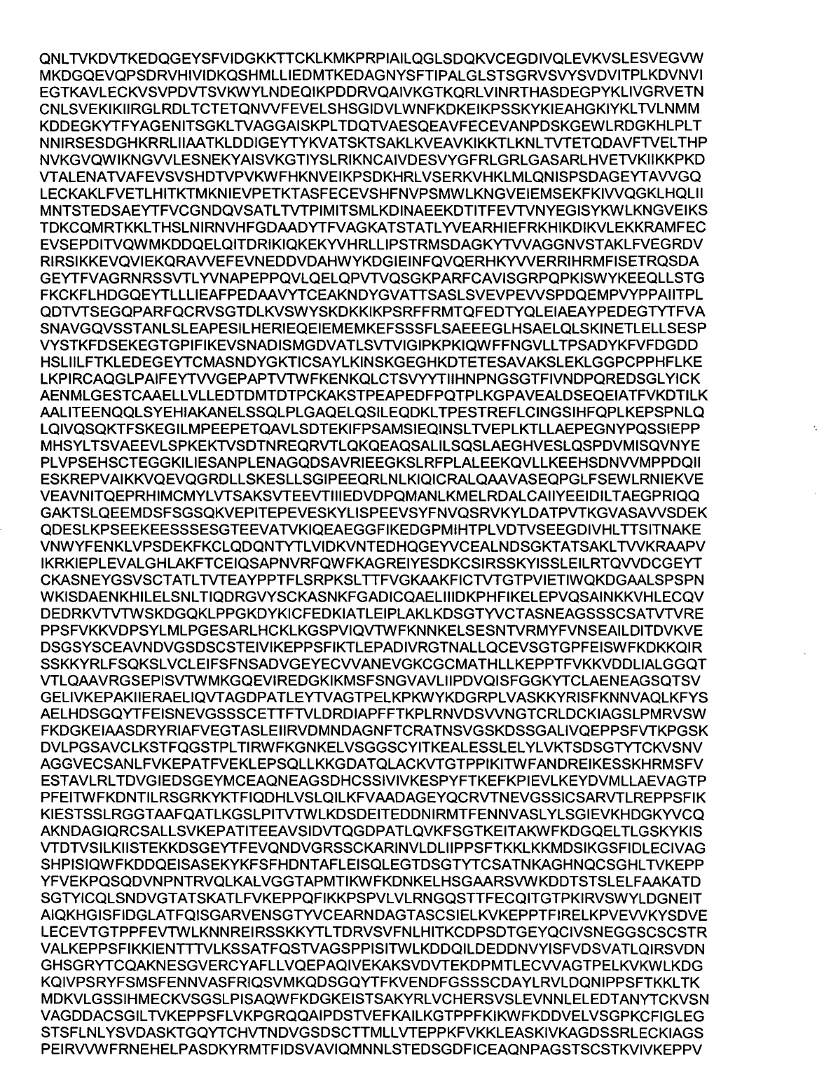

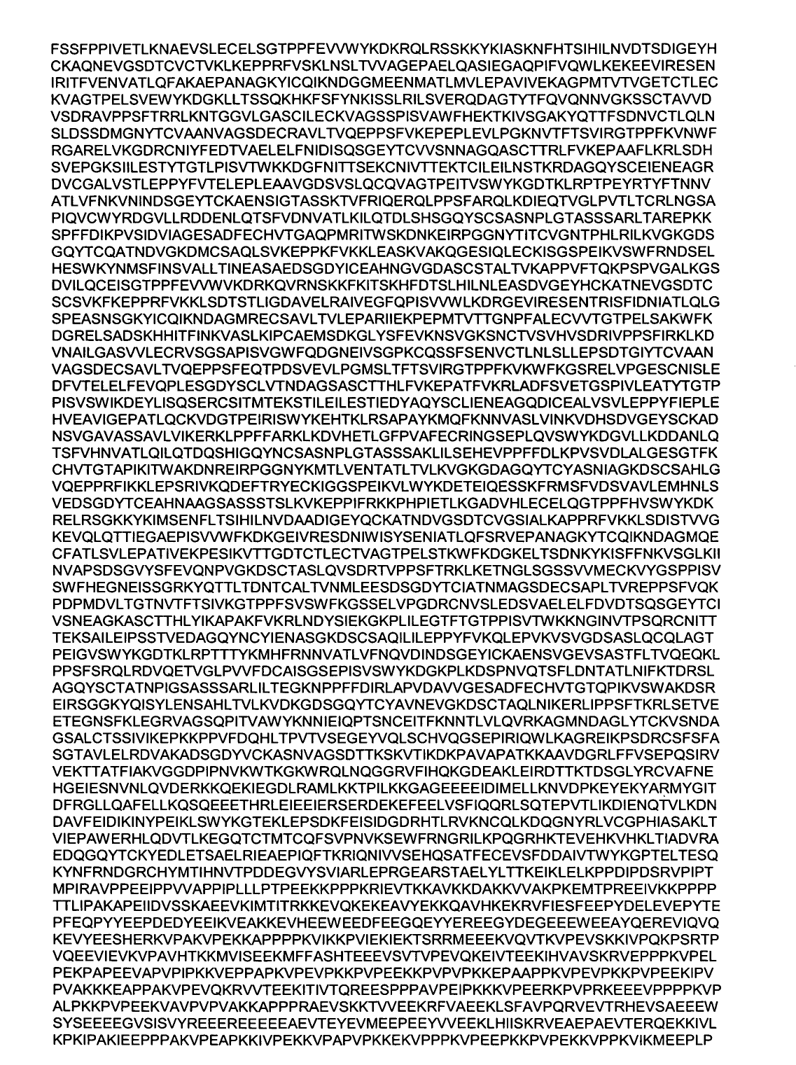

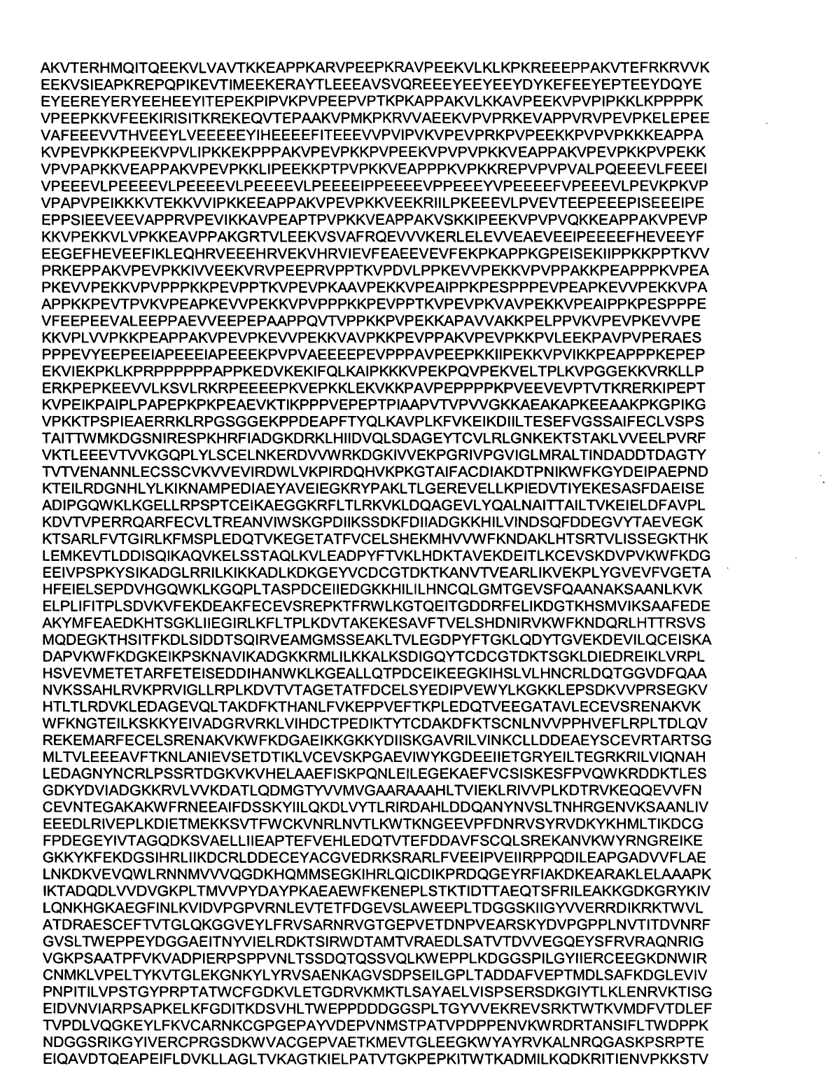

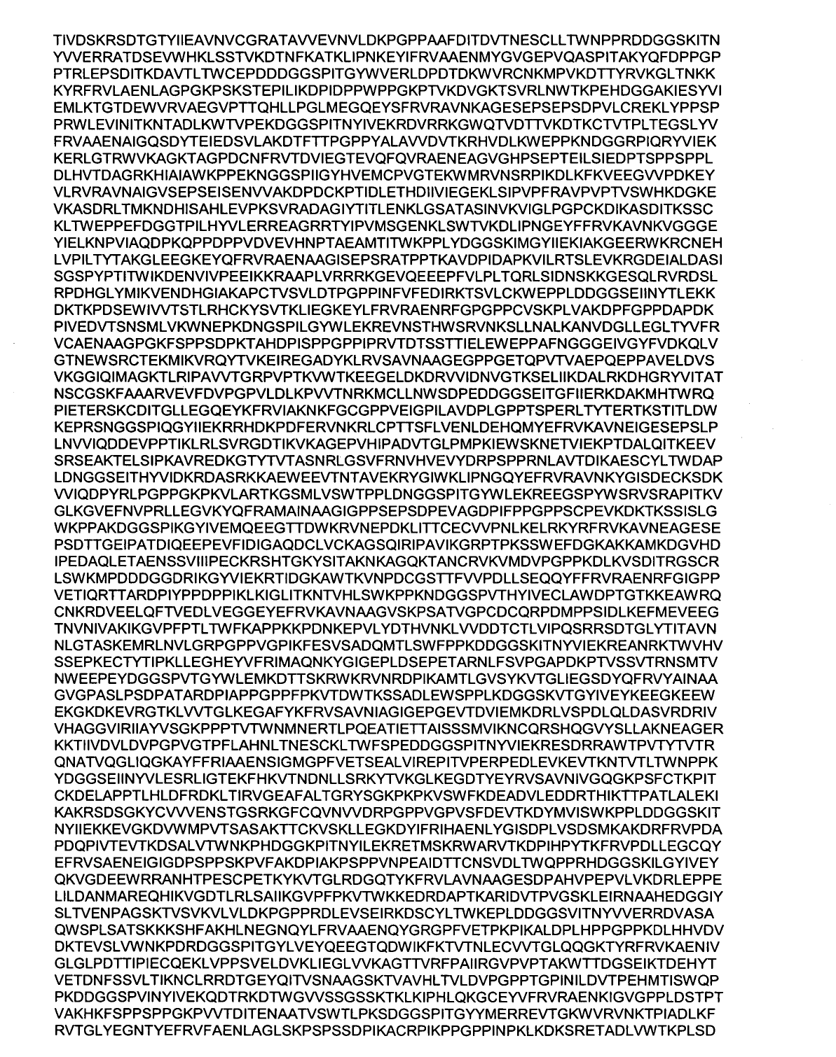

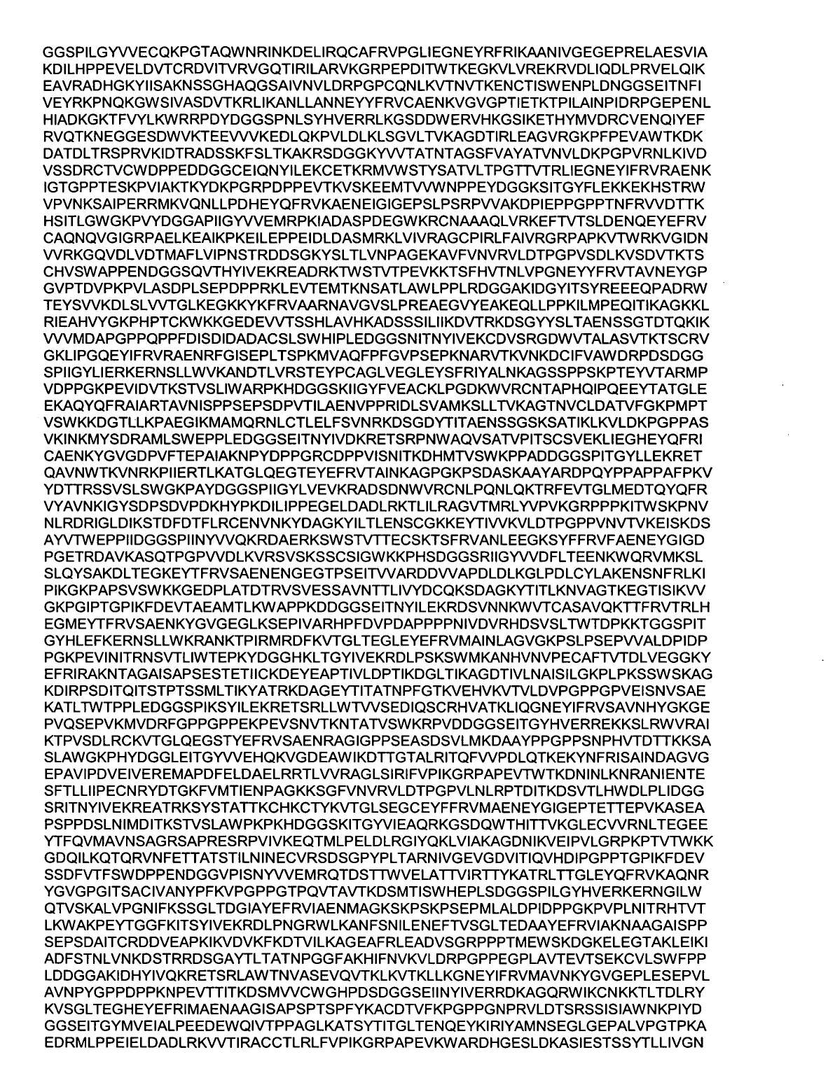

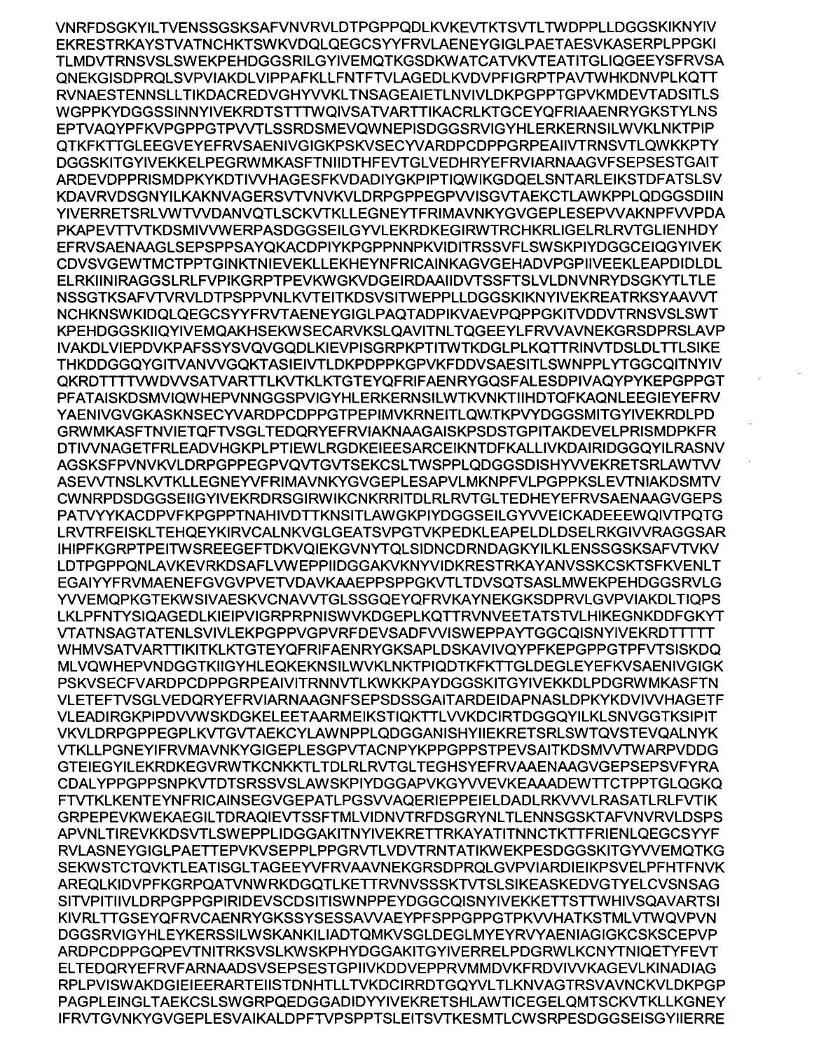

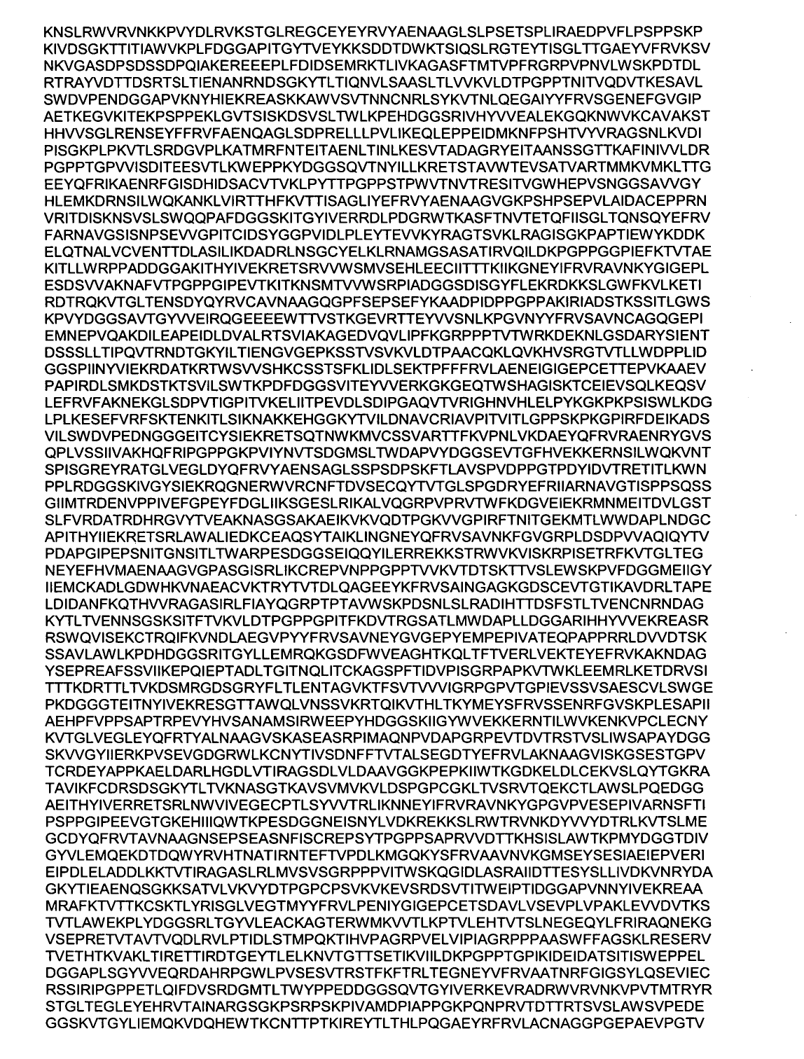

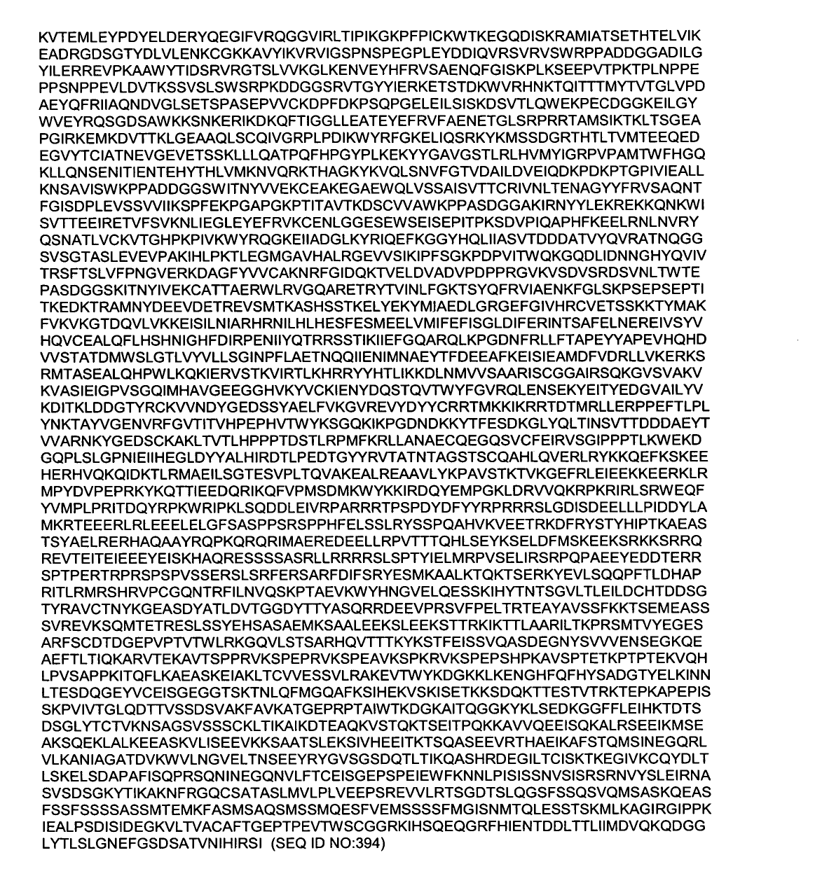

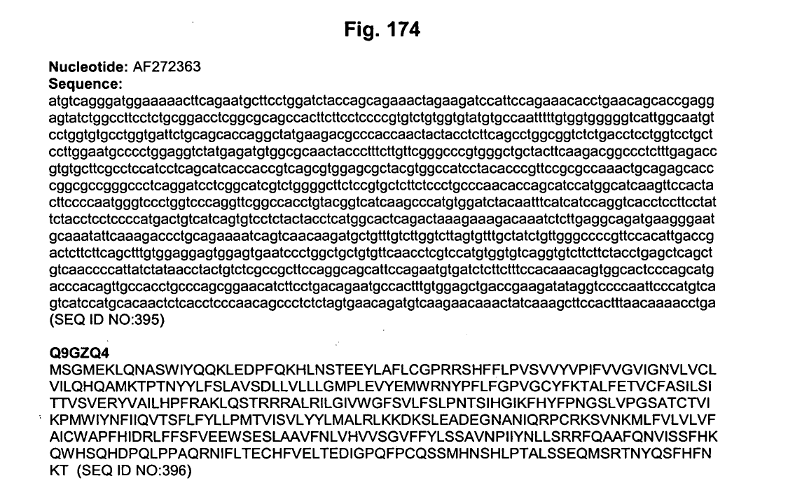

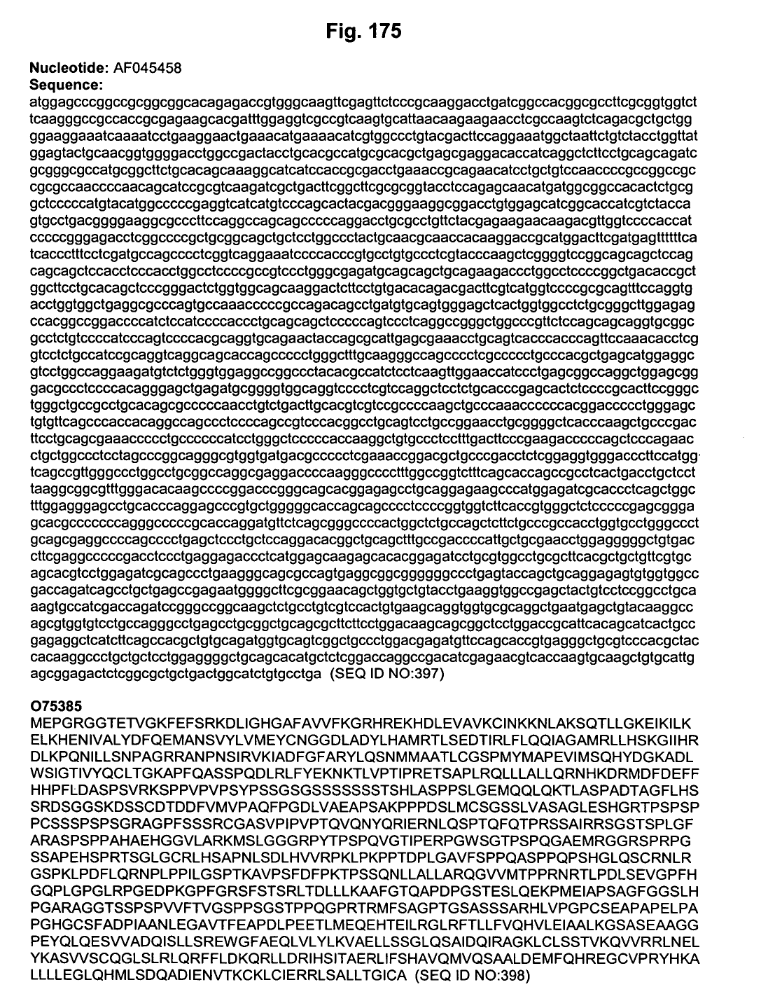

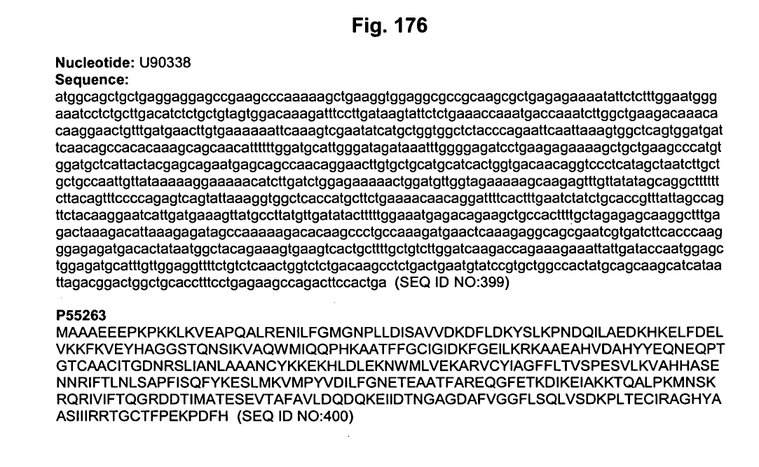

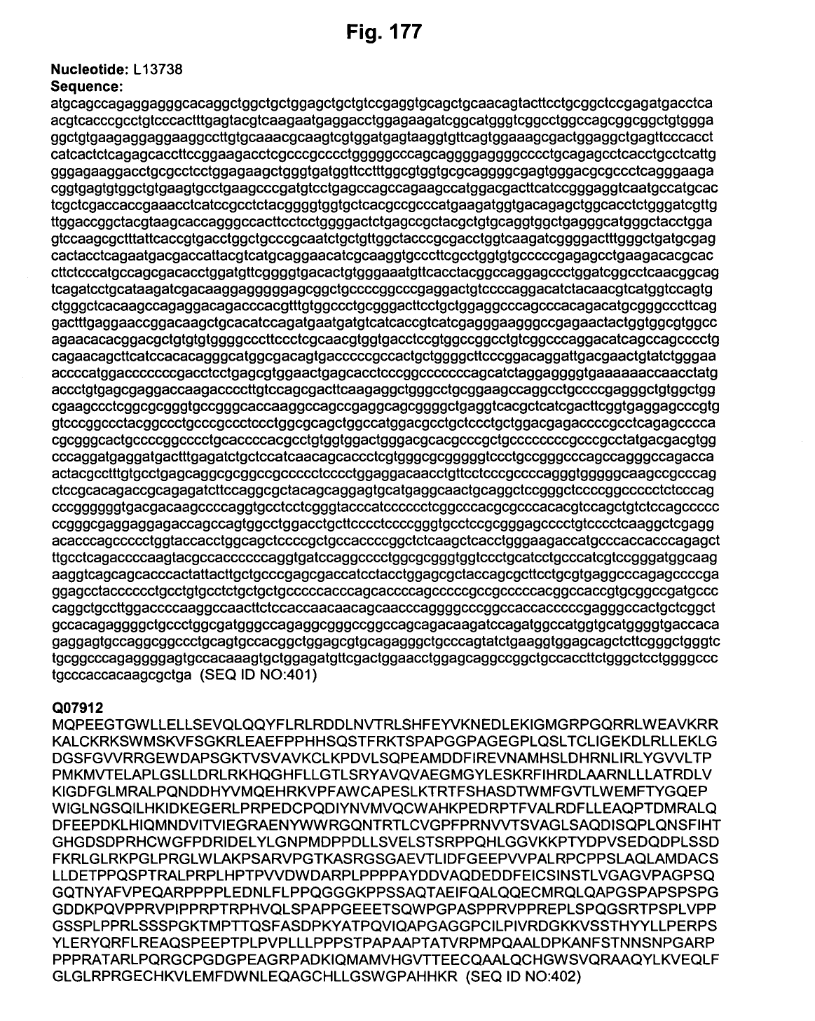

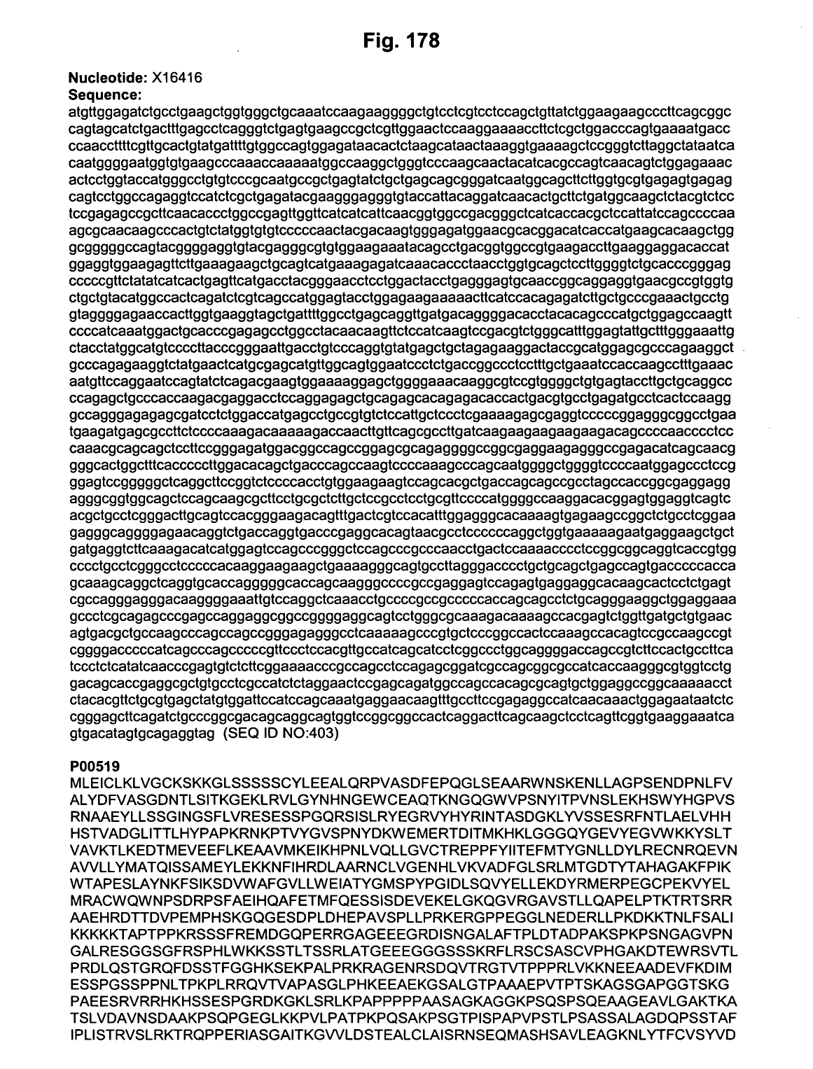

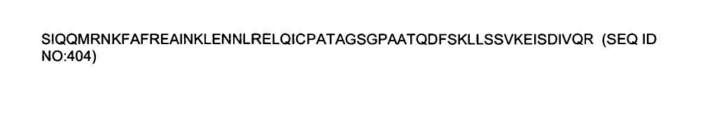

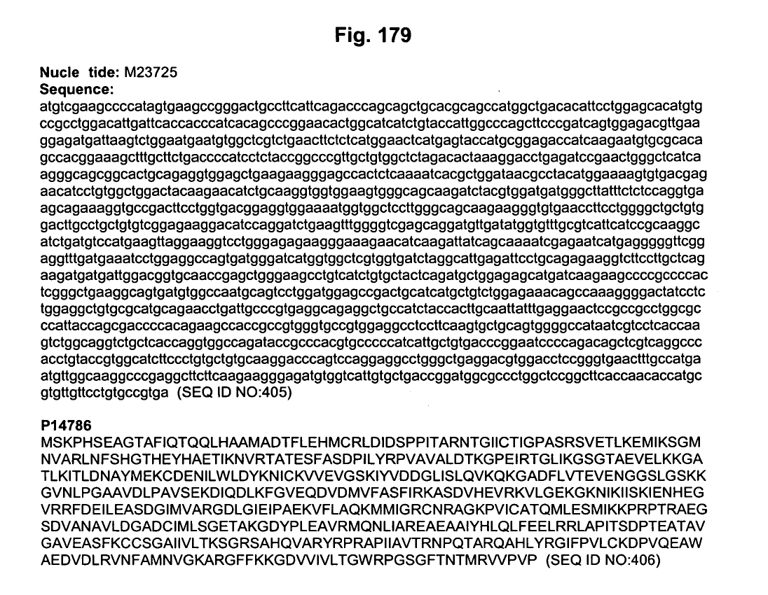

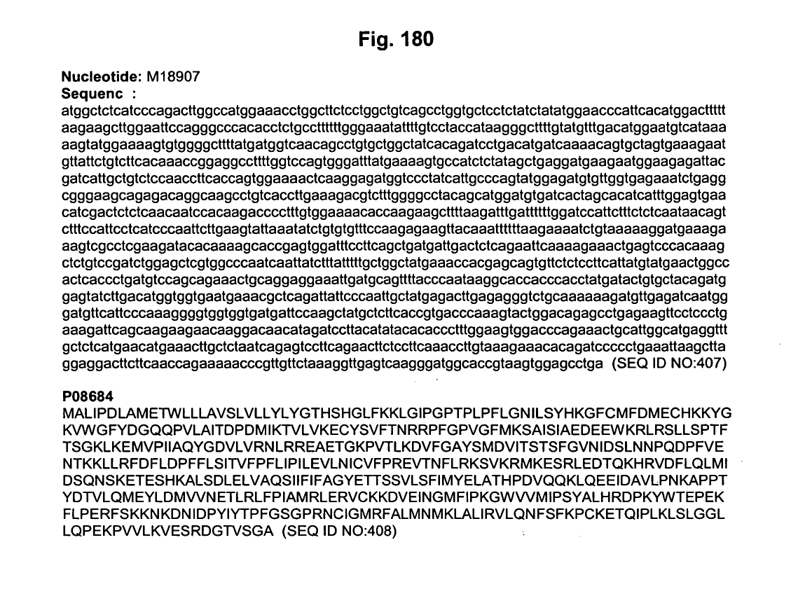

### DETAILED DESCRIPTION OF THE INVENTION

The invention describes human genes involved in cell cycle progression. Such genes can be used in assays described herein to determine if a candidate substance is an inhibitor of cell cycle progression. These same assays can be used to determine if the substance is an enhancer of cell cycle progression.

Such assays include binding asays, where the degree of binding of the substance to the polypeptides described herein is determined. Such assays also include determining if the substance prevents the binding between a polypeptide described herein and another substance known to bind that polypeptide.

The assays also include assays to determine the presence or absence of a polypeptide described herein in a sample, by adding to the sample a substance known to bind that polypeptide, and determining if binding takes place. Alternatively, a nucleic acid probe, the sequence of which is based on the nucleic acid that encodes the polypeptide, can be used to determine the presence or absence in the sample of the nucleic acid encoding the polypeptide. The probe can be added to the sample and incubated under hybridizing conditions to cause binding to the nucleic acid, if it is present in the sample. Such a probe can be labelled to more easily determine if binding has occurred. Alternatively, instead of a nucleic acid probe, an antibody specific for either the nucleic acid of the polypeptide can be used.

In situations where one wishes to know if a mutation has occurred in a nucleic acid encoding a polypeptide described herein, one can obtain a sample containing the nucleic acid, and use a nucleic acid probe specific for the region in which the mutation is believed to occur. Lack of binding, relative to a sample containing the equivalent nucleic acid without the mutation, indicates that the nucleic acid contains a mutation at that location. If it is not known where in the nucleic acid the mutation has occurred, then a sequential series of probes can be used which cover the entire length of the nucleic acid.

These assays can also be used to determine if a polypeptide described herein is being overexpressed in a tissue, e.g., a tumor, or tissue suspected of harboring overproliferating cells. An amount of a known ligand can be added to a sample from the tissue, where the ligand binds the polypeptide. If the amount of bound ligand-polypeptide complex in the sample is greater than that found normally, then the polypeptide is overexpressed. “Normally” can mean the amount of polypeptide found in a sample of another tissue from the same organism, or the equivalent tissue from another organism, or relative to previously-determined baseline levels of expression.

Any of the assays described above can be incorporated into a kit for determining presence/absence of a nucleic acid or polypeptide described herein, or the level of expression of a polypeptide described herein.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, IRL Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

By “modulating cell cycle progression” is meant that for when a given cell is treated, its normal tendency to progress through the cell cycle is changed, either increased or decreased, compared to an untreated cell, where otherwise the environmental conditions are the same.

Mitosis can be measured by FACS analysis, microscopy or by assays based on the status of cell cycle proteins. Standard assays include, but are not limited to, those protocols used in the molecular biological arts to assess cell cycle arrest, cell cycle analysis, cell proliferation of various cell types, detection of apoptosis, e.g., by apoptotic cell morphology or Annexin V-FITC assay, and inhibition of cancer cell growth or tumor growth in various animal models. Such assays are well-known to those of ordinary skill in those fields. Examples of assays are described herein.

The “functional activity” of a protein in the context of the present invention describes the function the protein performs in its native environment. Altering the functional activity of a protein includes within its scope increasing, decreasing or otherwise altering the native activity of the protein itself. In addition, it also includes within its scope increasing or decreasing the level of expression and/or altering the intracellular distribution of the nucleic acid encoding the protein, and/or altering the intracellular distribution of the protein itself.

The term “expression” refers to the transcription of a gene's DNA template to produce the corresponding mRNA and translation of this mRNA to produce the corresponding gene product (i.e., a peptide, polypeptide, or protein).

By “polynucleotide” or “polypeptide” is meant the DNA and protein sequences disclosed herein. The terms also include close variants of those sequences, where the variant possesses the same biological activity as the reference sequence. Such variant sequences include “alleles” (variant sequences found at the same genetic locus in the same or closely-related species), “homologs” (a gene related to a second gene by descent from a common ancestral DNA sequence, and separated by either speciation (“ortholog”) or genetic duplication (“paralog”)), so long as such variants retain the same biological activity as the reference sequence(s) disclosed herein.

The invention is also intended to include silent polymorphisms and conservative substitutions in the polynucleotides and polypeptides disclosed herein, so long as such variants retain the same biological activity as the reference sequence(s) as disclosed herein.

Polypeptides

It will be understood that polypeptides identified herein are not limited to polypeptides identified in Table 1 or those polypeptides having the amino acid sequence encoded by the nucleic acid sequences identified in Table 1 or fragments thereof but also include homologous sequences obtained from any source, for example related viral/bacterial proteins, cellular homologues and synthetic peptides, as well as variants or derivatives thereof.

Thus, reference herein to “polypeptides” also includes those sequences encoding homologues from other species including animals such as mammals (e.g., mice, rats or rabbits), especially primates. Particularly preferred polypeptides include homologous human sequences.

The term also covers variants, homologues or derivatives of the amino acid sequences encoded by the nucleic acids identified in Table 1, as well as variants, homologues or derivatives of the nucleotide sequences coding for the amino acid sequences.

In the context of the present invention, a homologous sequence is taken to include an amino acid sequence which is at least 15, 20, 25, 30, 40, 50, 60, 70, 80 or 90% identical, preferably at least 95 or 98% identical at the amino acid level over at least 50 or 100, preferably 200, 300, 400 or 500 amino acids with any one of the polypeptide sequences disclosed herein. In particular, homology should typically be considered with respect to those regions of the sequence known to be essential for protein function rather than non-essential neighbouring sequences. This is especially important when considering homologous sequences from distantly related organisms.

Although homology can also be considered in terms of functional similarity (i.e., amino acid residues having similar chemical properties/functions), in the context of the present invention it is preferred to express homology in terms of sequence identity.

Homology comparisons can be conducted by eye, or more usually, with the aid of readily available sequence comparison programs. These publicly and commercially available computer programs can calculate percent homology between two or more sequences.

Percent homology may be calculated over contiguous sequences, i.e., one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum percent homology therefore firstly requires the production of an optimal alignment, taking into consideration gap penalties. A suitable computer program for carrying out such an alignment is the GCG Wisconsin Bestfit package (University of Wisconsin, U.S.A; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Altschul et al., 1990, J. Mol. Biol. 215:403-410) and the GENEWORKS suite of comparison tools. Both BLAST and FASTA are available for offline and online searching (see Ausubel et al., 1999 ibid, pages 7-58 to 7-60). However it is preferred to use the GCG Bestfit program.

Although the final percent homology can be measured in terms of identity, the alignment process itself is typically not based on an all-or -nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate percent homology, preferably percent sequence identity. The software typically does this as part of the sequence comparison and generates a numerical result.

The terms “variant” or “derivative” in relation to amino acid sequences includes any substitution of, variation of, modification of, replacement of, deletion of or addition of one (or more) amino acids from or to the sequence providing the resultant amino acid sequence retains substantially the same activity as the unmodified sequence, preferably having at least the same activity as the polypeptides identified in Table 1 or the polypeptides encoded by the nucleic acid sequences identified in Table 1.

Polypeptides having the amino acid sequence encoded by the nucleic acid sequences identified in Table 1, or fragments or homologues thereof may be modified for use in the present invention. Typically, modifications are made that maintain the biological activity of the sequence. Amino acid substitutions may be made, for example from 1, 2 or 3 to 10, 20 or 30 substitutions provided that the modified sequence retains the biological activity of the unmodified sequence. Alternatively, modifications may be made to deliberately inactivate one or more functional domains of the polypeptides of the invention. Amino acid substitutions may include the use of non-naturally occurring analogues, for example to increase blood plasma half-life of a therapeutically administered polypeptide.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Polypeptides for use in the invention also include fragments of the full length sequences mentioned above. Preferably said fragments comprise at least one epitope. Methods of identifying epitopes are well known in the art. Fragments will typically comprise at least 6 amino acids, more preferably at least 10, 20, 30, 50 or 100 amino acids. Proteins are typically made by recombinant means, for example as described below. However they may also be made by synthetic means using techniques well known to skilled persons such as solid phase synthesis. Proteins may also be produced as fusion proteins, for example to aid in extraction and purification. Examples of fusion protein partners include glutathione-S-transferase (GST), 6×His, GAL4 (DNA binding and/or transcriptional activation domains) and β-galactosidase. It may also be convenient to include a proteolytic cleavage site between the fusion protein partner and the protein sequence of interest to allow removal of fusion protein sequences. Preferably the fusion protein will not hinder the function of the protein of interest sequence. Proteins may also be obtained by purification of cell extracts from animal cells.

Multimeric proteins comprising the cell cycle progression proteins are also intended to be encompassed by the present invention. By “multimer” is meant a protein comprising two or more copies of a subunit protein. The subunit protein may be one of the proteins of the present invention, e.g., a cell cycle progression protein as disclosed herein repeated two or more times. Such a multimer may also be a fusion or chimeric protein, e.g., a repeated cell cycle progression protein may be combined with polylinker sequence, and/or one or more cell cycle progression peptides, which may be present in a single copy, or may also be tandemly repeated, e.g., a protein may comprise two or more multimers within the overall protein.

Proteins may be in a substantially isolated form. It will be understood that the protein may be mixed with carriers or diluents which will not interfere with the intended purpose of the protein and still be regarded as substantially isolated. A protein for use in the invention may also be in a substantially purified form, in which case it will generally comprise the protein in a preparation in which more than 90%, e.g., 95%, 98% or 99% of the protein in the preparation is a protein as identified herein.

A polypeptide may be labeled with a revealing label. The revealing label may be any suitable label which allows the polypeptide to be detected. Suitable labels include radioisotopes, e.g., ¹²⁵I, enzymes, antibodies, polynucleotides and linkers such as biotin. Labeled polypeptides of the invention may be used in diagnostic procedures such as immunoassays to determine the amount of a polypeptide of the invention in a sample. Polypeptides or labeled polypeptides of the invention may also be used in serological or cell-mediated immune assays for the detection of immune reactivity to said polypeptides in animals and humans using standard protocols.

A polypeptide or labeled polypeptide or fragment thereof may also be fixed to a solid phase, for example the surface of a microarray, an immunoassay well or dipstick. Such labeled and/or immobilised polypeptides may be packaged into kits in a suitable container along with suitable reagents, controls, instructions and the like. Such polypeptides and kits may be used in methods of detection of antibodies to the polypeptides or their allelic or species variants by immunoassay.

Immunoassay methods are well known in the art and will generally comprise: (a) providing a polypeptide comprising an epitope bindable by an antibody against said protein; (b) incubating a biological sample with said polypeptide under conditions which allow for the formation of an antibody-antigen complex; and (c) determining whether antibody-antigen complex comprising said polypeptide is formed.

Immunoassays may be used for detecting polypeptides for examples in detecting a modulation of protein expression or function.

Polypeptides identified herein may be used in in vitro or in vivo cell culture systems to study the role of their corresponding genes and homologues thereof in cell function, including their function in disease. For example, truncated or modified polypeptides may be introduced into a cell to disrupt the normal functions which occur in the cell. The polypeptides of the invention may be introduced into the cell by in situ expression of the polypeptide from a recombinant expression vector (see below). The expression vector optionally carries an inducible promoter to control the expression of the polypeptide.

The use of appropriate host cells, such as insect cells or mammalian cells, is expected to provide for such post-translational modifications (e.g., myristolation, glycosylation, truncation, lapidation and tyrosine, serine or threonine phosphorylation) as may be needed to confer optimal biological activity on recombinant expression products of the invention. Such cell culture systems in which polypeptides of the invention are expressed may be used in assay systems to identify candidate substances which interfere with or enhance the functions of the polypeptides of the invention in the cell.

Polynucleotides

We demonstrate here that knockdown of genes as disclosed in the Examples causes a cell cycle defect, and that accordingly these genes and the proteins encoded by them are responsible for cell cycle function.

Polynucleotides or nucleic acids of the invention include polynucleotides identified in Table 1 or any one or more of the nucleic acid sequences encoding the polypeptides which are encoded by the nucleic acids identified in Table 1 and fragments thereof. Fragments will typically comprise at least 15 consecutive nucleotides of the full-length polynucleotide, more preferably at least 20, 30, 50 or 100 consecutive nucleotides of the full-length polynucleotide. It is straightforward to identify a nucleic acid sequence which encodes such a polypeptide, by reference to the genetic code. Furthermore, computer programs are available which translate a nucleic acid sequence to a polypeptide sequence, and/or vice versa. The disclosure of a nucleic acid and its corresponding polypeptide sequence includes a disclosure of all nucleic acids (and their sequences) which encode that polypeptide sequence.

It will be understood by a skilled person that numerous different polynucleotides can encode the same polypeptide as a result of the degeneracy of the genetic code. In addition, it is to be understood that skilled persons may, using routine techniques, make nucleotide substitutions that do not affect the polypeptide sequence encoded by the polynucleotides of the invention to reflect the codon usage of any particular host organism in which the polypeptides of the invention are to be expressed.

In preferred embodiments of the invention, nucleic acids of the invention comprise those polynucleotides, such as cDNA, mRNA, and genomic DNA. Such polynucleotides may typically comprise Drosophila cDNA, mRNA, and genomic DNA, Homo sapiens cDNA, mRNA, and genomic DNA, etc. Accession numbers are provided in the Examples for the nucleic acid sequences, and the polypeptides they encode can be derived by use of such accession numbers in a relevant database, such as a Drosophila sequence database, a human sequence database, including a Human Genome Sequence database, GadFly, FlyBase, etc. in particular, the annotated Drosophila sequence database of the Berkeley Drosophila Genome Project (GadFly: Genome Annotation Database of Drosophila at the world wide web site “fruitfly.org”, in the directory “/annot/”) may be used to identify such Drosophila and human polynucleotide or polypeptide sequences. Relevant sequences may also be obtained by searching sequence databases such as BLAST with the polypeptide sequences. In particular, a search using TBLASTN may be employed.

Nucleic acids for use in the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides described herein may be modified by any method available in the art. Such modifications may be carried out in order to enhance the in vivo activity or life span of polynucleotides of the invention.

The terms “variant”, “homologue” or “derivative” in relation to the nucleotide sequence for use in the present invention include any substitution of, variation of, modification of, replacement of, deletion of or addition of one (or more) nucleic acid from or to the sequence. Preferably said variant, homologues or derivatives code for a polypeptide having biological activity.

As indicated above, with respect to sequence homology, preferably there is at least 50 or 75%, more preferably at least 85%, more preferably at least 90% homology to the sequences shown in the sequence listing herein. More preferably there is at least 95%, more preferably at least 98%, homology. Nucleotide homology comparisons may be conducted as described above. A preferred sequence comparison program is the GCG Wisconsin Bestfit program described above. The default scoring matrix has a match value of 10 for each identical nucleotide and −9 for each mismatch. The default gap creation penalty is −50 and the default gap extension penalty is −3 for each nucleotide.

The present invention also encompasses the use of nucleotide sequences that are capable of hybridising selectively to the sequences presented herein, or any variant, fragment or derivative thereof, or to the complement of any of the above. Nucleotide sequences are preferably at least 15 nucleotides in length, more preferably at least 20, 30, 40 or 50 nucleotides in length.

The term “hybridization” as used herein shall include “the process by which a strand of nucleic acid joins with a complementary strand through base pairing” as well as the process of amplification as carried out in polymerase chain reaction technologies.

Polynucleotides capable of selectively hybridising to the nucleotide sequences presented herein, or to their complement, will be generally at least 70%, preferably at least 80 or 90% and more preferably at least 95% or 98% homologous to the corresponding nucleotide sequences presented herein over a region of at least 20, preferably at least 25 or 30, for instance at least 40, 60 or 100 or more contiguous nucleotides.

The term “selectively hybridizable” means that a nucleic acid is found to hybridize to the nucleic acid having a sequence identified in Table 1 at a level significantly above background. Background implies a level of signal generated by interaction between the test nucleic acid and a non-specific DNA member in a sample which is less than 10 fold, preferably less than 100 fold as intense as the specific interaction observed with the target DNA. The intensity of interaction may be measured, for example, by radiolabelling the probe, e.g., with ³²P.

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below. Conditions for stringency are also described in U.S. Pat. No. 5,976,838, the teachings of which are incorporated herein by reference in its entirety. In particular, examples of highly stringent, stringent, reduced and least stringent conditions are provided in U.S. Pat. No. 5,976,838, in the Table on page 15. Examples of stringency conditions for solutions during and after hybridization are shown, and highly stringent conditions are those that are at least as stringent as, for example, conditions A-F; stringent conditions are at least as stringent as, for example, conditions G-L; and reduced stringency conditions are at least as stringent as, for example, conditions M-R of that table.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical polynucleotide sequences while an intermediate (or low) stringency hybridization can be used to identify or detect similar or related polynucleotide sequences.

Stringency conditions for hybridization refers to conditions of temperature and buffer composition which permit hybridization of a first nucleic acid sequence to a second nucleic acid sequence, wherein the conditions determine the degree of identity between those sequences which hybridize to each other. Therefore, “high stringency conditions” are those conditions wherein only nucleic acid sequences which are very similar to each other will hybridize. The sequences may be less similar to each other if they hybridize under moderate stringency conditions. Still less similarity is needed for two sequences to hybridize under low stringency conditions. By varying the hybridization conditions from a stringency level at which no hybridization occurs, to a level at which hybridization is first observed, conditions can be determined at which a given sequence will hybridize to those sequences that are most similar to it. The precise conditions determining the stringency of a particular hybridization include not only the ionic strength, temperature, and the concentration of destabilizing agents such as formamide, but also on factors such as the length of the nucleic acid sequences, their base composition, the percent of mismatched base pairs between the two sequences, and the frequency of occurrence of subsets of the sequences (e.g., small stretches of repeats) within other non-identical sequences. Washing is the step in which conditions are set so as to determine a minimum level of similarity between the sequences hybridizing with each other. Generally, from the lowest temperature at which only homologous hybridization occurs, a 1% mismatch between two sequences results in a 1° C. decrease in the melting temperature (Tₘ) for any chosen SSC concentration. Generally, a doubling of the concentration of SSC results in an increase in the Tₘ of about 17° C. Using these guidelines, the washing temperature can be determined empirically, depending on the level of mismatch sought. Hybridization and wash conditions are explained in Current Protocols in Molecular Biology (Ausubel, F. M. et al., eds., John Wiley & Sons, Inc., 1995, with supplemental updates) on pages 2.10.1 to 2.10.16, and 6.3.1 to 6.3.6.

High stringency conditions can employ hybridization at either (1) 1×SSC (10×SSC=3 M NaCl, 0.3 M Na₃-citrate.2H₂O (88 g/liter), pH to 7.0 with 1 M HCl), 1% SDS (sodium dodecyl sulfate), 0.1-2 mg/ml denatured salmon sperm DNA at 65° C., (2) 1×SSC, 50% formamide, 1% SDS, 0.1-2 mg/ml denatured salmon sperm DNA at 42° C., (3) 1% bovine serum albumen (fraction V), 1 mM Na₂.EDTA, 0.5 M NaHPO₄ (pH 7.2) (1 M NaHPO₄=134 g Na₂HPO₄.7H₂O, 4 ml 85% H₃PO₄ per liter), 7% SDS, 0.1-2 mg/ml denatured salmon sperm DNA at 65° C., (4) 50% formamide, 5×SSC, 0.02 M Tris-HCl (pH 7.6), 1× Denhardt's solution (100X=10 g Ficoll 400, 10 g polyvinylpyrrolidone, 10 g bovine serum albumin (fraction V), water to 500 ml), 10% dextran sulfate, 1% SDS, 0.1-2 mg/ml denatured salmon sperm DNA at 42° C., (5) 5×SSC, 5× Denhardt's solution, 1% SDS, 100:g/ml denatured salmon sperm DNA at 65° C., or (6) 5×SSC, 5× Denhardt's solution, 50% formamide, 1% SDS, 100:g/ml denatured salmon sperm DNA at 42° C., with high stringency washes of either (1) 0.3-0.1×SSC, 0.1% SDS at 65° C., or (2) 1 mM Na₂EDTA, 40 mM NaHPO₄ (pH 7.2), 1% SDS at 65° C. The above conditions are intended to be used for DNA-DNA hybrids of 50 base pairs or longer. Where the hybrid is believed to be less than 18 base pairs in length, the hybridization and wash temperatures should be 5-101C below that of the calculated Tm of the hybrid, where Tₘ in ° C.=(2× the number of A and T bases)+(4× the number of G and C bases). For hybrids believed to be about 18 to about 49 base pairs in length, the Tₘ in ° C.=(81.5° C.+16.6(log₁₀M)+0.41(% G+C)−0.61 (% formamide)−500/L), where “M” is the molarity of monovalent cations (e.g., Na⁺), and “L” is the length of the hybrid in base pairs.

Moderate stringency conditions can employ hybridization at either (1) 4×SSC, (10×SSC=3 M NaCl, 0.3 M Na₃-citrate.2H₂O (88 g/liter), pH to 7.0 with 1 M HCl), 1% SDS (sodium dodecyl sulfate), 0.1-2 mg/ml denatured salmon sperm DNA at 65° C., (2) 4×SSC, 50% formamide, 1% SDS, 0.1-2 mg/ml denatured salmon sperm DNA at 42° C., (3) 1% bovine serum albumen (fraction V), 1 mM Na₂EDTA, 0.5 M NaHPO₄ (pH 7.2) (1 M NaHPO₄=134 g Na₂HPO₄.7H₂O, 4 ml 85% H₃PO₄ per liter), 7% SDS, 0.1-2 mg/ml denatured salmon sperm DNA at 65° C., (4) 50% formamide, 5×SSC, 0.02 M Tris-HCl (pH 7.6), 1× Denhardt's solution (100×=10 g Ficoll 400, 10 g polyvinylpyrrolidone, 10 g bovine serum albumin (fraction V), water to 500 ml), 10% dextran sulfate, 1% SDS, 0.1-2 mg/ml denatured salmon sperm DNA at 42° C., (5) 5×SSC, 5× Denhardt's solution, 1% SDS, 1000g/ml denatured salmon sperm DNA at 65° C., or (6) 5×SSC, 5× Denhardt's solution, 50% formamide, 1% SDS, 100:g/ml denatured salmon sperm DNA at 42° C., with moderate stringency washes of 1×SSC, 0.1% SDS at 65° C. The above conditions are intended to be used for DNA-DNA hybrids of 50 base pairs or longer. Where the hybrid is believed to be less than 18 base pairs in length, the hybridization and wash temperatures should be 5-110° C. below that of the calculated Tₘ of the hybrid, where Tₘ in ° C.=(2× the number of A and T bases)+(4× the number of G and C bases). For hybrids believed to be about 18 to about 49 base pairs in length, the Tₘ in ° C.=(81.5° C.+16.6(log₁₀M)+0.41(% G+C)−0.61 (% formamide)−500/L), where “M” is the molarity of monovalent cations (e.g., Na⁺), and “L” is the length of the hybrid in base pairs.

Low stringency conditions can employ hybridization at either (1) 4×SSC, (10×SSC=3 M NaCl, 0.3 M Na₃-citrate.2H₂O (88 g/liter), pH to 7.0 with 1 M HCl), 1% SDS (sodium dodecyl sulfate), 0.1-2 mg/ml denatured salmon sperm DNA at 50° C., (2) 6×SSC, 50% formamide, 1% SDS, 0.1-2 mg/ml denatured salmon sperm DNA at 40° C., (3) 1% bovine serum albumen (fraction V), 1 mM Na₂EDTA, 0.5 M NaHPO₄ (pH 7.2) (1 M NaHPO₄=134 g Na₂HPO₄.7H₂O, 4 ml 85% H₃PO₄ per liter), 7% SDS, 0.1-2 mg/ml denatured salmon sperm DNA at 50° C., (4) 50% formamide, 5×SSC, 0.02 M Tris-HCl (pH 7.6), 1× Denhardt's solution (100×=10 g Ficoll 400, 10 g polyvinylpyrrolidone, 10 g bovine serum albumin (fraction V), water to 500 ml), 10% dextran sulfate, 1% SDS, 0.1-2 mg/ml denatured salmon sperm DNA at 40° C., (5) 5×SSC, 5× Denhardt's solution, 1% SDS, 100:g/ml denatured salmon sperm DNA at 50° C., or (6) 5×SSC, 5× Denhardt's solution, 50% formamide, 1% SDS, 100:g/ml denatured salmon sperm DNA at 40° C., with low stringency washes of either 2×SSC, 0.1% SDS at 50° C., or (2) 0.5% bovine serum albumin (fraction V), 1 mM Na₂EDTA, 40 mM NaHPO₄ (pH 7.2), 5% SDS. The above conditions are intended to be used for DNA-DNA hybrids of 50 base pairs or longer. Where the hybrid is believed to be less than 18 base pairs in length, the hybridization and wash temperatures should be 5-10° C. below that of the calculated Tₘ of the hybrid, where Tₘ in ° C.=(2× the number of A and T bases)+(4× the number of G and C bases). For hybrids believed to be about 18 to about 49 base pairs in length, the Tₘ in ° C.=(81.5° C.+16.6(log₁₀M)+0.41(% G+C)−0.61 (% formamide)−500/L), where “M” is the molarity of monovalent cations (e.g., Na⁺), and “L” is the length of the hybrid in base pairs.

In a preferred aspect, the present invention covers the use of nucleotide sequences that can hybridise to the nucleotide sequence of the present invention under stringent conditions (e.g., 65° C. and 0.1×SSC (1×SSC=0.15 M NaCl, 0.015 M Na₃ Citrate pH 7.0)).

Where the polynucleotide is double-stranded, both strands of the duplex, the use of either individually or in combination, is encompassed by the present invention. Where the polynucleotide is single-stranded, it is to be understood that the use of the complementary sequence of that polynucleotide is also included within the scope of the present invention.

Polynucleotides which are not 100% homologous to the sequences in Table 1 but the use of which falls within the scope of the invention can be obtained in a number of ways. Other variants of the sequences described herein may be obtained for example by probing DNA libraries made from a range of individuals, for example individuals from different populations. In addition, other viral/bacterial, or cellular homologues particularly cellular homologues found in mammalian cells (e.g., rat, mouse, bovine and primate cells), may be obtained and such homologues and fragments thereof in general will be capable of selectively hybridising to sequences identified in Table 1. Such sequences may be obtained by probing cDNA libraries made from or genomic DNA libraries from other animal species, and probing such libraries with probes comprising all or part of any on of the sequences under conditions of medium to high stringency. The nucleotide sequences of or which encode the human homologues identified in column 3 of Table 1, may preferably be used to identify other primate/mammalian homologues or allelic variants.

Variants and strain/species homologues may also be obtained using degenerate PCR which will use primers designed to target sequences within the variants and homologues of the sequences of Table 1. Conserved sequences can be predicted, for example, by aligning the amino acid sequences from several variants/homologues. Sequence alignments can be performed using computer software known in the art. For example the GCG Wisconsin PileUp program is widely used.

The primers used in degenerate PCR will contain one or more degenerate positions and will be used at stringency conditions lower than those used for cloning sequences with single sequence primers against known sequences. It will be appreciated by the skilled person that overall nucleotide homology between sequences from distantly related organisms is likely to be very low and thus in these situations degenerate PCR may be the method of choice rather than screening libraries with labeled fragments.

In addition, homologous sequences may be identified by searching nucleotide and/or protein databases using search algorithms such as the BLAST suite of programs. This approach is described below and in the Examples.

Alternatively, such polynucleotides may be obtained by site directed mutagenesis of characterised sequences. This may be useful where for example silent codon changes are required to sequences to optimise codon preferences for a particular host cell in which the polynucleotide sequences are being expressed. Other sequence changes may be desired in order to introduce restriction enzyme recognition sites, or to alter the property or function of the polypeptides encoded by the polynucleotides. For example, further changes may be desirable to represent particular coding changes found in nucleic acid sequences which give rise to mutant genes which have lost their regulatory function. Probes based on such changes can be used as diagnostic probes to detect such mutants.

Polynucleotides may be used to produce a primer, e.g., a PCR primer, a primer for an alternative amplification reaction, a probe, e.g., labeled with a revealing label by conventional means using radioactive or non-radioactive labels, or the polynucleotides may be cloned into vectors. Such primers, probes and other fragments will be at least 8, 9, 10, or 15, preferably at least 20, for example at least 25, 30 or 40 nucleotides in length, and are also encompassed by the term polynucleotides as used herein.

Polynucleotides such as a DNA polynucleotides and probes for use in the invention may be produced recombinantly, synthetically, or by any means available to those of skill in the art. They may also be cloned by standard techniques. The invention also encompasses a composition comprising one or more isolated polynucleotides encoding a cell cycle progression protein, e.g., a vector containing a polynucleotide encoding a cell cycle progression protein, and also host cells containing such a vector. By “host cell” is meant a cell which has been or can be used as the recipient of transferred nucleic acid by means of a vector. Host cells can prokaryotic or eukaryotic, mammalian, plant, or insect, and can exist as single cells, or as a collection, e.g., as a culture, or in a tissue culture, or in a tissue or an organism. Host cells can also be derived from normal or diseased tissue from a multicellular organism, e.g., a mammal. Host cell, as used herein, is intended to include not only the original cell which was transformed with a nucleic acid, but also descendants of such a cell, which still contain the nucleic acid. The exogenous polynucleotide may be maintained as a non-integrated vector, for example, a plasmid, or alternatively, may be integrated into the host genome. The vector can also contain regulatory sequences, e.g., sequences permitting expression of the polynucleotide.

In general, primers will be produced by synthetic means, involving a stepwise manufacture of the desired nucleic acid sequence one nucleotide at a time. Techniques for accomplishing this using automated techniques are readily available in the art.

Longer polynucleotides will generally be produced using recombinant means, for example using a PCR (polymerase chain reaction) cloning techniques. This will involve making a pair of primers (e.g., of about 15 to 30 nucleotides) flanking a region of the lipid targeting sequence which it is desired to clone, bringing the primers into contact with mRNA or cDNA obtained from an animal or human cell, performing a polymerase chain reaction under conditions which bring about amplification of the desired region, isolating the amplified fragment (e.g., by purifying the reaction mixture on an agarose gel) and recovering the amplified DNA. The primers may be designed to contain suitable restriction enzyme recognition sites so that the amplified DNA can be cloned into a suitable cloning vector.

Polynucleotides or primers for use in the invention may carry a revealing label. Suitable labels include radioisotopes such as ³²P or ³⁵S, enzyme labels, or other protein labels such as biotin. Such labels may be added to polynucleotides or primers of the invention and may be detected by using techniques well known by those in the art.

Polynucleotides or primers for use in the invention or fragments thereof labeled or unlabeled may be used by a person skilled in the art in nucleic acid-based tests for detecting or sequencing polynucleotides of the invention in the human or animal body.

Such tests for detecting generally comprise bringing a biological sample containing DNA or RNA into contact with a probe comprising a polynucleotide or primer of the invention under hybridising conditions and detecting any duplex formed between the probe and nucleic acid in the sample. Such detection may be achieved using techniques such as PCR or by immobilising the probe on a solid support, removing nucleic acid in the sample which is not hybridised to the probe, and then detecting nucleic acid which has hybridised to the probe. Alternatively, the sample nucleic acid may be immobilised on a solid support, and the amount of probe bound to such a support can be detected. Suitable assay methods of this and other formats can be found in for example WO 89/03891 and WO 90/13667.

Tests for sequencing nucleotides for use in the invention include bringing a biological sample containing target DNA or RNA into contact with a probe comprising a polynucleotide or primer of the invention under hybridising conditions and determining the sequence by, for example the Sanger dideoxy chain termination method (see Sambrook et al.).

Such a method generally comprises elongating, in the presence of suitable reagents, the primer by synthesis of a strand complementary to the target DNA or RNA and selectively terminating the elongation reaction at one or more of an A, C, G or T/U residue; allowing strand elongation and termination reaction to occur; separating out according to size the elongated products to determine the sequence of the nucleotides at which selective termination has occurred. Suitable reagents include a DNA polymerase enzyme, the deoxynucleotides dATP, dCTP, dGTP and dTTP, a buffer and ATP. Dideoxynucleotides are used for selective termination.

Tests for detecting or sequencing nucleotides identified in Table 1 in a biological sample may be used to determine particular sequences within cells in individuals who have, or are suspected to have, an altered gene sequence, for example within cancer cells including leukaemia cells and solid tumours such as breast, ovary, lung, colon, pancreas, testes, liver, brain, muscle and bone tumours. Cells from patients suffering from a proliferative disease may also be tested in the same way.

In addition, the identification of the genes described in the Examples will allow the role of these genes in hereditary diseases to be investigated. In general, this will involve establishing the status of the gene (e.g., using PCR sequence analysis), in cells derived from animals or humans with, for example, neoplasms.

The probes for use in the invention may conveniently be packaged in the form of a test kit in a suitable container. In such kits the probe may be bound to a solid support where the assay format for which the kit is designed requires such binding. The kit may also contain suitable reagents for treating the sample to be probed, hybridising the probe to nucleic acid in the sample, control reagents, instructions, and the like.

Homology Searching

Sequence homology (or identity) may be determined using any suitable homology algorithm, using for example default parameters.

Advantageously, the BLAST algorithm is employed, with parameters set to default values. The BLAST algorithm is described in detail at the world wide web site (“www”) of the National Center for Biotechnology Information (“.ncbi”) of the National Institutes of Health (“nih”) of the U.S. government (“.gov”), in the “/Blast/” directory, in the “blast_help.html” file. The search parameters are defined as follows, and are advantageously set to the defined default parameters.

Advantageously, “substantial homology” when assessed by BLAST equates to sequences which match with an EXPECT value of at least about 7, preferably at least about 9 and most preferably 10 or more. The default threshold for EXPECT in BLAST searching is usually 10.

BLAST (Basic Local Alignment Search Tool) is the heuristic search algorithm employed by the programs blastp, blastn, blastx, tblastn, and tblastx; these programs ascribe significance to their findings using the statistical methods of Karlin and Altschul, 1990, Proc. Natl. Acad. Sci. USA 87(6):2264-8 (see the “blast_help.html” file, as described above) with a few enhancements. The BLAST programs were tailored for sequence similarity searching, for example to identify homologues to a query sequence. The programs are not generally useful for motif-style searching. For a discussion of basic issues in similarity searching of sequence databases, see Altschul et al. (1994).

The five BLAST programs available at the National Center for Biotechnology Information web site perform the following tasks:

“blastp” compares an amino acid query sequence against a protein sequence database;

“blastn” compares a nucleotide query sequence against a nucleotide sequence database;

“blastx” compares the six-frame conceptual translation products of a nucleotide query sequence (both strands) against a protein sequence database;

“tblastn” compares a protein query sequence against a nucleotide sequence database dynamically translated in all six reading frames (both strands).

“tblastx” compares the six-frame translations of a nucleotide query sequence against the six-frame translations of a nucleotide sequence database.

BLAST uses the following search parameters:

HISTOGRAM Display a histogram of scores for each search; default is yes. (See parameter H in the BLAST Manual).

DESCRIPTIONS Restricts the number of short descriptions of matching sequences reported to the number specified; default limit is 100 descriptions. (See parameter V in the manual page). See also EXPECT and CUTOFF.

ALIGNMENTS Restricts database sequences to the number specified for which high-scoring segment pairs (HSPs) are reported; the default limit is 50. If more database sequences than this happen to satisfy the statistical significance threshold for reporting (see EXPECT and CUTOFF below), only the matches ascribed the greatest statistical significance are reported. (See parameter B in the BLAST Manual).

EXPECT The statistical significance threshold for reporting matches against database sequences; the default value is 10, such that 10 matches are expected to be found merely by chance, according to the stochastic model of Karlin and Altschul (1990). If the statistical significance ascribed to a match is greater than the EXPECT threshold, the match will not be reported. Lower EXPECT thresholds are more stringent, leading to fewer chance matches being reported. Fractional values are acceptable. (See parameter E in the BLAST Manual).

CUTOFF Cutoff score for reporting high-scoring segment pairs. The default value is calculated from the EXPECT value (see above). HSPs are reported for a database sequence only if the statistical significance ascribed to them is at least as high as would be ascribed to a lone HSP having a score equal to the CUTOFF value. Higher CUTOFF values are more stringent, leading to fewer chance matches being reported. (See parameter S in the BLAST Manual). Typically, significance thresholds can be more intuitively managed using EXPECT.

MATRIX Specify an alternate scoring matrix for BLASTP, BLASTX, TBLASTN and TBLASTX. The default matrix is BLOSUM62 (Henikoff & Henikoff, 1992, Proc. Natl. Aacad. Sci. USA 89(22):10915-9). The valid alternative choices include: PAM40, PAM120, PAM250 and IDENTITY. No alternate scoring matrices are available for BLASTN; specifying the MATRIX directive in BLASTN requests returns an error response.

STRAND Restrict a TBLASTN search to just the top or bottom strand of the database sequences; or restrict a BLASTN, BLASTX or TBLASTX search to just reading frames on the top or bottom strand of the query sequence.

FILTER Mask off segments of the query sequence that have low compositional complexity, as determined by the SEG program of Wootton & Federhen (1993) Computers and Chemistry 17:149-163, or segments consisting of short-periodicity internal repeats, as determined by the XNU program of Clayerie & States, 1993, Computers and Chemistry 17:191-201, or, for BLASTN, by the DUST program of Tatusov and Lipman (see the world wide web site of the NCBI). Filtering can eliminate statistically significant but biologically uninteresting reports from the blast output (e.g., hits against common acidic-, basic- or proline-rich regions), leaving the more biologically interesting regions of the query sequence available for specific matching against database sequences.

Low complexity sequence found by a filter program is substituted using the letter “N” in nucleotide sequence (e.g., “N” repeated 13 times) and the letter “X” in protein sequences (e.g., “X” repeated 9 times).

Filtering is only applied to the query sequence (or its translation products), not to database sequences. Default filtering is DUST for BLASTN, SEG for other programs.

It is not unusual for nothing at all to be masked by SEG, XNU, or both, when applied to sequences in SWISS-PROT, so filtering should not be expected to always yield an effect. Furthermore, in some cases, sequences are masked in their entirety, indicating that the statistical significance of any matches reported against the unfiltered query sequence should be suspect.

NCBI-gi Causes NCBI gi identifiers to be shown in the output, in addition to the accession and/or locus name.

Most preferably, sequence comparisons are conducted using the simple BLAST search algorithm provided at the NCBI world wide web site described above, in the “/BLAST” directory.

Antibodies

The invention also provides the use of monoclonal or polyclonal antibodies to polypeptides encoded by the nucleic acids identified in Table 1 or fragments thereof.

Methods for production of antibodies are known by those skilled in the art. If polyclonal antibodies are desired, a selected mammal (e.g., mouse, rabbit, goat, horse, etc.) is immunised with an immunogenic polypeptide bearing an epitope(s) from a polypeptide. Serum from the immunised animal is collected and treated according to known procedures. If serum containing polyclonal antibodies to an epitope from a polypeptide contains antibodies to other antigens, the polyclonal antibodies can be purified by immunoaffinity chromatography. Techniques for producing and processing polyclonal antisera are known in the art. In order to generate a larger immunogenic response, polypeptides or fragments thereof maybe haptenised to another polypeptide for use as immunogens in animals or humans.

Monoclonal antibodies directed against epitopes in polypeptides can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against epitopes in the polypeptides of the invention can be screened for various properties; i.e., for isotype and epitope affinity.

An alternative technique involves screening phage display libraries where, for example the phage express scFv fragments on the surface of their coat with a large variety of complementarity determining regions (CDRs). This technique is well known in the art.

Antibodies, both monoclonal and polyclonal, which are directed against epitopes from polypeptides encoded by the nucleic acids identified in Table 1 are particularly useful in diagnosis, and those which are neutralising are useful in passive immunotherapy. Monoclonal antibodies, in particular, may be used to raise anti-idiotype antibodies. Anti-idiotype antibodies are immunoglobulins which carry an “internal image” of the antigen of the agent against which protection is desired.

Techniques for raising anti-idiotype antibodies are known in the art. These anti-idiotype antibodies may also be useful in therapy.

For the purposes of this invention, the term “antibody”, unless specified to the contrary, includes fragments of whole antibodies which retain their binding activity for a target antigen. Such fragments include Fv, F(ab′) and F(ab′)₂ fragments, as well as single chain antibodies (scFv). Furthermore, the antibodies and fragments thereof may be humanised antibodies, for example as described in EP-A-239400.

Antibodies may be used in detecting cell cycle progression polypeptides identified herein in biological samples by a method which comprises: (a) providing an antibody of the invention; (b) incubating a biological sample with said antibody under conditions which allow for the formation of an antibody-antigen complex; and (c) determining whether antibody-antigen complex comprising said antibody is formed.

Suitable samples include extracts tissues such as brain, breast, ovary, lung, colon, pancreas, testes, liver, muscle and bone tissues or from neoplastic growths derived from such tissues.

Antibodies that specifically bind to the cell cycle progression proteins can be used in diagnostic methods and kits that are well known to those of ordinary skill in the art to detect or quantify the cell cycle progression proteins in a body fluid or tissue. Results from these tests can be used to diagnose or predict the occurrence or recurrence of a cancer and other cell cycle progression-mediated diseases.

The invention also includes use of the cell cycle progression proteins, antibodies to those proteins, and compositions comprising those proteins and/or their antibodies in diagnosis or prognosis of diseases characterized by proliferative activity. As used herein, the term “prognostic method” means a method that enables a prediction regarding the progression of a disease of a human or animal diagnosed with the disease, in particular, a cell cycle progression-dependent disease. The term “diagnostic method” as used herein means a method that enables a determination of the presence or type of cell cycle progression-dependent disease in or on a human or animal.

The cell cycle progression proteins can be used in a diagnostic method and kit to detect and quantify antibodies capable of binding the proteins. These kits would permit detection of circulating antibodies to the cell cycle progression proteins which indicates, e.g., the proliferation of cancer cells. Patients that have such circulating anti-protein antibodies may be more likely to develop multiple tumors and cancers, and may be more likely to have recurrences of cancer after treatments or periods of remission.

Antibodies for use in the invention may be bound to a solid support and/or packaged into kits in a suitable container along with suitable reagents, controls, instructions and the like.

When used in a diagnostic composition, an antibody is preferably provided together with means for detecting the antibody, which can be enzymatic, fluorescent, radioisotopic or other means. The antibody and the detection means can be provided for simultaneous, simultaneous separate or sequential use, in a diagnostic kit intended for diagnosis.

Measuring Expression of Cell Cycle Progression Genes

Levels of gene expression may be determined using a number of different techniques.

a) at the RNA Level

Gene expression can be detected at the RNA level. RNA may be extracted from cells using RNA extraction techniques including, for example, using acid phenol/guanidine isothiocyanate extraction (RNAzol B; Biogenesis), or RNeasy RNA preparation kits (Qiagen).Typical assay formats utilising ribonucleic acid hybridisation include nuclear run-on assays, RT-PCR and RNase protection assays (Melton et al., Nuc. Acids Res. 12:7035. Methods for detection which can be employed include radioactive labels, enzyme labels, chemiluminescent labels, fluorescent labels and other suitable labels.

Typically, RT-PCR is used to amplify RNA targets. In this process, the reverse transcriptase enzyme is used to convert RNA to complementary DNA (cDNA) which can then be amplified to facilitate detection.

Many DNA amplification methods are known, most of which rely on an enzymatic chain reaction (such as a polymerase chain reaction, a ligase chain reaction, or a self-sustained sequence replication) or from the replication of all or part of the vector into which it has been cloned.

Many target and signal amplification methods have been described in the literature, for example, general reviews of these methods in Landegren, U. et al., Science 242:229-237 (1988) and Lewis, R., Genetic Engineering News 10:1, 54-55 (1990).

PCR is a nucleic acid amplification method described inter alia in U.S. Pat. Nos. 4,683,195 and 4,683,202. PCR can be used to amplify any known nucleic acid in a diagnostic context (Mok et al., 1994, Gynaecologic Oncology 52:247-252). Self-sustained sequence replication (3SR) is a variation of TAS, which involves the isothermal amplification of a nucleic acid template via sequential rounds of reverse transcriptase (RT), polymerase and nuclease activities that are mediated by an enzyme cocktail and appropriate oligonucleotide primers (Guatelli et al., 1990, Proc. Natl. Acad. Sci. USA 87:1874). Ligation amplification reaction or ligation amplification system uses DNA ligase and four oligonucleotides, two per target strand. This technique is described by Wu, D. Y. and Wallace, R. B., 1989, Genomics 4:560. In the Qβ Replicase technique, RNA replicase for the bacteriophage Qβ, which replicates single-stranded RNA, is used to amplify the target DNA, as described by Lizardi et al., 1988, Bio/Technology 6:1197.

Alternative amplification technology can be exploited in the present invention. For example, rolling circle amplification (Lizardi et al., 1998, Nat Genet 19:225) is an amplification technology available commercially (RCAT™) which is driven by DNA polymerase and can replicate circular oligonucleotide probes with either linear or geometric kinetics under isothermal conditions. A further technique, strand displacement amplification (SDA; Walker et al., 1992, Proc. Natl. Acad. Sci. USA 80:392) begins with a specifically defined sequence unique to a specific target.

b) at the Polypeptide Level

Gene expression may also be detected by measuring the cell cycle progression polypeptides. This may be achieved by using molecules which bind to the cell cycle progression polypeptides. Suitable molecules/agents which bind either directly or indirectly to the cell cycle progression polypeptides in order to detect the presence of the protein include naturally occurring molecules such as peptides and proteins, for example antibodies, or they may be synthetic molecules.

Standard laboratory techniques such as immunoblotting as described above can be used to detect altered levels of cell cycle progression proteins, as compared with untreated cells in the same cell population.

Gene expression may also be determined by detecting changes in post-translational processing of polypeptides or post-transcriptional modification of nucleic acids. For example, differential phosphorylation of polypeptides, the cleavage of polypeptides or alternative splicing of RNA, and the like may be measured. Levels of expression of gene products such as polypeptides, as well as their post-translational modification, may be detected using proprietary protein assays or techniques such as 2D polyacrylamide gel electrophoresis.

Antibodies can be assayed for immunospecific binding by any method known in the art. The immunoassays which can be used include but are not limited to competitive and non-competitive assay systems using techniques such as western blots, radioimmunoassays, ELISA, sandwich immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays. Such assays are routine in the art (see, for example, Ausubel et al., eds, 1994, Current Protocols in Molecular Biology, Vol. 1, John Wiley & Sons, Inc., New York, which is incorporated by reference herein in its entirety).

Arrays

Array technology and the various techniques and applications associated with it is described generally in numerous textbooks and documents. These include Lemieux et al., 1998, Molecular Breeding 4:277-289; Schena and Davis. Parallel Analysis with Biological Chips. in PCR Methods Manual (eds. M. Innis, D. Gelfand, J. Sninsky); Schena and Davis, 1999, Genes, Genomes and Chips. In DNA Microarrays: A Practical Approach (ed. M. Schena), Oxford University Press, Oxford, UK, 1999); The Chipping Forecast (Nature Genetics special issue; January 1999 Supplement); Mark Schena (Ed.), Microarray Biochip Technology, (Eaton Publishing Company); Cortes, 2000, The Scientist 14(17):25; Gwynne and Page, Microarray analysis: the next revolution in molecular biology, Science, 1999, August 6; Eakins and Chu, 1999, Trends in Biotechnology, 17:217-218, and also at various world wide web sites.

Array technology overcomes the disadvantages with traditional methods in molecular biology, which generally work on a “one gene in one experiment” basis, resulting in low throughput and the inability to appreciate the “whole picture” of gene function. Currently, the major applications for array technology include the identification of sequence (gene/gene mutation) and the determination of expression level (abundance) of genes. Gene expression profiling may make use of array technology, optionally in combination with proteomics techniques (Celis et al., 2000, FEBS Lett, 480(1):2-16; Lockhart and Winzeler, 2000, Nature 405(6788):827-836; Khan et al., 1999, 20(2):223-9). Other applications of array technology are also known in the art; for example, gene discovery, cancer research (Marx, 2000, Science 289: 1670-1672; Scherf et al et al., 2000, Nat Genet 24(3):236-44; Ross et al., 2000, Nat Genet 2000, 24(3):227-35), SNP analysis (Wang et al., 1998, Science 280(5366):1077-82), drug discovery, pharmacogenomics, disease diagnosis (for example, utilising microfluidics devices: Chemical & Engineering News, Feb. 22, 1999, 77(8):27-36), toxicology (Rockett and Dix (2000), Xenobiotica 30(2):155-77; Afshari et al., 1999, Cancer Res 59(19):4759-60) and toxicogenomics (a hybrid of functional genomics and molecular toxicology). The goal of toxicogenomics is to find correlations between toxic responses to toxicants and changes in the genetic profiles of the objects exposed to such toxicants (Nuwaysir et al., 1999, Molecular Carcinogenesis 24:153-159).

In the context of the present invention, array technology can be used, for example, in the analysis of the expression of one or more of the cell cycle progression proteins identified herein. In one embodiment, array technology may be used to assay the effect of a candidate compound on a number of the cell cycle progression proteins identified herein simultaneously. Accordingly, another aspect of the present invention is to provide microarrays that include at least one, at least two or at least several of the nucleic acids identified in Table 1, or fragments thereof, or protein or antibody arrays.

In general, any library or group of samples may be arranged in an orderly manner into an array, by spatially separating the members of the library or group. Examples of suitable libraries for arraying include nucleic acid libraries (including DNA, cDNA, oligonucleotide, etc. libraries), peptide, polypeptide and protein libraries, as well as libraries comprising any molecules, such as ligand libraries, among others. Accordingly, where reference is made to a “library” in this document, unless the context dictates otherwise, such reference should be taken to include reference to a library in the form of an array. In the context of the present invention, a “library” may include a sample of cell cycle progression proteins as identified herein.

The samples (e.g., members of a library) are generally fixed or immobilised onto a solid phase, preferably a solid substrate, to limit diffusion and admixing of the samples. In a preferred embodiment, libraries of DNA binding ligands may be prepared. In particular, the libraries may be immobilised to a substantially planar solid phase, including membranes and non-porous substrates such as plastic and glass. Furthermore, the samples are preferably arranged in such a way that indexing (i.e., reference or access to a particular sample) is facilitated. Typically the samples are applied as spots in a grid formation. Common assay systems may be adapted for this purpose. For example, an array may be immobilised on the surface of a microplate, either with multiple samples in a well, or with a single sample in each well. Furthermore, the solid substrate may be a membrane, such as a nitrocellulose or nylon membrane (for example, membranes used in blotting experiments). Alternative substrates include glass, or silica based substrates. Thus, the samples are immobilised by any suitable method known in the art, for example, by charge interactions, or by chemical coupling to the walls or bottom of the wells, or the surface of the membrane. Other means of arranging and fixing may be used, for example, pipetting, drop-touch, piezoelectric means, ink-jet and bubble jet technology, electrostatic application, etc. In the case of silicon-based chips, photolithography may be utilised to arrange and fix the samples on the chip.

The samples may be arranged by being “spotted” onto the solid substrate; this may be done by hand or by making use of robotics to deposit the sample. In general, arrays may be described as macroarrays or microarrays, the difference being the size of the sample spots. Macroarrays typically contain sample spot sizes of about 300 microns or larger and may be easily imaged by existing gel and blot scanners. The sample spot sizes in microarrays are typically less than 200 microns in diameter and these arrays usually contain thousands of spots. Thus, microarrays may require specialized robotics and imaging equipment, which may need to be custom made. Instrumentation is described generally in a review by Cortese, 2000, The Scientist 14(11):26.

Techniques for producing immobilised libraries of DNA molecules have been described in the art. Generally, most prior art methods described how to synthesise single-stranded nucleic acid molecule libraries, using for example masking techniques to build up various permutations of sequences at the various discrete positions on the solid substrate. U.S. Pat. No. 5,837,832, the contents of which are incorporated herein by reference, describes an improved method for producing DNA arrays immobilised to silicon substrates based on very large scale integration technology. In particular, U.S. Pat. No. 5,837,832 describes a strategy called “tiling” to synthesize specific sets of probes at spatially-defined locations on a substrate which may be used to produced the immobilised DNA libraries of the present invention. U.S. Pat. No. 5,837,832 also provides references for earlier techniques that may also be used.

Arrays of peptides (or peptidomimetics) may also be synthesised on a surface in a manner that places each distinct library member (e.g., unique peptide sequence) at a discrete, predefined location in the array. The identity of each library member is determined by its spatial location in the array. The locations in the array where binding interactions between a predetermined molecule (e.g., a target or probe) and reactive library members occur is determined, thereby identifying the sequences of the reactive library members on the basis of spatial location. These methods are described in U.S. Pat. No. 5,143,854; WO 90/15070 and WO 92/10092; Fodor et al., 1991, Science 251:767; Dower and Fodor, 1991, Ann. Rep. Med. Chem. 26:271.

To aid detection, targets and probes may be labelled with any readily detectable reporter, for example, a fluorescent, bioluminescent, phosphorescent, radioactive, etc reporter. Such reporters, their detection, coupling to targets/probes, etc are discussed elsewhere in this document. Labelling of probes and targets is also disclosed in Shalon et al., 1996, Genome Res 6(7):639-45.

Specific examples of DNA arrays include the following:

Format I: probe cDNA (500-˜5,000 bases long) is immobilized to a solid surface such as glass using robot spotting and exposed to a set of targets either separately or in a mixture. This method is widely considered as having been developed at Stanford University (Ekins and Chu, 1999, Trends in Biotechnology, 17:217-218).

Format II: an array of oligonucleotide (˜20-˜25-mer oligos) or peptide nucleic acid (PNA) probes is synthesized either in situ (on-chip) or by conventional synthesis followed by on-chip immobilization. The array is exposed to labeled sample DNA, hybridized, and the identity/abundance of complementary sequences are determined. Such a DNA chip is sold by Affymetrix, Inc., under the GeneChipg trademark.

Examples of some commercially available microarray formats are set out, for example, in Marshall and Hodgson, 1998, Nature Biotechnology 16(1):27-31.

Data analysis is also an important part of an experiment involving arrays. The raw data from a microarray experiment typically are images, which need to be transformed into gene expression matrices—tables where rows represent for example genes, columns represent for example various samples such as tissues or experimental conditions, and numbers in each cell for example characterize the expression level of the particular gene in the particular sample. These matrices have to be analyzed further, if any knowledge about the underlying biological processes is to be extracted. Methods of data analysis (including supervised and unsupervised data analysis as well as bioinformatics approaches) are disclosed in Brazma and Vilo J, 2000, FEBS Lett 480(1): 17-24.

As disclosed above, proteins, polypeptides, etc may also be immobilised in arrays. For example, antibodies have been used in microarray analysis of the proteome using protein chips (Borrebaeck Calif., 2000, Immunol Today 21(8):379-82). Polypeptide arrays are reviewed in, for example, MacBeath and Schreiber, 2000, Science, 289(5485):1760-1763.

Modifying the Functional Activity of a Cell Cycle Progression Protein

The functional activity of a cell cycle progression protein may be modified by suitable molecules/agents which bind either directly or indirectly to a cell cycle progression protein, or to the nucleic acid encoding it. Agents may be naturally occurring molecules such as peptides and proteins, for example antibodies, or they may be synthetic molecules. Methods of modulating the level of expression of a cell cycle progression protein include, for example, using antisense techniques.

Antisense constructs, i.e., nucleic acid, preferably RNA, constructs complementary to the sense nucleic acid or mRNA, are described in detail in U.S. Pat. No. 6,100,090 (Monia et al.), and Neckers et al., 1992, Crit Rev Oncog 3(1-2):175-231, the teachings of which document are specifically incorporated by reference. Other methods of modulating gene expression are known to those skilled in the art and include dominant negative approaches as well as introducing peptides or small molecules which inhibit gene expression or functional activity.

RNA interference (RNAi) is a method of post transcriptional gene silencing (PTGS) induced by the direct introduction of double-stranded RNA (dsRNA) and has emerged as a useful tool to knock out expression of specific genes in a variety of organisms. RNAi is described by Fire et al., Nature 391:806-811 (1998). Other methods of PTGS are known and include, for example, introduction of a transgene or virus. Generally, in PTGS, the transcript of the silenced gene is synthesised but does not accumulate because it is rapidly degraded. Methods for PTGS, including RNAi are described, for example, in the Ambion.com world wide web site, in the directory “/hottopics/”, in the “mai” file.

Suitable methods for RNAi in vitro are described herein. One such method involves the introduction of siRNA (small interfering RNA). Current models indicate that these 21-23 nucleotide dsRNAs can induce PTGS. Methods for designing effective siRNAs are described, for example, in the Ambion web site described above. RNA precursers can also be encoded by all or a part of one of the cell cycle progression nucleic acid sequences described herein.

Assays

The present invention provides assays that are suitable for identifying substances which bind to polypeptides of the invention and which affect, for example, formation of the nuclear envelope, exit from the quiescent phase of the cell cycle (G0), G1 progression, chromosome decondensation, nuclear envelope breakdown, START, initiation of DNA replication, progression of DNA replication, termination of DNA replication, centrosome duplication, G2 progression, activation of mitotic or meiotic functions, chromosome condensation, centrosome separation, microtubule nucleation, spindle formation and function, interactions with microtubule motor proteins, chromatid separation and segregation, inactivation of mitotic functions, formation of contractile ring, cytokinesis functions, chromatin binding, formation of replication complexes, replication licensing, phosphorylation or other secondary modification activity, proteolytic degradation, microtubule binding, actin binding, septin binding, microtubule organising centre nucleation activity and binding to components of cell cycle signalling pathways.

In general, a substance which inhibits one or more of these aspects of cell cycle progression either inhibits it completely, or leads to a significant (i.e., greater than 50%) reduction in protein activity at concentrations of 500 mM or less, relative to controls. Preferably, the inhibition is by 75% relative to controls, more preferably by 90%, and most preferably by 95% or 100% relative to controls. A substance which enhances or increases one or more of these aspects of cell cycle progression leads to a significant (i.e., greater than 50%) increase in protein activity at concentrations of 500 mM or less, relative to controls. Preferably, the increase is by 75% relative to controls, more preferably by 90%, and most preferably by 95% or 100% relative to controls.

In addition, assays can be used to identify substances that interfere with binding of polypeptides of the invention, where appropriate, to components of cell division cycle machinery. Such assays are typically in vitro. Assays are also provided that test the effects of candidate substances identified in preliminary in vitro assays on intact cells in whole cell assays. The assays described below, or any suitable assay as known in the art, may be used to identify these substances.

According to one aspect of the invention, therefore, we provide one or more substances identified by any of the assays described below, viz, mitosis assays, meiotic assays, polypeptide binding assays, microtubule binding/polymerisation assays, microtubule purification and binding assays, microtubule organising centre (MTOC) nucleation activity assays, motor protein assay, assay for spindle assembly and function, assays for DNA replication, chromosome condensation assays, kinase assays, kinase inhibitor assays, and whole cell assays, each as described in further detail below.

Modulator Screening Assays

Compounds having inhibitory, activating, or modulating activity can be identified using in vitro and in vivo assays for cell cycle progression protein activity and/or expression, e.g., ligands, agonists, antagonists, and their homologs and mimetics. Modulator screening may be performed by adding a putative modulator test compound to a tissue or cell sample, and monitoring the effect of the test compound on the function and/or expression of a cell cycle progression protein. A parallel sample which does not receive the test compound is also monitored as a control. The treated and untreated cells are then compared by any suitable phenotypic criteria, including but not limited to microscopic analysis, viability testing, ability to replicate, histological examination, the level of a particular RNA or polypeptide associated with the cells, the level of enzymatic activity expressed by the cells or cell lysates, and the ability of the cells to interact with other cells or compounds.

Methods for inducing cell cycle progression are well known in the art and include, without limitation, exposure to growth factors. Differences between treated and untreated cells indicates effects attributable to the test compound.

A substance that inhibits cell cycle progression as a result of an interaction with a polypeptide of the invention may do so in several ways. For example, if the substance inhibits cell division, mitosis and/or meiosis, it may directly disrupt the binding of a polypeptide of the invention to a component of the spindle apparatus by, for example, binding to the polypeptide and masking or altering the site of interaction with the other component. A substance which inhibits DNA replication may do so by inhibiting the phosphorylation or de-phosphorylation of proteins involved in replication. For example, it is known that the kinase inhibitor 6-DMAP (6-dimethylaminopurine) prevents the initiation of replication (Blow, J. J., 1993, J Cell Biol 122:993-1002). Candidate substances of this type may conveniently be preliminarily screened by in vitro binding assays as, for example, described below and then tested, for example in a whole cell assay as described below. Examples of candidate substances include antibodies which recognise a polypeptide of the invention.

A substance which can bind directly to a polypeptide of the invention may also inhibit its function in cell cycle progression by altering its subcellular localisation and hence its ability to interact with its normal substrate. The substance may alter the subcellular localisation of the polypeptide by directly binding to it, or by indirectly disrupting the interaction of the polypeptide with another component. For example, it is known that interaction between the p68 and p180 subunits of DNA polymerase alpha-primase enzyme is necessary in order for p180 to translocate into the nucleus (Mizuno et al., 1998, Mol Cell Biol 18:3552-62), and accordingly, a substance which disrupts the interaction between p68 and p180 will affect nuclear translocation and hence activity of the primase. A substance which affects mitosis may do so by preventing the polypeptide and components of the mitotic apparatus from coming into contact within the cell.

These substances may be tested using, for example the whole cells assays described below. Non-functional homologues of a polypeptide of the invention may also be tested for inhibition of cell cycle progression since they may compete with the wild type protein for binding to components of the cell division cycle machinery whilst being incapable of the normal functions of the protein or block the function of the protein bound to the cell division cycle machinery. Such non-functional homologues may include naturally occurring mutants and modified sequences or fragments thereof.

Alternatively, instead of preventing the association of the components directly, the substance may suppress the biologically available amount of a polypeptide of the invention. This may be by inhibiting expression of the component, for example at the level of transcription, transcript stability, translation or post-translational stability. An example of such a substance would be antisense RNA or double-stranded interfering RNA sequences which suppresses the amount of mRNA biosynthesis.

Suitable candidate substances include peptides, especially of from about 5 to 30 or 10 to 25 amino acids in size, based on the sequence of the polypeptides described in the Examples, or variants of such peptides in which one or more residues have been substituted. Peptides from panels of peptides comprising random sequences or sequences which have been varied consistently to provide a maximally diverse panel of peptides may be used.

Suitable candidate substances also include antibody products (for example, monoclonal and polyclonal antibodies, single chain antibodies, chimeric antibodies and CDR-grafted antibodies) which are specific for a polypeptide of the invention. Furthermore, combinatorial libraries, peptide and peptide mimetics, defined chemical entities, oligonucleotides, and natural product libraries may be screened for activity as inhibitors of binding of a polypeptide of the invention to the cell division cycle machinery, for example mitotic/meiotic apparatus (such as microtubules). The candidate substances may be used in an initial screen in batches of, for example 10 substances per reaction, and the substances of those batches which show inhibition tested individually. Candidate substances which show activity in in vitro screens such as those described below can then be tested in whole cell systems, such as mammalian cells which will be exposed to the inhibitor and tested for inhibition of any of the stages of the cell cycle.

A substance is identified as a modulator of cell cycle progression activity when it is found to inhibit, decrease, increase, enhance, or activate such activity. In general, a substance which inhibits one or more of these aspects of cell cycle progression either inhibits it completely, or leads to a significant (i.e., greater than 50%) reduction in protein activity at concentrations of 500 mM or less, relative to controls (i.e., substance known to not modulate one or more aspects of cell cycle progression). Preferably, the inhibition is by 75% relative to controls, more preferably by 90%, and most preferably by 95% or 100% relative to controls. The inhibition may prevent cell cycle progression, or may simply delay or prolong cell cycle progression. A substance which enhances or increases one or more of these aspects of cell cycle progression leads to a significant (i.e., greater than 50%) increase in protein activity at concentrations of 500 mM or less, relative to controls. Preferably, the increase is by 75% relative to controls, more preferably by 90%, and most preferably by 95% or 100% relative to controls.

Polypeptide Binding Assays

One type of assay for identifying substances that bind to a polypeptide of the invention involves contacting a polypeptide of the invention, which is immobilised on a solid support, with a non-immobilised candidate substance determining whether and/or to what extent the polypeptide of the invention and candidate substance bind to each other. Alternatively, the candidate substance may be immobilised and the polypeptide of the invention non-immobilised.

The binding of the substance to the cell cycle progression polypeptide can be transient, reversible or permanent. Preferably the substance binds to the polypeptide with a Kd value which is lower than the Kd value for binding to control polypeptides (i.e., polypeptides known to not be cell cycle progression polypeptides). Preferably the Kd value of the substance is 2 fold less than the Kd value for binding to control polypeptides, more preferably with a Kd value 100 fold less, and most preferably with a Kd 1000 fold less than that for binding to the control polypeptide.

In a preferred assay method, the polypeptide of the invention is immobilised on beads such as agarose beads. Typically this is achieved by expressing the component as a GST-fusion protein in bacteria, yeast or higher eukaryotic cell lines and purifying the GST-fusion protein from crude cell extracts using glutathione-agarose beads (Smith and Johnson, 1988; Gene 67(10):31-40). As a control, binding of the candidate substance, which is not a GST-fusion protein, to the immobilised polypeptide of the invention is determined in the absence of the polypeptide of the invention. The binding of the candidate substance to the immobilised polypeptide of the invention is then determined. This type of assay is known in the art as a GST pulldown assay. Again, the candidate substance may be immobilised and the polypeptide of the invention non-immobilised.

It is also possible to perform this type of assay using different affinity purification systems for immobilising one of the components, for example Ni-NTA agarose and histidine-tagged components.

Binding of the polypeptide of the invention to the candidate substance may be determined by a variety of methods well-known in the art. For example, the non-immobilised component may be labeled (with for example, a radioactive label, an epitope tag or an enzyme-antibody conjugate). Alternatively, binding may be determined by immunological detection techniques. For example, the reaction mixture can be Western blotted and the blot probed with an antibody that detects the non-immobilised component. ELISA techniques may also be used.

Candidate substances are typically added to a final concentration of from 1 to 1000 nmol/ml, more preferably from 1 to 100 nmol/ml. In the case of antibodies, the final concentration used is typically from 100 to 500 μg/ml, more preferably from 200 to 300 μg/ml.

Microtubule Binding/Polymerisation Assays

In the case of cell cycle progression polypeptides that bind to microtubules, another type of in vitro assay involves determining whether a candidate substance modulates binding of a polypeptide of the invention to microtubules. Such an assay typically comprises contacting a polypeptide with microtubules in the presence or absence of the candidate substance and determining if the candidate substance has an affect on the binding of the polypeptide to the microtubules. This assay can also be used in the absence of candidate substances to confirm that a polypeptide does indeed bind to microtubules.

The binding of the substance to the cell cycle progression polypeptide can be transient, reversible or permanent. Preferably the substance binds to the polypeptide with a Kd value which is lower than the Kd value for binding to control polypeptides (i.e., polypeptides known to not be cell cycle progression polypeptides). Preferably the Kd value of the substance is 2 fold less than the Kd value for binding to control polypeptides, more preferably with a Kd value 100 fold less, and most preferably with a Kd 1000 fold less than that for binding to the control polypeptide.

Microtubules may be prepared and assays conducted as follows.

Microtubule Purification and Binding Assays

Microtubules are purified from 0-3 h-old Drosophila embryos essentially as described previously (Saunders et al., 1997, J. Cell Biol. 137(4):881-90). About 3 ml of embryos are homogenized with a Dounce homogenizer in 2 volumes of ice-cold lysis buffer (0.1 M Pipes/NaOH, pH6.6, 5 mM EGTA, 1 mM MgSO₄, 0.9 M glycerol, 1 mM DTT, 1 mM PMSF, 1 μg/ml aprotinin, 1 μg/ml leupeptin and 1 μg/ml pepstatin). The microtubules are depolymerized by incubation on ice for 15 min, and the extract is then centrifuged at 16,000 g for 30 minutes at 4° C. The supernatant is recentrifuged at 135,000 g for 90 minutes at 4° C. Microtubules in this later supernatant are polymerized by addition of GTP to 1 mM and taxol to 20 μM and incubation at room temperature for 30 minutes A 3 ml aliquot of the extract is layered on top of 3 ml 15% sucrose cushion prepared in lysis buffer. After centrifuging at 54,000g for 30 minutes at 20° C. using a swing out rotor, the microtubule pellet is resuspended in lysis buffer.

Microtubule overlay assays are performed as previously described (Saunders et al., 1997). 500 ng per lane of recombinant Asp, recombinant polypeptide, and bovine serum albumin (BSA, Sigma) are fractionated by 10% SDS-PAGE and blotted onto PVDF membranes (Millipore). The membranes are preincubated in TBST (50 mM Tris pH 7.5, 150 mM NaCl, 0.05% Tween 20) containing 5% low fat powdered milk (LFPM) for 1 h and then washed 3 times for 15 minutes in lysis buffer. The filters are then incubated for 30 minutes in lysis buffer containing either 1 mM GDP, 1 mM GTP, or 1 mM GTP-γ-S. MAP-free bovine brain tubulin (Molecular Probes) is polymerised at a concentration of 2 μg/ml in lysis buffer by addition of GTP to a final concentration of 1 mM and incubated at 37° C. for 30 minutes. The nucleotide solutions are removed and the buffer containing polymerised microtubules added to the membanes for incubation for 1 h at 37° C. with addition of taxol at a final concentration of 10 μM for the final 30 minutes. The blots are then washed 3 times with TBST and the bound tubulin detected using standard Western blot procedures using anti-β-tubulin antibodies (Boehringer Mannheim) at 2.5 μg/ml and the Super Signal detection system (Pierce).

It may be desirable in one embodiment of this type of assay to deplete the polypeptide of interest from cell extracts used to produce polymerise microtubules. This may, for example, be achieved by the use of suitable antibodies.

A simple extension to this type of assay would be to test the effects of a purified polypeptide upon the ability of tubulin to polymerise in vitro (for example, as used by Andersen and Karsenti, 1997, J. Cell. Biol. 139(4):975-83) in the presence or absence of a candidate substance (typically added at the concentrations described above). Xenopus cell-free extracts may conveniently be used, for example as a source of tubulin.

Microtubule Organising Centre (MTOC) Nucleation Activity Assays

Candidate substances, for example those identified using the binding assays described above, may be screening using a microtubule organising centre nucleation activity assay to determine if they are capable of disrupting MTOCs as measured by, for example, aster formation. This assay in its simplest form comprises adding the candidate substance to a cellular extract which in the absence of the candidate substance has microtubule organising centre nucleation activity resulting in formation of asters.

A substance is identified as inhibiting cell cycle progression activity when it is found to inhibit or decrease such activity. In general, a substance which inhibits one or more of these aspects of cell cycle progression either inhibits it completely, or leads to a significant (i.e., greater than 50%) reduction in protein activity at concentrations of 500 mM or less, relative to controls (i.e., substance known to not modulate one or more aspects of cell cycle progression). Preferably, the inhibition is by 75% relative to controls, more preferably by 90%, and most preferably by 95% or 100% relative to controls. The inhibition may prevent cell cycle progression, or may simply delay or prolong cell cycle progression.

In a preferred embodiment, the assay system comprises (i) a polypeptide of interest and (ii) components required for microtubule organising centre nucleation activity except for functional polypeptide of intereset, which is typically removed by immunodepletion (or by the use of extracts from mutant cells). The components themselves are typically in two parts such that microtubule nucleation does not occur until the two parts are mixed. The polypeptide of interest may be present in one of the two parts initially or added subsequently prior to mixing of the two parts.

Subsequently, the polypeptide of interest and candidate substance are added to the component mix and microtubule nucleation from centrosomes measured, for example by immunostaining for the polypeptide of interest and visualising aster formation by immuno-fluorescence microscopy. The polypeptide of interest may be preincubated with the candidate substance before addition to the component mix. Alternatively, both the polypeptide of interest and the candidate substance may be added directly to the component mix, simultaneously or sequentially in either order.

The components required for microtubule organising centre formation typically include salt-stripped centrosomes prepared as described in Moritz et al., 1998, J. Cell Biol. 142(3):775-86). Stripping centrosome preparations with 2M KI removes the centrosome proteins CP60, CP190, CNN and γ-tubulin. Of these, neither CP60 nor CP190 appear to be required for microtubule nucleation. The other minimal components are typically provided as a depleted cellular extract, or conveniently, as a cellular extract from cells with a non-functional variant of a polypeptide of interest. Typically, labeled tubulin (usually β-tubulin) is also added to assist in visualising aster formation.

Alternatively, partially purified centrosomes that have not been salt-stripped may be used as part of the components. In this case, only tubulin, preferably labeled tubulin is required to complete the component mix.

Candidate substances are typically added to a final concentration of from 1 to 1000 nmol/ml, more preferably from 1 to 100 nmol/ml. In the case of antibodies, the final concentration used is typically from 100 to 500 μg/ml, more preferably from 200 to 300 μg/ml.

The degree of inhibition of aster formation by the candidate substance may be determined by measuring the number of normal asters per unit area for control untreated cell preparation and measuring the number of normal asters per unit area for cells treated with the candidate substance and comparing the result. Typically, a candidate substance is considered to be capable of disrupting MTOC integrity if the treated cell preparations have less than 50%, preferably less than 40, 30, 20 or 10% of the number of asters found in untreated cells preparations. It may also be desirable to stain cells for γ-tubulin to determine the maximum number of possible MTOCs present to allow normalisation between samples.

Motor Protein Assay

Polypeptides of interest may interact with motor proteins such as the Eg5-like motor protein in vitro. The effects of candidate substances on such a process may be determined using assays wherein the motor protein is inmobilised on coverslips. Rhodamine labeled microtubules are then added and their translocation can be followed by fluorescent microscopy. The effect of candidate substances may thus be determined by comparing the extent and/or rate of translocation in the presence and absence of the candidate substance. Generally, candidate substances known to bind to a polypeptide of interest, would be tested in this assay. Alternatively, a high throughput assay may be used to identify modulators of motor proteins and the resulting identified substances tested for affects on a polypeptide of interest as described above.

Typically this assay uses microtubules stabilised by taxol (e.g., Howard and Hyman 1993, Chandra and Endow, 1993—both chapters in “Motility Assays for Motor Proteins” Ed. Jon Scholey, pub. Academic Press). If however, a polypeptide of interest were to promote stable polymerisation of microtubules (see above) then these microtubules could be used directly in motility assays.

Simple protein-protein binding assays as described above, using a motor protein and a polypeptide of interest may also be used to confirm that the polypeptide of the invention binds to the motor protein, typically prior to testing the effect of candidate substances on that interaction.

The binding of the substance to the cell cycle progression polypeptide can be transient, reversible or permanent. Preferably the substance binds to the polypeptide with a Kd value which is lower than the Kd value for binding to control polypeptides (i.e., polypeptides known to not be cell cycle progression polypeptides). Preferably the Kd value of the substance is 2 fold less than the Kd value for binding to control polypeptides, more preferably with a Kd value 100 fold less, and most preferably with a Kd 1000 fold less than that for binding to the control polypeptide.

Assay for Spindle Assembly and Function

A further assay to investigate the function of a polypeptide of interest and the effect of candidate substances on those functions is an assay which measures spindle assembly and function. Typically, such assays are performed using Xenopus cell free systems, where two types of spindle assembly are possible. In the “half spindle” assembly pathway, a cytoplasmic extract of CSF arrested oocytes is mixed with sperm chromatin. The half spindles that form subsequently fuse together. A more physiological method is to induce CSF arrested extracts to enter interphase by addition of calcium, whereupon the DNA replicates and kinetochores form. Addition of fresh CSF arrested extract then induces mitosis with centrosome duplication and spindle formation (for discussion of these systems see Tournebize and Heald, 1996, Nature 382(6590):420-5).

Again, generally, candidate substances known to bind to a polypeptide of the invention, or non-functional polypeptide variants of the invention, would be tested in this assay. Alternatively, a high throughput assay may be used to identify modulators of spindle formation and function and the resulting identified substances tested for affects binding of the polypeptide of interest as described above.

The binding of the substance to the cell cycle progression polypeptide can be transient, reversible or permanent. Preferably the substance binds to the polypeptide with a Kd value which is lower than the Kd value for binding to control polypeptides (i.e., polypeptides known to not be cell cycle progression polypeptides). Preferably the Kd value of the substance is 2 fold less than the Kd value for binding to control polypeptides, more preferably with a Kd value 100 fold less, and most preferably with a Kd 1000 fold less than that for binding to the control polypeptide.

Assays for DNA Replication

Another assay to investigate the function of a polypeptide of interest and the effect of candidate substances on those functions is as assay for replication of DNA. A number of cell free systems have been developed to assay DNA replication. These can be used to assay the ability of a substance to prevent or inhibit DNA replication, by conducting the assay in the presence of the substance. Suitable cell-free assay systems include, for example the SV-40 assay (Li and Kelly, 1984, Proc. Natl. Acad. Sci USA 81:6973-6977; Waga and Stillman, 1994, Nature 369:207-212). A Drosophila cell free replication system, for example as described by Crevel and Cotteril, 1991, EMBO J. 10:4361-4369, may also be used. A preferred assay is a cell free assay derived from Xenopus egg low speed supernatant extracts described in Blow and Laskey (1986, Cell 47:577-587) and Sheehan et al. (1988, J. Cell Biol. 106:1-12), which measures the incorporation of nucleotides into a substrate consisting of Xenopus sperm DNA or HeLa nuclei. The nucleotides may be radiolabelled and incorporation assayed by scintillation counting. Alternatively and preferably, bromo-deoxy-uridine (BrdU) is used as a nucleotide substitute and replication activity measured by density substitution. The latter assay is able to distinguish genuine replication initiation events from incorporation as a result of DNA repair. The human cell-free replication assay reported by Krude et al., 1997, Cell 88:109-19 may also be used to assay the effects of substances on the polypeptides of interest.

A substance is identified as inhibiting cell cycle progression activity when it is found to inhibit or decrease such activity. In general, a substance which inhibits one or more of these aspects of cell cycle progression either inhibits it completely, or leads to a significant (i.e., greater than 50%) reduction in protein activity at concentrations of 50 mM or less, relative to controls (i.e., substance known to not modulate one or more aspects of cell cycle progression). Preferably, the inhibition is by 75% relative to controls, more preferably by 90%, and most preferably by 95% or 100% relative to controls. The inhibition may prevent cell cycle progression, or may simply delay or prolong cell cycle progression.

Other In vitro Assays

Other assays for identifying substances that bind to a polypeptide of interest are also provided. For example, substances which affect chromosome condensation may be assayed using the in vitro cell free system derived from Xenopus eggs, as known in the art.

The binding of the substance to the cell cycle progression polypeptide can be transient, reversible or permanent. Preferably the substance binds to the polypeptide with a Kd value which is lower than the Kd value for binding to control polypeptides (i.e., polypeptides known to not be cell cycle progression polypeptides). Preferably the Kd value of the substance is 2 fold less than the Kd value for binding to control polypeptides, more preferably with a Kd value 100 fold less, and most preferably with a Kd 1000 fold less than that for binding to the control polypeptide.

Substances which affect kinase activity or proteolysis activity are of interest. It is known, for example, that temporal control of ubiquitin-proteasome mediated protein degradation is critical for normal G1 and S phase progression (reviewed in Krek, 1998, Curr Opin Genet Dev 8:36-42). A number of E3 ubiquitin protein ligases, designated SCFs (Skpl-cullin-F-box protein ligase complexes), confer substrate specificity on ubiquitination reactions, while protein kinases phosphorylate substrates destined for destruction and convert them into preferred targets for ubiquitin modification catalyzed by SCFs. Furthermore, ubiquitin-mediated proteolysis due to the anaphase-promoting complex/cyclosome (APC/C) is essential for separation of sister chromatids during mitosis, and exit from mitosis (Listovsky et al., 2000, Exp Cell Res 255:184-191).

Substances which inhibit or affect kinase activity may be identified by means of a kinase assay as known in the art, for example, by measuring incorporation of ³²P into a suitable peptide or other substrate in the presence of the candidate substance. Similarly, substances which inhibit or affect proteolytic activity may be assayed by detecting increased or decreased cleavage of suitable polypeptide substrates. Assays for these and other protein or polypeptide activities are known to those skilled in the art, and may suitably be used to identify substances which bind to a polypeptide of interest and affect its activity.

Whole Cell Assays

Candidate substances may also be tested on whole cells for their effect on cell cycle progression, including mitosis and/or meiosis. Preferably the candidate substances have been identified by the above-described in vitro methods. Alternatively, rapid throughput screens for substances capable of inhibiting cell division, typically mitosis, may be used as a preliminary screen and then used in the in vitro assay described above to confirm that the affect is on a particular polypeptide of interest.

The candidate substance, i.e., the test compound, may be administered to the cell in several ways. For example, it may be added directly to the cell culture medium or injected into the cell. Alternatively, in the case of polypeptide candidate substances, the cell may be transfected with a nucleic acid construct which directs expression of the polypeptide in the cell. Preferably, the expression of the polypeptide is under the control of a regulatable promoter.

Typically, an assay to determine the effect of a candidate substance identified by the method of the invention on a particular stage of the cell division cycle comprises administering the candidate substance to a cell and determining whether the substance inhibits that stage of the cell division cycle. Techniques for measuring progress through the cell cycle in a cell population are well known in the art. The extent of progress through the cell cycle in treated cells is compared with the extent of progress through the cell cycle in an untreated control cell population to determine the degree of inhibition, if any. For example, an inhibitor of mitosis or meiosis may be assayed by measuring the proportion of cells in a population which are unable to undergo mitosis/meiosis and comparing this to the proportion of cells in an untreated population.

The concentration of candidate substances used will typically be such that the final concentration in the cells is similar to that described above for the in vitro assays.

A candidate substance is typically considered to be an inhibitor of a particular stage in the cell division cycle (for example, mitosis) if the proportion of cells undergoing that particular stage (i.e., mitosis) is reduced to below 50%, preferably below 40, 30, 20 or 10% of that observed in untreated control cell populations.

Suitably a polypeptide of interest in the context of the above assays is a polypeptide encoded by any nucleic acid sequence identified in Table 1.

Therapeutic Uses

Many tumours are associated with defects in cell cycle progression, for example loss of normal cell cycle control. Tumour cells may therefore exhibit rapid and often aberrant mitosis. One therapeutic approach to treating cancer is therefore to inhibit mitosis in rapidly dividing cells. Such an approach may also be used for therapy of any proliferative disease in general. In general, a proliferative disease is defined as being “treated” if the cell proliferation associated with the disease or condition is significantly inhibited (i.e., by 50% or more) relative to controls. Preferably, the inhibition is by 75% relative to controls, more preferably by 90%, and most preferably by 95% or 100% relative to controls. The inhibition may prevent cell proliferation, may simply delay or prolong proliferation.

Thus, since the polypeptides of the invention appear to be required for normal cell cycle progression, they represent targets for inhibition of their functions, particularly in tumour cells and other proliferative cells. Another therapeutic approach to treating cancer may be an anti-angiogenic approach in which angiogenesis is targeted and thus growth of tumour blood vessels inhibited thereby depriving a tumour of blood supply.

The term proliferative disorder is used herein in a broad sense to include any disorder that requires control of the cell cycle, for example, cardiovascular disorders such as restenosis and cardiomyopathy, auto-immune disorders such as glomerulonephritis and rheumatoid arthritis, dermatological disorders such as psoriasis, anti-inflammatory, anti-fungal, antiparasitic disorders such as malaria, emphysema and alopecia.

Proliferative disorders also include malignant and pre-neoplastic disorders. The present invention is especially useful in relation to treatment or diagnosis of adenocarcinomas such as: small cell lung cancer, and cancer of the kidney, uterus, prostrate, bladder, ovary, colon and breast. For example, malignancies which may be treatable according to the present invention include acute and chronic leukemias, lymphomas, myelomas, sarcomas such as Fibrosarcoma, myxosarcoma, liposarcoma, lymphangioendotheliosarcoma, angiosarcoma, endotheliosarcoma, chondrosarcoma, osteogenic sarcoma, chordoma, lymphangiosarcoma, synovioma, mesothelioma, leimyosarcoma, rhabdomyosarcoma, colon carcinoma, ovarian cancer, prostate cancer, pancreatic cancer, breast cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, choriocarcinoma, renal cell carcinoma, hepatoma, bile duct carcinoma seminoma, embryonal carcinoma, cervical cancer, testicular tumour, lung carcinoma, small cell lung carcinoma, bladder carcinoma, epithelial carcinoma, glioma, astrocytoma, ependymoma, pinealoma, hemangioblastoma, acoustic neuoma, medulloblastoma, craniopharyngioma, oligodendroglioma, menangioma, melanoma, neutroblastoma and retinoblastoma.

One possible approach is to express anti-sense constructs directed against polynucleotides of the invention, preferably selectively in tumour cells, to inhibit gene function and prevent the tumour cell from progressing through the cell cycle. Anti-sense constructs may also be used to inhibit gene function to prevent cell cycle progression in a proliferative cell. Another approach is to use non-functional variants of polypeptides of the invention that compete with the endogenous gene product for cellular components of cell cycle machinery, resulting in inhibition of function. Alternatively, compounds identified by the assays described above as binding to a polypeptide of the invention may be administered to tumour or proliferative cells to prevent the function of that polypeptide. This may be performed, for example, by means of gene therapy or by direct administration of the compounds. Suitable antibodies of the invention may also be used as therapeutic agents.

Alternatively, double-stranded (ds) RNA is a powerful way of interfering with gene expression in a range of organisms that has recently been shown to be successful in mammals (Wianny and Zemicka-Goetz, 2000, Nat Cell Biol 2:70-75). Double stranded RNA corresponding to the sequence of a polynucleotide according to the invention can be introduced into or expressed in oocytes and cells of a candidate organism to interfere with cell division cycle progression.

In addition, a number of the mutations described herein exhibit aberrant meiotic phenotypes. Aberrant meiosis is an important factor in infertility since mutations that affect only meiosis and not mitosis will lead to a viable organism but one that is unable to produce viable gametes and hence reproduce. Consequently, the elucidation of genes involved in meiosis is an important step in diagnosing and preventing/treating fertility problems. Thus the polypeptides of the invention identified in mutant Drosophila having meiotic defects (as is clearly indicated in the Examples) may be used in methods of identifying substances that affect meiosis. In addition, these polypeptides, and corresponding polynucleotides, may be used to study meiosis and identify possible mutations that are indicative of infertility. This will be of use in diagnosing infertility problems.

Administration

Substances identified or identifiable by the assay methods of the invention may preferably be combined with various components to produce compositions of the invention. Preferably the compositions are combined with a pharmaceutically acceptable carrier or diluent to produce a pharmaceutical composition (which may be for human or animal use). Suitable carriers and diluents include isotonic saline solutions, for example phosphate-buffered saline. The composition of the invention may be administered by direct injection. The composition may be formulated for parenteral, intramuscular, intravenous, subcutaneous, intraocular or transdermal administration. Typically, each protein may be administered at a dose of from 0.01 to 30 mg/kg body weight, preferably from 0.1 to 10 mg/kg, more preferably from 0.1 to 1 mg/kg body weight.

Polynucleotides/vectors encoding polypeptide components (or antisense constructs) for use in inhibiting cell cycle progression, for example, inhibiting mitosis or meiosis, may be administered directly as a naked nucleic acid construct. They may further comprise flanking sequences homologous to the host cell genome. When the polynucleotides/vectors are administered as a naked nucleic acid, the amount of nucleic acid administered may typically be in the range of from 1 μg to 10 mg, preferably from 100 μg to 1 mg. It is particularly preferred to use polynucleotides/vectors that target specifically tumour or proliferative cells, for example by virtue of suitable regulatory constructs or by the use of targeted viral vectors.

Uptake of naked nucleic acid constructs by mammalian cells is enhanced by several known transfection techniques for example those including the use of transfection agents. Example of these agents include cationic agents (for example calcium phosphate and DEAE-dextran) and lipofectants (for example lipofectam™ and transfectam™). Typically, nucleic acid constructs are mixed with the transfection agent to produce a composition.

Preferably the polynucleotide, polypeptide, compound or vector described here may be conjugated, joined, linked, fused, or otherwise associated with a membrane translocation sequence.

Preferably, the polynucleotide, polypeptide, compound or vector, etc described here may be delivered into cells by being conjugated with, joined to, linked to, fused to, or otherwise associated with a protein capable of crossing the plasma membrane and/or the nuclear membrane (i.e., a membrane translocation sequence). Preferably, the substance of interest is fused or conjugated to a domain or sequence from such a protein responsible for the translocational activity. Translocation domains and sequences for example include domains and sequences from the HIV-1-trans-activating protein (Tat), Drosophila Antennapedia homeodomain protein and the herpes simplex-1 virus VP22 protein. In a highly preferred embodiment, the substance of interest is conjugated with penetratin protein or a fragment of this. Penetratin comprises the sequence RQIKIWFQNRRMKWKK and is described in Derossi et al., 1994, J. Biol. Chem. 269:10444-50; use of penetratin-drug conjugates for intracellular delivery is described in WO 00/01417. Truncated and modified forms of penetratin may also be used, as described in WO 00/2927.

Preferably the polynucleotide, polypeptide, compound or vector according to the invention is combined with a pharmaceutically acceptable carrier or diluent to produce a pharmaceutical composition. Suitable carriers and diluents include isotonic saline solutions, for example phosphate-buffered saline. The composition may be formulated for parenteral, intramuscular, intravenous, subcutaneous, intraocular or transdermal administration.

Use of timed release or sustained release delivery systems are also included in the invention. Such systems are highly desirable in situations where surgery is difficult or impossible, e.g., patients debilitated by age or the disease course itself, or where the risk-benefit analysis dictates control over cure.

A sustained-release matrix, as used herein, is a matrix made of materials, usually polymers, which are degradable by enzymatic or acid/base hydrolysis or by dissolution. Once inserted into the body, the matrix is acted upon by enzymes and body fluids. The sustained-release matrix desirably is chosen from biocompatible materials.

The routes of administration and dosages described are intended only as a guide since a skilled practitioner will be able to determine readily the optimum route of administration and dosage for any particular patient and condition.

The invention will now be further described by way of Examples, which are meant to serve to assist one of ordinary skill in the art in carrying out the invention and are not intended in any way to limit the scope of the invention.

### EXAMPLES

Introduction

In order to identify new human cell cycle regulatory genes, candidate genes were identified by establishing the role of their Drosophila counterparts in cell cycle progression through an RNAi-based knockdown approach in cultured Drosophila cells followed by mitotic index evaluation (Cellomics Arrayscan) and confirming the role of the human gene through RNAi in human cells followed by FACS analysis and microscopy.

A list of Human cell cycle progression genes and their Drosophila counterparts for which data is described is presented in Table 1. Multiple protein sequence gi numbers are listed where alternative transcripts or splice variants for a Drosophila gene are listed in the data base, These Human and Drosophila genes and proteins are useful, for example, for screening for anti-proliferative molecules.

The homologies (Drosophila and human) are clustered homologies within a region of the gene and do not relate to comparison of the whole human gene with the whole Drosophila gene. The similarities quoted relate to the region of greatest homology for that gene. The score also indicates confidence in homology, with higher scores indicating higher confidence. The score relates not only to the percentage similarity, but also to the length of sequence over which the similarity extends.

The homologues are identified by performing a BLAST search and identifying the human protein with the best homology, i.e., the one nearest the top of the search results list, where genes are ranked in descending order according to the likelihood of a match not being due to chance. In some cases, there are other sections of the Drosophila and human genes with homology.

TABLE 1

| Drosophila Gene | | | | | | |
|---|---|---|---|---|---|---|
| | | Protein | Human homologue(s) | | | |
| | Nucleotide | sequence | | SWISS- | | |
| | sequence | gi | | PROT | BLAST results | |
| Gene Name | gi numbers | numbers | Gene Name | reference | Similarity | Score |
| CG3632 | 10728281 | 7293200 | Similar to myotubularin related protein 3. | Q9UEG3 | 365/575 | 546 |
| | | 7293201 | Hypothetical FYVE domain-containing dual | | (62%) | |
| | | 7293199 | specificity protein phosphatase FYVE- | | | |
| | | 7293198 | DSP1C. | | | |
| Pp1-87B | 7299572 | 7299625 | Serine/threonine protein phosphatase PP1- | P36873 | 289/299 | 590 |
| | | | gamma catalytic subunit (EC 3.1.3.16) (PP- | | (96%) | |
| | | | 1G). | | | |
| CG3524 | 10727365 | 7295849 | Fatty acid synthase (EC 2.3.1.85). | Q16702 | 748/1174 | 1011 |
| | | | | | (62%) | |
| CG9311 | 10727923 | 7294357 | Protein tyrosine phosphatase HD-PTP | Q9H3S7 | 423/738 | 480 |
| | | | (Protein tyrosine phosphatase TD14). | | (56%) | |
| CG9092 | 7297037 | 7297052 | Beta-galactosidase precursor (EC 3.2.1.23) | P16278 | 364/672 | 402 |
| | | | (Lactase) (Acid beta-galactosidase). | | (53%) | |
| Arr1 | 10728850 | 7298421 | Beta-Arrestin 1. A regulator of GPCR | P49407 | 228/356 | 306 |
| | | | activity | | (63%) | |
| CG9150 | 7297037 | 7297069 | Hypothetical protein with putative | Q9BUC7 | 101/176 | 115 |
| | | | dehydrogenase/reductase domains | | (56%) | |
| CG11102 | 10728232 | 7292929 | KIAA0659 protein (Fragment). | BAA31634 | 330/562 | 422 |
| | | | | | (58%) | |
| Smr | 7292788 | 7292802 | Nuclear receptor co-repressor 1 (NCR-1). | O75376 | 206/379 | 226 |
| | | | | | (53%) | |
| CG8045 | 7300335 | 7300358 | 14-3-3 protein epsilon (Mitochondrial import | P42655 | 237/258 | 424 |
| | | 7300360 | stimulation factor L subunit) (Protein kinase C | | (91%) | |
| | | 7300364 | inhibitor protein-1) (KCIP-1) (14-3-3E). | | | |
| CG10420 | 7301280 | 7301293 | SIL1 protein precursor (Endoplasmic | Q9H173 | 159/318 | 135 |
| | | | reticulum chaperone SIL1, homolog of yeast). | | (49%) | |
| Hsc70-2 | 10726497 | 7299717 | Heat shock cognate 71 kDa protein. | P11142 | 507/606 | 879 |
| | | | | | (82%) | |
| CG10805 | 7297167 | 7297181 | Protein BAP28. | Q9H583 | 629/1324 | 465 |
| | | | | | (47%) | |
| eIF-4a | 7297037 | 7297048 | Similar to eukaryotic translation initiation | Q96EA8 | 342/402 | 576 |
| | | | factor 4A2 | | (84%) | |
| ACXA | 7297983 | 10728753 | CYA2 Adenylate cyclase, type II (EC 4.6.1.1) | Q08462 | 420/931 | 306 |
| | | | (ATP pyrophosphate-lyase) (Adenylyl | | (44%) | |
| | | | cyclase) (Fragment). | | | |
| | | | CYA8 Adenylate cyclase, type VIII (EC | P40145 | 446/1037 | 296 |
| | | | 4.6.1.1) (ATP pyrophosphate-lyase) | | (42%) | |
| | | | (Ca(2+)/calmodulin activated adenylyl | | | |
| | | | cyclase). | | | |
| CG15117 | 10727456 | 7302519 | Beta-glucuronidase precursor (EC 3.2.1.31) | P08236 | 357/638 | 431 |
| | | | (Beta-G1). | | (55%) | |
| BG:DS01759.2 | 7298121 | 7298138 | No human homologue | | | |
| TepIII | 7297264 | 7297279 | Cell surface antigen CD109. | Q8TDJ3 | 686/1478 | 500 |
| | | | | | (46%) | |
| Hsc70-4 | 10726541 | 7299978 | HS7C Heat shock cognate 71 kDa protein. | P11142 | 539/612 | 994 |
| | | | | | (87%) | |
| CG7069 | 10726692 | 10726696 | Pyruvate kinase, M2 isozyme (EC 2.7.1.40). | P14786 | 280/412 | 400 |
| | | | | | (67%) | |
| ACXE | 7297983 | 7297987 | CYA8 Adenylate cyclase, type VIII (EC | P40145 | 454/968 | 331 |
| | | | 4.6.1.1) (ATP pyrophosphate-lyase) | | (46%) | |
| | | | (Ca(2+)/calmodulin activated adenylyl | | | |
| | | | cyclase). | | | |
| | | | CYA7 Adenylate cyclase, type VII (EC | P51828 | 211/397 | 212 |
| | | | 4.6.1.1) (ATP pyrophosphate-lyase) | | (52%) | |
| | | | (Adenylyl cyclase). | | | |
| EG:52C10.5 | 10727480 | 7302711 | FYVE finger-containing phosphoinositide | Q9Y217 | 193/348 | 253 |
| | | | kinase (EC 2.7.1.68) (1-phosphatidylinositol- | | (54%) | |
| | | | 4-phosphate kinase) (PIP5K) (PtdIns(4)P-5- | | | |
| | | | kinase) (p235) (Fragment). | | | |
| gatA | 10726610 | 7300468 | Hypothetical protein | Q9H0R6 | 320/507 | 465 |
| | | | | | (62%) | |
| CG17149 | 10727803 | 7293682 | DJ184J9.1 (Hypothetical protein KIAA0601) | Q9NUH3 | 552/856 | 793 |
| | | 7293681 | (Fragment) | | (63%) | |
| CG2905 | 10728163 | 7302249 | TRRAP protein/PI3 PI4 kinase, a novel ATM- | Q9Y6H4 | 2393/3582 | 3391 |
| | | | related protein. | | (65%) | |
| CG2336 | 10727121 | 7298905 | No human homologue | | | |
| TER94 | 10727672 | 7303816 | Transitional endoplasmic reticulum ATPase | P55072 | 692/803 | 1248 |
| | | 7303817 | (TER ATPase) (15S Mg(2+)- ATPase p97 | | (86%) | |
| | | | subunit) (Valosin containing protein) (VCP) | | | |
| | | | [Contains: Valosin]. | | | |
| CG6313 | 10726739 | 7301133 | KIAA0697 protein (Fragment). | BAA31672 | 451/575 | 737 |
| | | | | | (78%) | |
| aur | 10726473 | 7299536 | AURORA-related kinase 1 (DJ1167H4.2) | O60445 | 207/262 | 346 |
| | | | (Serine/threonine kinase 15). | | (78%) | |
| | | | AURORA-related kinase 2 (Serine/threonine | O60446 | 205/261 | 337 |
| | | | kinase 12). | | (78%) | |
| Pk91C | 10799498 | 7300437 | hypothetical protein FLJ14813 | Q96SJ5 | | |
| Top2 | 10728874 | 7298585 | DNA topoisomerase II, alpha isozyme (EC | P11388 | 651/898 | 1026 |
| | | | 5.99.1.3). | | (71%) | |
| | | | DNA topoisomerase II, beta isozyme (EC | Q02880 | 903/1200 | 1470 |
| | | | 5.99.1.3). | | (74%) | |
| alpha-Est1 | 10727101 | 10727102 | Acetylcholinesterase precursor (EC 3.1.1.7) | P22303 | 251/542 | 208 |
| | | | (AChE). | | (45%) | |
| Nrk | 10727582 | 7303361 | Muscle specific tyrosine kinase receptor. | O15146 | 228/303 | 378 |
| | | | | | (74%) | |
| otk | 10727617 | 7303541 | PTK7 Tyrosine-protein kinase-like 7 | Q13308 | 156/245 | 208 |
| | | | precursor (Colon carcinoma kinase-4) (CCK- | | (63%) | |
| | | | 4). | | | |
| cad | 10799497 | 7298711 | Homeobox protein CDX-2 (Caudal-type | Q99626 | 67/81 | 123 |
| | | | homeobox protein 2) (CDX-3). | | (82%) | |
| Rut | 10728252 | 7293001 | CYA6 Adenylate cyclase, type VI (EC | O43306 | 293/441 | 405 |
| | | | 4.6.1.1) (ATP pyrophosphate-lyase) (Ca(2+)- | | (66%) | |
| | | | inhibitable adenylyl cyclase). | | | |
| CG8002 | 10728334 | 7293569 | KIAA1999 protein (Fragment). | BAC02708 | 270/526 | 260 |
| | | | | | (51%) | |
| CG10335 | 10727968 | 7294597 | HEM2 Delta-aminolevulinic acid dehydratase | P13716 | 243/318 | 392 |
| | | | (EC 4.2.1.24) (Porphobilinogen synthase) | | (75%) | |
| | | | (ALADH). | | | |
| CG8070 | 10727693 | 7303942 | Hypothetical protein FLJ10498 | Q9NVU7 | 301/458 | 447 |
| | | | | | (65%) | |
| CG7460 | 10727853 | 7293951 | Polyamine oxidase isoform-1. | Q96QT3 | 209/521 | 137 |
| | | | | | (39%) | |
| CG17735 | 10727172 | 7296816 | Thyroid receptor interacting protein 12 | Q14669 | 438/663 | 652 |
| | | | (TRIP12). | | (65%) | |
| Gycalpha99B | 7301790 | 7301807 | CYG4 Guanylate cyclase soluble, alpha-2 | P33402 | 363/670 | 394 |
| | | | chain (EC 4.6.1.2) (GCS-alpha-2). | | (54%) | |
| CG13893 | 7291959 | 7291980 | SEC14-like protein 2 (Alpha-tocopherol | O76054 | 179/316 | 194 |
| | | | associated protein) (TAP) (hTAP) | | (56%) | |
| | | | (Supernatant protein factor) (SPF) (Squalene | | | |
| | | | transfer protein). | | | |
| CG18176 | 10728019 | 7294884 | DKFZP434B168 protein. | AAH33918 | 517/994 | 502 |
| | | | | | (51%) | |
| CG8858 | 10727617 | 7303499 | Hypothetical protein KIAA0368 (Fragment). | O15074 | 752/1552 | 607 |
| | | | | | (47%) | |
| Ac76E | 10733346 | 10733351 | CYA2 Adenylate cyclase, type II (EC 4.6.1.1) | Q08462 | 194/267 | 294 |
| | | | (ATP pyrophosphate-lyase) (Adenylyl | | (71%) | |
| | | | cyclase) (Fragment). | | | |
| | | | Adenylate cyclase, type VII (EC 4.6.1.1) | P51828 | 257/463 | 291 |
| | | | (ATP pyrophosphate-lyase) (Adenylyl | | (55%) | |
| | | | cyclase). | | | |
| CG17010 | 7297915 | 7297937 | Ribokinase (EC 2.7.1.15). | Q9H477 | 155/270 | 164 |
| | | | | | (57%) | |
| Tkv | 7296952 | 22945650 | Bone morphogenetic protein receptor type IB | O00238 | 313/489 | 460 |
| | | | precursor (EC 2.7.1.37). | | (63%) | |
| | | | Bone morphogenetic protein receptor type IA | P36894 | 307/490 | 454 |
| | | | precursor (EC 2.7.1.37) (Serine/threonine- | | (62%) | |
| | | | protein kinase receptor R5) (SKR5) (Activin | | | |
| | | | receptor-like kinase 3) (ALK-3). | | | |
| | | | Activin receptor type I precursor (EC | Q04771 | 309/521 | 394 |
| | | | 2.7.1.37) (ACTR-I) (Serine/threonine-protein | | (59%) | |
| | | | kinase receptor R1) (SKR1) (Activin receptor- | | | |
| | | | like kinase 2) (ALK-2) (TGF-B superfamily | | | |
| | | | receptor type I) (TSR-I). | | | |
| Dnt | 10728868 | 7298565 | Tyrosine-protein kinase RYK precursor (EC | P34925 | 305/558 | 343 |
| | | | 2.7.1.112). | | (54%) | |
| ACXD | 10727242 | 7292211 | CYA2 Adenylate cyclase, type II (EC 4.6.1.1) | Q08462 | 444/976 | 333 |
| | | | (ATP pyrophosphate-lyase) (Adenylyl | | (45%) | |
| | | | cyclase) (Fragment). | | | |
| | | | CYA8 Adenylate cyclase, type VIII (EC | P40145 | 423/897 | 320 |
| | | | 4.6.1.1) (ATP pyrophosphate-lyase) | | (46%) | |
| | | | (Ca(2+)/calmodulin activated adenylyl | | | |
| | | | cyclase). | | | |
| Aats-ala-m | 10728137 | 7295490 | Alanyl-tRNA synthetase (EC 6.1.1.7) | P49588 | 511/1047 | 489 |
| | | | (Alanine-tRNA ligase) (AlaRS). | | (48%) | |
| Gek | 7291737 | 7291742 | CDC42-binding protein kinase beta. | Q9Y5S2 | 977/1678 | 1155 |
| | | | | | (57%) | |
| CG3216 | 10726992 | 7291217 | Atrial natriuretic peptide receptor A precursor | P16066 | 588/1046 | 682 |
| | | | (ANP-A) (ANPRA) (GC-A) (Guanylate | | (55%) | |
| | | | cyclase) (EC 4.6.1.2) (NPR-A) (Atrial | | | |
| | | | natriuretic peptide A-type receptor). | | | |
| | | | Atrial natriuretic peptide receptor B precursor | P20594 | 146/224 | 187 |
| | | | (ANP-B) (ANPRB) (GC-B) (Guanylate | | (64%) | |
| | | | cyclase) (EC 4.6.1.2) (NPR-B) (Atrial | | | |
| | | | natriuretic peptide B-type receptor). | | | |
| CG5653 | 10728037 | 7295017 | Polyamine oxidase isoform-1. | Q96QT3 | 105/234 | 99 |
| | | | | | (44%) | |
| CG17740 | 10727606 | 21627360 | VSGP/F (vascular smooth muscle cell growth | Q9HCB6 | 261/527 | 259 |
| | | | promoting factor)-spondin. | | (49%) | |
| Tepl | 7298255 | 10728811 | Cell surface antigen CD109. | Q8TDJ3 | 644/1355 | 497 |
| | | | | | (46%) | |
| for | 10727349 | 10727350 | cGMP-dependent protein kinase 1, alpha | Q13976 | 518/706 | 827 |
| | | 10727351 | isozyme (EC 2.7.1.37) (CGK 1 alpha) (cGKI- | | (73%) | |
| | | 10727352 | alpha). Exhibits a substrate specificity similar | | | |
| | | 10727353 | but not identical to CAK | | | |
| | | 10727354 | | | | |
| Ac13E | 10728265 | 7293083 | Adenylate cyclase, type IX (EC 4.6.1.1) (ATP | O60503 | 286/521 | 351 |
| | | | pyrophosphate-lyase) (Adenylyl cyclase). | | (54%) | |
| | | | Adenylate cyclase type III (EC 4.6.1.1) | O60266 | 174/307 | 208 |
| | | | (Adenylate cyclase, olfactive type) (ATP | | (55%) | |
| | | | pyrophosphate-lyase) (Adenylyl cyclase) (AC- | | | |
| | | | III) (AC3). | | | |
| CG2667 | 7298935 | 7298950 | Diacylglycerol kinase, delta (EC 2.7.1.107) | Q16760 | 327/526 | 421 |
| | | | (Diglyceride kinase) (DGK-delta) (DAG | | (61%) | |
| | | | kinase delta) (130 kDa diacylglycerol kinase) | | | |
| | | | (Fragment). | | | |
| CG7842 | 10727872 | 7294021 | BK1191B2.3.1 (Putative novel acyl | O95510 | 203/310 | 299 |
| | | | transferase similar to C. ELEGANS C50D2.7) | | (65%) | |
| | | | (Isoform 1) (Fragment). | | | |
| CG17486 | 7289853 | 7289856 | Hypothetical protein FLJ20752 | Q9NWL6 | 310/632 | 273 |
| | | | | | (48%) | |
| CG6969 | 10726705 | 7300903 | MYELOBLAST KIAA0230 [Fragment], a | Q92626 | 295/574 | 317 |
| | | | human melanoma associated gene | | (51%) | |
| CG12262 | 10728071 | 7295201 | Acyl-CoA dehydrogenase, medium-chain | P11310 | 337/411 | 587 |
| | | | specific, mitochondrial [Precursor] | | (81%) | |
| Fray | 10726601 | 10726604 | Oxidative-stress responsive 1, a Ser/Thr | O95747 | 338/524 | 513 |
| | | | pkinase | | (63%) | |
| CG6879 | 10726756 | 7301203 | MYELOBLAST KIAA0230 [Fragment], a | Q92626 | 298/594 | 318 |
| | | | human melanoma associated gene | | (49%) | |
| CG11594 | 10727290 | 7292419 | Hypothetical protein with FGGY carbohydrate | Q96C11 | 286/442 | 402 |
| | | | kinase domain | | (64%) | |
| S6kII | 10803726 | 7295638 | Ribosomal protein S6 kinase alpha 3/Insulin- | P51812 | 520/744 | 803 |
| | | | stimulated protein kinase 1 (ISPK-1). | | (69%) | |
| CG11714 | 10727982 | 7294716 | Hypothetical protein | Q9HAB2 | 82/207 | 56 |
| | | | | | (39%) | |
| | | | speckle-type POZ protein [SPOP]/BTB | O43791 | 61/137 | 54 |
| | | | domain protein (Fragment) [BDPL] | | (44%) | |
| CG3534 | 7300193 | 7300206 | Xylulokinase [XYLB] | O75191 | 299/489 | 395 |
| | | | | | (60%) | |
| CG7335 | 10727803 | 7293697 | Ketohexokinase (EC 2.7.1.3) (Hepatic | P50053 | 145/330 | 102 |
| | | | fructokinase) [KHK] | | (43%) | |
| CG11275 | 7291355 | 7291386 | Hypothetical protein | Q9HAB2 | 83/181 | 71 |
| | | | | | (45%) | |
| | | | Speckle-type POZ protein [SPOP]/BTB | O43791 | 58/93 | 60 |
| | | | domain protein (Fragment) [BDPL] | | (61%) | |
| CG16726 | 10728019 | 7294899 | Thyrotropin-releasing hormone receptor | P34981 | 81/166 | 75 |
| | | | (TRH-R) (Thyroliberin receptor) [TRHR]. | | (48%) | |
| CG7514 | 10727313 | 7292529 | Mitochondrial 2-oxoglutarate/malate carrier | Q02978 | 199/296 | 301 |
| | | | protein | | (66%) | |
| CG17283 | 7300241 | 7300253 | Cathepsin E [Precursor] | P14091 | 178/332 | 218 |
| | | | | | (53%) | |
| BcDNA:GH07626 | 10727365 | 7295848 | Fatty acid synthase (EC 2.3.1.85) | Q16702 | 743/1140 | 1077 |
| | | | | | (65%) | |
| CG16752 | 10728478 | 7290587 | Putative G-protein coupled receptor [GPCR] | Q8TDU8 | 66/144 | 49 |
| | | | | | (45%) | |
| Rpt1 | 10727757 | 7304183 | MSS1 protein-26S protease regulatory | P35998 | 391/433 | 743 |
| | | | subunit 7. | | (89%) | |
| Wts | 7301969 | 7301980 | Large tumor suppressor 1 [LATS1] | O95835 | 362/428 | 630 |
| | | | | | (83%) | |
| | | | Large tumor suppressor 2 (Fragment) | Q9P2X1 | 346/414 | 604 |
| | | | [HSLATS2] | | (83%) | |
| CG1582 | 7292554 | 7292573 | Putative DEAH-box RNA/DNA helicase | AAM73547 | 584/866 | 776 |
| | | | | | (66%) | |
| CG12289 | 10727982 | 7294728 | Ketohexokinase (EC 2.7.1.3) (Hepatic | P50053 | 141/300 | 147 |
| | | | fructokinase) [KHK] | | (49%) | |
| Pepck | 10727469 | 7302595 | Phosphoenolpyruvate (EC 4.1.1.32) | Q16822 | 453/616 | 789 |
| | | | carboxykinase, mitochondrial precursor [GTP] | | (73%) | |
| | | | (Phosphoenolpyruvate carboxylase) | | | |
| | | | (PEPCK-M) [PCK2] | | | |
| CG5665 | 10726906 | 7296289 | Lipoprotein lipase precursor (EC 3.1.1.34) | P06858 | 119/259 | 105 |
| | | | (LPL) [LPL] | | (45%) | |
| CG7285 | 10727839 | 7293895 | Somatostatin receptor 2B [SSTR2] | Q96TF2 | 174/335 | 201 |
| | | | | | (51%) | |
| Bt | 10726313 | 10726323 | Titin | Q8WZ42 | 2478/5772 | 1796 |
| | | | | | (42%) | |
| CG8795 | 10726505 | 7299748 | Neuromedin U receptor-type 2 | Q9GZQ4 | 167/302 | 177 |
| | | | G protein-coupled receptor TGR-1 | | (55%) | |
| CG10967 | 10727955 | 7294537 | Serine/threonine-protein kinase ULK1 (EC | O75385 | 255/504 | 327 |
| | | | 2.7.1.—) (Unc-51-like kinase 1) [ULK1] | | (50%) | |
| CG3809 | 10726480 | 7299571 | Adenosine kinase (EC 2.7.1.20) (AK) | P55263 | 203/343 | 251 |
| | | | (Adenosine | | (58%) | |
| | | | 5′-phosphotransferase) [ADK] | | | |
| Ack | 10727290 | 10727294 | Activated p21cdc42 Hs kinase [ACK1] | Q07912 | 309/438 | 489 |
| | | | | | (69%) | |
| AbI | 10727878 | 7294076 | Proto-oncogene tyrosine-protein kinase ABL1 | P00519 | 407/470 | 742 |
| | | | (EC 2.7.1.112) (p150) (c-ABL) [ABL1] | | (86%) | |
| CG7362 | 10726534 | 7299922 | Pyruvate kinase, M2 isozyme (EC 2.7.1.40) | P14786 | 166/273 | 214 |
| | | | [PKM2] | | (59%) | |
| Cyp9f2 | 7299572 | 7299618 | Cytochrome P450 3A4 (EC 1.14.14.1) | P08684 | 282/505 | 270 |
| | | | (CYPIIIA4) (Nifedipine oxidase) (NF-25) | | (55%) | |
| | | | (P450-PCN1) [CYP3A4] | | | |

1) Synthesis of D.MEL-2 Genomic DNA and cDNA
i) Genomic DNA

D.Mel-2 cells from an established culture were grown in a 500 ml Erlenmeyer flask in Drosophila-SFM/glutamine/Pen-Strep at 28° C. until the culture reached 2×10⁷ cells/ml.

Cells were pelleted and washed twice with 50 ml PBS, then the pellet resuspended in 1 volume (10 ml) digestion buffer (100 mM NaCl, 10 mM Tris pH8, 25 mM EDTA pH8, 0.5% SDS, 0.1 mg/ml proteinase K) and incubated at 50° C. for 15 hrs (in Hybaid rotisserie). DNA was extracted 3 times with an equal volume of Phenol/Chloroform and twice with an equal volume of Chloroform.

DNA was then precipitated by adding ½ volume of 7.5M ammonium acetate and 2 volumes of 97% ethanol, incubating at −20° C. for 15 minutes, then centrifuging at 10,000 rpm, 4° C. for 20 minutes. The pellet was washed with 70% ethanol, allowed to dry, then dissolved in 12 ml 10 mM Tris (pH 8.5).

For sufficient genomic DNA to amplify all the Drosophila genes requiring a genomic DNA template in the PCR reaction, this procedure was repeated a further 7 times, pooling the genomic DNA and the concentration assessed by measuring A260/A280 values.

ii) cDNA

D.Mel-2 total RNA was prepared as follows:

Cells were pelleted from 50 ml of a DMEL-2 culture at 5×10⁶ cells/ml and washed twice with 50 ml PBS, then resuspended in 4 ml denaturing solution (4 M guanidine thiocyanate, 25 mM sodium citrate, 0.5% N-laurylsarcosine (Sarkosyl), 0.1M 2-mercaptoethanol) in a corex tube.

400 μl 2 M sodium acetate pH 4.0 was added and mixed by inversion before adding 4 ml water-saturated phenol followed by 800 μl 49:1 chloroform/isoamylalcohol, then the suspension incubated on ice for 15 mins. The mixture was then spun in a centrifuge at 10,000 rpm for 30 mins and the aqueous phase transferred to a fresh tube. The RNA was precipitated by adding 4 ml isopropanol and incubating at 20° C. for 20 mins. The RNA was centrifuged at 10,000 rpm for 30 minutes at 4° C. to pellet the RNA and the pellet dissolved in 1200 μl denaturing solution. The RNA was precipitated a second time by adding 1200 μl isopropanol and incubating at −20° C. for 20 mins. The mixture was then centrifuged at 10,000 rpm for 30 minutes at 4° C. to pellet the RNA which was then washed with 1 ml 75% ethanol, incubating at room temperature for 10 mins, then centrifuging at 4° C. for 5 mins. The supernatant was removed and the pellet allowed to dry before resuspending in 800 μl RNase- and DNase-free water. The concentration of the total RNA preparation was assessed from A₂₆₀/A₂₈₀ values.

D.Mel-2 cDNA was prepared using SuperScript™ First-strand synthesis system for RT-PCR (Invitrogen Ltd, 3 Fountain Drive, Inchinnan Business Park, Paisley PA4 9RF, UK).

To prepare sufficient cDNA to amplify all the Drosophila genes requiring a cDNA template in the PCR reaction, 96 50 μl cDNA preparations reactions in a 96-well plate were carried out as follows:

Reaction Mix (1) (800 μg D.MeI-2 RNA, 250 μl 10 mM dNTP mixture, 250 pt 500 μg/ml oligo dT primer, RNase- and DNase-free water to a final volume of 2.6 ml) was prepared and 26 μl was aliquoted into each well of a 96-well plate, then incubated at 65° C. for 5 minutes.

Reaction Mix (2) was prepared (500 μl 10×RT buffer, 1000 μl 25 mM MgCl₂, 500 μl 0.1 M DTT, 250 μl RNase OUT and 250 μl SuperScript™ II RT) was prewarmed to 42° C. for 2 minutes, then 25 μl added to each well of the 96-well plate containing the 26 μl Reaction Mix (1).

The plate was heat sealed and incubated at 42° C. for 1 hour, then at 70° C. for 15 minutes on PTC-225 thermocycler (MJ Research, 590 Lincoln Street, Waltham, Mass. 02451-1003, USA). The reactions were placed on ice for 5 minutes, then 2.5 μl RNase H added to each well and incubated at 37° C. for 20 minutes. The reactions were pooled, mixed and stored as 200 μl aliquots at −20° C.

2) Generating Drosophila Gene Specific dsRNA

i) Designing and ordering RNAi primers.

Primers for the amplification of specific sequences of groups of Drosophila genes to be used as templates for the preparation of dsRNA were designed using PhilaSys primer design program (PhilaSysAmis Software Pvt. Ltd 120-1A Elephant Rock Road, 3 Brock Jaayenagar, Bangalore 560011, India). This program is designed to identify suitable primers for each of the Drosophila genes in the NCBI database, tagging a T7 RNA polymerase binding site sequence (TAATACGACTCACTATAGGGAGA) to the 5′ end of each primer.

The following parameters were used in the primer design process:
Length of final PCR product=between 500 to 550 base pairs
Length of primer=25-35 base pairs
Primer melting temperature=from 50.0 to 80.0C
Minimum % GC=35.0
Maximum permitted length of palindromes=8 base pairs
Maximum permitted value of free energy of haripin loops=−1.0 kcal/mol
Minimum permitted value of primer-primer duplex free energy=−15.0 kcal/mol
Minimum permitted value for primer-primer 3′ end duplex free energy=−3.0 kcal/mol
Maximum difference in the melting temperatures between primer pairs=5° C.
Maximum value for the difference in the % GC between the primers=10.0
Primer concentrations=250 pMol
Salt concentration=50 mM

Primer pairs amplifying an intra-exon sequence were tagged as genomic (indicating that genomic DNA could be used in the PCR reaction) while sequences spanning 2 or more exons were tagged as cDNA (indicating a requirement for cDNA template for PCR amplification of the gene specific sequence).

Where no suitable primer pair for a gene was identified by the primer design program, the parameters were modified and the search process repeated. The minimum parameters settings were as follows:
Length of the final PCR product=between 150 to 200 base pairs
Length of the primer=25-35 base pairs
Primer melting temperature=from 50.0 to 65.0C
Minimum % GC of the primers=30.0
Maximum permitted length of palindromes=10 base pairs
Maximum permitted value of free energy of hairpin loops=−2.5 kcal/mol
Minimum permitted value of primer-primer duplex free energy=−10.0 kcal/mol
Minimum permitted value for primer-primer 3′ end duplex free energy=−5.0 kcal/mol
Maximum difference in the melting temperatures between primer pairs=8° C.
Maximum value for the difference in the % GC between the primers=15.0
Primer concentrations=250 pMol
Salt concentration=50 mM

Primers (Table 2) were ordered from MWG (MWG Biotech (UK) Ltd, Mill Court, Featherstone Road, Wolverton Mill South, Milton Keynes, MK12 5RD, UK) pre-mixed at a concentration of 50 μM in 96 well Thermosprint plates.

ii) The primers were then used to synthesis Drosophila Gene Specific PCR products for dsRNA production.

The details of the primers for each plate were imported into the MWG AG Biotech RoboSeq 4204SE samples database.

The PCR reactions were aliquoted in a 96-well plate format using the RoboSeq 4204SE following the manufacturer's instructions. Each reaction comprised of 3 μl 17 μM gene specific primer pair, 2 μl D.MeI-2 genomic DNA or cDNA (2 μg/ml) as appropriate and 45 μl 1.1× thermo-start PCR master mix with 2.5 mM MgCl₂ (ABgene House, Blenheim Road, Epsom, Surrey KT19 9AP, UK) following the manufacturer's instructions. The RoboSeq 4204SE assigned barcode for each plate was recorded and the plates were stored at −80° C.

Once the RoboSeq 4204SE completed the PCR reaction set up, the plates were sealed and transferred to the PTC-225 thermocycler, running the programme THERMOPC, with steps 2 and 3 repeated 35 times:
1: 95° C. 00:15
2: 95° C. 00:30
3: 55° C. 00:45
4: 72° C. 01:00
5: 72° C. 05:00
6: 4° C. ∞

The PCR products were purified using the RoboSeq 4204SE together with Millipore's Montage™ PCR₉₆ Cleanup Kit (Millipore (U.K.) Ltd, Units 3&5, The Courtyards, Hatters Lane, Watford, WD18 8YH, UK).

The RoboSeq 4204SE assigned barcode for each plate was again recorded and the purified PCR product plates were stored at −20° C.

Each PCR product plate was thawed and sequences verified by running a sequencing reaction using ordered arrayed sequencing primers corresponding to the forward primers used in the original amplification reactions, but without the 5′ T7 RNA polymerase binding site sequence. Sequencing reactions were performed by Lark Technologies, Inc. (Radwinter Road, Saffron Walden, Essex, CB13HY, UK).

The resulting sequences were verified to ensure that they corresponded to the sequences of the genes associated with each of the test wells, by BLAST searching the NCBI database.

iii) Gene specific PCR products were used as templates in in vitro transcription reactions to produce gene specific dsRNA.

Reactions were set up in a 96-well plate format using the RoboSeq 4204SE, with each reaction comprised of 8 μl gene specific PCR product, 6 μl 3.3×T7 RNA polymerase buffer (130.7 mM HEPES (pH 8.1), 16.3 mM DTT, 13.1% PEG 8000, 0.03% Triton X-100, 65.3 mM Mg(OAc)), 4 μl 25 mM rNTP mix, 0.5 μl 50 U/μl yeast inorganic pyrophosphatase, 1 μl 20 U/μl Rnasin, 0.5 μl 80 U/μl T7 RNA polymerase. The reactions were incubated for 4 hours at 37° C. on the PTC-225 thermocycler.

Transcribed RNA was treated with DNase I (2U/μl) at 37° C. for 15 mins and diluted by the addition of 80 μl RNase-free Milli-Q water again with the aid of the RoboSeq 4204SE. To anneal the RNA, the samples were heated to 95° C. for 10 mins and then cooled slowly to room temperature overnight.

The RoboSeq 4204SE assigned barcode for each plate was recorded and the dsRNA plates were stored at −20° C.

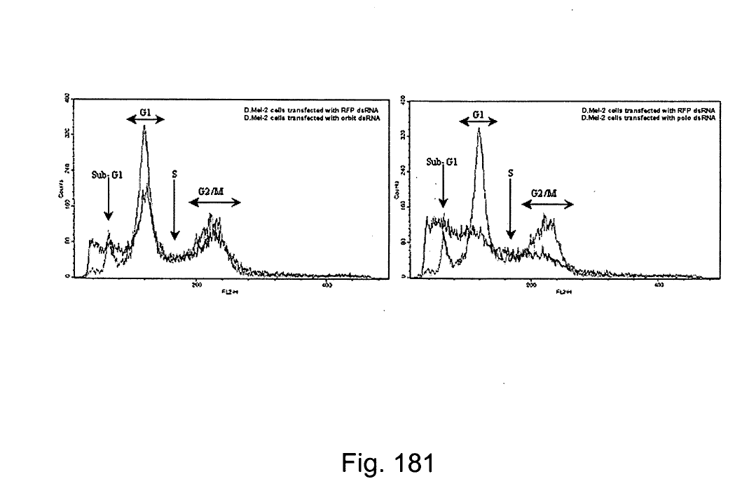

iv) Synthesis of control sequences from Red Fluorescent Protein (RFP) (Matz et al., 1999, Nat Biotechnol. 17:969-973), Drosophila orbit and Drosophila polo dsRNA. RFP dsRNA was used as a negative control throughout, due to this gene being absent from D.Mel-2 cells. dsRNAs targeting 2 well established Drosophila cell cycle genes, orbit (Inoue et al., 2000, J. Cell Biol. 149:153-166, Maiato et al., 2002, J. Cell Biol. 157:749-760.) and polo (Sunkel and Glover, 1988, J Cell Sci. 89:25-38, Fenton and Glover, 1993, Nature 363:637-640, Carmena et al., 1998, J. Cell Biol. 143:659-671), were used as positive controls throughout, as experiments had shown that RNAi of orbit or polo gene expression has a significant affect on cell cycle progression (See FIG. 181 of FACS profiles for D.Mel-2 cells).

These control dsRNAs were prepared in 96 reactions in a 96-well plate format essentially as before, with some minor differences:

For PCR amplification of RFP gene specific sequence 0.1 μl 0.5 mg/ml 716 bp RFP sequence cloned into pGEM-T Easy vector (Invitrogen) together with primers RFP-1 and RFP-2 as follows:

RFP-1
TAATACGACTCACTATAGGGCGAATTGGGCCCGACGT
RFP-2
TAATACGACTCACTATAGGGCGATGCAGGCGGCCGCGAATTCACTAGT

polo specific sequence was amplified from D.MeI-2 cDNA using primers RNAi7 and RNAi8:

RNAi7
GAATTAATACGACTCACTATAGGGAGAGGCCGCGAAGCCCGAGGATAAGA
GCA
RNAi8
GAATTAATACGACTCACTATAGGGAGAGATGACCATATCCGCCGCCGGTT
TCCTT

orbit specific sequence was amplified from D.Mel-2 genomic DNA using primers RNAi192 and RNAi193:

RNAi192
TAATACGACTCACTATAGGGAGACCCGCATTGGCCGAACACCTGGAACC)
RNAi193
TAATACGACTCACTATAGGGAGAACGTCGAGACCCCGCACCTGTAGAGT

The 96 dsRNA preparations for polo, orbit or RFP were pooled and the concentrations assessed from A₂₆₀/A₂₈₀ values. The samples were aliquoted and stored at −20° C.

v) The quality and concentration of dsRNA in the samples was assessed by agarose gel electrophoresis and by fluorometry using PicoGreen reagent (Molecular Probes, PoortGebouw, Rijnsburgerweg 10, 2333 AA Leiden, The Netherlands) and a KC4 fluorometer (Bio-Teks Instruments, Inc., Winooski, Vt.) linked to the RoboSeq 4204SE, following manufacturers' recommendations. The standard curve used to assess dsRNA concentrations was generated using orbit dsRNA prepared essentially as above, with the exception that the dsRNA (200 μg) was treated with 20 μg/ml RNaseA in the presence of 0.3 M NaCl for 30 minutes at 30° C., to remove ssRNA. The reaction was terminated with 0.1 mg/ml proteinase K and the dsRNA purified by extracting once with phenol/chloroform, once with chloroform and precipitating with 0.1 volume NaOAc (pH5.2) and 2.5 volumes absolute ethanol (−20° C.). Following centrifuging at 13000 rpm for 30 minutes at 4° C., the dsRNA pellet was washed with 70% EtOH (−20° C.), and resuspended in 200 μl RNase-free, DNase-free Milli-Q H₂O. The concentration of this standard dsRNA was assessed using A₂₆₀/A₂₈₀ readings.

vi) Each Drosophila gene specific dsRNA was diluted and arrayed into 3 wells of a 96-well Packard Viewplate, using 1 μg dsRNA per well, ready for transfection.

3) RNA Interference in Drosophila D.Mel-2 Cells

i) D.Mel-2 cells were cultured as follows ready for transfection with dsRNA.

A cryovial containing 1 ml cryopreserved D.Mel-2 cells (Passage #8) in 10% DMSO, Drosophila-SFM (GIbco #15240), 1% 200 mM L-glutamine (GIbco #25030) and 1% Penicllin/Streptomycin Solution (GIbco #15140) was rapidly thawed and the entire contents transferred into a 25 cm² tissue culture flask containing 5 ml Drosophila-SFM/glutamine/Pen-Strep. The cells were grown at 28° C. for 4-5 days until they approached confluency, when they were transferred to a 75 cm² tissue culture flask containing 10 ml Drosophila-SFM/glutamine/Pen-Strep. The cells were again grown at 28° C. for 3-4 days, until they approached confluency. Cells were split 1:10 and into fresh medium every 3-4 days with the date and passage number recorded on the flask. After thawing, the D.Mel-2 cells were ready for transfection following 3 passages.

ii) For each new batch of D.Mel-2 cells, the efficiency of dsRNA transfection of the cells was assessed, using fluorescently labelled dsRNA as follows.

Using 8 μl RFP, Drosophila polo or Drosophila orbit specific PCR products, including a 5′ T7 RNA polymerase binding site on each strand, dsRNA was synthesised using amino-allyl substituted UTP, setting up 3 reactions with each template. Each reaction comprised of 6.12 μl 3.27×T7 RNA polymerase buffer (130.7 mM HEPES (pH 8.1), 16.3 mM DTT, 13.1% PEG 8000, 0.03% Triton X-100, 65.3 mM Mg(OAc)), 1 μl 100 mM ATP, 1 μl 100mM CTP, 1 μl 100 mM GTP, 1 μl 100 mM amino-allyl-UTP (Sigma A5660), 0.5 μl 50 U/μl yeast inorganic pyrophosphatase, 1 μl 20 U/μl Rnasin and 0.5 μl 80 U/μl T7 RNA polymerase.

Unlabelled RFP, Drosophila polo or Drosophila orbit dsRNA was also synthesised in the same way, only using 1 μl 100 mM UTP in each reaction instead of the amino-allyl substituted UTP.

The reactions were incubated at 37° C. for 4 hours in the PTC-225 thermocycler, and then treated with DNase 1 (2 U/μl) at 37° C. for 15 mins and annealed by heating to 95° C. for 10 minutes and then cooling to 4° C. over a 15 hour period. The 3 equivalent reactions containing labelled or unlabelled RFP, Drosophila polo or Drosophila orbit dsRNA were pooled and 140 μl RNase- and DNase-free water added to each.

18 BioRad P30 MicroBiospin columns were buffer exchanged to 100 mM NaHCO₃ (pH7.5) by washing the columns through 3 times with 0.5 ml buffer per wash. Each of the pooled dsRNA preparations were divided into 3 and passed through a column (65 μl per column) following manufacturer's recommendations, then re-pooled. To each of the purified dsRNA preparations, 33 μl AlexaFluor 594-succinimidyl ester (Molecular Probes A-20004) resuspended at 10 mg/ml in DMSO, and 8 μl 500 mM NaHCO₃ (pH7.5) was added. The dsRNA and label were incubated at room temperature in the dark overnight.

Once again, each of the dsRNA preparations was purifed by splitting them in three and passing each through a BioRad P30 MicroBiospin column (10 mM Tris pH7.4). The 3 purified dsRNA preparations were re-pooled and precipitated by the addition of 0.1 volumes NaOAc (pH5.2) and 2.5 volumes absolute ethanol (−20° C.). The dsRNA was centrifuged at 13000 rpm for 30 minutes at 4° C. and the supernatant removed. The pellet was washed twice with 70% EtOH (−20° C.), then allowed to air dry, and resuspended in 200 μl 10 mM Tris (pH8.0).

The concentration and quality of dsRNA in the samples was assessed from A₂₆₀/A₂₈₀ readings and by agarose gel electrophoresis. The concentrations of the dsRNA preparations were adjusted to 67 ng/μl and 15 μl of each aliquoted into 16 wells of a Packard viewplate:
Labeled polo dsRNA in columns 1 and 2
Labeled orbit dsRNA in columns 3 and 4
Labeled RFP dsRNA in columns 5 and 6
Unlabeled polo dsRNA in columns 7 and 8
Unlabeled orbit dsRNA in columns 9 and 10
Unlabeled RFP dsRNA in columns 11 and 12

To each well 35 μl of logarithmically growing D.Mel-2 cells diluted to 2.3×10⁵ cells/ml in fresh Drosophila-SFM/glutamine/Pen-Strep pre-warmed to 28° C. was added. The cells were incubated with the dsRNA (60 nM) in a humid chamber at 28° C. for 1 hr, then 100 II Drosophila-SFM/glutamine/Pen-Strep pre-warmed to 28° C. was added and the cells containing the dsRNA returned to the humid chamber at 28° C. for 72 hrs. The medium was removed and the cells incubated with 100 μl Fixation Solution (3.7% formaldehyde, 1.33 mM CaCl₂, 2.69 mM KCl, 1.47 mM KH₂PO₄, 0.52 mM MgCl₂-6H₂O, 137 mM NaCl, 8.50 mM Na₂HPO₄.7H₂O) pre-warmed to 28° C. 15 minutes. The Fixation Solution was removed and the cells washed with 100 μl Wash Buffer (1.33 mM CaCl₂, 2.69 mM KCl, 1.47 mM KH₂PO₄, 0.52 mM MgCl₂-6H₂O, 137 mM NaCl, 8.50 mM Na₂HPO₄.7H₂O). The cells were treated with 100 μl Permeabilisation Buffer (30.8 mM NaCl, 0.31 mM KH₂PO₄, 0.57 mM Na₂HPO₄.7H₂O, 0.02% Triton X-100) for 15 minutes and once more with 100 μl Wash Buffer, prior to the addition of 50 μl Staining Solution (1 μg/ml Hoechst 33258, 1.33 mM CaCl₂, 2.69 mM KCl, 1.47 mM KH₂PO₄, 0.52 mM MgCl₂-6H₂O, 137 mM NaCl, 8.50 mM Na₂HPO₄.7H₂O) per well. The cells were incubated with the Staining solution for 1 hour protected from the light. The Staining Solution was removed and the cells washed twice with 100 μl Wash Buffer. 200 μL Wash Buffer containing 0.02% sodium azide was finally added to the cells, the plates were sealed and the transfection efficiency analysed using the Cellomics ArrayScan HCS System (Cellomics Europe, St. Mary's Court, The Broadway, Old Amersham, Bucks, HP7 OUT, UK), with the 10× objective and the QuadBGRFR filter set following manufacturer's recommendations.

iii) Transfection of D.Mel-2 cells with gene specific dsRNA was carried out as for fluorescently labelled control dsRNA detailed above.

Following incubation of D.Mel-2 cells with the dsRNA for 72 hrs at 28° C., the cells were fixed and stained using the Cellomics Mitotic Index Hitkit, essentially following the manufacturer's recommendations, with the exception that the Fixation solution was pre-warmed to 28° C. and only half the specified volumes of primary and secondary antibody were added to the Primary Antibody Solution and to the Staining Solution, respectively. The kit provides a fixed endpoint assay based on immunofluorescence detection and localisation of a phosphorylated core histone protein that is abundant in the nuclei of dividing cells, to enable a determination of the Mitotic Index. The Mitotic Index (MI) represents the fraction of cells within a population under-going cell division and is a valuable means of characterising cell proliferation. The calculated value for mitotic index is often higher in cancerous cells due to uncontrolled cell proliferation.

The Mitotic Indices were analysed following manufacturer's recommendations, using the Cellomics Array Scan HCS System, with the 10× objective and the DualBGlp filter set.

2,500 cells were assessed for each well as confirmed, by viewing cell counts for the wells of each plate within Data Viewer.

The average MI value, the standard deviation and Ttest scores (assuming a two-tailed distribution with two samples of equal variance) for each gene, compared to the RFP control on the plate containing the gene was calculated from Mitotic Index data generated by the Arrayscan. The Ttest scores for each gene, compared to the water control on the plate containing the gene was also calculated, again assuming a two-tailed distribution with two samples of equal variance and the two Ttest scores for each gene combined to give an aggregate Ttest score. The Mitotic Index for D.Mel-2 cells transfected with each gene specific dsRNA was also expressed as a percentage of the mitotic index for cells transfected with the RFP control dsRNA for that plate. The percentage change in MI for each gene, relative to the RFP control was also calculated where:

$$\begin{matrix}change in \% MI \\ of gene X\end{matrix} = ( ( \frac{\begin{matrix}MI for cells transfected with \\ gene X specific dsRNA\end{matrix}}{\begin{matrix}MI for cells transfected with \\ RFP specific dsRNA\end{matrix}} ) \times 100 ) - 100$$

and from this the absolute change in MI was identified.

Genes with an aggregate Ttest score less than 0.1 were viewed as significant and were ranked according to their absolute change in MI relative to the RFP control for further analysis (See Table 2 below).

TABLE 2

Mitotic Index Data

| | | | |
|---|---|---|---|
| | | | MI as % |
| Drosophila | | Aggregate | Change |
| Gene | Froward and reverse primer sequences | T Test | Relative |
| Name | (including T7 RNA polyerase binding site) | Score | to RFP |

| | | | |
|---|---|---|---|
| CG3632 | TAATACGACTCACTATAGGGAGAAGGTGCGAGATCTGTTCCAGCTGATT | 0.0740 | 126.6 |
| | TAATACGACTCACTATAGGGAGAAGGGCACAAGCAATTTGGGTGGATAC | | |
| Pp1-87B | TAATACGACTCACTATAGGGAGATCCGCCGGAATCGAATTACCTGTTC | 0.0048 | 107.8 |
| | TAATACGACTCACTATAGGGAGAACTTGATGGGCTCGGCAGATGAGAT | | |
| CG3524 | TAATACGACTCACTATAGGGAGAGGCAAAGAAGCACCAACAGAAGTTA | 0.0914 | 82.5 |
| | TAATACGACTCACTATAGGGAGATCTGGGCTAGCATATTCACAGAGTA | | |
| CG9311 | TAATACGACTCACTATAGGGAGACTACCAAACAGCGGCAGGTTATTCAG | 0.0980 | 74.6 |
| | TAATACGACTCACTATAGGGAGAGTTGCTGCTGATATGCCAGTGGTTGT | | |
| CG9092 | TAATACGACTCACTATAGGGAGATTAGCAAGGGTTGGGGCAGCACACT | 0.0212 | 61.4 |
| | TAATACGACTCACTATAGGGAGACACTGCAGGTAGATCCTCGCCAGTT | | |
| Arr1 | TAATACGACTCACTATAGGGAGACAATTTCCGATATGGGCGCGAGGAC | 0.0402 | −54.4 |
| | TAATACGACTCACTATAGGGAGACTTCACCACCTTGTTGGAGTTGTTG | | |
| CG9150 | TAATACGACTCACTATAGGGAGAGGTGGCAGAACAAACTGGCTGTGGT | 0.0843 | −53.0 |
| | TAATACGACTCACTATAGGGAGAATGGCGGTGATGGCAAACTTGGTGG | | |
| CG11102 | TAATACGACTCACTATAGGGAGAATGCTTTGGCTGCTGTTGCGGTACAAGT | 0.0049 | 51.0 |
| | TAATACGACTCACTATAGGGAGAGCGGAGAGTAACTGAAGCACTGGAG | | |
| Smr | TAATACGACTCACTATAGGGAGACCGCCGGAGACCATAATCTACAATG | 0.0606 | 50.3 |
| | TAATACGACTCACTATAGGGAGACGGGCACTGGTACTGGTTGTAGATA | | |
| CG8045 | TAATACGACTCACTATAGGGAGACTGCTGTCGGTGGCGTACAAGAATG | 0.0065 | −49.5 |
| | TAATACGACTCACTATAGGGAGACTCAGTGTATCCAACTCGGCAATGG | | |
| CG10420 | TAATACGACTCACTATAGGGAGAGGAGTCTATACGGCGGGTCAAGGAG | 0.0509 | 49.4 |
| | TAATACGACTCACTATAGGGAGAGAGCCTGTGTACCCGAGGTGGAAAG | | |
| Hsc70-2 | TAATACGACTCACTATAGGGAGAGAAGTTCGACGACAAGAAGATACAG | 0.0116 | −47.4 |
| | TAATACGACTCACTATAGGGAGACGCTTGAATTCCTCGGCAAAATGAT | | |
| CG10805 | TAATACGACTCACTATAGGGAGAATTAATCAGTAATCGCAAGCTGGTG | 0.0746 | 46.1 |
| | TAATACGACTCACTATAGGGAGATAGCTTCTGTTCAAGTTGGACATAG | | |
| eIF-4a | TAATACGACTCACTATAGGGAGACTGCCACCTTCTCGATTGCTATCCT | 0.0263 | −45.7 |
| | TAATACGACTCACTATAGGGAGACTCCTGCTTCACGTTGACGTAAAAC | | |
| ACXA | TAATACGACTCACTATAGGGAGATCCAACTGGCCTCTTAATACCTTAT | 0.0166 | 44.8 |
| | TAATACGACTCACTATAGGGAGACATAAAGGATACTGACATCGTTGTG | | |
| CG15117 | TAATACGACTCACTATAGGGAGATGTACGATAAGGATGGCATATTGGT | 0.0690 | 44.1 |
| | TAATACGACTCACTATAGGGAGAGAAAATCGAAGCCAAGAGCATTAGT | | |
| BG:DS01759.2 | TAATACGACTCACTATAGGGAGAGAACTCAAAACGATGCTGGTCAAGT | 0.0521 | 43.6 |
| | TAATACGACTCACTATAGGGAGAAAATCAATAGGTCCAGTAGATGGGG | | |
| TepIII | TAATACGACTCACTATAGGGAGAGCGTTCATACGAACTGAGCGATGTG | 0.0165 | −41.4 |
| | TAATACGACTCACTATAGGGAGAGAGTAGGGCAACTCGGTGCTTATGT | | |
| Hsc70-4 | TAATACGACTCACTATAGGGAGACTATCTGGGCAAGACTGTGACCAAC | 0.0711 | −40.9 |
| | TAATACGACTCACTATAGGGAGAGGCACGAGTAATCGAGGTGTAGAAG | | |
| CG7069 | TAATACGACTCACTATAGGGAGACCAAGAAGGAGATGGCAGACAAGAG | 0.0292 | −40.3 |
| | TAATACGACTCACTATAGGGAGACAATCGATTTCTGGGCTAGGGGAAC | | |
| ACXE | TAATACGACTCACTATAGGGAGATCCACTTCATCAGCGGTGCAGTCCT | 0.0433 | 39.2 |
| | TAATACGACTCACTATAGGGAGAGAGATCGTGCAGGACCTTCACCAAC | | |
| EG:52C10.5 | TAATACGACTCACTATAGGGAGAGCAGAACTTCGATCTAAGCGACCAA | 0.0813 | −37.9 |
| | TAATACGACTCACTATAGGGAGATCAGCTAGCTTCGACTGAATGTCTC | | |
| gatA | TAATACGACTCACTATAGGGAGAAAGGTGGCCGATTTGCTGGAGTGTA | 0.0851 | 37.1 |
| | TAATACGACTCACTATAGGGAGAGAATCGGTATAGACACGGCGGGAAT | | |
| CG17149 | TAATACGACTCACTATAGGGAGAAGAGGCCTGCTTTCCGGACATCAGT | 0.0283 | 36.6 |
| | TAATACGACTCACTATAGGGAGAGTGGACATGTCTGCTGGATGGGAAC | | |
| CG2905 | TAATACGACTCACTATAGGGAGAGATAAAGTTCTTGCTACAGTGGAAA | 0.0314 | −35.8 |
| | TAATACGACTCACTATAGGGAGAAAAGGTAAAGCATTTGAATCAGGAG | | |
| CG2336 | TAATACGACTCACTATAGGGAGATCAGTCTAGAAGAGGAGGTTCAATA | 0.0419 | 35.7 |
| | TAATACGACTCACTATAGGGAGACAACTATTCTCTTTGCCTGTAAGTG | | |
| TER94 | TAATACGACTCACTATAGGGAGAGGTCTGGAGAGCGTCAAGAAGGAAT | 0.0623 | −35.6 |
| | TAATACGACTCACTATAGGGAGACGGGCAGCGGAATATAGATCAACTG | | |
| CG6313 | TAATACGACTCACTATAGGGAGACCGCAATCTAGCCAACAAGCTCTCA | 0.0752 | −35.2 |
| | TAATACGACTCACTATAGGGAGAGGCGCAGTACGTTATTAGCATCCAC | | |
| aur | TAATACGACTCACTATAGGGAGAACGTGCGCATATATCTGATCTTGGA | 0.0147 | 35.1 |
| | TAATACGACTCACTATAGGGAGAATTAAGGACCAGCAGCTTGGAAATG | | |
| Pk91C | TAATACGACTCACTATAGGGAGAGCGATAGCAAGATATCTGGTGTTTC | 0.0560 | 34.9 |
| | TATACGACTCACTATAGGGAGAGCTTAGGTTGTCCACATTCTTCTCA | | |
| Top2 | TAATACGACTCAOTATAGGGAGAAGGTGGTTTCTACCGAGTGTTCAAA | 0.0071 | 34.3 |
| | TAATACGACTCACTATAGGGAGATCTGTCAACATCCACATGGACATAC | | |
| Alpha-Est1 | TAATACGACTCACTATAGGGAGATCCGAAATGGCCGCACAGGGTATTA | 0.0185 | 33.4 |
| | TAATACGACTCACTATAGGGAGAGATTGAAATGGGGCGAGTCCACATC | | |
| Nrk | TAATACGACTCACTATAGGGAGAAGATCTACTAGTCGCTGTTAAGATG | 0.0453 | 33.1 |
| | TAATACGACTCACTATAGGGAGAAAGCGAGAACTTGTTGTACAGTATG | | |
| otk | TAATACGACTCACTATAGGGAGACAAGCCGACAATTCAGTGGGACAAG | 0.0506 | 33.1 |
| | TAATACGACTCACTATAGGGAGACTGCAGGCTGTGTCATCGGATTTCT | | |
| cad | TAATACGACTCACTATAGGGAGAGGCGGATAACTTCGTTCAGAATGTG | 0.0587 | 32.4 |
| | TAATACGACTCACTATAGGGAGAGATAGGCGGGCTTCTTCATCCAGTC | | |
| rut | TAATACGACTCACTATAGGGAGAGCGCCAAAGTACGAGCCACCACGTTACA | 0.0343 | 30.7 |
| | TAATACGACTCACTATAGGGAGACATGTCGACGACCGGAGAGGTGGGA | | |
| CG8002 | TAATACGACTCACTATAGGGAGAAGCTGCACATTGACGATACGGAGAG | 0.0560 | 30.4 |
| | TAATACGACTCACTATAGGGAGAATGCCGACAAGCTCTTGGTCACAGT | | |
| CG10335 | TAATACGACTCACTATAGGGAGACACAATCTCATGTATCCGGTGTTCA | 0.0678 | 30.3 |
| | TAATACGACTCACTATAGGGAGAAATTGGATGTGAACTTGGCGGAGTA | | |
| CG8070 | TAATACGACTCACTATAGGGAGAGGGTGCTTCTCTAAGGTAACAAAAG | 0.0493 | 30.2 |
| | TAATACGACTCACTATAGGGAGATTAGGATAGGCTCGATGATGTGTCC | | |
| CG7460 | TAATACGACTCACTATAGGGAGACCTTTAGATGCGTTCGATCCAACAA | 0.0568 | 29.3 |
| | TAATACGACTCACTATAGGGAGAATGACATGATCCGCTGTGATTACCT | | |
| CG17735 | TAATACGACTCACTATAGGGAGACCTTCAGAGCTGTACGAACTTACCT | 0.0658 | −29.1 |
| | TAATACGACTCACTATAGGGAGAAGATGGACGGCGCTTTACTTGATTG | | |
| Gycalpha99B | TAATACGACTCACTATAGGGAGAGCTCTACAAGGTGGACGTGAACATC | 0.0720 | 28.4 |
| | TAATACGACTCACTATAGGGAGAAGAGCAACGAATTGGACTCGGGACA | | |
| CG13893 | TAATACGACTCACTATAGGGAGAACGATCAGAGTCAAAAGCACGGATGG | 0.0943 | 28.3 |
| | TAATACGACTCACTATAGGGAGATGACCTTAAAGTGCAGCTTCAACTT | | |
| CG18176 | TAATACGACTCACTATAGGGAGAAAAAGGTTGTTCAGTGCTTTGAGAA | 0.0751 | −28.3 |
| | TAATACGACTCACTATAGGGAGAACATGACCAAACTTTTGCAGATAGT | | |
| CG8858 | TAATACGACTCACTATAGGGAGAACATGAGCTGGAACCTGCTGGAAAA | 0.0617 | 27.8 |
| | TAATACGACTCACTATAGGGAGACTCAGOCGCTTGATCAGCATAAGAA | | |
| Ac76E | TAATACGACTCACTATAGGGAGAGTGCCACCGAAACCAGCATACACCT | 0.0907 | 27.0 |
| | TAATACGACTCACTATAGGGAGATCCGCTGTTGGAGATGCCGCTCTCT | | |
| CG17010 | TAATACGACTCACTATAGGGAGAACGTGGCTGGTGCGAATGTATTTCT | 0.0034 | −26.0 |
| | TAATACGACTCACTATAGGGAGATGCCGCACTGATATGATGTTCTGTG | | |
| tkv | TAATACGACTCACTATAGGGAGAGAACCATTGCCAAGCAGATTCAGAT | 0.0863 | 25.8 |
| | TAATACGACTCACTATAGGGAGATGAATGACATCCAGTTCCGAGTTGT | | |
| dnt | TAATACGACTCACTATAGGGAGAGACCGGCGATCAATGTGTCACACAG | 0.0886 | 25.6 |
| | TAATACGACTCACTATAGGGAGAACTGGAACTTTCCGTGGCAAGGAGG | | |
| ACXD | TAATACGACTCACTATAGGGAGAATGTTTACCACGCCACCAGCCACAA | 0.0673 | 25.1 |
| | TAATACGACTCACTATAGGGAGAATGGTCCGGTTGTCGCCACCTTTTA | | |
| Aats-ala-m | TAATACGACTCACTATAGGGAGACTATTGTGGTGGAGACACTTGGTGA | 0.0676 | 25.0 |
| | TAATACGACTCACTATAGGGAGAGGAGTAGGTGTACTTGTGACTGTTG | | |
| gek | TAATACGACTCACTATAGGGAGAGCAACAAACACAGGAAAGGCTGAAG | 0.0959 | −24.9 |
| | TAATACGACTCACTATAGGGAGAGGATATGAGGTCCGATCTGGTTTGA | | |
| CG3216 | TAATACGACTCACTATAGGGAGATCTACCAAATCCTGCCGCGTCCTGT | 0.0999 | 24.7 |
| | TAATACGACTCACTATAGGGAGAGGTGGCCGAGGACACATGTATCTTG | | |
| CG5653 | TAATACGACTCACTATAGGGAGAGTACCACGAATGTGATGGCGACAAG | 0.0703 | 24.1 |
| | TAATACGACTCACTATAGGGAGACCATCAACATTCGGGGCTGACAAGT | | |
| GG17740 | TAATACGACTCACTATAGGGAGACGAGGCAGAGAACTTTGGTGACTAC | 0.0969 | −23.6 |
| | TAATACGACTCACTATAGGGAGAAGCATGCACTCATCCGCACAGAAGT | | |
| Tepl | TAATACGACTCACTATAGGGAGAAATGGATGTGAAGGCGAAAGTATTA | 0.0572 | 23.4 |
| | TAATACGACTCACTATAGGGAGAGTATACTGGGCACAAAGTTAAACAT | | |
| for (CT43154 | TAATACGACTCACTATAGGGAGACGTTCAGCAGAAGTGTGGTCAGGTC | 0.0213 | 22.5 |
| & CT43158) | TAATACGACTCACTATAGGGAGACCGTCCGCTGGCAGTTGTACAGGAT | | |
| Ac13E | TAATACGACTCACTATAGGGAGAACTCAACCAGACGGCGATTAGTCAG | 0.0992 | 21.3 |
| | TAATACGACTCACTATAGGGAGAAAGCAGGAGAACAAGGTCACCCACA | | |
| CG2667 | TAATACGACTCACTATAGGGAGATGCCGGAACTGCAGGGAATTGTGAT | 0.0180 | 21.2 |
| | TAATACGACTCACTATAGGGAGAGAGCTGACGGCTTCGATGAAGTTGA | | |
| CG7842 | TAATACGACTCACTATAGGGAGAGGAGGGACCACGTGAGAAACTGAAT | 0.0397 | 21.2 |
| | TAATACGACTCACTATAGGGAGAACGGCGCTTTGCATTAGAGGAGTGT | | |
| CG17486 | TAATACGACTCACTATAGGGAGAACGCACTTGGAAGAAATTCACTATT | 0.0501 | 20.3 |
| | TAATACGACTCACTATAGGGAGATAGAATACACAATTTTGCGTGTCTG | | |
| CG6969 | TAATACGACTCACTATAGGGAGAACAGAAGATCCACCGGGGTGTGTAA | 0.0226 | 20.2 |
| | TAATACGACTCACTATAGGGAGACTGATTTTGCCGCTGCTCCAGATTG | | |
| CG12262 | TAATACGACTCACTATAGGGAGAGGTTCATTGTGGAGCGCGACAGTCC | 0.0966 | 19.6 |
| | TAATACGACTCACTATAGGGAGACTCCACGGGATACTCGCTGTTGAAG | | |
| fray | TAATACGACTCACTATAGGGAGAATTAAGCGCATCAACCTGGAGAAGT | 0.0749 | 19.5 |
| | TAATACGACTCACTATAGGGAGACCAAATGTCCGCCTTAAAGTCATAG | | |
| CG6879 | TAATACGACTCACTATAGGGAGACCGGATACAACGTGACCTCACTGGA | 0.0645 | 19.5 |
| | TAATACGACTCACTATAGGGAGACACCTCTTCCGCCAATCTGGTAGGT | | |
| CG11594 | TAATACGACTCACTATAGGGAGAGAGGAATCTGTCACAGACTTTTGGA | 0.0076 | 19.0 |
| | TAATACGACTCACTATAGGGAGACGTTTAGGAAGTAGCCGGGAATAAT | | |
| S6kll | TAATACGACTCACTATAGGGAGAATTTTGCCGCTGATTGGTGGAGTTT | 0.0168 | 18.9 |
| | TAATACGACTCACTATAGGGAGACAGCAGGAATAGGAGCTATACTATG | | |
| CG11714 | TAATACGACTCACTATAGGGAGAGAGTTACTTCGTCTATTCCAGTGTG | 0.0346 | 18.4 |
| | TAATACGACTCACTATAGGGAGATACTTCTCAGCCAGGCTAAGGAAAT | | |
| CG3534 | TAATACGACTCACTATAGGGAGACCACCACCAAACAGTGCCTAGAGAT | 0.0877 | 17.9 |
| | TAATACGACTCACTATAGGGAGACCTTCAAGCTCATCATTAGGGTGTC | | |
| CG7335 | TAATACGACTCACTATAGGGAGAAGGAGCTCACCTACCAGCAGTTTGT | 0.0094 | 17.8 |
| | TAATACGACTCACTATAGGGAGACCACTAATTTGGGCGGAATCTGGGA | | |
| CG11275 | TAATACGACTCACTATAGGGAGAAGCTGTACTACGCCGCTGAGAAGTA | 0.0040 | 17.7 |
| | TAATACGACTCACTATAGGGAGAACAGCGGAGTTTCTGCATCCACTGT | | |
| CG16726 | TAATACGACTCACTATAGGGAGAGCGAATCCAATGTCACGGAATACAA | 0.0359 | 17.3 |
| | TAATACGACTCACTATAGGGAGATGGCAATGACTATATAGGGCAGACA | | |
| CG7514 | TAATACGACTCACTATAGGGAGACCACTGTGTTGGCCAGCATGGGTAT | 0.0939 | 17.0 |
| | TAATACGACTCACTATAGGGAGAGTGTGCGGTCCTAAGCGACACAAAT | | |
| CG17283 | TAATACGACTCACTATAGGGAGAAAATTAGGGCCAAGACCGAGTCAAT | 0.0921 | 16.8 |
| | TAATACGACTCACTATAGGGAGACGAAATTTGAGGTCACGAAAGTGGT | | |
| BcDNA:GH07 | TAATACGACTCACTATAGGGAGAGGCCACCGAGCAGAACTTTAACTGG | 0.0885 | 16.7 |
| 626 | TAATACGACTCACTATAGGGAGACGGCCAGCTTACCAGAGGTCAACAT | | |
| CG16752 | TAATACGACTCACTATAGGGAGAGTATATTGCATTGCTGGCGTTTCTG | 0.0333 | 16.3 |
| | TAATACGACTCACTATAGGGAGATGCCGCAGTAAATGCCGAAGTTGAT | | |
| Rpt1 | TAATACGACTCACTATAGGGAGATGAGCTGGTGCAAAAGTACGTGGGT | 0.0418 | −15.8 |
| | TAATACGACTCACTATAGGGAGAGGCTTCGAGGAAATCCTTCTCTGTG | | |
| wts | TAATACGACTCACTATAGGGAGAAACAGCAACTGCAGGCCTTGAGGGT | 0.0630 | 15.7 |
| | TAATACGACTCACTATAGGGAGAATACGTGCGCTGGCGATACGACTTG | | |
| CG1582 | TAATACGACTCACTATAGGGAGATCAACTGCCAGCAAAGGAGAACTTG | 0.0531 | −15.7 |
| | TAATACGACTCACTATAGGGAGATAAGCTTGCGGCAGTACTTAGTGTC | | |
| CG12289 | TAATACGACTCACTATAGGGAGACACCAAGTATATAGCCGAGTCCAGA | 0.0349 | −14.6 |
| | TAATACGACTCACTATAGGGAGATCAGTGGGTGCAATGGAGGACTTTT | | |
| Pepck | TAATACGACTCACTATAGGGAGAGTTGCACAAGCTGCGCCAGGACAAT | 0.0124 | −14.2 |
| | TAATACGACTCACTATAGGGAGACACCACGTAAGCAGAGTCCGTCAGT | | |
| CG5665 | TAATACGACTCACTATAGGGAGAATTCACTAGGAGCTCACATTATGGG | 0.0576 | 14.0 |
| | TAATACGACTCACTATAGGGAGACACGTTTTAAGCCTAGGATCAACAG | | |
| CG7285 | TAATACGACTCACTATAGGGAGAACACGAACGAGAGCTTATATACCAC | 0.0930 | 13.6 |
| | TAATACGACTCACTATAGGGAGAAGACCACTTTGGCAATATGCAGAGT | | |
| bt | TAATACGACTCACTATAGGGAGACGGACCACTTCAAATATCAGATGTG | 0.0177 | 13.4 |
| | TAATACGACTCACTATAGGGAGAGGAAACTCGGAATTTGTAGGTTTGG | | |
| CG8795 | TAATACGACTCACTATAGGGAGAATGGCAGTGGCAATGGAACGACAAC | 0.0865 | 13.3 |
| | TAATACGACTCACTATAGGGAGAGGTTTGTGGCTGCCTTTGCGTCTGG | | |
| CG10967 | TAATACGACTCACTATAGGGAGAAGCGCAAGAGCAGTGTGAGCAGTGA | 0.0498 | 12.4 |
| | TAATACGACTCACTATAGGGAGACGCCAGCACAAAGTTCAGCTTGGAC | | |
| CG3809 | TAATACGACTCACTATAGGGAGACTTCTTTCTGGCCGTTTGTCCACCT | 0.0959 | 11.8 |
| | TAATACGACTCACTATAGGGAGAGCTTATCGATCTGAACACCGACCAC | | |
| Ack | TAATACGACTCACTATAGGGAGATCTACTCGAATTTCAACCAGTCTCT | 0.0319 | 10.0 |
| | TAATACGACTCACTATAGGGAGATCATACCAAATACGATTCACCACAC | | |
| Abl | TAATACGACTCACTATAGGGAGACACGGGCGATAGTCTGGAGCAGAGT | 0.0449 | 9.2 |
| | TAATACGACTCACTATAGGGAGACGGAATGGGGCTGGCCTTCGGATTT | | |
| CG7362 | TAATACGACTCACTATAGGGAGACCGCGAATCCGATGGCATAATGGTG | 0.0886 | −9.0 |
| | TAATACGACTCACTATAGGGAGATAGGACACGGCGGCCTCATTTGACT | | |
| Cyp9f2 | TAATACGACTCACTATAGGGAGAATGAAATACCGACAGGAGCACAATA | 0.0438 | 6.4 |
| | TAATACGACTCACTATAGGGAGACGAACTTCATAGGGTTCTCAAAGTA | | |

4) Analysis Of The Concentration-Dependent Effect of Transfected dsRNA upon Drosophila D.MEL-2 Cells Mitotic Index.

To confirm the initial effect of gene specific dsRNA on the mitotic index of transfected D.Mel-2 cells and also to demonstrate a dosage dependant effect on MI the concentration dependent effect of dsRNA is tested.

Drosophila gene specific dsRNA ranging in concentration from 10 ng per well to 4 μg per well is transfected into D.Mel-2 cells and the Mitotic Index of the transfected cells assayed using the Cellomics Mitotic Index Hit Kit as described previously. Each Cellomics assay plate includes the range of gene specific dsRNA samples and also an equivalent range of RFP control dsRNA samples for comparison.

The resulting data is analysed essentially as before, except that for each gene a bar graph showing the Mitotic Index value for D.Mel-2 cells transfected with the gene specific dsRNA at each concentration and for cells transfected with the control dsRNA at each concentration is constructed. Error bars corresponding to +/−1 standard deviation are also included.

5) Human Homologues

An automated web query system was used to identify matching BLAST hits from SWISS-PROT for Drosophila genes where RNAi resulted in a significant change in mitotic index in D.Mel-2 cells.

The Drosophila gene name was used to find matching entries in SWISS-PROT, using the get-entries service (“/cgi-bin/get-entries”) at the EXpert Protein Analysis SYstem world wide web site (“expasy.org”). The search used an exact string match in the gene name field and “Drosophila” in the organism field. One or more accession numbers from the SWISS-PROT and trEMBL datasets were returned by this search.

Each accession number was used to identify the corresponding full SWISS-PROT entry, which was returned by requesting the accession number from “/cgi-bin/hub” at expasy.org. Each entry was used as a blastp query against the SWISS-PROT database via the BLAST service at expasy.org (“/cgi-bin/blast.pl”). All blast hits were stored, but only those matching rat, mouse or human were included in the output file. The SWISS-PROT files for hits from the above organisms were requested from expasy.org (“/cgi-bin/hub”).

Human homologues with best homology (>25% similarity and BLAST score >10) over the majority of the Drosophila protein were earmarked for further validation. In some cases, several human proteins were identified as homologues of the Drosophila gene.

The sequence references for the human homologues are set out in Table 1.

6) Validating Human Mitotic Gene Function by RNAi

The identified human gene homologues are initially validated by RNA interference of gene expression in U2OS cells, using FACS analysis to identify human genes required for normal cell cycle progression.

i) Designing and Ordering Human Gene Specific siRNA

siRNA sequences for gene specific RNAi are identified using the siRNA FINDER at the Ambion.com site, in the “/techlib/misc/” directory, in the file “siRNA_finder.html”. siRNAs are identified at the middle and 3′ end of the open reading frame and with a GC content of between 42.9% and 52.4%. BLAST searches are performed with prospective siRNAs to verify that they are unique to the selected human target gene. Where an siRNA shares more than 81% identity with another human gene, the next adjacent siRNA is selected. For each selected sequence, 0.2 Rmole of purified, annealed, duplex siRNA is ordered from Dharmacon (Dharmacon Research, Inc., 1376 Miners Drive, #101, Lafayette, Colo. 80026, USA). siRNAs are resuspended in RNase- and DNase-free water and used to prepare 20 mM stock solutions.

ii) Culturing Human Cell Lines for RNAi analysis

Human Hela and U20S cell lines are cultured for transfection with siRNA in the following media: 500 ml DMEM with L-glutamine, sodium pyruvate and pyridoxine (GIbco: #41966-029 (InVitrogen Corporation, Carlsbad, Calif., USA)), 50 ml FBS (E.U. approved, Gibco; #10106-169), 5 ml Pen/Strep 5000 IU (GIbco: #15070-063).

hTERT-BJ1 cell line is cultured in: 400 ml DMEM with L-glutamine, sodium pyruvate and pyridoxine (GIbco: #41966-029), 100 ml M199 with Earle's salts, L-glutamine, NaHCO₃ (Sigma; #M4530), 50 ml FBS (E.U. approved, Gibco; #10106-169).

To passsage the cells, the culture medium from a 75 cm² flask of cells is removed and the cells washed with pre-warmed (37° C.) PBS (Dulbecco, W/O Calcium and Magnesium, W/O Sodium Bicarbonate; Gibco #14190-094). 0.5 ml pre-warmed (37° C.) Trypsin/EDTA (GIbco #25300-054) is applied and spread evenly on the surface of the cells and incubated for 5 minutes at 37° C. The trypsin is neutralised with 4.5 ml of the pre-warmed (37° C.) appropriate culture medium and the cells washed from the surface of the flask by pipetting.

Cell densities are measured using a haemocytometer.

iii) FACS analysis of U2OS cells transfected with Gene Specific siRNA is Carried out as Follows.

U2O S cells are transfected with 240 nM of each gene specific siRNA, using 2 ml 1×10⁵ cells/ml cell suspension per well of the 6-well plates for each transfections and leaving the cells to grow overnight at 37° C./5% CO₂. siRNAs are thawed, the Oligofectamine Reagent is pre-warmed to room temperature and all culture media is pre-warmed to 37° C. In RNase-free, sterile Eppendorf tubes 12 μl of each 20 mM siRNA solution is added to 200 μl of OptiMEM Serum-free medium (GIbco #31985-039). For each transfection reaction, 8 μl of Oligofectamine™ Reagent (Invitrogen #12252-011) is mixed with 52 μl of OptiMEM and incubated for 10 minutes at room temperature. This is prepared as a batch for all the transfections.

60 μl Oligofectamine/OptiMEM is mixed with each siRNA and incubate for 15-20 minutes at room temperature. 128 μl of OptiMEM is then added to each siRNA/Oligofectamine mix. After removing the culture medium from the cells, washing briefly with PBS and re-feeding with 600 μl of the appropriate cell culture medium without antibiotics and without FBS, 400 μl siRNA/Oligofectamine mix is added to the cells in the 6-well dish and left to incubate for 4 hours at 37° C./5% CO₂. The culture is subsequently supplemented with 1 ml of the appropriate cell culture medium containing 20% FBS and antibiotics and the cells incubated at 37° C./5% CO₂ for 48 hours.

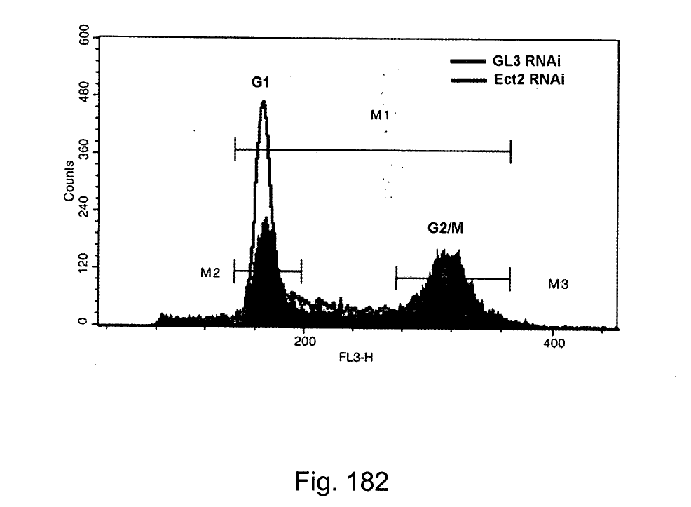

For each experiment cells transfected with siRNA (aaCUUACGCUGAGUACUUCGA) targeting the Photinus pyralis (GL3) luciferase gene (Accession no. U47296) (Harborth et al., 2001, J Cell Sci. 114:4557-4565) and cells transfected with no siRNA are included as negative controls. Cells transfected with Ect2 siRNA COD1513 (aaGUGGGCUUUGUAAAGAUGG) shown previously by us to result in a block in mitosis are included as the positive control (See FACS profiles in FIG. 182).

Following incubation, the supernatant from each well is transferred to a 15 ml centrifuige tube. The well is rinsed with 0.5 ml pre-warmed (37° C.) Trypsin/EDTA and this is combined with the 2 ml of supernatant from the same well. A further 0.5 ml pre-warmed (37° C.) Trypsin/EDTA is added to the remaining cells, spread evenly over the surface of the cells and incubated for 5 minutes at 37° C. The trypsinised cells are added to the 2.5 ml supernatant from the same well and the cells pelleted by centrifuging at 2000 rpm for 5 minutes. The supernatant is removed and the cells resuspended in 1 ml PBS, transferring the resuspended cells to an Eppendorf tube. The cells are pelleted at 2000 rpm for 1 minute in a micofuge, most of the PBS removed and the cells resuspended in the remaining PBS by flicking the tube. 1 ml 70% ethanol is added drop-wise to the cells while vortexing the tube and the fixed cells stored at −20° C. overnight.

The cells are pelleted by centrifuging at 1000 rpm for 1 minute most of the ethanol removed and the cells resuspended in the remaining ethanol by flicking the tube as before. 0.5 ml PBS is added drop-wise to the cells while vortexing the tube. The cells are incubated with 3 μl 6 mg/ml RNase A and the DNA stained with 12.5 μl 5 mg/ml propidium iodide at 37° C. for 30 minutes, then stored on ice until ready for analysis.

The DNA content of the cells is analysed with BD FACSCalibur™ System (BD Biosciences, European Office, Denderstraat 24, 9320 Erembodegem, Belgium) using the FL3 channel and CellQuest software.

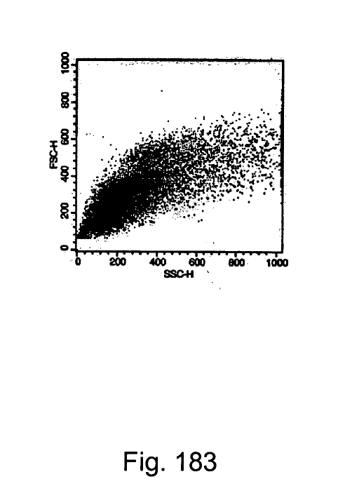

Voltages on FSC and SSC channels are altered until the profile appears as shown in FIG. 183.

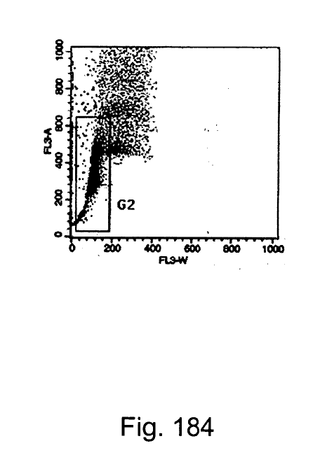

The voltage on FL3 channel is also altered to enable analysis of cells in GI, S and G2/M, setting a gate (G2) to exclude doublets resulting from cell clumping, as shown in FIG. 184.

10,000 gated events are collected for analysis and dot plots of FSC-H against SSC-H and of FL3-A against FL3-W are generated, as demonstrated above.

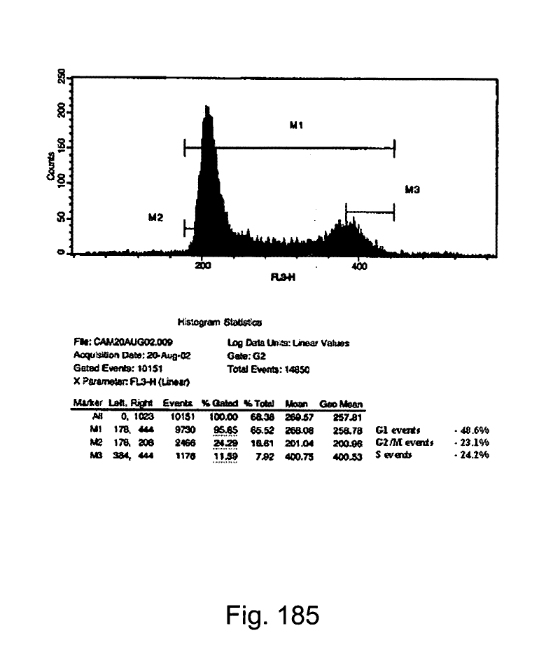

A histogram showing counts against FL3-H within the gated region (G2) and the associated histogram statistics is also generated. Using the histogram statistics, events in G1, in S and in G2/M are calculated, where the number of G1 events was equal to M2×2, the number of G2/M events is equal to M3×2 and the number of S events equated to M1-[(M2×2)+(M3×2)]. See FIG. 185 for example.

The cell cycle profile for cells transfected with the gene specific siRNA are compared with control cells transfected with GL3 siRNA, by comparison of the statistics and also by overlaying the histogram profiles, to identify human genes required for normal cell cycle progression.

Changes in cell cycle profile are deemed significant where the FACS profile is noticeably different when compared, by eye, to the negative control profile.

7) Abnormal Human Cell Phenotypes Resulting from siRNA Transfection are Assessed by Immunostaining siRNA-Treated Cells and Microscopic Analysis.

U2OS and HeLa human cell lines cultured as before are transfected with 240 nM gene specific siRNA, selecting siRNAs previously shown to induce in an abnormal FACS profile in transfected cells. The cells are transfected essentially as detailed above, except that a clean, sterile 22×22 mm coverslip is first placed in each well of the 6-well plates used for transfections.

i) Immunostaining of siRNA-treated Human Cells for Microscopic Analysis is Carried out as Follows:

The medium is gently removed and the cells incubated with 1 ml Fixation Solution (60 mM PIPES, 25 mM Hepes, 10 mM EGTA, 4 mM MgSO₄, pH to 6.8 with KOH, 3.7% formaldehyde) pre-warmed to 37° C. for 10 minutes at room temperature. The fixation solution is removed and the cells permeablised with 1 ml PBT (PBS+0.1% Triton X-100) for 2 minutes at room temperature. The permeablisation solution is removed and the cells incubated with 1 ml blocking solution (1% BSA/PBS/0.1% Triton X-100), for 1 hour at room temperature. The blocking solution is replaced with 0.5 ml primary antibody solution (1% BSA/PBS/0.1% Triton X-100/1:300 dilution rat YL½ anti-α-tubulin antibody (SeroTec #MCA77G)/1:750 dilution rabbit anti-γ-tubulin antibody (Sigma #DQ-19) and the cells incubated with the primary antibody solution either at room temperature for 2 hours or at 4° C. overnight. The cells are washed 3 times for 5 minutes with 1 ml PBT, then incubated with 0.5 ml secondary antibody solution (1% BSA/PBS/0.1% Triton X-100/1:300 dilution TRITC-donkey anti-rat IgG (Jackson Immunoresearch # 712-026-150)/1:300 dilution AlexaFluor 488-goat anti-rabbit (Molecular Probes #A-11008) at room temperature for 45-60 minutes. The cells are once more washed 3 times for 5 minutes with 1 ml PBT and once for 5 minutes with 1 ml PBS. Finally, the coverslips are mounted, cells-side down on clean microscope slides using mounting medium containing DAPI (1.25% (w/v) n-propylgallate in 75% glycerol/0.5 μg/ml DAPI, stored at 4° C.), sealed with nail enamel and stored, protected from light at 4° C.

ii) Analysis of the Mitotic Phenotype of the Immunostained, siRNA-Treated Cells.

Staining of DNA (Zeiss filter set 01), α-tubulin (Zeiss filter set 15) and γ-tubulin (Zeiss filter set 10) is observed using Plan-neofluar 40×/1.30 oil Ph3 and Plan-Apochromat 63×/1.40 oil Ph3 objectives on the Zeiss Axioskop 2 plus (Carl Zeiss Ltd., Woodfield Road, Welwyn Garden City, Herts. AL7 1LU, UK) using Axiocam HR CCD camera and AxioVision 3.0 software to acquire images.

For each experiment, the number of normal looking mitotic cells in prophase/prometaphase, metaphase, anaphase and telophase is quantified as well as the abnormal cells in each stage of the cell cycle. For each experiment, 200-250 mitotic cells are assessed. Of the abnormal cells, the percentage with misaligned chromosomes and lagging chromosomes and the number showing abnormal spindle morphology is quantitated. The number of centrosomes associated with each nucleus is also noted. For a more complete characterisation of the phenotype the ploidy and cell viability (cell confluency and number of apoptotic cells), the number of multinucleated interphase cells and the nuclear and overall cell morphology is assessed. To determine if a particular gene specific siRNA caused an abnormal cell cycle phenotype, comparisons are made with the phenotype of cells transfected with the GL3 siRNA control.

Positive results are judged as those where the overall mitotic defects were increased from the negative control by about 10% or more. There are also instances, however, where the number of overall mitotic defects does not increase significantly, yet there is a significant increase in the percentage of cells with one particular defect and this would also be viewed as significant.

8) Disease Association

The level of expression of a gene(s) in tumour and normal cells is determined by assessing the levels of hybridisation of a gene specific radioactive probe to a cancer profile array. The array consists of 240 total cDNAs from tumour cells and from equivalent normal cells from 13 tissue types (Cat no. 7841-1-Clontech Laboratories, Inc., 1020 East Meadow Circle, Palo Alto, Calif. 94303-4230, USA). A gene specific DNA sequence of approximately 500 bp is amplified from a plasmid containing the gene or a part of the gene and the PCR product purified following a standard PCR purification protocol (QIAquick PCR purification—QIAGEN GmbH, Max-Volmer-Strasse 4, 40724 Hilden, Germany). The purified PCR fragment is then radioactively labelling using 6000 Ci/mmol α³²P [dCTP] (Amersham Biosciences UK Ltd., Amersham Place, Little Chalfont, Buckinghamshire HP7 9NA) following a standard random hexamer labelling protocol (High prime—Roche Diagnostics Ltd., Bell Lane, Lewes, East Sussex BN7 1LG, UK).

Changes in gene expression level between normal and cancer cells are detected by probing the cancer profile array with 100 ng of radiolabelled probe, following the standard manufacturer's protocol (Clontech). Briefly, the cancer profile array is pre-hybridised for 90 minutes at 65° C. in 10 ml ExpressHyb (Clonetech) containing 1.5 mg of denatured sheared salmon testis DNA. The pre-hybridisation solution discarded and the cancer profile array hybridised overnight at 65° C. in 5 ml ExpressHyb containing the labelled DNA probe, 30 kg Cₒt-1 DNA, 150 μg sheared salmon testis DNA and 1% 20×SSC. After hybridisation, the cancer profile array is washed four times in 40 ml 2×SSC, 1% SDS for 30 minutes at 65° C. before 1 wash in 0.1×SSC, 0.5% SDS for 30 minutes at 65° C. A final wash is performed in 5×SSC for 5 minutes at room temperature.

The gene specific radiolabelled DNA bound to the cancer profile array is detected by exposing the array to X-ray film (Kodak Biomax MR) for 6-14 days at −70° C. using intensifying screens.

The expression pattern for the gene in the cancer profile array is assessed, by noting the number of cases where gene expression is increased and the number where there is a decrease. Changes in gene expression in tumour cells are deemed significant where more than 50% of the samples in a tissue sample result in a decrease or an increase in gene expression in 4 or more tissue types.

All patents, patent applications, and published references cited herein are hereby incorporated by reference in their entirety. While this invention has been particularly shown and described with references to preferred embodiments thereof, it will be understood by those of ordinary skill in the art that various changes in form and details may be made herein without departing from the scope of the invention encompassed by the appended claims.

1. A method of identifying an agent that modulates the function of a cell cycle progression polypeptide of SEQ ID NOs: 1-204 (even numbered sequences), the method comprising:
(a) providing a sample containing a cell cycle progression polypeptide of SEQ ID NOs: 1-204 (even numbered sequences), and a candidate agent;
(b) measuring the binding of the cell cycle progression polypeptide of SEQ ID NOs: 1-204 (even numbered sequences) to the candidate agent in the sample; and
(c) comparing the binding of the cell cycle progression polypeptide of SEQ ID NOs: 1-204 (even numbered sequences) to the candidate agent in'the sample with the binding of the polypeptide of SEQ ID NOs: 1-204 (even numbered sequences) to a control agent, wherein the control agent is known to not bind to the polypeptide of SEQ ID NOs: 1-204 (even numbered sequences;
wherein an increase in the binding of the cell cycle progression polypeptide of SEQ ID NOs: 1-204 (even numbered sequences) to the candidate agent in the sample relative to the binding of the cell cycle progression polypeptide of SEQ ID NOs: 1-204 (even numbered sequences) to the control agent indicates that the candidate agent modulates the function of the cell cycle progression polypeptide of SEQ ID NOs:1-204 (even numbered sequences).
